(19) United States
(12) Patent Application Publication
(10) Pub. No.: US 2025/0188078 A1
(43) Pub. Date: Jun. 12, 2025

(54) SMALL MOLECULE INHIBITORS OF THE CRL4 UBIQUITIN LIGASE

(71) Applicant: Cornell University, Ithaca, NY (US)

(72) Inventors: Pengbo Zhou, Princeton Junction, NJ (US); Chenyi Yang, Rego Park, NY (US); Lei Shi, Clarkville, MD (US)

(73) Assignee: Cornell University, Ithaca, NY (US)

(21) Appl. No.: 18/846,154

(22) PCT Filed: Mar. 13, 2023
(86) PCT No.: PCT/US2023/064232

§ 371 Date: Sep. 11, 2024

Related U.S. Application Data
(60) Provisional application No. 63/319,142, filed on Mar. 11, 2022.

(51) Int. Cl. C07D 471/04; A61K 31/437; A61K 31/444; A61K 31/496; A61K 31/635; A61P 35/00

(52) U.S. Cl. CPC C07D 471/04; A61K 31/437; A61K 31/444; A61K 31/496; A61K 31/635; A61P 35/00

(57) ABSTRACT

Compounds of formula (I), compounds of formula (II), and compounds of formula (III) are disclosed. Also disclosed are compounds, compositions, and methods to inhibit CUL4A expression or activity, CUL4B expression or activity, and/or DDB1 expression or activity for the treatment or prevention of cancer, DNA damage, or related conditions. In some aspects, the interaction between CUL4A and/or CUL4B and the beta-propeller B of DDB1 is disrupted with the compounds, compositions, or methods.

### CROSS-REFERENCE TO RELATED APPLICATIONS

This patent application claims the benefit of U.S. Provisional Patent Application No. 63/319,142, filed Mar. 11, 2022, the entire disclosure of which is hereby incorporated by reference.

### BACKGROUND

The gene encoding the CUL4A ubiquitin ligase (also referred to in the art as Cullin Ring Ligase 4 (CRL4) and Cullin-4A) is frequently amplified or overexpressed in a wide variety of cancer types, such as breast cancer, hepatocellular carcinoma, colon or colorectal cancer, squamous cell carcinoma, adrenocortical carcinoma, childhood medulloblastoma, primary malignant pleural mesothelioma, lung cancer, brain cancer, and many other tumor types. CUL4A functions as a component of a multimeric protein complex wherein the N-terminus of CUL4A interacts with damage-specific DNA binding protein 1 (DDB1). DDB1, in turn, acts as an adaptor, binding to DDB1 CUL4A associated factors (DCAFs), which serve as specific substrate receptors. Both CUL4A and DDB1 are directly involved in repairing damaged DNA. Accordingly, proper expression or activity of CUL4A is desirable for prevention and treatment of cancers.

CUL4B, another member of the CUL4 family, is a scaffold of the Cullin4B-Ring E3 ligase complex, which is known to participate in proteolysis and has tumorigenesis implications. Abnormal expression or activity of CUL4B has been observed in various diseases or conditions, including various cancers (cervical, osteosarcoma, colorectal, ovarian, pancreatic, stomach, thyroid, esophageal, kidney, liver, and lung). As mentioned above, CUL4B also plays a role in DNA damage repair. Accordingly, proper functioning of CUL4B is desirable to treat or prevent diseases or conditions associated with cancer or DNA damage. In addition, CUL4B mutations have been associated with X-linked mental retardation.

Thus, there is a need for compounds, compositions, and methods that facilitate proper functioning of CUL4A expression or activity, CUL4B expression or activity, and/or DDB1 expression or activity for the treatment or prevention of cancer, DNA damage, or related conditions. This invention provides such compositions and methods.

### BRIEF SUMMARY

Disclosed herein are compounds of formula (I):

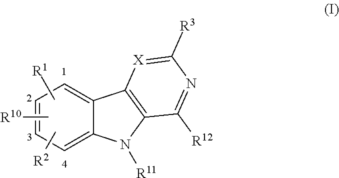

wherein:
X is C—H or N;
R¹, R², and R¹⁰ independently are —H, —OH, —CN, -haloalkyl, —O—(C₁-C₆ alkyl), halogen, —C₁-C₆ alkyl, or phenyl;
R³ is —COOH, —COO—(C₁-C₆ alkyl), —CONH₂, —CON(C₁-C₆ alkyl)₂, —CONH(C₁-C₆ alkyl), —CONHSO₂(C₁-C₆ alkyl), —CON(C₁-C₆ alkyl)SO₂(C₁-C₆ alkyl), halogen, —SO₂NH₂, —NHSO₂(C₁-C₆ alkyl), —CN, or tetrazole;
R¹¹ is —H or —C₁-C₆ alkyl;
wherein any alkyl, phenyl, or tetrazole moiety of an R¹, R², R³, R¹⁰, or R¹¹ group can be further substituted with —C₁-C₆ alkyl, —O—(C₁-C₆ alkyl), —SO₂(C₁-C₆ alkyl), halogen, —CN, haloalkyl, —OH, pyridinyl, —NO₂, —N-piperazinyl-N—(C₁-C₆ alkyl), phenyl, Bn, or any combination thereof, and
R¹² is phenyl, a 5-membered heterocycle, or a 6-membered heterocycle, each of which is substituted with R⁴, R⁵, and R⁶ on a carbon atom and/or heteroatom,
wherein:
   R⁴, R⁵, and R⁶ independently are —H, —C₁-C₆ alkyl, halogen, —OH, —O(C₁-C₆ alkyl), —CN, —NO₂, —(C₁-C₆ alkyl)-OH, SO₂(C₁-C₆ alkyl), -haloalkyl, —CO₂(C₁-C₆ alkyl), —NHSO₂(C₁-C₆ alkyl), —NHBn, phenyl, —SBn, —OBn, —SO₂NH₂, —SO₂(C₁-C₆ alkyl), —SO₂Bn, SO₂N(C₁-C₆ alkyl)₂, —OSO₂(C₁-C₆ alkyl), —C≡C-phenyl, —C≡CCH₂(OCH₂CH₂)₁₋₄OH, —C≡CCH₂(OCH₂CH₂)₁₋₄OMe, —NHSO₂-pyridinyl, —NBnSO₂-pyridinyl, —NHSO₂-phenyl, —NHSO₂(N-piperazinyl-N—(C₁-C₆ alkyl)), —N(C₁-C₆ alkyl)SO₂-pyridinyl, —N(C₁-C₆ alkyl)SO₂-phenyl, —N(C₁-C₆ alkyl)SO₂(N-piperazinyl-N—(C₁-C₆ alkyl)), —NBnSO₂(C₁-C₆ alkyl), —NBnSO₂(N-piperazinyl-N—(C₁-C₆ alkyl)), —NBnSO₂-phenyl, or —N(C₁-C₆ alkyl)SO₂(C₁-C₆ alkyl); or
   R⁴ and R⁵ taken together form a fused pyrazole ring, a fused pyrrole ring, or a fused phenyl ring, and R⁶ is as defined above;
wherein any alkyl, phenyl, Bn, pyridinyl, fused pyrazole ring, fused pyrrole ring, or fused phenyl ring moiety of an R⁴, R⁵, or R⁶ group can be further substituted with —C₁-C₆ alkyl, —O—(C₁-C₆ alkyl), —SO₂(C₁-C₆ alkyl), halogen, —CN, haloalkyl, —OH, pyridinyl, furanyl, tetrahydrofuranyl, tetrahydropyranyl, thiophenyl, thiazolyl, pyrrolyl, pyrrolidinyl, piperidinyl, morpholinyl, pyrimidinyl, triazolyl, imidazolyl, —NO₂, —N-piperazinyl-N—(C₁-C₆ alkyl), phenyl, -Bn, or any combination thereof;
   provided that:
when R¹² is phenyl; R¹, R², R⁴, R⁵, and R¹⁰ are —H; and R³ is tetrazole or tetrazole with its N—H group substituted with N-Bn; then R⁶ is not —H, 4-methoxy, or 4-fluoro;
when R¹² is phenyl; R¹, R², R⁴, R⁵, and R¹⁰ are —H; and R³ is —COOH; then R⁶ is not —H, 4-nitro, 4-chloro, 4-methoxyl, 4-CF₃, 4-bromo, 4-isopropyl, or 4-hydroxyl;
when R¹² is phenyl; R¹, R², R⁴, and R¹⁰ are —H; R³ is —COOH; and R⁵ is 4-hydroxyl; then R⁶ is not CO₂Me or methoxyl;
when R¹² is phenyl; R¹ R², and R¹⁰ are —H; and R³ is —COOH, —COOMe, or —COOEt; then R⁴, R⁵, and R⁶ are not 3-methoxyl, 4-methoxyl, and 5-methoxyl, respectively;
when R¹² is phenyl; R¹, R², R⁴, R⁵, and R¹⁰ are —H; and R³ is —COOMe; then R⁶ is not —H, 4-methoxyl, 2-chloro, 4-chloro, 4-hydroxyl, 3-nitro, 4-nitro, 4-bromo, 4-methoxyl, or 4-CF₃; and
when R¹² is phenyl; R¹, R⁴, R⁵, and R¹⁰ are —H; R² is methoxyl or methyl; and R³ is —COOEt; then R⁶ is not —H, 4-methoxyl, 2-chloro, 4-hydroxyl, or 3-nitro;
or a salt or ester thereof.

Also disclosed herein is a compound of formula (II):

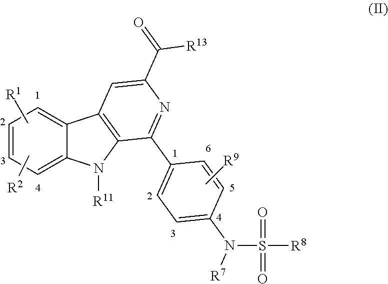

wherein:
R¹ and R² independently are —H, —OH, —C₁-C₆ alkyl, —O—(C₁-C₆ alkyl), halogen, —CN, haloalkyl, or phenyl;
R¹¹ is —H or —C₁-C₆ alkyl;
R¹³ is —OH, —O—(C₁-C₆ alkyl), —NH₂, —NHSO₂—(C₁-C₆ alkyl), or —N(C₁-C₆ alkyl)SO₂(C₁-C₆ alkyl);
R⁷ is —H, —C₁-C₆ alkyl, or -Bn;
R⁸ is —C₁-C₆ alkyl, —N-piperazinyl-N—(C₁-C₆ alkyl), pyridinyl, or phenyl; and
R⁹ is —H, —OH, —C₁-C₆ alkyl, —O—(C₁-C₆ alkyl), haloalkyl, or a halogen;
wherein any alkyl phenyl, Bn, or pyridinyl moiety on an R¹, R², R⁷, R⁸, R⁹, R¹¹, or R³ group can be further substituted with —C₁-C₆ alkyl, —O—(C₁-C₆ alkyl), —SO₂(C₁-C₆ alkyl), halogen, —CN, haloalkyl, —OH, pyridinyl, furanyl, tetrahydrofuranyl, tetrahydropyranyl, thiophenyl, thiazolyl, pyrrolyl, pyrrolidinyl, piperidinyl, morpholinyl, pyrimidinyl, triazolyl, imidazolyl, —NO₂, —N-piperazinyl-N—(C₁-C₆ alkyl), phenyl, -Bn, or any combination thereof, or a salt or ester thereof.

Also disclosed herein is a compound of formula (III):

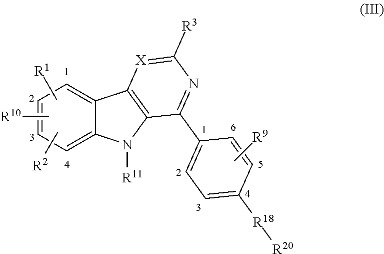

wherein:
X is C—H or N,
R¹, R², and R¹⁰ independently are —H, —OH, —CN, haloalkyl, —O—(C₁-C₆ alkyl), halogen, —C₁-C₆ alkyl, or phenyl;
R³ is —COOH, —COO—(C₁-C₆ alkyl), —CONH₂, —CON(C₁-C₆ alkyl)₂, —CONH(C₁-C₆ alkyl), —CONHSO₂(C₁-C₆ alkyl), —CON(C₁-C₆ alkyl)SO₂(C₁-C₆ alkyl), halogen, —SO₂NH₂, —NHSO₂(C₁-C₆ alkyl), —CN, or tetrazole;
R¹¹ is —H or C₁-C₆ alkyl;
R¹⁸ is —O—, —S—, —NR¹⁹—, —(C₁-C₆ alkyl)-, —(O—(C₁-C₆ alkyl))-, —(O—(C₁-C₆ alkyl)-O)—, —(OCH₂CH₂)ₙ—O—, —(NR¹⁹—(C₁-C₆ alkyl))-, —(NR¹⁹—(C₁-C₆ alkyl)-O)—, —(NR¹⁹—(C₁-C₆ alkyl)-NR¹⁹)—, —(OCH₂CH₂)ₙ—NR¹⁹—, —(S—(C₁-C₆ alkyl))-, —(S—(C₁-C₆ alkyl)-S)—, —(OCH₂CH₂)ₙ—S—, —(NR¹⁹—(C₁-C₆ alkyl)-S)—, —(S—(C₁-C₆ alkyl)-O)—, or —(CONR¹⁹)ₙ—;
wherein each n independently is an integer from 1 to 6;
wherein each R¹⁹ independently is H or C₁-C₆ alkyl; and
R⁹ is —H, —OH, —C₁-C₆ alkyl, —O—(C₁-C₆ alkyl), phenyl, haloalkyl, or halogen;
R²⁰ is C₃-C₆ cycloalkyl, phenyl, a 5-membered heterocycle, or a 6-membered heterocycle, each of which is substituted with R¹⁴, R¹⁵, and R¹⁶ on a carbon atom and/or heteroatom,
wherein:
   R¹⁴, R¹⁵, and R¹⁶ independently are —H, —C₁-C₆ alkyl, halogen, —OH, —O(C₁-C₆ alkyl), —CN, —NO₂, —(C₁-C₆ alkyl)-OH, SO₂(C₁-C₆ alkyl), haloalkyl, —CO₂(C₁-C₆ alkyl), —NHSO₂(C₁-C₆ alkyl), —NHBn, phenyl, —SBn, —OBn, —SO₂NH₂, —SO₂(C₁-C₆ alkyl), —SO₂Bn, SO₂N(C₁-C₆ alkyl)₂, —OSO₂(C₁-C₆ alkyl), —C≡C-phenyl, —C≡CCH₂(OCH₂CH₂)₁₋₄OH, —C≡CCH₂(OCH₂CH₂)₁₋₄OMe, —NHSO₂-pyridinyl, —NBnSO₂-pyridinyl, —NHSO₂-phenyl, —NHSO₂(N-piperazinyl-N—(C₁-C₆ alkyl)), —N(C₁-C₆ alkyl)SO₂-pyridinyl, —N(C₁-C₆ alkyl)SO₂-phenyl, —N(C₁-C₆ alkyl)SO₂(N-piperazinyl-N—(C₁-C₆ alkyl)), —NBnSO₂(C₁-C₆ alkyl), —NBnSO₂(N-piperazinyl-N—(C₁-C₆ alkyl)), —NBnSO₂-phenyl, or —N(C₁-C₆ alkyl)SO₂(C₁-C₆ alkyl); or
   R¹⁴ and R¹⁵ taken together form a fused pyrazole ring, a fused pyrrole ring, or a fused phenyl ring, and R¹⁶ is as defined above; and
wherein any -Bn, pyridinyl, alkyl, tetrazole, fused pyrazole ring, fused pyrrole ring, fused phenyl ring, or phenyl moiety of the foregoing groups can be substituted with C₁-C₆ alkyl, —O—(C₁-C₆ alkyl), —SO₂(C₁-C₆ alkyl), halogen, —CN, haloalkyl, —OH, pyridinyl, furanyl, tetrahydrofuranyl, tetrahydropyranyl, thiophenyl, thiazolyl, pyrrolyl, pyrrolidinyl, piperidinyl, morpholinyl, pyrimidinyl, triazolyl, imidazolyl, nitro, —N-piperazinyl-N—(C₁-C₆ alkyl), phenyl, Bn, or any combination thereof;
or a salt or ester thereof.

Additionally disclosed herein is a method of reducing CRL4A ubiquitin ligase expression or activity, CUL4A expression or activity, CUL4B expression or activity, and/or DDB1 expression or activity (e.g., by disrupting the interaction between CUL4A and/or CUL4B and the beta-propeller B of DDB1) in an animal, particularly a human, which method comprises administering to the animal a compound of formula (I), formula (II), and/or formula (III).

Further provided is a method of treating or preventing in an animal, particularly a human, a disease or condition associated with abnormal or pathogenic expression or activity of CRL4A ubiquitin ligase, CUL4A, CUL4B, and/or DDB1 (e.g., abnormal or pathogenic levels of interaction between CUL4A and/or CUL4B and the beta-propeller B of DDB1), the method comprising administering to the mammal a compound of formula (I), formula (II), and/or formula (III).

Also disclosed herein is a method of preventing or treating a cancer in an animal, particularly a human, which method comprises administering to an animal a compound of formula (I), formula (II), and/or formula (III).

Also provided is a method of inhibiting or slowing aging in an animal, particularly a human, optionally aging associated with DNA damage from external (e.g., UV radiation) or internal DNA damaging sources, which method comprises administering to the animal a compound of formula (I), formula (II), and/or formula (III).

Further disclosed herein is a composition comprising (a) a compound of formula (I), formula (II), and/or formula (III) and (b) a carrier, which composition is useful in any of the forgoing methods.

### BRIEF DESCRIPTION OF THE DRAWINGS

FIG. 1 is a line drawing showing a synthetic scheme for the preparation of compound PA99-1 (methyl 5-fluoro-1-(4-(N-methylphenylsulfonamido)phenyl)-9H-pyrido[3,4-b]indole-3-carboxylate) and compound PA99 (5-fluoro-1-(4-(N-methylphenylsulfonamido)phenyl)-9H-pyrido[3,4-b]indole-3-carboxylic acid), as described further in Example 1.

FIG. 2 is a line drawing showing a synthetic scheme for the preparation of compound 8[PA1-3] (methyl 7-fluoro-1-(4-(N-methylphenylsulfonamido)phenyl)-9H-pyrido[3,4-b]indole-3-carboxylate) and compound PA1-3 (7-fluoro-1-(4-(N-methylphenylsulfonamido)phenyl)-9H-pyrido[3,4-b]indole-3-carboxylic acid), as described further in Example 2.

FIGS. 3A-3B are line drawings showing synthetic schemes for the preparation of compound 8[PA1-9] (methyl 7-cyano-1-(4-(N-methylphenylsulfonamido)phenyl)-9H-pyrido[3,4-b]indole-3-carboxylate) and compound PA1-9 (7-cyano-1-(4-(N-methylphenylsulfonamido)phenyl)-9H-pyrido[3,4-b]indole-3-carboxylic acid), as described further in Example 3.

FIG. 4 is a line drawing showing a synthetic scheme for the preparation of compound 12[PA1-13A-7] (methyl 7-fluoro-1-(4-((N-methyl-4-(4-methylpiperazin-1-yl)phenyl)sulfonamido)phenyl)-9H-pyrido[3,4-b]indole-3-carboxylate) and compound PA1-13A-7 (7-fluoro-1-(4-((N-methyl-4-(4-methylpiperazin-1-yl)phenyl)sulfonamido)phenyl)-9H-pyrido[3,4-b]indole-3-carboxylic acid), as described further in Example 4.

FIG. 5 shows a compound of formula (I) and a specific compound (PA35) falling within the scope of formula (I), both of which have portions of the molecule designated “A,” “B,” and “C.” FIG. 5 also shows the crystal structure of the BPB beta-propeller of DDB1. The “C” portion of the molecule is believed to bind to the hydrophobic pocket formed by F458-I471-V500, and positively charged R589 is believed to interact with the “B” portion of the molecule (e.g., carboxylate).

FIGS. 6A-6D: FIG. 6A is a line graph showing that PA99 disrupts binding of CUL4A with DDB1-BPB, with IC₅₀ measured from AlphaLISA. FIG. 6B shows how a compound disclosed herein is believed to bind to DDB1-BPB (crystal structure) based on molecular docking and molecular dynamic simulation. FIGS. 6C-6D are western blots showing that compounds disclosed herein (PA78, PA93, PA99) specifically inhibit CUL4A but not CUL1 ubiquitin ligase. FIG. 6C demonstrates CUL4A-Vpr-dependent UNG2 degradation (robust inhibition). FIG. 6D demonstrates TNF-α induced CUL1-dependent IκB-α degradation (no inhibition).

FIG. 7 is a bar graph showing the concentration (μM) of compound PA99 in xenograft tumor tissue at various time intervals.

FIGS. 8A-8C are line graphs showing the suppression of CUL4A^{high} xenograft and PDX tumor growth by a compound disclosed herein (compound PA99). FIG. 8A is a breast cancer xenograft tumor model, and FIG. 8B is a colorectal cancer patient-derived xenograft tumor model.

FIGS. 9A-9C are line graphs showing the EC₅₀ (μM) of cell killing in CUL4^{high} cells (MDA-MB-468), CUL4^{low} cells (T47D), and MCF7 cells.

FIG. 10 is a line graph showing the selective killing of CUL4A^{high} tumors by compound PA99.

FIG. 11 is a line drawing showing a synthetic scheme for the preparation of compound 5[PA1-12] (methyl 1-[4-[benzenesulfonyl(methyl)amino]-2-fluoro-phenyl]-7-fluoro-9H-pyrido[3,4-b]indole-3-carboxylate) and PA1-12 (1-[4-[benzenesulfonyl(methyl)amino]-2-fluoro-phenyl]-7-fluoro-9H-pyrido[3,4-b]indole-3-carboxylic acid), as described further in Example 12. PA1-12A and its ester were also prepared using the synthetic scheme of FIG. 11, as described further in Example 17.

FIG. 12 is a line drawing showing a synthetic scheme for the preparation of compound 7[PA1-11] (methyl 1-(4-benzyloxyphenyl)-7-fluoro-9H-pyrido[3,4-b]indole-3-carboxylate) and PA1-11 (1-(4-benzyloxyphenyl)-7-fluoro-9H-pyrido[3,4-b]indole-3-carboxylic acid), as described further in Example 13.

FIG. 13 is a line drawing showing a synthetic scheme for the preparation of PA1-1, its methyl ester, and other various intermediates, as described further in Example 14.

FIG. 14 is a line drawing showing a synthetic scheme for the preparation of PA1-4, its methyl ester, and other various intermediates, as described further in Example 15. PA1-5, PA1-6, PA1-7, and each of their esters were also prepared using the synthetic scheme of FIG. 14, as described further in Example 16.

FIG. 15 is a line drawing showing a synthetic scheme for the preparation of PA1-13A-3, its methyl ester, and other various intermediates, as described further in Example 18.

FIG. 16 is a line drawing showing a synthetic scheme for the preparation of PA1-14, its methyl ester, and other various intermediates, as described further in Example 19.

FIG. 17 is a line drawing showing a synthetic scheme for the preparation of PA2-1, its methyl ester, and other various intermediates, as described further in Example 20. PA2-2, PA2-3, PA2-4, PA2-6, and PA1-13A-2 and each of their esters were also prepared using the synthetic scheme of FIG. 17, as described in Example 21.

FIG. 18 is a line drawing showing a synthetic scheme for the preparation of PA2-5, its methyl ester, and other various intermediates, as described further in Example 22.

FIG. 19 is a line drawing showing a synthetic scheme for the preparation of PA2-5, its t-butoxy ester, and other various intermediates, as described further in Example 23. PA24 and its t-butoxy ester was produced using the synthetic scheme of FIG. 19, as described further in Example 24.

FIG. 20 is a line drawing showing a synthetic scheme for the preparation of PA35, PA54, PA55, and other various intermediates, as described further in Example 25.

FIG. 21 is a line drawing showing a synthetic scheme for the preparation of PA53, its t-butoxy ester, and other various intermediates, as described further in Example 26.

FIG. 22 is a line drawing showing a synthetic scheme for the preparation of PA70, its ethyl ester, and other various intermediates, as described further in Example 27.

FIG. 23 is a line drawing showing a synthetic scheme for the preparation of PA73m PA74, the methyl ester of PA73, and other various intermediates, as described further in Example 28.

FIG. 24 is a line drawing showing a synthetic scheme for the preparation of PA2, PA3, PA5, PA6, PA7, PA8, PA19, PA21, PA22, PA23, PA25, PA26, PA27, PA29, PA32, PA33, PA36, PA71, and PA72, the methyl esters of each such compound, and other various intermediates, as described further in Example 30.

FIG. 25A, FIG. 25B, and FIG. 25C are line drawings showing a synthetic schemes for the preparation of PA9, PA14, PA30, PA38, PA40, PA41, PA42, PA48, PA57, PA68, PA75, PA76, PA77, and PA78, some esters thereof, and other various intermediates, as described further in Example 31.

FIG. 26A and FIG. 26B are line drawings showing a synthetic schemes for the preparation of PA45, PA49, PA50, PA58, PA59, PA60, PA61, PA62, PA63, PA64, PA65, PA66, and PA67, for some of these compounds the esters or carboxylic acids thereof, and other various intermediates, as described further in Example 32.

### DETAILED DESCRIPTION

Provided herein are compounds of formula (I), compounds of formula (II), and compounds of formula (III). Without wishing to be bound by any particular theory or mechanism of action, it is believed the compounds can interfere with CRL4A ubiquitin ligase expression or activity, CUL4A expression or activity, CUL4B expression or activity, DDB1 expression or activity (e.g., disrupting the interaction between CUL4A and/or CUL4B and the beta-propeller B of DDB1), or any combination thereof. The compounds are useful for the treatment or prevention of diseases or conditions associated with abnormal or otherwise pathogenic expression or activity (e.g., overexpression) of CRL4A ubiquitin ligase, CUL4A, CUL4B, or DDB1.

CUL4A is a ubiquitin ligase which functions as a component of a multimeric protein complex wherein the C-terminus of CUL4A interacts with the RING finger protein Rbx1 to recruit the E2 ubiquitin-conjugating enzyme, and the N-terminus of CUL4A interacts with DDB1. DDB1, in turn, acts as an adaptor, binding to DDB1 CUL4A associated factors (DCAFs), which serve as specific substrate receptors. Substrates for ubiquitination by CUL4A-containing complexes include c-Jun, DDB2, XPC, p21, PR-Set7/Set8, TSC2, HOXA9, HOXB4, the p12 subunit of DNA polymerase δ, CHK1 kinase, RORα, GRK5, androgen receptor, MCM10, RASSF1A, and REDD1. CUL4B, the other CUL4 family member, has extensive sequence homology with CUL4A and shares some redundant functions with CUL4A, including maintaining cell growth and mediating the ubiquitination of certain CUL4 targets.

Damage-specific DNA binding protein 2 (DDB2) has been shown to be subjected to CUL4A-dependent ubiquitination and degradation, which leads to an overall decrease in the ability to recognize DNA lesions. Other studies have shown that the cyclin-dependent kinase inhibitor p21 is also a substrate of the CUL4A ubiquitin ligase. Conditional CUL4A knockout mice exhibit increased accumulation of DDB2 and p21, resulting in both enhanced repair activity in the removal of strand-distorting DNA lesions induced by UV (e.g. cyclobutane pyrimidine dimers (CPDs) and 6,4-photoproducts (6,4-PPs)), and prolonged G1/S DNA damage checkpoint that allows additional time for the NER machinery to remove the DNA lesions. In addition, skin-specific deletion of CUL4A rendered the knockout mice resistant to UV-induced skin carcinogenesis. As such, inhibiting CUL4A expression or activity may be a potential therapeutic strategy for both prevention and treatment of human cancers, as well as for DNA damage repair.

In addition, a compound that interferes with the expression or activity of CUL4A also can interfere with the expression or activity of CUL4B, inasmuch as CUL4A and CUL4B exhibit redundant, isoform-specific activities. CUL4B is a scaffold of the Cullin4B-Ring E3 ligase complex, which is known to participate in proteolysis and has tumorigenesis implications. The ability to inhibit CUL4B is desirable in some aspects, since abnormal expression or activity of CUL4B has been observed in various diseases or conditions, including various cancers (cervical, osteosarcoma, colorectal, ovarian, pancreatic, stomach, thyroid, esophageal, kidney, liver, and lung). CUL4B also plays a role in DNA damage repair, and thus proper functioning of CUL4B is desirable to treat or prevent diseases or conditions associated with DNA damage.

Examples of biological functions of CUL4A include, without limitation, the regulation of cell proliferation, cell survival, DNA repair, and genomic integrity. Due to their high structural similarity, CUL4A and CUL4B have substantial overlap of function. Accordingly, in some aspects, the compound that interferes with the expression or activity of CUL4A, CUL4B, or both, causes an increase in DNA repair activity. In some aspects, the compound that interferes with the expression or activity of CUL4A, CUL4B, or both, causes an increase in nucleotide excision repair activity, thereby preventing or treating an ailment associated with DNA damage.

As used herein, as relating to the compounds of formulas (I), (II), and (III), the terms “formula (I) and/or formula (II) and/or formula (III),” “formula (I), formula (II), and/or formula (III),” “formula (I), formula (II), formula (III), or any combination thereof,” or similar phrases, means that any such compound can be employed alone or in any combination with one another. For example, a composition can be prepared that employs compounds of formulas (I), (II), or (III) as follows: (I) only; (II) only; (III) only; (I) and (II); (I) and (III); (II) and (III); or (I), (II), and (III). In addition, multiple compounds falling within each formula can also be employed in a composition if desired, such as within any of the combinations noted in this paragraph or elsewhere herein. For example, two compounds of formula (I) can be employed, or two compounds of formula (I) and one compound of formula (III), and so forth. All such combinations are contemplated.

As used herein, the term “animal” refers to any animal that would benefit from the treatment or prevention of cancer or related conditions, the treatment or prevention of DNA damage, and/or inhibition or slowing of aging. The animal may be any animal, but preferably is a mammal. In some aspects, the mammal is a mouse or other experimental mammal. In some aspects, the mammal is a human. In some aspects, the mammal is a primate, livestock, or a domestic pet (e.g., an equine, a canine, or a feline).

As used herein, the terms “substance,” “compound,” and “therapeutic agent” refer to a chemical compound, a mixture of chemical compounds, a biological macromolecule, or an extract made from biological materials such as bacteria, plants, fungi, or animal (particularly mammalian or human) cells or tissues that are suspected of having therapeutic properties. The substance, compound, or therapeutic agent can be purified, substantially purified or partially purified. The terms the terms “substance,” “compound,” and “therapeutic agent” generally are used interchangeably herein, unless context dictates a more specific meaning. Preferably the substance is a small molecule chemical compound. As used herein, the term “small molecule” refers to a non-biological substance or compound having a molecular weight of less than about 1,000 g/mol.

As used herein, the terms “interferes with CRL4A ubiquitin ligase expression or activity,” “interferes with CUL4A expression or activity,” “interferes with CUL4B expression or activity,” “interferes with DDB1 expression or activity,” “disrupts the interaction between CUL4A and/or CUL4B and the beta-propeller B of DDB1,” and similarly worded terms or phrases refer to the ability of a substance (e.g., the compound of formula (I), formula (II), and/or formula (III)) to inhibit the expression and/or biochemical or biological function of CRL4A, CUL4A, CUL4B, DDB1, or any combination thereof. Examples of biochemical functions of CUL4A include, without limitation, binding to DDB1 (e.g., disrupting the interaction between CUL4A and/or CUL4B and the beta-propeller B of DDB1), binding to CUL4A, binding to Rbx1, binding to DCAFs, having ubiquitin ligase activity (e.g., ubiquitinating and destabilizing p21, ubiquitinating and destabilizing DDB2, and ubiquitinating and destabilizing HOXB4), or any combination thereof. In some aspects, the substance that interferes with the expression or activity of CUL4A disrupts the binding of CUL4A to damaged DNA binding protein 1 (DDB1). In some aspects, the substance disrupts the interaction of the N-terminal α-helical region of CUL4A with the BPB P3-propeller domain of DDB1. The substance that disrupts the binding of CUL4A to DDB1 may interact directly with CUL4A and/or DDB1 or act indirectly (or allosterically) by binding to another component of a CUL4A-DDB1 containing complex. In some aspects, the substance that interferes with the expression or activity of CUL4A competitively inhibits the binding of an endogenous CUL4A to DDB1 in an animal, particularly a human. Examples of biological functions of CUL4A include, without limitation, the regulation of cell proliferation, cell survival, DNA repair, and genomic integrity. In some aspects, the substance that interferes with the expression or activity of CUL4A, CUL4B, and/or DDB1 causes an increase in DNA repair activity. In some aspects, the substance that interferes with the expression or activity of CUL4A, CUL4B, and/or DDB1 causes an increase in nucleotide excision repair activity. Thus, disclosed herein is a method of increasing DNA repair activity in an animal, particularly a human, which comprises administering to an animal (e.g., human) in need thereof an effective amount of a substance that interferes with the expression or activity of CUL4A, CUL4B, and/or DBB1. The substance that interferes with the expression or activity of CUL4A also can interfere with the expression or activity of CUL4B, inasmuch as CUL4A and CUL4B exhibit redundant, isoform-specific activities. The ability to inhibit CUL4B is desirable in some aspects, since abnormal expression or activity of CUL4B has been observed in various diseases or conditions, including various cancers (cervical, osteosarcoma, colorectal, ovarian, pancreatic, stomach, thyroid, esophageal, kidney, liver, and lung). CUL4B also plays a role in DNA damage repair, and thus proper functioning of CUL4B (e.g., downregulation of an overactive/overexpressed CUL4B) is desirable to treat or prevent diseases or conditions associated with DNA damage.

In some aspects, the substance (e.g., compound of formula (I), formula (II), and/or formula (III)) that interferes with the expression or activity of CUL4A, CUL4B, and/or DDB1 inhibits ubiquitin ligase activity by at least 25% (e.g., 25% or more, 35% or more, or 45% or more) compared to ubiquitin ligase activity in the absence of the interfering substance. In some aspects, the substance that interferes with the expression or activity of CUL4A, CUL4B, and/or DDB1 inhibits ubiquitin ligase activity by at least 50% (e.g., 50% or more, 60% or more, or 70% or more) compared to ubiquitin ligase activity in the absence of the interfering substance. In some aspects, the substance that interferes with the expression or activity of CUL4A, CUL4B, and/or DDB1 inhibits ubiquitin ligase activity by at least 75% (e.g., 75% or more, 85% or more, or 95% or more) compared to ubiquitin ligase activity in the absence of the interfering substance. When the substance interferes with the expression of CUL4A, CUL4B, DDB1, or a biochemical or biological function of CUL4A, CUL4B, and/or DDB1, the degree of inhibition may be partially complete (e.g., 10% or more, 25% or more, 50% or more, or 75% or more), substantially complete (e.g., 85% or more, 90% or more, or 95% or more), or fully complete (e.g., 98% or more, or 99% or more).

The term “DNA damage” is known to one of ordinary skill in the art and refers to any alteration of a DNA molecule relative to its native state. Examples of DNA damage include but are not limited to base pairing mismatches, spontaneous alterations in the chemistry of DNA bases (e.g., tautomeric shifts and deamination), loss of bases (i.e., depurination and depyrimidination), oxygen radical- and ionizing radiation-induced lesions (e.g., thymine damage due to the attack of C-5=C-6 double bond and DNA strand breaks), UV radiation-induced lesions (e.g., cyclobutane pyrimidine dimers and pyrimidine-pyrimidone (6-4) photoproducts), and chemical-induced lesions (e.g., alkylation and inter- or intra-strand crosslinks).

The terms “condition associated with DNA damage,” “disease associated with DNA damage,” or “ailment associated with DNA damage” refer to any ailment, condition, or disease wherein DNA damage is a causative or contributing factor. In some aspects, the condition associated with DNA damage is cancer.

In some aspects, the condition associated with DNA damage is aging. Aging includes the natural process of aging in an animal (e.g., a human) as well as accelerated aging which occurs in an animal (e.g., a human) having a heritable mutation in one or more genes that regulates the aging process. A main cause of the aging process in animals is somatic damage due to the effects of reactive oxygen species on cellular DNA. The reactive oxygen species are known to cause myriad DNA lesions such as base modifications, single- and double-strand DNA breaks and interstrand crosslinks. Accordingly, the invention provides methods, compounds, and compositions to enhance DNA repair activity in an animal, particularly a human, thereby preventing or treating aging.

In some aspects, the condition associated with DNA damage is prolonged exposure to UV radiation. As discussed above, it is well known that UV radiation induces cyclobutane pyrimidine dimers and pyrimidine-pyrimidone (6-4) photoproducts in DNA. Additional types of DNA damage caused by UV radiation include, without limitation, complex lesions involving purines (e.g., 8,8-adenine dehydrodimer), pyrimidine hydrates (e.g., 5,6-Dihydro-6-hydroxy-cytosine), thymine glycols, and strand breaks. Accordingly, the invention also provides methods, compounds, and compositions to enhance DNA repair activity in an animal, particularly a human, thereby preventing or treating a condition associated with prolonged exposure to UV radiation.

In some aspects, the condition associated with DNA damage is exposure to a chemical carcinogen. Chemical carcinogens are known to cause a variety of DNA lesions, including, without limitation, alkylation, inter- or intra-strand crosslinks, and adduct formation. One of ordinary skill in the art is aware of many common chemical carcinogens and is familiar with databases which contain information regarding the carcinogenicity of a given chemical (e.g., The Carcinogenic Potency Project maintained by the University of California-Berkeley and the National Toxicology Program maintained by the United States Department of Health and Human Services). In addition, one of ordinary skill in the art is aware of methods to assess the carcinogenicity of a given chemical (e.g., the Ames test). Accordingly, the invention also provides methods, compounds, and compositions to enhance DNA repair activity in an animal, particularly a human, thereby preventing or treating a condition associated with prolonged exposure to a chemical carcinogen. In some aspects, the chemical carcinogen is tobacco smoke. In some aspects, the chemical carcinogen is aflatoxin.

As used herein, “cancer” means any of various cellular diseases with malignant neoplasms characterized by the proliferation of anaplastic cells. It is not intended that the diseased cells must actually invade surrounding tissue and metastasize to new body sites. Cancer can involve any tissue of the body and have many different forms in each body area. Most cancers are named for the type of cell or organ in which they start. Various types of cancer are disclosed elsewhere herein. In some aspects, the cancer may result from a tumor generally found in humans and other mammals or a tumor that arises as the result of inoculation, such as in experimental mammals. Many types of cancer are encountered in humans and other animals, and the aspects described herein are not limited to any particular cancer type. In this respect, the methods disclosed herein are useful for tumor cells and associated stromal cells, solid tumors, and tumors associated with soft tissue, such as, soft tissue sarcoma, for example, in a human. The tumor or cancer can be located in the skin (e.g., melanoma), oral cavity, pharynx, respiratory system, digestive system, bones, joints (e.g., bony metastases), soft tissue, breast, genital system, urinary system, eye, orbit, brain (e.g., glioma or medulloblastoma), central nervous system, or endocrine system (e.g., thyroid or adrenal gland) and is not necessarily the primary tumor or cancer. Tissues associated with the oral cavity include, but are not limited to, the tongue and tissues of the mouth. Cancer can arise in tissues of the digestive system including, for example, the esophagus, stomach, small intestine, colon, rectum, anus, liver, gall bladder, and pancreas. Cancers of the respiratory system can affect the larynx, lung, and bronchus and include, for example, non small cell lung carcinoma. Tumors can arise in the uterine cervix, uterine corpus, ovary vulva, vagina, prostate, testis, and penis, which make up the male and female genital systems, and the urinary bladder, kidney, renal pelvis, and ureter, which comprise the urinary system. The tumor or cancer can be located in the head and/or neck (e.g., laryngeal cancer and parathyroid cancer). The tumor or cancer also can be located in the hematopoietic system or lymphoid system, and include, for example, lymphoma (e.g., Hodgkin's disease and Non Hodgkin's lymphoma), multiple myeloma, or leukemia (e.g., acute lymphocytic leukemia, chronic lymphocytic leukemia, acute myeloid leukemia, chronic myeloid leukemia, and the like). In some aspects, the tumor or cancer is skin cancer, breast cancer, colorectal cancer, lung cancer, or brain cancer. When the tumor or cancer is located in the brain, the tumor or cancer can be any known brain tumor or cancer, but in some aspects is a medulloblastoma. When the tumor or cancer is located in the adrenal gland, the tumor or cancer can be any known adrenal gland tumor or cancer, but in some aspects is an adrenocortical carcinoma.

As used herein, a “tumor” means an abnormal mass of tissue growth that may be classified as benign or malignant. Tumors, as is known, include an abnormal mass of tissue that results from uncontrolled and progressive cell division, and is also typically known as a “neoplasm.”

Within the context of certain aspects, whether cancer is “reduced” or is at least responding to treatment may be identified by a variety of diagnostic manners known to one skill in the art including, but not limited to, observation the reduction in size or number of tumor masses or if an increase of apoptosis of cancer cells observed, e.g., if more than a 5% increase in apoptosis of cancer cells is observed for a sample compound compared to a control without the compound. It may also be identified by a change in relevant biomarker or gene expression profile, such as PSA for prostate cancer, her2 for breast cancer, or others. For example, reduction of cancer may be identified in vitro using the following conditions for evaluation of apoptosis: i) Jurkat human T-cell leukemia cells are passed into flasks (250 mL, 75 cm²) with 20 mL of supporting media; ii) after incubation at 37° C. with 5% CO₂, sample compound (or absent control) is added to a flask to make final concentration at 1 mM, and cells are incubated for another day; iii) cells are treated with 10 μM camptothecin and incubated with SYTOX Green reagent and annexin V allophycocyanin (APC) conjugate (invitrogen) and iv) Flow cytometry at 488 nm and 633 nm excitation. In cells undergoing apoptosis, phosphatidylserine (PS) is transferred from the cytoplasmic surface of the cell membrane to the outer leaflet. Annexin V has a high affinity for PS and dye conjugates provide indication of apoptosis by phosphatidylserine exposure and membrane integrity.

The terms “condition associated with cancer,” “disease associated with cancer,” or “ailment associated with cancer” refer to any ailment, condition, or disease wherein cancer is a causative or contributing factor. In some aspects, the condition associated with cancer is DNA damage. In some aspects, the ailment, condition, or disease associated with DNA damage is a human hereditary disease or an experimental animal model of a human hereditary disease. Examples of human hereditary diseases that may be treated according to the methods provided herein include, but are not limited to, Xeroderma pigmentosum, Cockayne syndrome, Trichothiodystrophy, Fanconi anemia, Ataxia telangiectasia (Louis-Bar Syndrome), and Bloom syndrome. Preferably, the human hereditary disease is Xeroderma pigmentosum.

In some aspects, provided are compositions, compounds, and methods to enhance DNA repair activity in an animal, particularly a human, that has been exposed to chemotherapy or radiation therapy. Although a primary goal of chemotherapy or radiation therapy is to induce DNA damage in cancerous cells thereby leading to growth arrest and or cell death, it may be desirable to concurrently enhance DNA repair in non-cancerous cells in the animal (e.g., human) exposed to chemotherapy or radiation therapy. One of ordinary skill is aware of chemotherapeutic chemicals that induce DNA damage. Examples of such chemicals include, without limitation, platinum derivatives (e.g., cisplatin, carboplatin, and oxaliplatin), nitrogen mustards (e.g., mechlorethamine, cyclophosphamide, and chlorambucil), and nitrosoureas (e.g., carmustine, lomustine, and ethylnitrosourea).

The terms “preventing or treating,” “treating,” “treatment,” “therapy,” and “therapeutic treatment” as used herein refer to curative therapy, prophylactic therapy, or preventative therapy. Such terms are not limited to the case where the animal (e.g., human patient) is cured and the disease is eradicated. Rather, the present invention also contemplates treatment that merely reduces symptoms, and/or delays disease progression. The terms “prevent” and “preventing” include the prevention of the recurrence, spread or onset. It is not intended that the present invention be limited to complete prevention. In some aspects, the onset is delayed, or the severity of the disease is reduced. An example of “prevention” or “preventative therapy” is the prevention or lessening of the chance of acquiring a cancer or other proliferative disease, DNA damage, or related conditions thereto. Those in need of treatment include those animals (e.g., humans) already diagnosed with cancer or DNA damage as well as those animals (e.g., humans) prone to develop cancer or DNA damage. The terms “treating,” “treatment,” “therapy,” and “therapeutic treatment” as used herein also describe the management and care of an animal (e.g., human) for the purpose of combating cancer, DNA damage, or related conditions, and includes the administration of a composition to alleviate the symptoms, side effects, or other complications of the cancer, DNA damage, or related conditions.

By “effective amount” or “therapeutically effective amount” with regard to cancer, DNA damage, or related conditions, it is meant an amount of a compound or composition that relieves (to some extent, as judged by a skilled medical practitioner, for example, in relation to an animal (e.g., human) not administered a compound as disclosed herein) one or more symptoms of the cancer, DNA damage, or the related condition or ailment in an animal (e.g., human). Additionally, by “effective amount” or “therapeutically effective amount” with regard to cancer, DNA damage, or a related condition, it is meant an amount that returns to normal, either partially or completely, physiological or biochemical parameters associated with or causative of the cancer, DNA damage, or related condition. Moreover, by “effective amount” or “therapeutically effective amount” with regard to aging or DNA damage, it is meant an amount that inhibits or slows aging or DNA damage, or aging associated with DNA damage (to some extent, as judged by a skilled medical practitioner, for example, in relation to an animal (e.g., human) not administered a compound as disclosed herein). A clinician skilled in the art can determine the therapeutically effective amount of a composition in order to treat or prevent a particular cancer or DNA damage when it is administered. The precise amount of the composition required to be therapeutically effective will depend upon numerous factors, e.g., such as the specific activity of the active compound, the delivery device employed, physical characteristics of the compound, purpose for the administration, in addition to many patient specific considerations. The determination of amount of a composition that must be administered to be an effective amount or a therapeutically effective amount is routine in the art and within the skill of an ordinarily skilled clinician.

By “administering” or “administered” it is meant that the compound is delivered to an animal (e.g., human) in need thereof. The route of administration may be topical, oral, intranasal, parenteral, enteric, rectal, intravenous, intraperitoneal, subcutaneous, pulmonary, transdermal, intramuscular, buccal, sublingual, or ocular. The compound preferably is suitable for parenteral administration. The term “parenteral,” as used herein, includes intravenous, intramuscular, subcutaneous, rectal, vaginal, and intraperitoneal administration. In some aspects, the compound can be administered to a mammal using peripheral systemic delivery by intravenous, intraperitoneal, or subcutaneous injection.

As used herein, the terms “CUL4^{high},”, “CUL4-high,” “high level of CUL4 expression,” “CUL4^{high} tumor,” or similar terms/phrases mean a high level of the indicated expression or activity (e.g., CUL4, CUL4A, or CUL4B) as compared to a normal level of the same indicated expression or activity, as could be determined by one of ordinary skill in the art by known techniques in the art. Conversely, “CUL4^{low},” “CUL4-low,” “low level of CUL4 expression,” and similar terms mean the opposite as the “high” counterparts, namely, a low level of the indicated expression or activity (e.g., CUL4, CUL4A, or CUL4B) as compared to a normal level of the same indicated expression or activity, as could be determined by one of ordinary skill in the art by known techniques in the art.

As used herein, “alkyl” means a straight chain or branched, noncyclic or cyclic, unsaturated or saturated aliphatic hydrocarbon containing from 1 to 10 carbon atoms (e.g., 1, 2, 3, 4, 5, 6, 7, 8, 9, or 10 carbon atoms, or any range thereof, including 1-2, 1-3, 1-4, 1-5, 1-6, 1-7, 1-8, 1-9, 1-10, 2-3, 2-4, 2-5, 2-6, 2-7, 2-8, 2-9, 2-10, 3-4, 3-5, 3-6, 3-7, 3-8, 3-9, 3-10, 4-5, 4-6, 4- 7, 4-8, 4-9, 4-10, 5-6, 5-7, 5-8, 5-9, 5-10, 6-7, 6-8, 6-9, 6-10, 7-8, 7-9, 7-10, 8-9, 8-10, or 9-10 carbon atoms, and so forth). The term “lower alkyl” has the same meaning as alkyl but contains from 1 to 6 carbon atoms and is sometimes termed “C₁-C₆ alkyl” herein. The carbon atom ranges for the term “alkyl” are applicable to “lower alkyls” within the 1 to 6 carbon atom range. The term “higher alkyl” has the same meaning as alkyl but contains from 7 to 10 carbon atoms. The carbon atom ranges for the term “alkyl” are applicable to “higher alkyls” within the 7 to 10 carbon atom range. Representative saturated straight chain alkyls include methyl, ethyl, n-propyl, n-butyl, n-pentyl, n-hexyl, n-heptyl, n-octyl, n-nonyl, n-decyl, and the like; while saturated branched alkyls include isopropyl, sec-butyl, isobutyl, tert-butyl, isopentyl, and the like (each of which has a carbon atom count that can be readily determined). C₁-C₆ alkyls can include such straight chain alkyls, including methyl, ethyl, n-propyl, n-butyl, n-pentyl, and n-hexyl. Representative saturated cyclic alkyls include cyclopropyl, cyclobutyl, cyclopentyl, cyclohexyl, and the like; while unsaturated cyclic alkyls include cyclopropenyl, cyclobutenyl, cyclopentenyl, cyclohexenyl, and the like (each of which has a carbon atom count that can be readily determined). C₁-C₆ alkyls can include such saturated cyclic alkyls, including cyclopropyl, cyclobutyl, cyclopentyl, and cyclohexyl. C₁-C₆ alkyls can also include such unsaturated cyclic alkyls, including cyclopropenyl, cyclobutenyl, cyclopentenyl and cyclohexenyl, and the like. Unsaturated alkyls contain at least one double or triple bond between adjacent carbon atoms (referred to as an “alkenyl” or “alkynyl”, respectively) (each of which has a carbon atom count that can be readily determined). C₁-C₆ alkyls can include such unsaturated alkyls. Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like (each of which has a carbon atom count that can be readily determined). C₁-C₆ alkyls can include such straight chain and branched alkenyls, including ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like. C₁-C₆ alkyls can also include such straight chain and branched alkynyls, including acetylenyl, propynyl, 1-butynyl, 2-butynyl, 1-pentynyl, 2-pentynyl, 3-methyl-1-butynyl, and the like. In some aspects, any “C₁-C₆ alkyl” term throughout this disclosure can be substituted with the term “alkyl,” or any of the terms set forth in this paragraph.

As used herein, “alkoxyl” means an alkyl moiety attached through an oxygen bridge (i.e., —O-alkyl), such as methoxyl, ethoxyl, propoxyl, and the like.

As used herein, a ring structure that is “fused” means a molecular structure in which two or more aromatic rings have two carbon atoms in common. Examples of fused ring structures include, for example, (a) a phenyl ring with a fused pyrazole ring, (b) a phenyl ring with a fused pyrrole ring, and (c) a phenyl ring with a fused phenyl ring, as shown below (with substituents defined elsewhere herein):

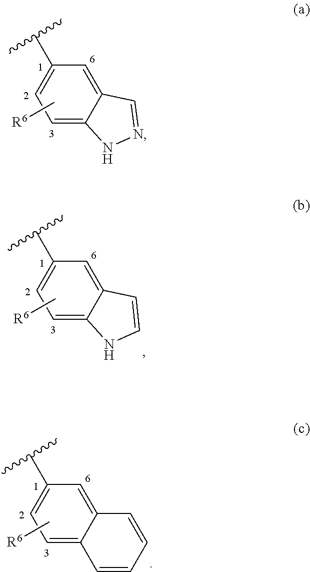

As used herein, “haloalkyl” means an alkyl group (e.g., C₁-C₆ alkyl) in which one or more hydrogen atoms is replaced with a halogen (e.g., —F, —Cl, —Br, —I, or any combination thereof. Examples of a haloalkyl group include, for example, —CF₃, —CH₂F, —CHF₂, —CCl₃, —CH₂CH₂F, —CH₂CHF₂, —CH₂CF₃, —CHFCH₃, —CF₂CH₃, —CF₂CF₃, —CCl₂CCl₃, —CCl₂CF₃, —CF₂CCl₃, —CH₂CH₂CF₃, and the like.

As used herein, “halogen” means —F, —Cl, —Br, and —I.

As used herein, “heteroatom” means a nitrogen atom, an oxygen atom, and a sulfur atom.

As used herein, “heteroaryl” means an aromatic heterocycle ring of 5- to 10 members (e.g., 5-, 6-, 7-, 8-, 9-, or 10-membered, or any range thereof) and having at least one heteroatom selected from nitrogen, oxygen and sulfur, and containing at least 1 carbon atom, including both mono- and bicyclic ring systems. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl.

As used herein, the term “heterocycle” (also referred to herein as a “heterocyclic ring”) means a 4- to 7-membered monocyclic (e.g., 4-, 5-, 6-, or 7-membered), or 7- to 10-membered bicyclic (e.g., 7-, 8-, 9-, or 10-membered, or any range thereof), heterocyclic ring which is either saturated, unsaturated, or aromatic, and which contains from 1 to 4 heteroatoms (e.g., 1, 2, 3, or 4 heteroatoms, or any range thereof, such as 1-2, 1-3, 1-4, 2-3, 2-4, or 3-4 heteroatoms) independently selected from nitrogen, oxygen and sulfur, and wherein the nitrogen and sulfur heteroatoms may be optionally oxidized, and the nitrogen heteroatom may be optionally quaternized, including bicyclic rings in which any of the above heterocycles are fused to a benzene ring. The heterocycle may be attached via any heteroatom or carbon atom. Heterocycles include heteroaryls as defined above. Thus, in addition to the heteroaryls listed above, heterocycles also include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, piperzinyl, dihydropyrimidinyl, and the like.

As used herein, the term “piperazine” means a 6-membered saturated ring containing two nitrogen atoms at opposite positions in the ring, including the structure shown below:

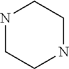

This structure can be substituted or unsubstituted in any position. Note one or more nitrogen atoms have an incomplete valence, indicating the nitrogen atom can be bonded to any group or atom, including a hydrogen, a carbon, or a more complex structure.

As used herein, the term “pyrrole” means an aromatic 5-membered heterocyle containing four carbon atoms and one nitrogen atom, including the structure shown below:

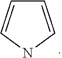

This structure can be substituted or unsubstituted in any position. Note one or more nitrogen atoms have an incomplete valence, indicating the nitrogen atom can be bonded to any group or atom, including a hydrogen, a carbon, or a more complex structure.

As used herein, the term “pyrazole” means an aromatic 5-membered heterocycle containing three carbon atoms and two adjacent nitrogen atoms, including the structure shown below:

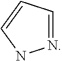

This structure can be substituted or unsubstituted in any position. Note one or more nitrogen atoms have an incomplete valence, indicating the nitrogen atom can be bonded to any group or atom, including a hydrogen, a carbon, or a more complex structure.

As used herein, the term “tetrazole” means an aromatic 5-membered heterocycle containing four nitrogen atoms and one carbon atom, including the structure shown below:

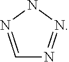

This structure can be substituted or unsubstituted in any position. Note one or more nitrogen atoms have an incomplete valence, indicating the nitrogen atom can be bonded to any group or atom, including a hydrogen, a carbon, or a more complex structure.

As used herein, the term “dihydropyrimidine” means a 6-membered heterocycle containing two double bonds and two nitrogen atoms in a meta arrangement, including the structure shown below:

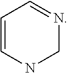

This structure can be substituted or unsubstituted in any position. Note one or more nitrogen atoms have an incomplete valence, indicating the nitrogen atom can be bonded to any group or atom, including a hydrogen, a carbon, or a more complex structure.

As used herein, the term “imidazole” means an aromatic 5-membered heterocycle containing two non-adjacent nitrogen atoms, including the structure shown below:

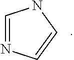

This structure can be substituted or unsubstituted in any position. Note one or more nitrogen atoms have an incomplete valence, indicating the nitrogen atom can be bonded to any group or atom, including a hydrogen, a carbon, or a more complex structure.

As used herein, the term “pyridine” means an aromatic 6-membered ring containing one nitrogen atom, including the structure shown below:

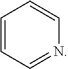

This structure can be substituted or unsubstituted in any position.

As used herein, the term “substituted” means a chemical group (e.g., alkyl, phenyl, tetrazole, heterocycle, benzyl, pyrazole, pyrrole, pyridinyl, etc.) wherein at least one hydrogen atom on the chemical group is replaced with a substituent. When the hydrogen on the chemical group is replaced (i.e., substituted), the entity that replaces the hydrogen is called a “substituent.” Substituents within the context of this invention include, for example, alkyl (e.g., —C₁-C₆ alkyl), alkoxyl (e.g., —O—C₁-C₆ alkyl), alkylsulfone (e.g., —SO₂(C₁-C₆ alkyl)), halogen (e.g., —F, —Cl, —Br, —I), cyano (i.e., —CN), haloalkyl (e.g., —CH₂F, —CHF₂, —CCl₃), hydroxyl (i.e., —OH), pyridinyl, nitro (i.e., —NO₂), —N-piperazinyl-N—(C₁-C₆ alkyl), phenyl (i.e., -Ph), and benzyl (i.e., -Bn). In addition, these substituents may be further substituted with one or more of the above substituents, such that the substituent is substituted alkyl, substituted phenyl, substituted tetrazole, substituted heterocycle, substituted benzyl, substituted pyrazole, substituted pyrrole, or substituted pyridinyl. In the context of some aspects, a compound may be described as “unsubstituted” meaning that the compound does not contain extra substituents attached to the compound (i.e., the hydrogens on the compound are not replaced with one or more substituents).

While some substituents herein may be referred to either with substituent terminology (i.e., with the suffix “yl”) or using the parent terminology (i.e., with the suffix “ane,” “ene,” “ine,” and so forth), it is intended that such different terminology has no difference in meaning, unless clearly contradicted by context. For example, although a substituent may be referred to as a “piperazine” substituent, it is meant that the substituent is “piperazinyl,” which is clear in the context of referring to piperazine as a substituent. This concept is applicable throughout this disclosure.

As used herein, the following terms are well known in the art and have the indicated meanings: “Me” (methyl), “Et” (ethyl), “Pr” (propyl), “Bu” (butyl). Additionally, such terms, and other terms herein, can be prefaced with certain terminology (e.g., “n,” “i,” “t,” “tert,” “2-,” etc.) that has well-known meanings in the art indicating connectivity or type of group. For example, “nPr” means “n-propyl,” “iBu” means “isobutyl,” and so forth.

Disclosed herein are compounds of formula (I):

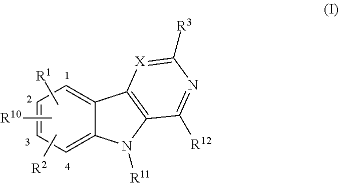

wherein:
X is C—H or N;
R¹, R², and R¹⁰ independently are —H, —OH, —CN, -haloalkyl (e.g., in which the alkyl portion is a C₁-C₆ alkyl), —O—(C₁-C₆ alkyl), halogen, —C₁-C₆ alkyl, or phenyl;
R³ is —COOH, —COO—(C₁-C₆ alkyl), —CONH₂, —CON(C₁-C₆ alkyl)₂, —CONH(C₁-C₆ alkyl), —CONHSO₂(C₁-C₆ alkyl), —CON(C₁-C₆ alkyl)SO₂(C₁-C₆ alkyl), halogen, —SO₂NH₂, —NHSO₂(C₁-C₆ alkyl), —CN, or tetrazole;
R¹ is —H or —C₁-C₆ alkyl;
wherein any alkyl, phenyl, or tetrazole moiety of an R¹, R², R³, R¹⁰, or R¹ group can be further substituted with —C₁-C₆ alkyl, —O—(C₁-C₆ alkyl), —SO₂(C₁-C₆ alkyl), halogen, —CN, haloalkyl, —OH, pyridinyl, —NO₂, —N-piperazinyl-N—(C₁-C₆ alkyl), phenyl, Bn, or any combination thereof, and
R¹² is phenyl, a 5-membered heterocycle, or a 6-membered heterocycle, each of which is substituted with R⁴, R⁵, and R⁶ on a carbon atom and/or heteroatom,
wherein:
   R⁴, R⁵, and R⁶ independently are —H, —C₁-C₆ alkyl, halogen, —OH, —O(C₁-C₆ alkyl), —CN, —NO₂, —(C₁-C₆ alkyl)-OH, SO₂(C₁-C₆ alkyl), -haloalkyl, —CO₂(C₁-C₆ alkyl), —NHSO₂(C₁-C₆ alkyl), —NHBn, phenyl, —SBn, —OBn, —SO₂NH₂, —SO₂(C₁-C₆ alkyl), —SO₂Bn, SO₂N(C₁-C₆ alkyl)₂, —OSO₂(C₁-C₆ alkyl), —C≡C-phenyl, —C≡CCH₂(OCH₂CH₂)₁₋₄OH, —C≡CCH₂(OCH₂CH₂)₁₋₄OMe, —NHSO₂-pyridinyl, —NBnSO₂-pyridinyl, —NHSO₂-phenyl, —NHSO₂(N-piperazinyl-N—(C₁-C₆ alkyl)), —N(C₁-C₆ alkyl)SO₂-pyridinyl, —N(C₁-C₆ alkyl)SO₂-phenyl, —N(C₁-C₆ alkyl)SO₂(N-piperazinyl-N—(C₁-C₆ alkyl)), —NBnSO₂(C₁-C₆ alkyl), —NBnSO₂(N-piperazinyl-N—(C₁-C₆ alkyl)), —NBnSO₂-phenyl, or —N(C₁-C₆ alkyl)SO₂(C₁-C₆ alkyl); or
   R⁴ and R⁵ taken together form a fused pyrazole ring, a fused pyrrole ring, or a fused phenyl ring, and R⁶ is as defined above;
wherein any alkyl, phenyl, Bn, pyridinyl, fused pyrazole ring, fused pyrrole ring, or fused phenyl ring moiety of an R⁴, R⁵, or R⁶ group can be further substituted with —C₁-C₆ alkyl, —O—(C₁-C₆ alkyl), —SO₂(C₁-C₆ alkyl), halogen, —CN, haloalkyl, —OH, pyridinyl, furanyl, tetrahydrofuranyl, tetrahydropyranyl, thiophenyl, thiazolyl, pyrrolyl, pyrrolidinyl, piperidinyl, morpholinyl, pyrimidinyl, triazolyl, imidazolyl, —NO₂, —N-piperazinyl-N—(C₁-C₆ alkyl)), phenyl, -Bn, or any combination thereof;
   provided that:
when R¹² is phenyl; R¹, R², R⁴, R⁵, and R¹⁰ are —H; and R³ is tetrazole or tetrazole with its N—H group substituted with N-Bn; then R⁶ is not —H, 4-methoxy, or 4-fluoro;
when R¹² is phenyl; R¹, R², R⁴, R⁵, and R¹⁰ are —H; and R³ is —COOH; then R⁶ is not —H, 4-nitro, 4-chloro, 4-methoxyl, 4-CF₃, 4-bromo, 4-isopropyl, or 4-hydroxyl;
when R¹² is phenyl; R¹, R², R⁴, and R¹⁰ are —H; R³ is —COOH; and R⁵ is 4-hydroxyl; then R⁶ is not CO₂Me or methoxyl;
when R¹² is phenyl; R¹ R², and R¹⁰ are —H; and R³ is —COOH, —COOMe, or —COOEt; then R⁴, R⁵, and R⁶ are not 3-methoxyl, 4-methoxyl, and 5-methoxyl, respectively;
when R¹² is phenyl; R¹, R², R⁴, R⁵, and R¹⁰ are —H; and R³ is —COOMe; then R⁶ is not —H, 2-chloro, 4-chloro, 4-hydroxyl, 3-nitro, 4-nitro, 4-bromo, 4-methoxyl, or 4-CF₃; and
when R¹² is phenyl; R¹, R⁴, R⁵, and R¹⁰ are —H; R² is methoxyl or methyl; and R³ is —COOEt; then R⁶ is not —H, 4-methoxyl, 2-chloro, 4-hydroxyl, or 3-nitro;
or a salt or ester thereof.

In some aspects, in formula (I):
R¹, R², and R¹⁰ independently are H, hydroxyl, methoxyl, a halogen, methyl, —CN, —CF₃, —CH₂F, —CHF₂, —CCl₃, or phenyl;
R³ is —COOH, —COO—(C₁-C₆ alkyl), —CONH₂, —CONHSO₂(C₁-C₆ alkyl), or tetrazole;
R¹¹ is H or —C¹-C⁶ alkyl;
wherein any alkyl, phenyl, or tetrazole moiety of an R¹, R², R³, R¹⁰, or R¹¹ group can be further substituted with —C₁-C₆ alkyl, —O—(C₁-C₆ alkyl), —SO₂(C₁-C₆ alkyl), halogen, —CN, haloalkyl, —OH, pyridinyl, —NO₂, —N-piperazinyl-N—(C₁-C₆ alkyl), phenyl, -Bn, or any combination thereof, and
R¹² is phenyl, imidazole, pyridine, or dihydropyrimidine, each of which is substituted with R⁴, R⁵, and R⁶;
wherein:
   R⁴, R⁵, and R⁶ independently are —H, methyl, fluoro, chloro, bromo, —OH, methoxyl, —CN, —NO₂, —CH₂OH, —SO₂Me, —CF₃, —CH₂F, —CHF₂, —CCl₃, —COOMe, —COOEt, —NHSO₂Me, —NHBn, phenyl, —SBn, —OBn, —SO₂NH₂, —SO₂Me, —SO₂Et, —SO₂Bn, —SO₂NMe₂, —OSO₂Me, —C≡C-phenyl, —C≡CCH₂(OCH₂CH₂)OH, or —C≡CCH₂(OCH₂CH₂)OMe, or
   R⁴ and R⁵ taken together form a fused pyrazole ring, a fused pyrrole ring, or a fused phenyl ring, and R⁶ is as defined above; and
wherein any alkyl, phenyl, -Bn, pyridinyl, fused pyrazole ring, fused pyrrole ring, or fused phenyl ring moiety of an R⁴, R⁵, or R⁶ group can be further substituted with —C₁-C₆ alkyl, —O—(C₁-C₆ alkyl), —SO₂(C₁-C₆ alkyl), halogen, —CN, haloalkyl, —OH, pyridinyl, furanyl, tetrahydrofuranyl, tetrahydropyranyl, thiophenyl, thiazolyl, pyrrolyl, pyrrolidinyl, piperidinyl, morpholinyl, pyrimidinyl, triazolyl, imidazolyl, —NO₂, —N-piperazinyl-N—(C₁-C₆ alkyl), phenyl, Bn, or any combination thereof, optionally wherein any alkyl, phenyl, Bn, pyridinyl, fused pyrazole ring, fused pyrrole ring, or fused phenyl ring moiety of an R⁴, R⁵, or R⁶ group can be further substituted with —C₁-C₆ alkyl, —O—(C₁-C₆ alkyl), —SO₂(C₁-C₆ alkyl), halogen, —CN, haloalkyl, —OH, pyridinyl, —NO₂, —N— piperazinyl-N—(C₁-C₆ alkyl), phenyl, -Bn, or any combination thereof;
provided that:
when R¹² is phenyl; R¹, R², R⁴, R⁵, and R¹⁰ are —H; and R³ is tetrazole or tetrazole with its N—H group substituted with N-Bn; then R⁶ is not —H, 4-methoxy, or 4-fluoro;
when R¹² is phenyl; R¹, R², R⁴, R⁵, and R¹⁰ are —H; and R³ is —COOH; then R⁶ is not —H, 4-nitro, 4-chloro, 4-methoxyl, 4-CF₃, 4-bromo, 4-isopropyl, or 4-hydroxyl;
when R¹² is phenyl; R¹, R², R⁴, and R¹⁰ are —H; R³ is —COOH; and R⁵ is 4-hydroxyl; then R⁶ is not CO₂Me or methoxyl;
when R¹² is phenyl; R¹, R², and R¹⁰ are —H; and R³ is —COOH, —COOMe, or —COOEt; then R⁴, R⁵, and R⁶ are not 3-methoxyl, 4-methoxyl, and 5-methoxyl, respectively;
when R¹² is phenyl; R¹, R², R⁴, R⁵, and R¹⁰ are —H; and R³ is —COOMe; then R⁶ is not —H, 4-methoxyl, 2-chloro, 4-chloro, 4-hydroxyl, 3-nitro, 4-nitro, 4-bromo, 4-methoxyl, or 4-CF₃; and
when R¹² is phenyl; R¹, R⁴, R⁵, and R¹⁰ are —H; R² is methoxyl or methyl; and R³ is —COOEt; then R⁶ is not —H, 4-methoxyl, 2-chloro, 4-hydroxyl, or 3-nitro;
or a salt or ester thereof.

In some aspects in formula (I), X is C—H. In some aspects in formula (I), X is N.

In some aspects in formula (I), R¹, R², and R¹⁰ independently are —H, —OH, —CN, -haloalkyl, —O—(C₁-C₆ alkyl), halogen, —C₁-C₆ alkyl, or phenyl. A compound of formula (I) can have any combination of the R¹, R², and R¹⁰ groups as defined herein. For example, in some aspects, R¹ is —H, R² is —H, and R¹⁰ is —H. In some aspects, R¹ is —OH, R² is —CN, and R¹⁰ is -haloalkyl (e.g., —CF₃, —CH₂F, —CHF₂, —CCl₃, —CH₂CH₂F, —CH₂CHF₂, —CH₂CF₃, —CHFCH₃, —CF₂CH₃, —CF₂CF₃, —CCl₂CCl₃, —CCl₂CF₃, —CF₂CCl₃, or —CH₂CH₂CF₃). In some aspects, R¹ is —H, R² is -halogen (e.g., —F, —Cl, —Br, or —I), and R¹⁰ is —O—(C₁-C₆ alkyl) (e.g., —OMe, —OEt, —O-nPr, —O-iPr, —O-nBu, —O-tBu, —O-sBu, —O-iBu, —O-n-pentyl, —O-isopentyl, —O-n-hexyl, or any other C₁-C₆ alkyl as defined elsewhere herein). In some aspects, R¹ is —CN, R² is phenyl, and R¹⁰ is —(C₁-C₆ alkyl) (e.g., -Me, -Et, -nPr, -iPr, -nBu, -tBu, -sBu, -iBu, -n-pentyl, -isopenyl, -n-hexyl, or any other C₁-C₆ alkyl as defined elsewhere herein). In some aspects, R¹, R², and R¹⁰ independently are H, hydroxyl, methoxyl, a halogen, methyl, —CN, —CF₃, —CH₂F, —CHF₂, —CCl₃, or phenyl. In some aspects, R¹ and R² are —H, and R¹⁰ is H, hydroxyl, methoxyl, a halogen, methyl, —CN, —CF₃, —CH₂F, —CHF₂, —CCl₃, or phenyl.

In some aspects in formula (I), R³ is —COOH, —COO—(C₁-C₆ alkyl), —CONH₂, —CON(C₁-C₆ alkyl)₂, —CONH(C₁-C₆ alkyl), —CONHSO₂(C₁-C₆ alkyl), —CON(C₁-C₆ alkyl)SO₂(C₁-C₆ alkyl), halogen, —SO₂NH₂, —NHSO₂(C₁-C₆ alkyl), —CN, or tetrazole. For example, in some aspects, R³ is —COOH. In some aspects, R³ is —CONH₂. In some aspects, R³ is —SO₂NH₂. In some aspects, R³ is —CN. In some aspects, R³ is —COO—(C₁-C₆ alkyl) (e.g., —COOMe, —COOEt, —COOPr, —COO-nBu, —COO-tBu, or any other C₁-C₆ alkyl as defined elsewhere herein). In some aspects, R³ is —CON(C₁-C₆ alkyl)₂ (e.g., —CON(Me)(Et), —CON(Me)(Me), —CON(Et)₂, —CON(tBu)(tBu), and so forth, or any other C₁-C₆ alkyl or combination thereof as defined elsewhere herein). In some aspects, R³ is —CONH(C₁-C₆ alkyl) (e.g., —CONH(Me), —CONH(Et), and so forth, or any other C₁-C₆ alkyl as defined elsewhere herein). In some aspects, R³ is —CONHSO₂(C₁-C₆ alkyl) (e.g., —CONHSO₂Me, —CONHSO₂Et, —CONHSO₂-tBu, or any other C₁-C₆ alkyl as defined elsewhere herein). In some aspects, R³ is —NHSO₂(C₁-C₆ alkyl) (e.g., —NHSO₂Me, or any other C₁-C₆ alkyl as defined elsewhere herein). In some aspects, R³ is —COOH or an ester thereof, in which the —COOH group is replaced with —COO—(C₁-C₆ alkyl). In some aspects, R³ is —COOH, —COO—(C₁-C₆ alkyl), —CONH₂, —CONHSO₂(C₁-C₆ alkyl), or tetrazole. In some aspects, R³ is —COOH, —COOMe, —COOEt, —COO(n-propyl), —COO(i-propyl), —COO(n-butyl), —COO(i-butyl), —COO(s-butyl), or —COO(t-butyl). In some aspects, R³ is tetrazole having the following structure:

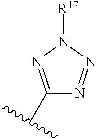

in which R¹⁷ is —H, C₁-C₆ alkyl, —O—(C₁-C₆ alkyl), —SO₂(C₁-C₆ alkyl), halogen, —CN, —OH, phenyl, or Bn; in which any phenyl, -Bn, or alkyl moiety of an R¹⁷ group can be further substituted with C₁-C₆ alkyl, —O—(C₁-C₆ alkyl), —SO₂(C₁-C₆ alkyl), halogen, —CN, haloalkyl, —OH, pyridinyl, nitro, —N-piperazinyl-N—(C₁-C₆ alkyl), phenyl, -Bn, or any combination thereof. In some aspects, R¹⁷ is —H. The —N-piperazinyl-N—(C₁-C₆ alkyl) structure is shown below and is applicable to any disclosure herein where this term is used:

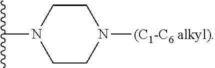

In some aspects in formula (I), R¹¹ is —H. In some aspects in formula (I), R¹¹ is —C₁-C₆ alkyl. R¹¹ can be any C₁-C₆ alkyl as defined herein. In some aspects in formula (I), Ru is methyl, ethyl, isopropyl, n-propyl, n-butyl, isobutyl, t-butyl, sec-butyl, n-pentyl, isopentyl, n-hexyl, or the like. In some aspects in formula (I), R¹¹ is methyl.

In some aspects in formula (I), any alkyl, phenyl, or tetrazole moiety of an R¹, R², R³, R¹⁰, or R¹¹ group can be further substituted with —C₁-C₆ alkyl, —O—(C₁-C₆ alkyl), —SO₂(C₁-C₆ alkyl), halogen, —CN, haloalkyl, —OH, pyridinyl, —NO₂, —N-piperazinyl-N—(C₁-C₆ alkyl), phenyl, Bn, or any combination thereof. For example, in some aspects, R¹ and R² are —H, R¹¹ is —H or -Me, R¹⁰ is phenyl, and the R¹⁰ phenyl is substituted with halogen (e.g., —F, —Cl, —Br, or —I). The —C₁-C₆ alkyl, halogen, haloalkyl, and other groups are as defined elsewhere herein. In some aspects, any alkyl, phenyl, or tetrazole moiety of an R¹, R², R³, R¹⁰, or R¹¹ group is not further substituted with any group. In some aspects, any alkyl, phenyl, or tetrazole moiety of an R¹, R², R³, R¹⁰, or R¹¹ group can be further substituted with —C₁-C₆ alkyl, —O—(C₁-C₆ alkyl), —SO₂(C₁-C₆ alkyl), halogen, —CN, haloalkyl, —OH, pyridinyl, —NO₂, —N-piperazinyl-N—(C₁-C₆ alkyl), phenyl, -Bn, or any combination thereof.

In some aspects in formula (I), R¹² is phenyl, a 5-membered heterocycle, or a 6-membered heterocycle, each of which is substituted with R⁴, R⁵, and R⁶ on a carbon atom and/or heteroatom. In some aspects, the 5-membered heterocycle is pyrrole, pyrazole, tetrazole, imidazole, or furan. In some aspects, the 6-membered heterocycle is piperazine, dihydropyrimidine, pyrimidine, pyridine, or pyrazine. In some aspects, R² is phenyl, imidazole, pyridine, or dihydropyrimidine, each of which is substituted with R⁴, R⁵, and R⁶. In some aspects, R¹² is:

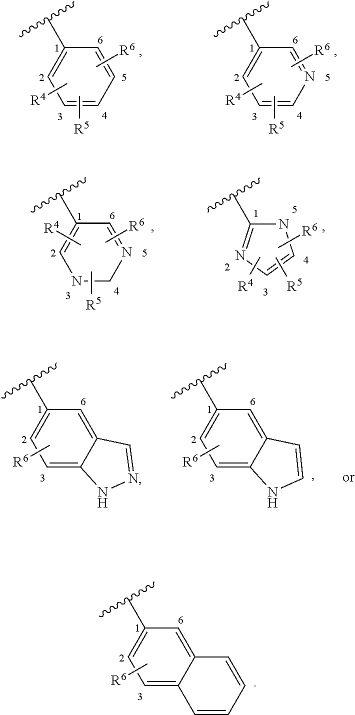

In some aspects in formula (I), R⁴, R⁵, and R⁶ independently are —H, —C₁-C₆ alkyl, halogen, —OH, —O(C₁-C₆ alkyl), —CN, —NO₂, —(C₁-C₆ alkyl)-OH, SO₂(C₁-C₆ alkyl), -haloalkyl, —CO₂(C₁-C₆ alkyl), —NHSO₂(C₁-C₆ alkyl), —NHBn, phenyl, —SBn, —OBn, —SO₂NH₂, —SO₂(C₁-C₆ alkyl), —SO₂Bn, SO₂N(C₁-C₆ alkyl)₂, —OSO₂(C₁-C₆ alkyl), —C≡C-phenyl, —C≡CCH₂(OCH₂CH₂)₁₋₄OH, —C≡CCH₂(OCH₂CH₂)₁₋₄OMe, —NHSO₂-pyridinyl, —NBnSO₂-pyridinyl, —NHSO₂-phenyl, —NHSO₂(N-piperazinyl-N—(C₁-C₆ alkyl)), —N(C₁-C₆ alkyl)SO₂-pyridinyl, —N(C₁-C₆ alkyl)SO₂-phenyl, —N(C₁-C₆ alkyl)SO₂(N-piperazinyl-N—(C₁-C₆ alkyl)), —NBnSO₂(C₁-C₆ alkyl), —NBnSO₂(N-piperazinyl-N—(C₁-C₆ alkyl)), —NBnSO₂-phenyl, or —N(C₁-C₆ alkyl)SO₂(C₁-C₆ alkyl. In some aspects, R⁴, R⁵, and R⁶ independently are —H, methyl, fluoro, chloro, bromo, —OH, methoxyl, —CN, —NO₂, —CH₂OH, —SO₂Me, —CF₃, —CH₂F, —CHF₂, —CCl₃, —COOMe, —COOEt, —NHSO₂Me, —NHBn, phenyl, —SBn, —OBn, —SO₂NH₂, —SO₂Me, —SO₂Et, —SO₂Bn, —SO₂NMe₂, —OSO₂Me, —C≡C-phenyl, —C≡CCH₂(OCH₂CH₂)OH, or —C≡CCH₂(OCH₂CH₂)OMe. In some aspects, R⁴, R⁵, and R⁶ independently are —H; methyl; fluoro; chloro; bromo; hydroxyl; methoxyl; —CN; —NO₂; —CH₂OH; —SO₂Me; —CF₃; —CH₂F; —CHF₂; —CCl₃; —CO₂Me; —CO₂Et; —NHSO₂Me; —NHBn; phenyl; —SBn; —OBn; —SO₂NH₂; —SO₂Me; —SO₂Et; —SO₂Bn; —SO₂NMe₂; —OSO₂Me; —C≡C-phenyl; —C≡CCH₂(OCH₂CH₂)OH; —C≡CCH₂(OCH₂CH₂)OMe; phenyl substituted with at least one of methyl, methoxyl, —SO₂Me, or halogen; or —NHBn wherein -Bn is substituted with at least one of —C₁-C₆ alkyl, —O—(C₁-C₆ alkyl), —SO₂(C₁-C₆ alkyl), halogen, —CN, haloalkyl, —OH, pyridinyl, —NO₂, —N-piperazinyl-N—(C₁-C₆ alkyl), or phenyl.

In some aspects in formula (I), R⁴ and R⁵ taken together form a fused pyrazole ring, a fused pyrrole ring, or a fused phenyl ring, and R⁶ is as defined above. In some aspects, R⁴ and R⁵ taken together form a fused pyrazole ring, a fused pyrrole ring, or a fused phenyl ring, and R⁶ is as defined above. In some aspects, R⁴ and R⁵ are taken together to form a structure as follows:

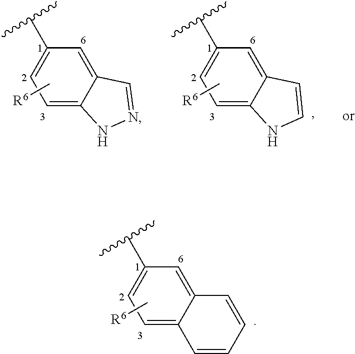

In some aspects in formula (I), R¹² is phenyl; R⁴ is —H; R⁵ and R⁶ are as defined above; R¹ and R² independently are —H, —OH, —C₁-C₆ alkyl, —O—(C₁-C₆ alkyl), halogen, —CN, haloalkyl, or phenyl; R³ is —COOH, —COO(C₁-C₆ alkyl), or tetrazole; and R¹¹ is —H or —C₁-C₆ alkyl. In some aspects, R¹² is phenyl; R⁴ and R⁵ are —H; R⁶ is as defined above; R¹ and R² independently are —H, —OH, —C₁-C₆ alkyl, —O—(C₁-C₆ alkyl), halogen, —CN, haloalkyl, or phenyl; R³ is —COOH, —COO(C₁-C₆ alkyl), or tetrazole; and R¹¹ is —H or —C₁-C₆ alkyl. In some aspects, R¹² is phenyl; R⁴ and R⁵ are —H; R⁶ is as defined above; R¹ and R² independently are —H, halogen, —CN, or haloalkyl; R³ is —COOH, —COO(C₁-C₆ alkyl), or tetrazole; and R¹¹ is —H or —C₁-C₆ alkyl. In some aspects, R¹² is phenyl; R⁴ and R⁵ taken together form a fused pyrazole ring, a fused pyrrole ring, or a fused phenyl ring, and R⁶ is as defined above; R^{t} and R² independently are —H, —OH, —C₁-C₆ alkyl, —O—(C₁-C₆ alkyl), halogen, —CN, haloalkyl, or phenyl; R³ is —COOH, —COO(C₁-C₆ alkyl), or tetrazole; and R¹¹ is —H or —C₁-C₆ alkyl. In some aspects, R¹² is phenyl; R⁴ and R⁵ taken together form a fused pyrazole ring, a fused pyrrole ring, or a fused phenyl ring, and R⁶ is as defined above; R¹ and R² independently are —H, —OH, —C₁-C₆ alkyl, —O—(C₁-C₆ alkyl), halogen, —CN, haloalkyl, or phenyl; R³ is —COOH, —COO(C₁-C₆ alkyl), or tetrazole; and R¹¹ is —H or —C₁-C₆ alkyl. In some aspects, R¹² is phenyl; R⁴ and R⁵ taken together form a fused pyrazole ring, a fused pyrrole ring, or a fused phenyl ring, and R⁶ is as defined above; R¹ and R² independently are —H, halogen, —CN, or haloalkyl; R³ is —COOH, —COO(C₁-C₆ alkyl), or tetrazole; and R¹¹ is —H or —C₁-C₆ alkyl.

In some aspects in formula (I), any alkyl, phenyl, Bn, pyridinyl, fused pyrazole ring, fused pyrrole ring, or fused phenyl ring moiety of an R⁴, R⁵, or R⁶ group can be further substituted with —C₁-C₆ alkyl, —O—(C₁-C₆ alkyl), —SO₂(C₁-C₆ alkyl), halogen, —CN, haloalkyl, —OH, pyridinyl, furanyl, tetrahydrofuranyl, tetrahydropyranyl, thiophenyl, thiazolyl, pyrrolyl, pyrrolidinyl, piperidinyl, morpholinyl, pyrimidinyl, triazolyl, imidazolyl, —NO₂, —N-piperazinyl-N—(C₁-C₆ alkyl), phenyl, -Bn, or any combination thereof. In some aspects in formula (I), any alkyl, phenyl, Bn, pyridinyl, fused pyrazole ring, fused pyrrole ring, or fused phenyl ring moiety of an R⁴, R⁵, or R⁶ group can be further substituted with —C₁-C₆ alkyl, —O—(C₁-C₆ alkyl), —SO₂(C₁-C₆ alkyl), halogen, —CN, haloalkyl, —OH, pyridinyl, —NO₂, —N-piperazinyl-N—(C₁-C₆ alkyl), phenyl, -Bn, or any combination thereof. As used herein, “fused pyrazole ring, fused pyrrole ring, or fused phenyl ring moiety of an R⁴, R⁵, or R⁶ group” includes the fused structures that can be formed by taking together R⁴ and R⁵, as described elsewhere herein. For clarity, the R¹² phenyl, 5-membered heterocycle, or 6-membered heterocycle is not further substituted as described above, since R⁴, R⁵, and R⁶ define the substitution on such R¹² phenyl, 5-membered heterocycle, or 6-membered heterocycle, as described elsewhere herein. In some aspects, R⁴ and R⁵ are —H, R⁶ is phenyl, and this R⁶ phenyl is further substituted as defined above.

In some aspects in formula (I), when R¹² is phenyl; R¹, R², R⁴, R⁵, and R¹⁰ are —H; and R³ is tetrazole or tetrazole with its N—H group substituted with N-Bn; then R⁶ is not —H, 4-methoxy, or 4-fluoro (i.e., proviso 1).

In some aspects in formula (I), when R¹¹ is phenyl; R¹, R², R⁴, R⁵, and R¹⁰ are —H; and R³ is —COOH; then R⁶ is not —H, 4-nitro, 4-chloro, 4-methoxyl, 4-CF₃, 4-bromo, 4-isopropyl, or 4-hydroxyl (i.e., proviso 2).

In some aspects in formula (I), when R¹² is phenyl; R¹, R², R⁴, and R¹⁰ are —H; R³ is —COOH; and R⁵ is 4-hydroxyl; then R⁶ is not CO₂Me or methoxyl (i.e., proviso 3).

In some aspects in formula (I), when R¹² is phenyl; R¹, R², and R¹⁰ are —H; and R³ is —COOH, —COOMe, or —COOEt; then R⁴, R⁵, and R⁶ are not 3-methoxyl, 4-methoxyl, and 5-methoxyl, respectively (i.e., proviso 4).

In some aspects in formula (I), when R¹² is phenyl; R¹, R², R⁴, R⁵, and R¹⁰ are —H; and R³ is —COOMe; then R⁶ is not —H, 4-methoxyl, 2-chloro, 4-chloro, 4-hydroxyl, 3-nitro, 4-nitro, 4-bromo, 4-methoxyl, or 4-CF₃ (i.e., proviso 5).

In some aspects in formula (I), when R¹² is phenyl; R¹, R⁴, R³, and R¹⁰ are —H; R² is methoxyl or methyl; and R³ is —COOEt; then R⁶ is not —H, 4-methoxyl, 2-chloro, 4-hydroxyl, or 3-nitro (i.e., proviso 5).

In some aspects, neither formula (I) nor formula (II) (described elsewhere herein) includes the compounds specified by all of provisos 1-5.

In some aspects in formula (I):
R² is phenyl, imidazole, pyridine, or dihydropyrimidine, each of which is substituted with R⁴, R³, and R⁶;
R¹, R², R⁴, R⁵, and R¹⁰ are H;
R³ is —COOH or —COO—(C₁-C₆ alkyl);
R⁶ is methyl; fluoro; chloro; bromo; hydroxyl; methoxyl; —CN; —NO₂; —CH₂OH; —SO₂Me; —CF₃; —CH₂F; —CHF₂; —CCl₃; —CO₂Me; —NHSO₂Me; —SBn; —OBn; —SO₂NH₂; —SO₂Me; —SO₂Bn; —SO₂NMe₂; —OSO₂Me; —C≡C-phenyl; —C≡CCH₂(OCH₂CH₂)OH; —C≡CCH₂(OCH₂CH₂)OMe; —NHBn; —NHBn wherein -Bn is substituted with at least one of —C₁-C₆ alkyl, —O—(C₁-C₆ alkyl), —SO₂(C₁-C₆ alkyl), halogen, —CN, —CF₃, —OH, pyridinyl, —NO₂, —N-piperazinyl-N—(C₁-C₆ alkyl), or phenyl; phenyl; or phenyl substituted with at least one of methyl, methoxyl, —SO₂Me, or halogen; and
R¹¹ is H or —C¹-C⁶ alkyl;
optionally wherein X is C—H;
provided that:
when R¹² is phenyl, then R⁶ is not 4-nitro, 4-chloro, 4-methoxyl, 4-CF₃, 4-bromo, 4-isopropyl, 2-chloro, 4-chloro, 4-hydroxyl, 3-nitro, or 4-hydroxyl.

In some aspects in formula (I):
R¹² is phenyl, imidazole, pyridine, or dihydropyrimidine, each of which is substituted with R⁴, R⁵, and R⁶;
R¹, R², R⁴, R⁵, and R¹⁰ are H;
R³ is —COOH, —COOMe, —COOEt, —COO(n-propyl), —COO(i-propyl), —COO(n-butyl), —COO(i-butyl), —COO(s-butyl), —COO(t-butyl), or tetrazole (e.g., the tetrazole structure defined elsewhere herein);
R⁶ is methyl; fluoro; chloro; bromo; hydroxyl; methoxyl; —CN; —NO₂; —CH₂OH; —SO₂Me; —CF₃; —CH₂F; —CHF₂; —CCl₃; —CO₂Me; —NHSO₂Me; —SBn; —OBn; —SO₂NH₂; —SO₂Me; —SO₂Bn; —SO₂NMe₂; —OSO₂Me; —C≡C-phenyl; —C≡CCH₂(OCH₂CH₂)OH; —C≡CCH₂(OCH₂CH₂)OMe; —NHBn; —NHBn wherein -Bn is substituted with at least one of —C₁-C₆ alkyl, —O—(C₁-C₆ alkyl), —SO₂(C₁-C₆ alkyl), halogen, —CN, —CF₃, —OH, pyridinyl, —NO₂, —N-piperazinyl-N—(C₁-C₆ alkyl), or phenyl; phenyl; or phenyl substituted with at least one of methyl, methoxyl, —SO₂Me, or halogen; and
R¹¹ is H or —C¹-C⁶ alkyl;
optionally wherein X is C—H;
provided that:
when R¹² is phenyl, then R⁶ is not 4-nitro, 4-chloro, 4-methoxyl, 4-CF₃, 4-bromo, 4-isopropyl, 2-chloro, 4-chloro, 4-hydroxyl, 3-nitro, or 4-hydroxyl.

In some aspects in formula (I):
R¹² is phenyl, imidazole, pyridine, or dihydropyrimidine, each of which is substituted with R⁴, R⁵, and R⁶;
R¹, R², R⁴, R⁵, and R¹⁰ are H;
R³ is —COOH, —COOMe, —COOEt, —COO(n-propyl), —COO(i-propyl), —COO(n-butyl), —COO(i-butyl), —COO(s-butyl), —COO(t-butyl), or tetrazole (e.g., the tetrazole structure defined elsewhere herein);
R⁶ is methyl; fluoro; chloro; bromo; hydroxyl; methoxyl; —CN; —NO₂; —CH₂OH; —SO₂Me; —CF₃; —CH₂F; —CHF₂; —CCl₃; —CO₂Me; —NHSO₂Me; —SBn; —OBn; —SO₂NH₂; —SO₂Me; —SO₂Bn; —SO₂NMe₂; —OSO₂Me; —C≡C-phenyl; —C≡CCH₂(OCH₂CH₂)OH; —C≡CCH₂(OCH₂CH₂)OMe; —NHBn; —NHBn wherein -Bn is substituted with at least one of —C₁-C₆ alkyl, —O—(C₁-C₆ alkyl), —SO₂(C₁-C₆ alkyl), halogen, —CN, —CF₃, —OH, pyridinyl, —NO₂, —N-piperazinyl-N—(C₁-C₆ alkyl), or phenyl; phenyl; or phenyl substituted with at least one of methyl, methoxyl, —SO₂Me, or halogen; and
R¹¹ is —H, -Me, Et, -nPr, or -iPr;
optionally wherein X is C—H;
provided that:
when R¹² is phenyl, then R⁶ is not 4-nitro, 4-chloro, 4-methoxyl, 4-CF₃, 4-bromo, 4-isopropyl, 2-chloro, 4-chloro, 4-hydroxyl, 3-nitro, or 4-hydroxyl.

In some aspects in formula (I):
R¹² is phenyl, imidazole, pyridine, or dihydropyrimidine, each of which is substituted with R⁴, R⁵, and R⁶;
R¹, R², R⁴, R⁵, and R¹⁰ are H;
R³ is —COOH or —COO—(C₁-C₆ alkyl);
R⁶ is 4-methyl, 4-fluoro, 3-fluoro, 3-chloro, 3-methyl, 3-methoxy, 4-cyano, 4-(CH₂OH), 4-(SO₂Me), 3-nitro, 3-hydroxyl, 4-(CO₂Me), 3-cyano, 4-(NHSO₂Me), 4-(4-methylphenyl), 4-(4-methoxyphenyl), 4-(4-(SO₂Me)phenyl), 4-(SBn), 4-(NHCH₂(4-fluorophenyl)), 4-(SO₂NH₂), 4-(4-fluorophenyl), 4-(SO₂Bn), 3-(CO₂Me), 4-(SO₂Et), 4-(SO₂NMe₂), 4-(OSO₂Me), 4-(C≡C-phenyl), 4-(C≡CCH₂OCH₂CH₂OCH₃), 4-(NHSO₂-phenyl), 4-(NBnSO₂Me), or 4-(NMeSO₂Me); and
R¹¹ is H or —C¹-C⁶ alkyl;
optionally wherein X is C—H.

In some aspects in formula (I):
R¹² is phenyl, imidazole, pyridine, or dihydropyrimidine, each of which is substituted with R⁴, R⁵, and R⁶;
R¹, R², R⁴, R⁵, and R¹⁰ are H;
R³ is —COOH, —COOMe, —COOEt, —COO(n-propyl), —COO(i-propyl), —COO(n-butyl), —COO(i-butyl), —COO(s-butyl), —COO(t-butyl), or tetrazole (e.g., the tetrazole structure defined elsewhere herein);
R⁶ is 4-methyl, 4-fluoro, 3-fluoro, 3-chloro, 3-methyl, 3-methoxy, 4-cyano, 4-(CH₂OH), 4-(SO₂Me), 3-nitro, 3-hydroxyl, 4-(CO₂Me), 3-cyano, 4-(NHSO₂Me), 4-(4-methylphenyl), 4-(4-methoxyphenyl), 4-(4-(SO₂Me)phenyl), 4-(SBn), 4-(NHCH₂(4-fluorophenyl)), 4-(SO₂NH₂), 4-(4-fluorophenyl), 4-(SO₂Bn), 3-(CO₂Me), 4-(SO₂Et), 4-(SO₂NMe₂), 4-(OSO₂Me), 4-(C≡C-phenyl), 4-(C≡CCH₂OCH₂CH₂OCH₃), 4-(NHSO₂-phenyl), 4-(NBnSO₂Me), or 4-(NMeSO₂Me); and
R¹¹ is H or —C¹-C⁶ alkyl;
optionally wherein X is C—H.

In some aspects in formula (I):
R¹² is phenyl, imidazole, pyridine, or dihydropyrimidine, each of which is substituted with R⁴, R⁵, and R⁶;
R¹, R², R⁴, R⁵, and R¹⁰ are H;
R³ is —COOH, —COOMe, —COOEt, —COO(n-propyl), —COO(i-propyl), —COO(n-butyl), —COO(i-butyl), —COO(s-butyl), —COO(t-butyl), or tetrazole (e.g., the tetrazole structure defined elsewhere herein);
R⁶ is 4-methyl, 4-fluoro, 3-fluoro, 3-chloro, 3-methyl, 3-methoxy, 4-cyano, 4-(CH₂OH), 4-(SO₂Me), 3-nitro, 3-hydroxyl, 4-(CO₂Me), 3-cyano, 4-(NHSO₂Me), 4-(4-methylphenyl), 4-(4-methoxyphenyl), 4-(4-(SO₂Me)phenyl), 4-(SBn), 4-(NHCH₂(4-fluorophenyl)), 4-(SO₂NH₂), 4-(4-fluorophenyl), 4-(SO₂Bn), 3-(CO₂Me), 4-(SO₂Et), 4-(SO₂NMe₂), 4-(OSO₂Me), 4-(C≡C-phenyl), 4-(C≡CCH₂OCH₂CH₂OCH₃), 4-(NHSO₂-phenyl), 4-(NBnSO₂Me), or 4-(NMeSO₂Me); and
R¹¹ is —H, -Me, Et, -nPr, or -iPr;
optionally wherein X is C—H.

In some aspects in formula (I):
R¹² is phenyl, imidazole, pyridine, or dihydropyrimidine, each of which is substituted with R⁴, R⁵, and R⁶;
R¹, R², R⁴, and R⁵ are H;
R¹⁰ is H, hydroxyl, methoxyl, a halogen, methyl, —CN, —CF₃, —CH₂F, —CHF₂, —CCl₃, or phenyl;
R³ is —COOH or —COO—(C₁-C₆ alkyl);
R⁶ is methyl; fluoro; chloro; bromo; hydroxyl; methoxyl; —CN; —NO₂; —CH₂OH; —SO₂Me; —CF₃; —CH₂F; —CHF₂; —CCl₃; —CO₂Me; —NHSO₂Me; —SBn; —OBn; —SO₂NH₂; —SO₂Me; —SO₂Bn; —SO₂NMe₂; —OSO₂Me; —C≡C-phenyl; —C≡CCH₂(OCH₂CH₂)OH; —C≡CCH₂(OCH₂CH₂)OMe; —NHBn; —NHBn wherein -Bn is substituted with at least one of —C₁-C₆ alkyl, —O—(C₁-C₆ alkyl), —SO₂(C₁-C₆ alkyl), halogen, —CN, —CF₃, —OH, pyridinyl, —NO₂, —N-piperazinyl-N—(C₁-C₆ alkyl), or phenyl; phenyl; or phenyl substituted with at least one of methyl, methoxyl, —SO₂Me, or halogen; and
R¹¹ is H or —C¹-C⁶ alkyl;
optionally wherein X is C—H;
provided that:
when R¹² is phenyl, then R⁶ is not 4-nitro, 4-chloro, 4-methoxyl, 4-CF₃, 4-bromo, 4-isopropyl, 2-chloro, 4-chloro, 4-hydroxyl, 3-nitro, or 4-hydroxyl.

In some aspects in formula (I):
R¹² is phenyl, imidazole, pyridine, or dihydropyrimidine, each of which is substituted with R⁴, R⁵, and R⁶;
R¹, R², R⁴, and R⁵ are H;
R¹⁰ is H, hydroxyl, methoxyl, a halogen, methyl, —CN, —CF₃, —CH₂F, —CHF₂, —CCl₃, or phenyl;
R³ is —COOH, —COOMe, —COOEt, —COO(n-propyl), —COO(i-propyl), —COO(n-butyl), —COO(i-butyl), —COO(s-butyl), —COO(t-butyl), or tetrazole (e.g., the tetrazole structure defined elsewhere herein);
R⁶ is methyl; fluoro; chloro; bromo; hydroxyl; methoxyl; —CN; —NO₂; —CH₂OH; —SO₂Me; —CF₃; —CH₂F; —CHF₂; —CCl₃; —CO₂Me; —NHSO₂Me; —SBn; —OBn; —SO₂NH₂; —SO₂Me; —SO₂Bn; —SO₂NMe₂; —OSO₂Me; —C≡C-phenyl; —C≡CCH₂(OCH₂CH₂)OH; —C≡CCH₂(OCH₂CH₂)OMe; —NHBn; —NHBn wherein -Bn is substituted with at least one of —C₁-C₆ alkyl, —O—(C₁-C₆ alkyl), —SO₂(C₁-C₆ alkyl), halogen, —CN, —CF₃, —OH, pyridinyl, —NO₂, —N-piperazinyl-N—(C₁-C₆ alkyl), or phenyl; phenyl; or phenyl substituted with at least one of methyl, methoxyl, —SO₂Me, or halogen; and
R¹¹ is H or —C¹-C⁶ alkyl;
optionally wherein X is C—H;
provided that:
when R¹² is phenyl, then R⁶ is not 4-nitro, 4-chloro, 4-methoxyl, 4-CF₃, 4-bromo, 4-isopropyl, 2-chloro, 4-chloro, 4-hydroxyl, 3-nitro, or 4-hydroxyl.

In some aspects in formula (I):
R¹² is phenyl, imidazole, pyridine, or dihydropyrimidine, each of which is substituted with R⁴, R⁵, and R⁶;
R¹, R², R⁴, and R⁵ are H;
R¹⁰ is H, hydroxyl, methoxyl, a halogen, methyl, —CN, —CF₃, —CH₂F, —CHF₂, —CCl₃, or phenyl;
R³ is —COOH, —COOMe, —COOEt, —COO(n-propyl), —COO(i-propyl), —COO(n-butyl), —COO(i-butyl), —COO(s-butyl), —COO(t-butyl), or tetrazole (e.g., the tetrazole structure defined elsewhere herein);
R⁶ is methyl; fluoro; chloro; bromo; hydroxyl; methoxyl; —CN; —NO₂; —CH₂OH; —SO₂Me; —CF₃; —CH₂F; —CHF₂; —CCl₃; —CO₂Me; —NHSO₂Me; —SBn; —OBn; —SO₂NH₂; —SO₂Me; —SO₂Bn; —SO₂NMe₂; —OSO₂Me; —C≡C-phenyl; —C≡CCH₂(OCH₂CH₂)OH; —C≡CCH₂(OCH₂CH₂)OMe; —NHBn; —NHBn wherein -Bn is substituted with at least one of —C₁-C₆ alkyl, —O—(C₁-C₆ alkyl), —SO₂(C₁-C₆ alkyl), halogen, —CN, —CF₃, —OH, pyridinyl, —NO₂, —N-piperazinyl-N—(C₁-C₆ alkyl), or phenyl; phenyl; or phenyl substituted with at least one of methyl, methoxyl, —SO₂Me, or halogen; and
R¹¹ is —H, -Me, Et, -nPr, or -iPr;
optionally wherein X is C—H;
provided that:
when R¹² is phenyl, then R⁶ is not 4-nitro, 4-chloro, 4-methoxyl, 4-CF₃, 4-bromo, 4-isopropyl, 2-chloro, 4-chloro, 4-hydroxyl, 3-nitro, or 4-hydroxyl.

In some aspects in formula (I):
R¹² is phenyl, imidazole, pyridine, or dihydropyrimidine, each of which is substituted with R⁴, R⁵, and R⁶;
R¹, R², R⁴, and R⁵ are H;
R¹⁰ is H, hydroxyl, methoxyl, a halogen, methyl, —CN, —CF₃, —CH₂F, —CHF₂, —CCl₃, or phenyl;
R³ is —COOH or —COO—(C₁-C₆ alkyl);
R⁶ is 4-methyl, 4-fluoro, 3-fluoro, 3-chloro, 3-methyl, 3-methoxy, 4-cyano, 4-(CH₂OH), 4-(SO₂Me), 3-nitro, 3-hydroxyl, 4-(CO₂Me), 3-cyano, 4-(NHSO₂Me), 4-(4-methylphenyl), 4-(4-methoxyphenyl), 4-(4-(SO₂Me)phenyl), 4-(SBn), 4-(NHCH₂(4-fluorophenyl)), 4-(SO₂NH₂), 4-(4-fluorophenyl), 4-(SO₂Bn), 3-(CO₂Me), 4-(SO₂Et), 4-(SO₂NMe₂), 4-(OSO₂Me), 4-(C≡C-phenyl), 4-(C≡CCH₂OCH₂CH₂OCH₃), 4-(NHSO₂-phenyl), 4-(NBnSO₂Me), or 4-(NMeSO₂Me); and
R¹¹ is H or —C¹-C⁶ alkyl;
optionally wherein X is C—H.

In some aspects in formula (I):
R¹² is phenyl, imidazole, pyridine, or dihydropyrimidine, each of which is substituted with R⁴, R⁵, and R⁶;
R¹, R², R⁴, and R⁵ are H;
R¹⁰ is H, hydroxyl, methoxyl, a halogen, methyl, —CN, —CF₃, —CH₂F, —CHF₂, —CCl₃, or phenyl;
R³ is —COOH, —COOMe, —COOEt, —COO(n-propyl), —COO(i-propyl), —COO(n-butyl), —COO(i-butyl), —COO(s-butyl), —COO(t-butyl), or tetrazole (e.g., the tetrazole structure defined elsewhere herein);
R⁶ is 4-methyl, 4-fluoro, 3-fluoro, 3-chloro, 3-methyl, 3-methoxy, 4-cyano, 4-(CH₂OH), 4-(SO₂Me), 3-nitro, 3-hydroxyl, 4-(CO₂Me), 3-cyano, 4-(NHSO₂Me), 4-(4-methylphenyl), 4-(4-methoxyphenyl), 4-(4-(SO₂Me)phenyl), 4-(SBn), 4-(NHCH₂(4-fluorophenyl)), 4-(SO₂NH₂), 4-(4-fluorophenyl), 4-(SO₂Bn), 3-(CO₂Me), 4-(SO₂Et), 4-(SO₂NMe₂), 4-(OSO₂Me), 4-(C≡C-phenyl), 4-(C≡CCH₂OCH₂CH₂OCH₃), 4-(NHSO₂-phenyl), 4-(NBnSO₂Me), or 4-(NMeSO₂Me); and
R¹¹ is H or —C¹-C⁶ alkyl;
optionally wherein X is C—H.

In some aspects in formula (I):
R² is phenyl, imidazole, pyridine, or dihydropyrimidine, each of which is substituted with R⁴, R⁵, and R⁶;
R¹, R², R⁴, and R⁵ are H;
R¹⁰ is H, hydroxyl, methoxyl, a halogen, methyl, —CN, —CF₃, —CH₂F, —CHF₂, —CCl₃, or phenyl;
R³ is —COOH, —COOMe, —COOEt, —COO(n-propyl), —COO(i-propyl), —COO(n-butyl), —COO(i-butyl), —COO(s-butyl), —COO(t-butyl), or tetrazole (e.g., the tetrazole structure defined elsewhere herein);
R⁶ is 4-methyl, 4-fluoro, 3-fluoro, 3-chloro, 3-methyl, 3-methoxy, 4-cyano, 4-(CH₂OH), 4-(SO₂Me), 3-nitro, 3-hydroxyl, 4-(CO₂Me), 3-cyano, 4-(NHSO₂Me), 4-(4-methylphenyl), 4-(4-methoxyphenyl), 4-(4-(SO₂Me)phenyl), 4-(SBn), 4-(NHCH₂(4-fluorophenyl)), 4-(SO₂NH₂), 4-(4-fluorophenyl), 4-(SO₂Bn), 3-(CO₂Me), 4-(SO₂Et), 4-(SO₂NMe₂), 4-(OSO₂Me), 4-(C≡C-phenyl), 4-(C≡CCH₂OCH₂CH₂OCH₃), 4-(NHSO₂-phenyl), 4-(NBnSO₂Me), or 4-(NMeSO₂Me); and
R¹¹ is —H, -Me, Et, -nPr, or -iPr;
optionally wherein X is C—H.

In some aspects in formula (I):
R¹² is phenyl, imidazole, pyridine, or dihydropyrimidine, each of which is substituted with R⁴, R⁵, and R⁶;
R¹ and R⁴ are H;
R² and R¹⁰ independently are —H, —OH, —CN, -haloalkyl, —O—(C₁-C₆ alkyl), halogen, —C₁-C₆ alkyl, or phenyl;
R³ is —COOH or —COO—(C₁-C₆ alkyl);
R¹ and R⁶ independently are methyl; fluoro; chloro; bromo; hydroxyl; methoxyl; —CN; —NO₂; —CH₂OH; —SO₂Me; —CF₃; —CH₂F; —CHF₂; —CCl₃; —CO₂Me; —NHSO₂Me; —SBn; —OBn; —SO₂NH₂; —SO₂Me; —SO₂Bn; —SO₂NMe₂; —OSO₂Me; —C≡C-phenyl; —C≡CCH₂(OCH₂CH₂)OH; —C≡CCH₂(OCH₂CH₂)OMe; —NHBn; —NHBn wherein -Bn is substituted with at least one of —C₁-C₆ alkyl, —O—(C₁-C₆ alkyl), —SO₂(C₁-C₆ alkyl), halogen, —CN, —CF₃, —OH, pyridinyl, —NO₂, —N-piperazinyl-N—(C₁-C₆ alkyl), or phenyl;
phenyl; or phenyl substituted with at least one of methyl, methoxyl, —SO₂Me, or halogen; and
R¹¹ is H or —C¹-C⁶ alkyl;
optionally wherein X is C—H;
provided that:
when R¹² is phenyl, then R⁶ is not 4-nitro, 4-chloro, 4-methoxyl, 4-CF₃, 4-bromo, 4-isopropyl, 2-chloro, 4-chloro, 4-hydroxyl, 3-nitro, or 4-hydroxyl.

In some aspects in formula (I):
R¹² is phenyl, imidazole, pyridine, or dihydropyrimidine, each of which is substituted with R⁴, R⁵, and R⁶;
R¹ and R⁴ are H;
R² and R¹⁰ independently are —H, —OH, —CN, -haloalkyl, —O—(C₁-C₆ alkyl), halogen, —C₁-C₆ alkyl, or phenyl;
R³ is —COOH, —COOMe, —COOEt, —COO(n-propyl), —COO(i-propyl), —COO(n-butyl), —COO(i-butyl), —COO(s-butyl), —COO(t-butyl), or tetrazole (e.g., the tetrazole structure defined elsewhere herein);
R⁵ and R⁶ independently are methyl; fluoro; chloro; bromo; hydroxyl; methoxyl; —CN; —NO₂; —CH₂OH; —SO₂Me; —CF₃; —CH₂F; —CHF₂; —CCl₃; —CO₂Me; —NHSO₂Me; —SBn; —OBn; —SO₂NH₂; —SO₂Me; —SO₂Bn; —SO₂NMe₂; —OSO₂Me; —C≡C-phenyl; —C≡CCH₂(OCH₂CH₂)OH; —C≡CCH₂(OCH₂CH₂)OMe; —NHBn; —NHBn wherein -Bn is substituted with at least one of —C₁-C₆ alkyl, —O—(C₁-C₆ alkyl), —SO₂(C₁-C₆ alkyl), halogen, —CN, —CF₃, —OH, pyridinyl, —NO₂, —N-piperazinyl-N—(C₁-C₆ alkyl), or phenyl;
phenyl; or phenyl substituted with at least one of methyl, methoxyl, —SO₂Me, or halogen; and
R¹¹ is H or —C¹-C⁶ alkyl;
optionally wherein X is C—H;
provided that:
when R¹² is phenyl, then R⁶ is not 4-nitro, 4-chloro, 4-methoxyl, 4-CF₃, 4-bromo, 4-isopropyl, 2-chloro, 4-chloro, 4-hydroxyl, 3-nitro, or 4-hydroxyl.

In some aspects in formula (I):
R¹² is phenyl, imidazole, pyridine, or dihydropyrimidine, each of which is substituted with R⁴, R⁵, and R⁶;
R¹ and R⁴ are H;
R² and R¹⁰ independently are —H, —OH, —CN, -haloalkyl, —O—(C₁-C₆ alkyl), halogen, —C₁-C₆ alkyl, or phenyl;
R³ is —COOH, —COOMe, —COOEt, —COO(n-propyl), —COO(i-propyl), —COO(n-butyl), —COO(i-butyl), —COO(s-butyl), —COO(t-butyl), or tetrazole (e.g., the tetrazole structure defined elsewhere herein);
R⁵ and R⁶ independently are methyl; fluoro; chloro; bromo; hydroxyl; methoxyl; —CN; —NO₂; —CH₂OH; —SO₂Me; —CF₃; —CH₂F; —CHF₂; —CCl₃; —CO₂Me; —NHSO₂Me; —SBn; —OBn; —SO₂NH₂; —SO₂Me; —SO₂Bn; —SO₂NMe₂; —OSO₂Me; —C≡C-phenyl; —C≡CCH₂(OCH₂CH₂)OH; —C≡CCH₂(OCH₂CH₂)OMe; —NHBn; —NHBn wherein -Bn is substituted with at least one of —C₁-C₆ alkyl, —O—(C₁-C₆ alkyl), —SO₂(C₁-C₆ alkyl), halogen, —CN, —CF₃, —OH, pyridinyl, —NO₂, —N-piperazinyl-N—(C₁-C₆ alkyl), or phenyl;
phenyl; or phenyl substituted with at least one of methyl, methoxyl, —SO₂Me, or halogen; and
R¹¹ is —H, -Me, Et, -nPr, or -iPr;
optionally wherein X is C—H;
provided that:
when R¹² is phenyl, then R⁶ is not 4-nitro, 4-chloro, 4-methoxyl, 4-CF₃, 4-bromo, 4-isopropyl, 2-chloro, 4-chloro, 4-hydroxyl, 3-nitro, or 4-hydroxyl.

In some aspects in formula (I):
R¹² is phenyl, imidazole, pyridine, or dihydropyrimidine, each of which is substituted with R⁴, R⁵, and R⁶;
R¹ and R⁴ are H;
R² and R¹⁰ independently are —H, —OH, —CN, -haloalkyl, —O—(C₁-C₆ alkyl), halogen, —C₁-C₆ alkyl, or phenyl;
R³ is —COOH or —COO—(C₁-C₆ alkyl);
R⁵ and R⁶ independently are 4-methyl, 4-fluoro, 3-fluoro, 3-chloro, 3-methyl, 3-methoxy, 4-cyano, 4-(CH₂OH), 4-(SO₂Me), 3-nitro, 3-hydroxyl, 4-(CO₂Me), 3-cyano, 4-(NHSO₂Me), 4-(4-methylphenyl), 4-(4-methoxyphenyl), 4-(4-(SO₂Me)phenyl), 4-(SBn), 4-(NHCH₂(4-fluorophenyl)), 4-(SO₂NH₂), 4-(4-fluorophenyl), 4-(SO₂Bn), 3-(CO₂Me), 4-(SO₂Et), 4-(SO₂NMe₂), 4-(OSO₂Me), 4-(C≡C-phenyl), 4-(C≡CCH₂OCH₂CH₂OCH₃), 4-(NHSO₂-phenyl), 4-(NBnSO₂Me), or 4-(NMeSO₂Me); and
R¹¹ is H or —C¹-C⁶ alkyl;
optionally wherein X is C—H.

In some aspects in formula (I):
R¹² is phenyl, imidazole, pyridine, or dihydropyrimidine, each of which is substituted with R⁴, R⁵, and R⁶;
R¹ and R⁴ are H;
R² and R¹⁰ independently are —H, —OH, —CN, -haloalkyl, —O—(C₁-C₆ alkyl), halogen, —C₁-C₆ alkyl, or phenyl;
R³ is —COOH, —COOMe, —COOEt, —COO(n-propyl), —COO(i-propyl), —COO(n-butyl), —COO(i-butyl), —COO(s-butyl), —COO(t-butyl), or tetrazole (e.g., the tetrazole structure defined elsewhere herein);
R⁵ and R⁶ independently are 4-methyl, 4-fluoro, 3-fluoro, 3-chloro, 3-methyl, 3-methoxy, 4-cyano, 4-(CH₂OH), 4-(SO₂Me), 3-nitro, 3-hydroxyl, 4-(CO₂Me), 3-cyano, 4-(NHSO₂Me), 4-(4-methylphenyl), 4-(4-methoxyphenyl), 4-(4-(SO₂Me)phenyl), 4-(SBn), 4-(NHCH₂(4-fluorophenyl)), 4-(SO₂NH₂), 4-(4-fluorophenyl), 4-(SO₂Bn), 3-(CO₂Me), 4-(SO₂Et), 4-(SO₂NMe₂), 4-(OSO₂Me), 4-(C≡C-phenyl), 4-(C≡CCH₂OCH₂CH₂OCH₃), 4-(NHSO₂-phenyl), 4-(NBnSO₂Me), or 4-(NMeSO₂Me); and
R¹¹ is H or —C¹-C⁶ alkyl;
optionally wherein X is C—H.

In some aspects in formula (I):
R¹² is phenyl, imidazole, pyridine, or dihydropyrimidine, each of which is substituted with R⁴, R⁵, and R⁶;
R¹ and R⁴ are H;
R² and R¹⁰ independently are —H, —OH, —CN, -haloalkyl, —O—(C₁-C₆ alkyl), halogen, —C₁-C₆ alkyl, or phenyl;
R³ is —COOH, —COOMe, —COOEt, —COO(n-propyl), —COO(i-propyl), —COO(n-butyl), —COO(i-butyl), —COO(s-butyl), —COO(t-butyl), or tetrazole (e.g., the tetrazole structure defined elsewhere herein);
R⁵ and R⁶ independently are 4-methyl, 4-fluoro, 3-fluoro, 3-chloro, 3-methyl, 3-methoxy, 4-cyano, 4-(CH₂OH), 4-(SO₂Me), 3-nitro, 3-hydroxyl, 4-(CO₂Me), 3-cyano, 4-(NHSO₂Me), 4-(4-methylphenyl), 4-(4-methoxyphenyl), 4-(4-(SO₂Me)phenyl), 4-(SBn), 4-(NHCH₂(4-fluorophenyl)), 4-(SO₂NH₂), 4-(4-fluorophenyl), 4-(SO₂Bn), 3-(CO₂Me), 4-(SO₂Et), 4-(SO₂NMe₂), 4-(OSO₂Me), 4-(C≡C-phenyl), 4-(C≡CCH₂OCH₂CH₂OCH₃), 4-(NHSO₂-phenyl), 4-(NBnSO₂Me), or 4-(NMeSO₂Me); and
R¹¹ is —H, -Me, Et, -nPr, or -iPr;
optionally wherein X is C—H.

Also disclosed is a compound of formula (II):

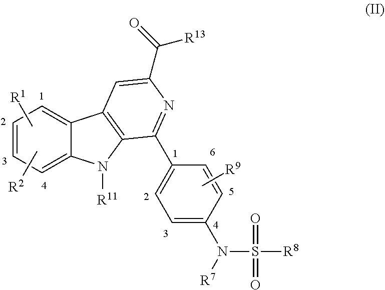

in which:
R¹ and R² independently are —H, —OH, —C₁-C₆ alkyl, —O—(C₁-C₆ alkyl), halogen, —CN, haloalkyl, or phenyl;
R¹¹ is —H or —C₁-C₆ alkyl;
R¹³ is —OH, —O—(C₁-C₆ alkyl), —NH₂, —NHSO₂—(C₁-C₆ alkyl), or —N(C₁-C₆ alkyl)SO₂(C₁-C₆ alkyl);
R⁷ is —H, —C₁-C₆ alkyl, or -Bn;
R⁸ is —C₁-C₆ alkyl, —N-piperazinyl-N—(C₁-C₆ alkyl), pyridinyl, or phenyl; and
R⁹ is —H, —OH, —C₁-C₆ alkyl, —O—(C₁-C₆ alkyl), haloalkyl, or a halogen;
wherein any alkyl phenyl, Bn, or pyridinyl moiety on an R¹, R², R⁷, R¹, R⁹, R¹¹, or R³ group can be further substituted with —C₁-C₆ alkyl, —O—(C₁-C₆ alkyl), —SO₂(C₁-C₆ alkyl), halogen, —CN, haloalkyl, —OH, pyridinyl, furanyl, tetrahydrofuranyl, tetrahydropyranyl, thiophenyl, thiazolyl, pyrrolyl, pyrrolidinyl, piperidinyl, morpholinyl, pyrimidinyl, triazolyl, imidazolyl, —NO₂, —N-piperazinyl-N—(C₁-C₆ alkyl), phenyl, -Bn, or any combination thereof, optionally wherein any alkyl phenyl, Bn, or pyridinyl moiety on an R¹, R², R⁷, R⁸, R⁹, R¹¹, or R¹³ group can be further substituted with —C₁-C₆ alkyl, —O—(C₁-C₆ alkyl), —SO₂(C₁-C₆ alkyl), halogen, —CN, haloalkyl, —OH, pyridinyl, furanyl, tetrahydrofuranyl, tetrahydropyranyl, thiophenyl, thiazolyl, pyrrolyl, pyrrolidinyl, piperidinyl, morpholinyl, pyrimidinyl, triazolyl, imidazolyl, —NO₂, —N-piperazinyl-N—(C₁-C₆ alkyl), phenyl, -Bn, or any combination thereof, or a salt or ester thereof.

In some aspects in formula (II), R¹ and R² independently are —H, —OH, —C₁-C₆ alkyl, —O—(C₁-C₆ alkyl), halogen, —CN, haloalkyl, or phenyl. The disclosures elsewhere herein in relation to R¹ and R² in the context of formula (I) are equally applicable here with respect to formula (II).

In some aspects in formula (II), R¹¹ is —H or —C₁-C₆ alkyl. The disclosures elsewhere herein in relation to R¹¹ in the context of formula (I) are equally applicable here with respect to formula (II).

In some aspects in formula (II), R¹³ is —OH, —O—(C₁-C₆ alkyl), —NH₂, —NHSO₂—(C₁-C₆ alkyl), or —N(C₁-C₆ alkyl)SO₂(C₁-C₆ alkyl). For example, in some aspects, R¹³ is —OH. In some aspects, R¹³ is —NH₂. In some aspects, R¹³ is —O—(C₁-C₆ alkyl) (e.g., —OMe, —OEt, and so forth, or any other C₁-C₆ alkyl as defined elsewhere herein). In some aspects, R¹³ is —NHSO₂—(C₁-C₆ alkyl) (e.g., —NHSO₂Me, or any other C₁-C₆ alkyl as defined elsewhere herein). In some aspects, R¹³ is —N(C₁-C₆ alkyl)SO₂(C₁-C₆ alkyl) (e.g., —NMeSO₂Me, or independently any other C₁-C₆ alkyl as defined elsewhere herein). In general, the R¹³ group in formula (II) corresponds to the R³ group in formula (I) in which R³ has the following structure: —COR¹³ (i.e., a carbonyl group connected to R¹³). Accordingly, any disclosure herein relating to an R³ group that fits the —COR¹³ structure is equally applicable with respect to the —COR¹³ group (e.g., since R³ in formula (I) is disclosed elsewhere herein to be —COMe in some aspects, and —COMe fits the —COR¹³ structure, solely in view of this structural fit R¹³ can also be -Me).

In some aspects in formula (II), R⁷ is —H, —C₁-C₆ alkyl, or -Bn. In some aspects, R⁷ is —H. In some aspects, R⁷ is —C₁-C₆ alkyl (e.g., -Me, -Et, or any other C₁-C₆ alkyl as defined elsewhere herein). In some aspects, R⁷ is -Bn.

In some aspects in formula (II), R⁸ is —C₁-C₆ alkyl, —N-piperazinyl-N—(C₁-C₆ alkyl), pyridinyl, or phenyl. In some aspects, R⁸ is —C₁-C₆ alkyl (e.g., -Me, -Et, or any other C₁-C₆ alkyl as defined elsewhere herein). In some aspects, R⁸ is —N-piperazinyl-N—(C₁-C₆ alkyl) (e.g., —N— piperazinyl-N-Me, —N-piperazinyl-N-Et, —N-piperazinyl-N-isopropyl, —N-piperazinyl-N-n-propyl, or any other C₁-C₆ alkyl as defined elsewhere herein). The —N-piperazinyl-N—(C₁-C₆ alkyl) structure is shown below and is applicable to any disclosure herein where this term is used:

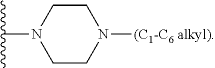

In some aspects in formula (II), R⁹ is —H, —OH, —C₁-C₆ alkyl, —O—(C₁-C₆ alkyl), haloalkyl, or a halogen. In some aspects, R⁹ is H. In some aspects, R⁹ is —OH. In some aspects, R⁹ is —C₁-C₆ alkyl (e.g., -Me, -Et, -isopropyl, -n-propyl, or any other C₁-C₆ alkyl as defined elsewhere herein). In some aspects, R⁹ is —O—(C₁-C₆ alkyl) (e.g., —OMe, —OEt, —O-isopropyl, —O-n-propyl, —O-t-butyl, or any other C₁-C₆ alkyl as defined elsewhere herein). In some aspects, R⁹ is haloalkyl (e.g., —CF₃, —CH₂F, —CHF₂, —CCl₃, or any other haloalkyl as defined elsewhere herein). In some aspects, R⁹ is halogen (e.g., —F, —Cl, —Br, or —I).

In some aspects in formula (II), any alkyl, phenyl, Bn, or pyridinyl moiety on an R¹, R², R⁷, R¹, R⁹, R¹¹, or R³ group in formula (II) can be further substituted with —C₁-C₆ alkyl, —O—(C₁-C₆ alkyl), —SO₂(C₁-C₆ alkyl), halogen, —CN, haloalkyl, —OH, pyridinyl, furanyl, tetrahydrofuranyl, tetrahydropyranyl, thiophenyl, thiazolyl, pyrrolyl, pyrrolidinyl, piperidinyl, morpholinyl, pyrimidinyl, triazolyl, imidazolyl, —NO₂, —N-piperazinyl-N—(C₁-C₆ alkyl), phenyl, -Bn, or any combination thereof, optionally wherein any alkyl phenyl, Bn, or pyridinyl moiety on an R¹, R², R⁷, R⁸, R⁹, R¹¹, or R³ group can be further substituted with —C₁-C₆ alkyl, —O—(C₁-C₆ alkyl), —SO₂(C₁-C₆ alkyl), halogen, —CN, haloalkyl, —OH, pyridinyl, furanyl, tetrahydrofuranyl, tetrahydropyranyl, thiophenyl, thiazolyl, pyrrolyl, pyrrolidinyl, piperidinyl, morpholinyl, pyrimidinyl, triazolyl, imidazolyl, —NO₂, —N-piperazinyl-N—(C₁-C₆ alkyl), phenyl, -Bn, or any combination thereof. For example, in some aspects, R⁸ is phenyl, and the R⁸ phenyl is further substituted with C₁-C₆ alkyl (e.g., -Me, -Et), —O—(C₁-C₆ alkyl) (e.g., —OMe), halogen, —CN, —N— piperazinyl-N—(C₁-C₆ alkyl) or pyridinyl, in which the substitution can be located in any position (e.g., in the 2-position, 3-position, or 4-position, in which position 1 on the phenyl is attached to the sulfone sulfur atom; in some aspects, the substitution is at the 4-position).

In some aspects in formula (II):
R¹ and R² independently are —H, —OH, methyl, methoxyl, fluoro, chloro, bromo, phenyl, —CF₃, —CH₂F, —CHF₂, —CCl₃, or —CN;
R¹³ is —OH, —NH₂, —O—(C₁-C₆ alkyl) (e.g., —OMe, —OEt, OtBu, and so forth), or —NHSO₂Me;
R⁷ is —H, methyl, or -Bn;
R⁸ is methyl, N-piperazinyl-N-methyl, pyridinyl, phenyl, or phenyl substituted with at least one of methyl, methoxyl, pyridinyl, fluoro, chloro, bromo, or N-piperazinyl-N-methyl; and
R⁹ is H, methyl, fluoro, chloro, bromo, haloalkyl, —O—(C₁-C₆ alkyl), or —OH, optionally in which R¹³ is —OH, methoxyl, ethoxyl, isopropoxyl, n-propoxyl, isobutoxyl, sec-butoxyl, n-butoxyl, or t-butoxyl.

In some aspects in formula (II):
R¹ is —H, fluoro, chloro, bromo, cyano, phenyl, methoxyl, hydroxyl, or methyl;
R² is fluoro, chloro, bromo, cyano, phenyl, methoxyl, hydroxyl, or methyl;
R¹³ is —OH or —O—(C₁-C₆ alkyl) (e.g., —OMe, —OEt, OtBu, and so forth);
R⁷ is —H, methyl, or -Bn;
R⁸ is methyl, N-piperazinyl-N-methyl, pyridinyl, phenyl, or phenyl substituted with at least one of methyl, methoxyl, pyridinyl, fluoro, chloro, bromo, or N-piperazinyl-N-methyl; and
R⁹ is —H, fluoro, or methyl, optionally in which R¹³ is —OH, methoxyl, ethoxyl, isopropoxyl, n-propoxyl, isobutoxyl, sec-butoxyl, n-butoxyl, or t-butoxyl.

In some aspects in formula (II):
R¹ is —H;
R² is fluoro or cyano;
R³ is —OH or —O—(C₁-C₆ alkyl) (e.g., —OMe, —OEt, OtBu, and so forth);
R⁷ is —H or methyl;
R⁸ is methyl, N-piperazinyl-N-methyl, pyridinyl, phenyl, or phenyl substituted with at least one of methyl, methoxyl, pyridinyl, fluoro, chloro, bromo, or N-piperazinyl-N-methyl;
R⁹ is —H,
optionally in which R¹³ is —OH, methoxyl, ethoxyl, isopropoxyl, n-propoxyl, isobutoxyl, sec-butoxyl, n-butoxyl, or t-butoxyl.

In some aspects in formula (II):
R¹ is —H;
R² is fluoro, chloro, bromo, cyano, or methyl;
R³ is —OH or —O—(C₁-C₆ alkyl) (e.g., —OMe, —OEt, OtBu, and so forth);
R⁷ is —H or methyl;
R⁸ is methyl, N-piperazinyl-N-methyl, pyridinyl, phenyl, or phenyl substituted with at least one of methyl, methoxyl, pyridinyl, fluoro, chloro, bromo, or N-piperazinyl-N-methyl;
R⁹ is —H or fluoro;
optionally in which R¹³ is —OH, methoxyl, ethoxyl, isopropoxyl, n-propoxyl, isobutoxyl, sec-butoxyl, n-butoxyl, or t-butoxyl.

In some aspects in formula (II):
R¹ is —H;
R² is fluoro, chloro, bromo, cyano, methyl, or —CF₃;
R³ is —OH or —O—(C₁-C₆ alkyl) (e.g., —OMe, —OEt, OtBu, and so forth);
R⁷ is —H or methyl;
R⁸ is methyl, N-piperazinyl-N-methyl, pyridinyl, phenyl, or phenyl substituted with at least one of methyl, methoxyl, pyridinyl, fluoro, chloro, bromo, or N-piperazinyl-N-methyl;
R⁹ is —H, fluoro, or —CF₃;
optionally in which R¹³ is —OH, methoxyl, ethoxyl, isopropoxyl, n-propoxyl, isobutoxyl, sec-butoxyl, n-butoxyl, or t-butoxyl.

In some aspects or embodiments, the compound of formula (I) and/or formula (II) is:

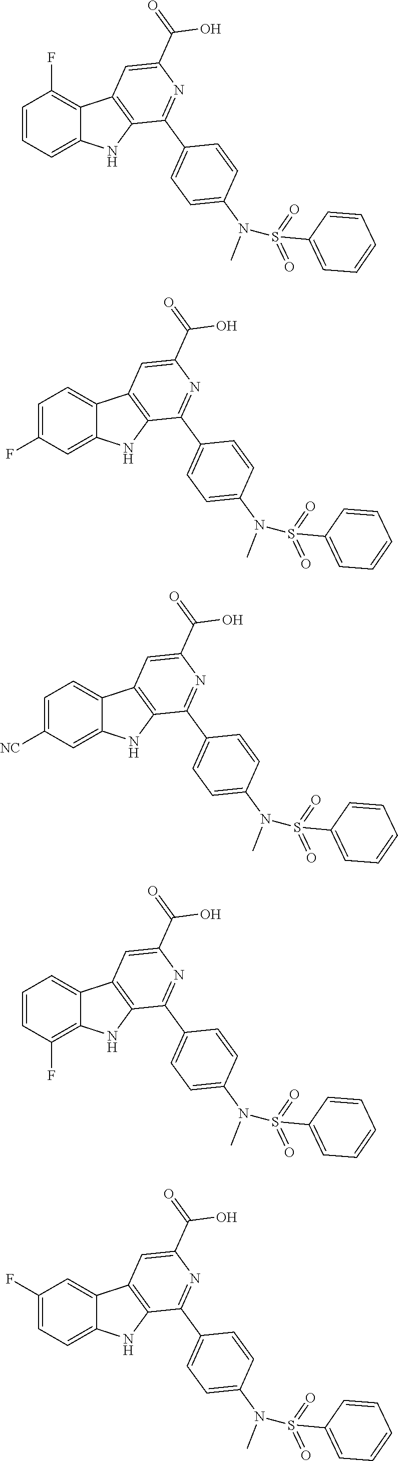 [SMILES: CN(c1ccc(-c2nc(C(=O)O)cc3c2[nH]c2cccc(F)c23)cc1)S(=O)(=O)c1ccccc1 ; CN(c1ccc(-c2nc(C(=O)O)cc3c2[nH]c2cc(F)ccc23)cc1)S(=O)(=O)c1ccccc1 ; CN(c1ccc(-c2nc(C(=O)O)cc3c2[nH]c2cc(C#N)ccc23)cc1)S(=O)(=O)c1ccccc1 ; CN(c1ccc(-c2nc(C(=O)O)cc3c2[nH]c2c(F)cccc23)cc1)S(=O)(=O)c1ccccc1 ; CN(c1ccc(-c2nc(C(=O)O)cc3c2[nH]c2ccc(F)cc23)cc1)S(=O)(=O)c1ccccc1]

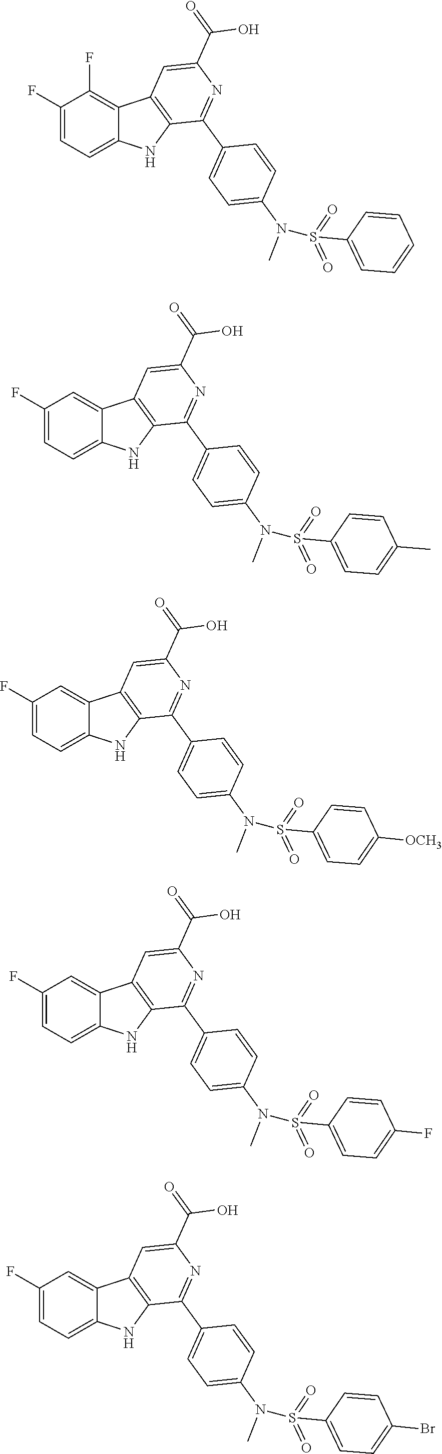 [SMILES: CN(c1ccc(-c2nc(C(=O)O)cc3c2[nH]c2ccc(F)c(F)c23)cc1)S(=O)(=O)c1ccccc1 ; Cc1ccc(S(=O)(=O)N(C)c2ccc(-c3nc(C(=O)O)cc4c3[nH]c3ccc(F)cc34)cc2)cc1 ; COc1ccc(S(=O)(=O)N(C)c2ccc(-c3nc(C(=O)O)cc4c3[nH]c3ccc(F)cc34)cc2)cc1 ; CN(c1ccc(-c2nc(C(=O)O)cc3c2[nH]c2ccc(F)cc23)cc1)S(=O)(=O)c1ccc(F)cc1 ; CN(c1ccc(-c2nc(C(=O)O)cc3c2[nH]c2ccc(F)cc23)cc1)S(=O)(=O)c1ccc(Br)cc1]

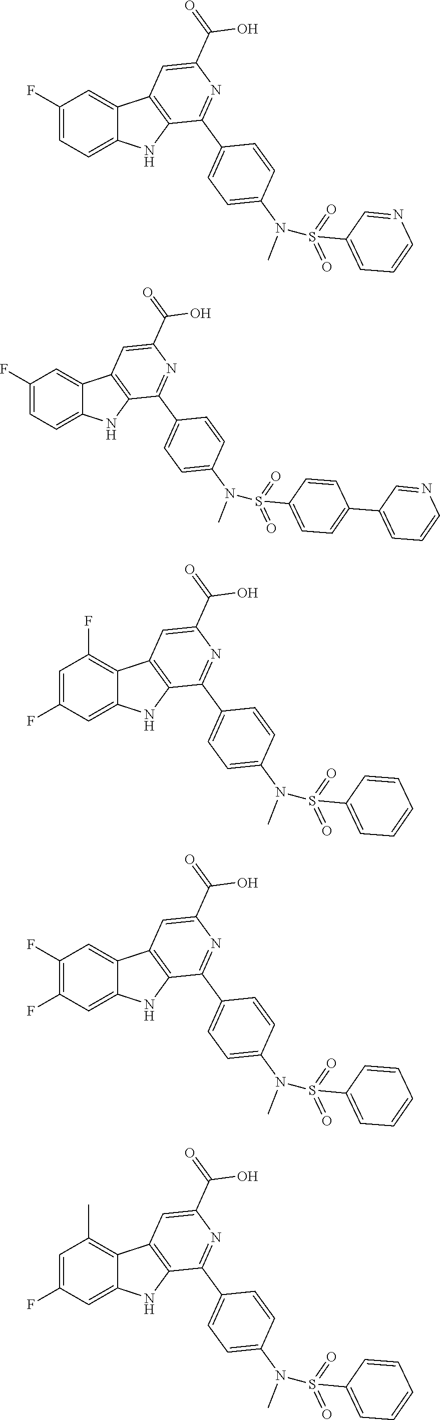 [SMILES: CN(c1ccc(-c2nc(C(=O)O)cc3c2[nH]c2ccc(F)cc23)cc1)S(=O)(=O)c1cccnc1 ; CN(c1ccc(-c2nc(C(=O)O)cc3c2[nH]c2ccc(F)cc23)cc1)S(=O)(=O)c1ccc(-c2cccnc2)cc1 ; CN(c1ccc(-c2nc(C(=O)O)cc3c2[nH]c2cc(F)cc(F)c23)cc1)S(=O)(=O)c1ccccc1 ; CN(c1ccc(-c2nc(C(=O)O)cc3c2[nH]c2cc(F)c(F)cc23)cc1)S(=O)(=O)c1ccccc1 ; Cc1cc(F)cc2[nH]c3c(-c4ccc(N(C)S(=O)(=O)c5ccccc5)cc4)nc(C(=O)O)cc3c12]

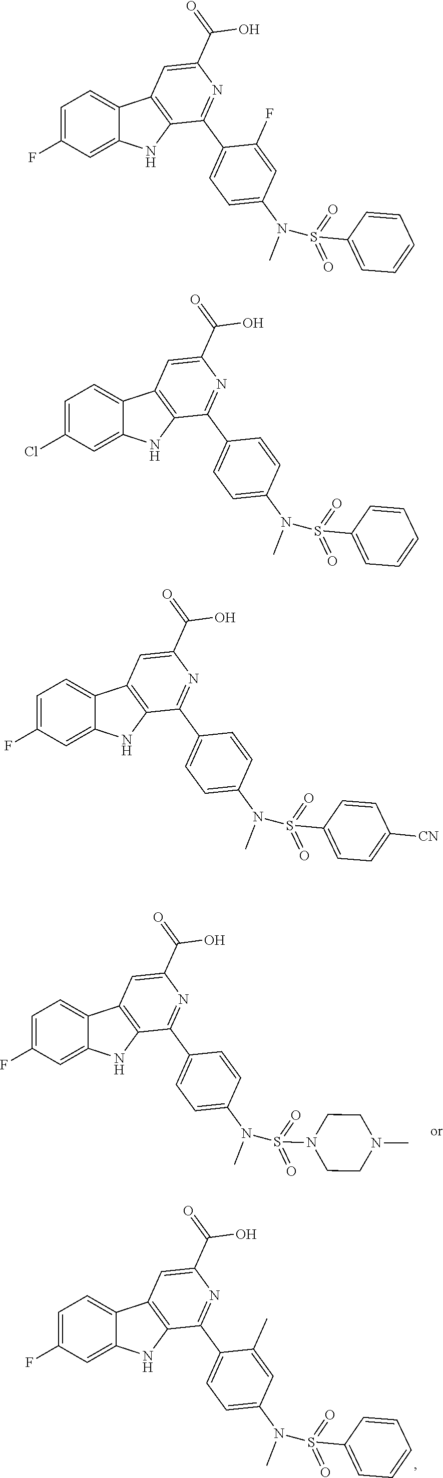 [SMILES: CN(c1ccc(-c2nc(C(=O)O)cc3c2[nH]c2cc(F)ccc23)c(F)c1)S(=O)(=O)c1ccccc1 ; CN(c1ccc(-c2nc(C(=O)O)cc3c2[nH]c2cc(Cl)ccc23)cc1)S(=O)(=O)c1ccccc1 ; CN(c1ccc(-c2nc(C(=O)O)cc3c2[nH]c2cc(F)ccc23)cc1)S(=O)(=O)c1ccc(C#N)cc1 ; CN1CCN(S(=O)(=O)N(C)c2ccc(-c3nc(C(=O)O)cc4c3[nH]c3cc(F)ccc34)cc2)CC1 ; Cc1cc(N(C)S(=O)(=O)c2ccccc2)ccc1-c1nc(C(=O)O)cc2c1[nH]c1cc(F)ccc12]

or a salt or ester thereof. In some aspects, the compound of formula (I) and/or formula (II) is:

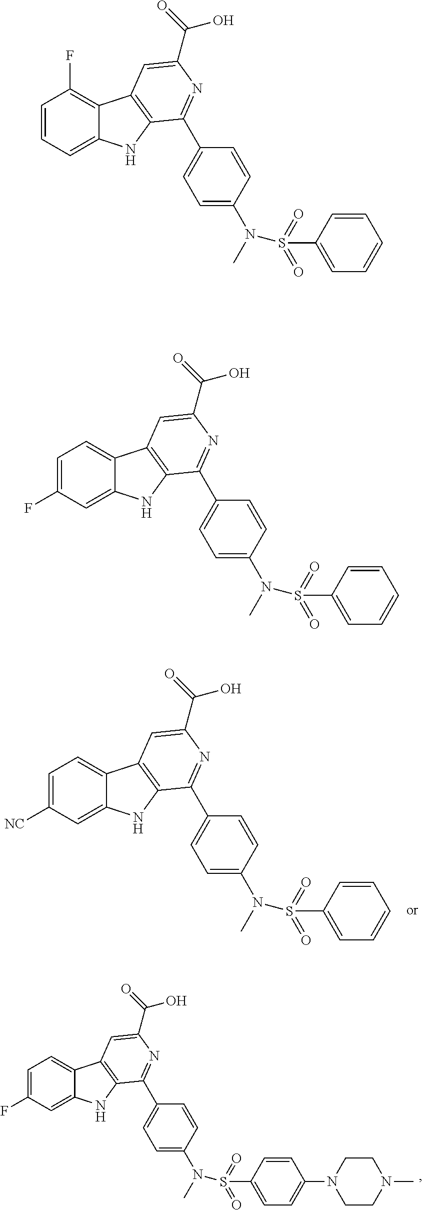 [SMILES: CN(c1ccc(-c2nc(C(=O)O)cc3c2[nH]c2cccc(F)c23)cc1)S(=O)(=O)c1ccccc1 ; CN(c1ccc(-c2nc(C(=O)O)cc3c2[nH]c2cc(F)ccc23)cc1)S(=O)(=O)c1ccccc1 ; CN(c1ccc(-c2nc(C(=O)O)cc3c2[nH]c2cc(C#N)ccc23)cc1)S(=O)(=O)c1ccccc1 ; CN1CCN(c2ccc(S(=O)(=O)N(C)c3ccc(-c4nc(C(=O)O)cc5c4[nH]c4cc(F)ccc45)cc3)cc2)CC1]

or a salt or ester thereof (e.g., a methyl ester thereof). Examples of such esters include, for instance:

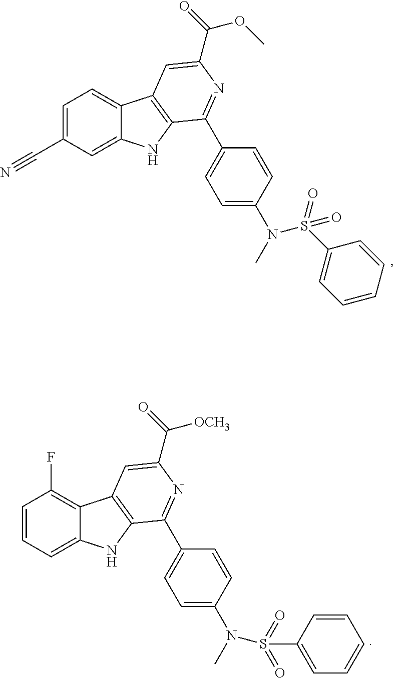 [SMILES: COC(=O)c1cc2c([nH]c3cc(C#N)ccc32)c(-c2ccc(N(C)S(=O)(=O)c3ccccc3)cc2)n1 ; COC(=O)c1cc2c([nH]c3cccc(F)c32)c(-c2ccc(N(C)S(=O)(=O)c3ccccc3)cc2)n1]

Also disclosed is a compound of formula (III):

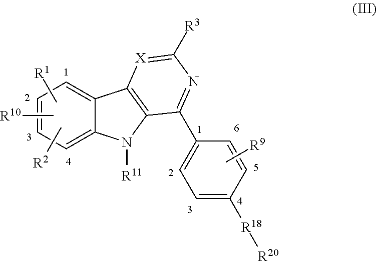

wherein:
X is C—H or N,
R¹, R², and R¹⁰ independently are —H, —OH, —CN, haloalkyl, —O—(C₁-C₆ alkyl), halogen, —C₁-C₆ alkyl, or phenyl;
R³ is —COOH, —COO—(C₁-C₆ alkyl), —CONH₂, —CON(C₁-C₆ alkyl)₂, —CONH(C₁-C₆ alkyl), —CONHSO₂(C₁-C₆ alkyl), —CON(C₁-C₆ alkyl)SO₂(C₁-C₆ alkyl), halogen, —SO₂NH₂, —NHSO₂(C₁-C₆ alkyl), —CN, or tetrazole;
R¹¹ is —H or C₁-C₆ alkyl;
R¹⁸ is —O—, —S—, —NR¹⁹—, —(C₁-C₆ alkyl)-, —(O—(C₁-C₆ alkyl))-, —(O—(C₁-C₆ alkyl)-O)—, —(OCH₂CH₂)ₙ—O—, —(NR¹⁹—(C₁-C₆ alkyl))-, —(NR¹⁹—(C₁-C₆ alkyl)-O)—, —(NR¹⁹—(C₁-C₆ alkyl)-NR¹⁹)—, —(OCH₂CH₂)ₙ—NR¹⁹—, —(S—(C₁-C₆ alkyl))-, —(S—(C₁-C₆ alkyl)-S)—, —(OCH₂CH₂)ₙ—S—, —(NR¹⁹—(C₁-C₆ alkyl)-S)—, —(S—(C₁-C₆ alkyl)-O)—, or —(CONR¹⁹)ₙ—;
wherein each n independently is an integer from 1 to 6;
wherein each R¹⁹ independently is H or C₁-C₆ alkyl; and
R⁹ is —H, —OH, —C₁-C₆ alkyl, —O—(C₁-C₆ alkyl), phenyl, haloalkyl, or halogen;
R²⁰ is C₃-C₆ cycloalkyl, phenyl, a 5-membered heterocycle, or a 6-membered heterocycle, each of which is substituted with R¹⁴, R¹⁵, and R¹⁶ on a carbon atom and/or heteroatom,
wherein:
   R¹⁴, R¹⁵, and R¹⁶ independently are —H, —C₁-C₆ alkyl, halogen, —OH, —O(C₁-C₆ alkyl), —CN, —NO₂, —(C₁-C₆ alkyl)-OH, SO₂(C₁-C₆ alkyl), haloalkyl, —CO₂(C₁-C₆ alkyl), —NHSO₂(C₁-C₆ alkyl), —NHBn, phenyl, —SBn, —OBn, —SO₂NH₂, —SO₂(C₁-C₆ alkyl), —SO₂Bn, SO₂N(C₁-C₆ alkyl)₂, —OSO₂(C₁-C₆ alkyl), —C≡C-phenyl, —C≡CCH₂(OCH₂CH₂)₁₋₄OH, —C≡CCH₂(OCH₂CH₂)₁₋₄OMe, —NHSO₂-pyridinyl, —NBnSO₂-pyridinyl, —NHSO₂-phenyl, —NHSO₂(N-piperazinyl-N—(C₁-C₆ alkyl)), —N(C₁-C₆ alkyl)SO₂-pyridinyl, —N(C₁-C₆ alkyl)SO₂-phenyl, —N(C₁-C₆ alkyl)SO₂(N-piperazinyl-N—(C₁-C₆ alkyl)), —NBnSO₂(C₁-C₆ alkyl), —NBnSO₂(N-piperazinyl-N—(C₁-C₆ alkyl)), —NBnSO₂-phenyl, or —N(C₁-C₆ alkyl)SO₂(C₁-C₆ alkyl); or
   R¹⁴ and R¹⁵ taken together form a fused pyrazole ring, a fused pyrrole ring, or a fused phenyl ring, and R¹⁶ is as defined above; and
wherein any -Bn, pyridinyl, alkyl, tetrazole, fused pyrazole ring, fused pyrrole ring, fused phenyl ring, or phenyl moiety of the foregoing groups can be substituted with C₁-C₆ alkyl, —O—(C₁-C₆ alkyl), —SO₂(C₁-C₆ alkyl), halogen, —CN, haloalkyl, —OH, pyridinyl, furanyl, tetrahydrofuranyl, tetrahydropyranyl, thiophenyl, thiazolyl, pyrrolyl, pyrrolidinyl, piperidinyl, morpholinyl, pyrimidinyl, triazolyl, imidazolyl, nitro, —N-piperazinyl-N—(C₁-C₆ alkyl), phenyl, Bn, or any combination thereof;
or a salt or ester thereof.

In some embodiments, the compound of formula (III) is of formula (IIIa):

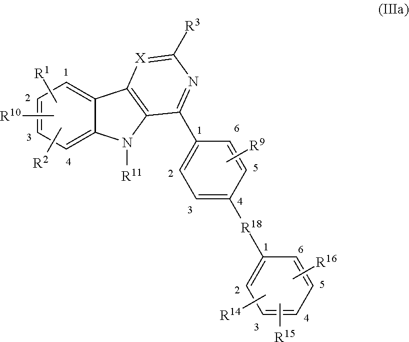

wherein:
X is C—H or N,
R¹, R², and R¹⁰ independently are —H, —OH, —CN, haloalkyl, —O—(C₁-C₆ alkyl), halogen, —C₁-C₆ alkyl, or phenyl;
R³ is —COOH, —COO—(C₁-C₆ alkyl), —CONH₂, —CON(C₁-C₆ alkyl)₂, —CONH(C₁-C₆ alkyl), —CONHSO₂(C₁-C₆ alkyl), —CON(C₁-C₆ alkyl)SO₂(C₁-C₆ alkyl), halogen, —SO₂NH₄₂, —N—SO₂(C₁-C₆ alkyl), —CN, or tetrazole;
R¹¹ is —H or C₁-C₆ alkyl;
R¹⁸ is —O—, —S—, —NR¹⁹—, —(C₁-C₆ alkyl)-, —(O—(C₁-C₆ alkyl))-, —(O—(C₁-C₆ alkyl)-O)—, —(OCH₂CH₂)ₙ—O—, —(NR¹⁹—(C₁-C₆ alkyl))-, —(NR¹⁹—(C₁-C₆ alkyl)-O)—, —(NR¹⁹—(C₁-C₆ alkyl)-NR¹⁹)—, —(OCH₂CH₂)ₙ—NR¹⁹—, —(S—(C₁-C₆ alkyl))-, —(S—(C₁-C₆ alkyl)-S)—, —(OCH₂CH₂)ₙ—S—, —(NR¹⁹—(C₁-C₆ alkyl)-S)—, —(S—(C₁-C₆ alkyl)-O)—, or —(CONR¹⁹)ₙ—;
wherein each n independently is an integer from 1 to 6;
wherein each R¹⁹ independently is H or C₁-C₆ alkyl; and
R⁹ is —H, —OH, —C₁-C₆ alkyl, —O—(C₁-C₆ alkyl), phenyl, haloalkyl, or halogen;
wherein:
   R¹⁴, R¹⁵, and R¹⁶ independently are —H, —C₁-C₆ alkyl, halogen, —OH, —O(C₁-C₆ alkyl), —CN, —NO₂, —(C₁-C₆ alkyl)-OH, SO₂(C₁-C₆ alkyl), haloalkyl, —CO₂(C₁-C₆ alkyl), —NHSO₂(C₁-C₆ alkyl), —NHBn, phenyl, —SBn, —OBn, —SO₂NH₂, —SO₂(C₁-C₆ alkyl), —SO₂Bn, SO₂N(C₁-C₆ alkyl)₂, —OSO₂(C₁-C₆ alkyl), —C≡C-phenyl, —C≡CCH₂(OCH₂CH₂)₁₋₄OH, —C≡CCH₂(OCH₂CH₂)₁₋₄OMe, —NHSO₂-pyridinyl, —NBnSO₂-pyridinyl, —NHSO₂-phenyl, —NHSO₂(N-piperazinyl-N—(C₁-C₆ alkyl)), —N(C₁-C₆ alkyl)SO₂-pyridinyl, —N(C₁-C₆ alkyl)SO₂-phenyl, —N(C₁-C₆ alkyl)SO₂(N-piperazinyl-N—(C₁-C₆ alkyl)), —NBnSO₂(C₁-C₆ alkyl), —NBnSO₂(N-piperazinyl-N—(C₁-C₆ alkyl)), —NBnSO₂-phenyl, or —N(C₁-C₆ alkyl)SO₂(C₁-C₆ alkyl); or
   R¹⁴ and R¹⁵ taken together form a fused pyrazole ring, a fused pyrrole ring, or a fused phenyl ring, and R¹⁶ is as defined above; and
wherein any -Bn, pyridinyl, alkyl, tetrazole, fused pyrazole ring, fused pyrrole ring, fused phenyl ring, or phenyl moiety of the foregoing groups can be substituted with C₁-C₆ alkyl, —O—(C₁-C₆ alkyl), —SO₂(C₁-C₆ alkyl), halogen, —CN, haloalkyl, —OH, pyridinyl, furanyl, tetrahydrofuranyl, tetrahydropyranyl, thiophenyl, thiazolyl, pyrrolyl, pyrrolidinyl, piperidinyl, morpholinyl, pyrimidinyl, triazolyl, imidazolyl, nitro, —N-piperazinyl-N—(C₁-C₆ alkyl), phenyl, Bn, or any combination thereof; optionally wherein any -Bn, pyridinyl, alkyl, tetrazole, fused pyrazole ring, fused pyrrole ring, fused phenyl ring, or phenyl moiety of the foregoing groups can be substituted with C₁-C₆ alkyl, —O—(C₁-C₆ alkyl), —SO₂(C₁-C₆ alkyl), halogen, —CN, haloalkyl, —OH, pyridinyl, furanyl, tetrahydrofuranyl, tetrahydropyranyl, thiophenyl, thiazolyl, pyrrolyl, pyrrolidinyl, piperidinyl, morpholinyl, pyrimidinyl, triazolyl, imidazolyl, nitro, —N— piperazinyl-N—(C₁-C₆ alkyl), phenyl, Bn, or any combination thereof;
or a salt or ester thereof.

In some aspects, the compound of formula (III) is:

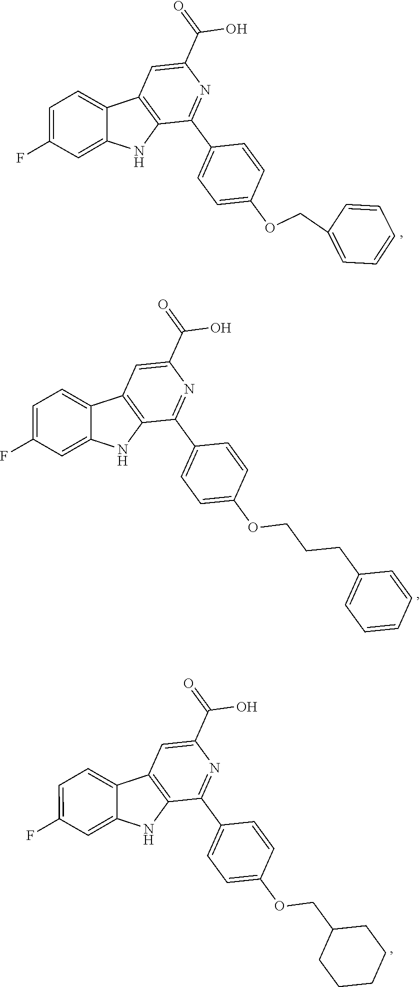 [SMILES: O=C(O)c1cc2c([nH]c3cc(F)ccc32)c(-c2ccc(OCc3ccccc3)cc2)n1 ; O=C(O)c1cc2c([nH]c3cc(F)ccc32)c(-c2ccc(OCCCc3ccccc3)cc2)n1 ; O=C(O)c1cc2c([nH]c3cc(F)ccc32)c(-c2ccc(OCC3CCCCC3)cc2)n1]

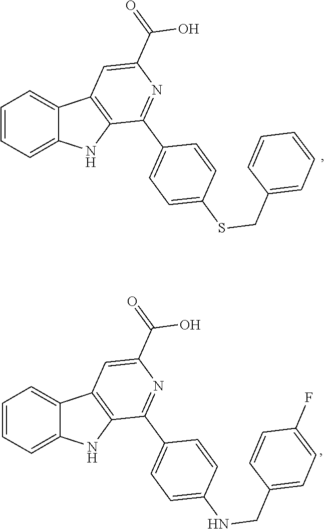 [SMILES: O=C(O)c1cc2c([nH]c3ccccc32)c(-c2ccc(SCc3ccccc3)cc2)n1 ; O=C(O)c1cc2c([nH]c3ccccc32)c(-c2ccc(NCc3ccc(F)cc3)cc2)n1]

or a salt or ester thereof.

Additional examples of compounds of formula (I), formula (II), or formula (III), are set forth in Table 1 (see Example 5), which are contemplated as embodiments of the disclosure along with any salt or ester thereof.

Salts and esters of the foregoing compounds of formula (I), formula (II), and/or formula (III) (including specific embodiments set forth above or in the examples) can be any salt or ester known in the art, such as a pharmaceutically acceptable salt or ester. The salt can be, for example, acid addition salts, such as those formed with inorganic acids such as hydrochloric acid, hydrobromic acid, sulfuric acid, nitric acid, phosphoric acid, and the like; or those formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like. In some aspects, the salt can be formed when an acidic proton present in a compound of formula (I), (II), and/or (III) either is replaced by a metal ion, e.g., an alkali metal ion, an alkaline earth ion, or an aluminum ion; or coordinates with an organic base such as ethanolamine, diethanolamine, triethanolamine, N-methylglucamine and the like. In some aspects, salts further include, for example, sodium, potassium, calcium, magnesium, ammonium, tetraalkylammonium, and the like; and when the compound of formula (I), (II), and/or (III) contains a basic functionality, salts of non toxic organic or inorganic acids, such as hydrochloride, hydrobromide, tartrate, mesylate, acetate, maleate, oxalate and the like, also are contemplated. The ester can be, for instance, a compound of formula (I), (II), or (III) (including specific embodiments set forth above or in the examples) in which a —COOH group is replaced with —COO—(C₁-C₆ alkyl), and/or wherein an N—H group is replaced with N—(C₁-C₆ alkyl). For example, in some embodiments, the —COOH group is replaced with —COOMe, —COOEt, —COO(iPr), —COO(nPr), —COO(tBu), —COO(sBu), —COO(nBu), —COO(n-pentyl), —COO(isopentyl), —COO(2-pentyl), —COO(n-hexyl), —COO(2-hexyl), —COO(3-hexyl), and so forth. Alternatively or additionally, an N—H group can be replaced with NMe, NEt, N(nPr), N(iPr), N(tBu), N(sBu), N(nBu), N (n-pentyl), N(isopentyl), N(2-pentyl), N(n-hexyl), N(2-hexyl), N(3-hexyl), or the like. For instance, for any compound set forth in Table 1, the position corresponding to R¹¹ of formula (I), formula (II), or formula (III) (as applicable) can be —H or —C₁-C₆ alkyl. The “ester thereof” can be —COOMe, —COOEt, —COO(iPr), —COO(nPr), —COO(tBu), —COO(sBu), —COO(nBu), —COO(n-pentyl), —COO(isopentyl), —COO(2-pentyl), —COO(n-hexyl), —COO(2-hexyl), —COO(3-hexyl), and so forth, or any other ester as described herein, including those that can be formed for a C₁-C₆ alkyl as defined herein.

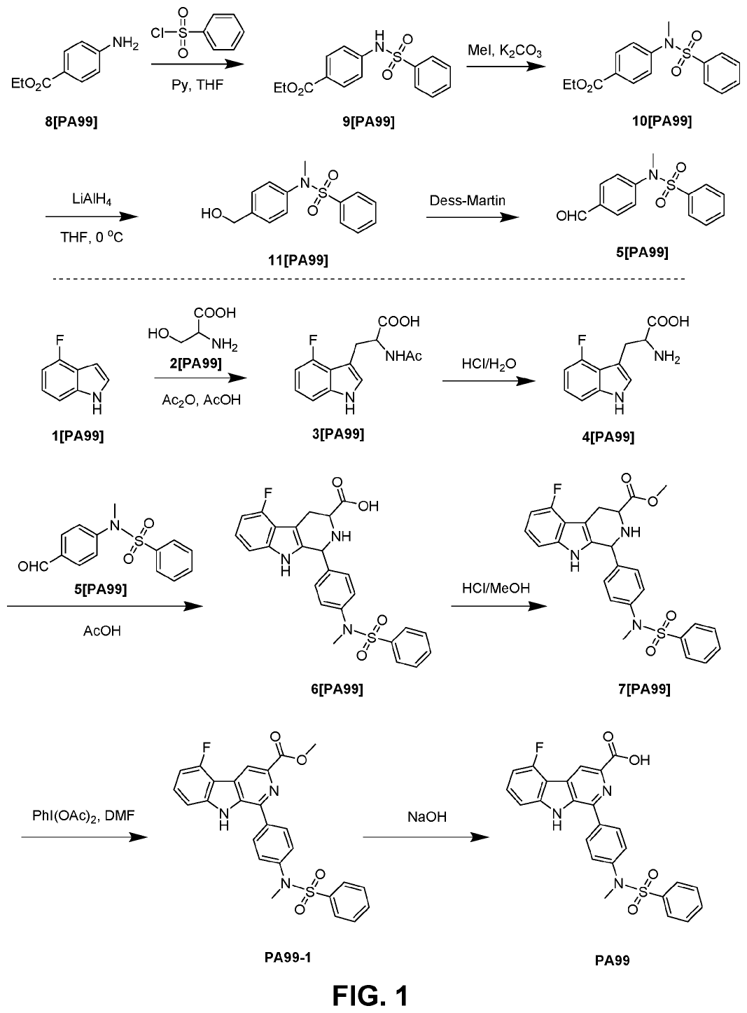

The compounds of formula (I), formula (II), formula (III), or salts or esters thereof, as provided herein including any embodiments thereof, can be made by any suitable method as illustrated in the Examples, which methods are considered part of the invention along with any intermediates described therein. Accordingly, the present disclosure also provides a method of making a compound of formula (I), (II), or (II), or a salt or ester thereof, as set forth in any one of FIG. 1-4 or 11-26B, or as otherwise described in the Examples. The present disclosure also provides an intermediate compound useful in the preparation of a compound of formula (I), (II), or (II), or a salt or ester thereof, which intermediate compound has a structure as disclosed in any one of FIG. 1-4 or 11-26B, or as otherwise described in the Examples.

Without wishing to be bound by any particular theory or mechanism of action, it is believed the compounds of formula (I), formula (II), formula (III), or any combination thereof, including those shown in Table 1 (see Example 5), are CRL4 inhibitors that specifically target the DDB1 adaptor, thereby disrupting the interaction between CUL4A and/or CUL4B and the beta-propeller B of DDB1. Thus, the compounds of formula (I), formula (II), and formula (III) are believed to be useful for treating or preventing a disease or condition that is responsive to such activity. In some aspects, the disease is cancer, particularly cancer associated with or characterized by CRL4A ubiquitin ligase expression or activity, CUL4A or CUL4B expression or activity, and/or DDB1 expression or activity. Non-limiting examples of cancers include cancers and tumors characterized by a high level of CUL4 expression, including breast cancer, colorectal cancer, lung cancer, and brain cancer, or any other cancer as disclosed herein. In some aspects, the cancer is resistant to one or more topoisomerase I-directed chemotherapy drugs (e.g., camptothecin, irinotecan, and topotecan). Accordingly, also provided is a method of using the compound or composition for the treatment of a disease or condition (e.g., in a mammal or human) associated with CRL4A ubiquitin ligase expression or activity, CUL4A or CUL4B expression or activity, and/or DDB1 expression or activity, which method is described more specifically elsewhere herein.

In some aspects, disclosed herein is a composition or formulation (e.g., a pharmaceutical composition) comprising (a) a compound of formula (I):

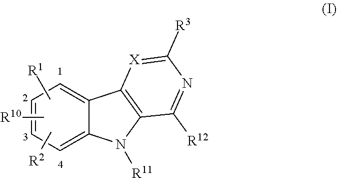

and/or a compound of formula (II):

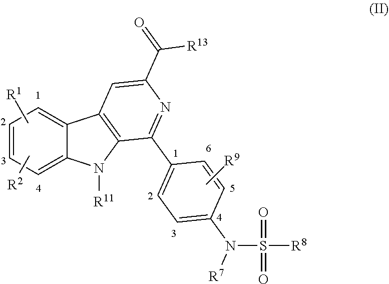

and/or a compound of formula (III):

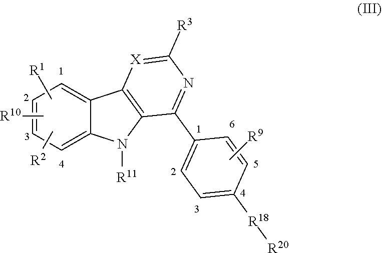

or any salt or ester thereof (including any of the foregoing embodiments provided herein) and (b) a carrier (e.g., a pharmaceutically acceptable carrier), wherein all aspects and embodiments of formula (I), formula (II), and formula (III) are as set forth herein with respect to the description of the compounds themselves.

Any suitable carrier can be used. The carrier typically will be liquid, but also can be solid, or a combination of liquid and solid components. The carrier desirably is physiologically acceptable (e.g., a pharmaceutically, pharmacologically, or cosmetically acceptable) carrier (e.g., excipient or diluent). Any suitable physiologically acceptable carrier can be used, and such carriers are well known in the art. The choice of carrier will be determined, at least in part, by the location of the target tissue and/or cells, and the particular method used to administer the composition.

In some embodiments, the composition or formulation comprises a single type of compound of formula (I), formula (II), and/or formula (III) in any suitable amount (e.g., in an effective amount). In other embodiments, a composition or formulation comprises two or more different compounds of formula (I), formula (II), and/or formula (III). For example, in some aspects the composition or formulation comprises PA1-13A-3 shown in Table 1. In other aspects, the composition or formulation comprises PA1-13A-7 and PA1-13A-2 shown in Table 1. Any combination of two or more compounds of formula (I), formula (II), and/or formula (III) can be made to form a composition or formulation (along with a carrier, if desired), and any two or more compounds shown in Table 1 can be made to form a composition or formulation (along with a carrier, if desired).

In some aspects, the composition containing the compound of formula (I), formula (II), and/or formula (III) can further comprise any other suitable components, especially for enhancing the stability of the composition and/or its end use. Accordingly, there is a wide variety of suitable formulations for use with the compounds disclosed herein. The following formulations and methods are merely exemplary and are in no way limiting. Generally, the term “composition” and “formulation” are used interchangeably herein.

Formulations containing the compound of formula (I), formula (II), formula (III), or any combination thereof suitable for parenteral administration include aqueous and nonaqueous, isotonic sterile injection solutions, which can contain antioxidants, buffers, bacteriostats, and solutes that render the formulation isotonic with the blood of the intended recipient, and aqueous and nonaqueous sterile suspensions that can include suspending agents, solubilizers, thickening agents, stabilizers, and preservatives. The formulations can be presented in unit dose or multi dose sealed containers, such as ampules and vials, and can be stored in a freeze dried (lyophilized) condition requiring only the addition of a sterile liquid excipient, for example, water, for injections, immediately prior to use. Extemporaneous injection solutions and suspensions can be prepared from sterile powders, granules, and tablets of the kind previously described. The formulation for parenteral administration can be formulated for intratumoral administration, intravenous injection, intraperitoneal injection, intraocular injection, subcutaneous injection, and the like.

Compositions containing the compound of formula (I), formula (II), formula (III), or any combination thereof suitable for enteric administration are formulated using pharmaceutically acceptable carriers well known in the art in dosages suitable for oral administration. Such carriers enable the pharmaceutical compositions to be formulated as tablets, pills, dragees, capsules, liquids, gels, syrups, slurries, suspensions and the like, for ingestion by the patient. Pharmaceutical preparations for oral use can be obtained through combining active compounds with solid excipients, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, or other plants; cellulose such as methyl cellulose, hydroxypropylmethyl-cellulose, or sodium carboxymethyl cellulose; and gums including arabic and tragacanth; and proteins such as gelatin and collagen. If desired, disintegrating or solubilizing agents may be added, such as cross-linked polyvinyl pyrrolidone, agar, alginic acid, or a salt thereof, such as sodium alginate. Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, i.e., dosage.

Formulations containing the compound of formula (I), formula (II), and/or formula (III) suitable for anal or rectal administration can be prepared as suppositories by mixing the active compound with a variety of bases such as emulsifying bases or water soluble bases. Formulations containing the compound of formula (I), formula (II), and/or formula (III) suitable for vaginal administration can be presented as pessaries, tampons, creams, gels, pastes, foams, or spray formulas containing, in addition to the active ingredient, such carriers as are known in the art to be appropriate.

Formulations containing the compound of formula (I), formula (II), and/or formula (III) suitable for ocular administration can be prepared as an injectables, drops, sprays, or films, by mixing the active compound with a variety of aqueous and nonaqueous, isotonic sterile injection solutions, which can contain anti-oxidants, buffers, bacteriostats, and solutes that render the formulation isotonic with the eye tissue of the intended recipient, and aqueous and nonaqueous sterile suspensions that can include suspending agents, solubilizers, thickening agents, stabilizers, and preservatives.

Formulations containing the compound of formula (I), formula (II), and/or formula (III) suitable for topical administration include creams, lotions, ointments, patches, oils, pastes, sprays (e.g., an aerosol spray), gels, mousse, roll-on liquids, solid sticks, etc. In some aspects, the topical formulation is a cream, a lotion, an ointment, or a patch.

In some aspects, the compound of formula (I), formula (II), and/or formula (III), alone or in combination with other suitable components, is made into an aerosol formulation to be administered via inhalation. A compound of formula (I), formula (II), and/or formula (III) is preferably supplied in finely divided form along with a surfactant and propellant. Typical percentages of the compound of formula (I), formula (II), and/or formula (III) can be about 0.01% to about 20% by weight, preferably about 1% to about 10% by weight. Such weight percents refer to the compound of formula (I) separately, the compound of formula (II) separately, the compound of formula (III) separately, or the combined total of all compounds of formula (I), formula (II), and formula (III) present. The surfactant generally is nontoxic at the concentrations employed, and in some aspects is soluble in the propellant. Representative of such surfactants are the esters or partial esters of fatty acids containing from 6 to 22 carbon atoms, such as caproic, octanoic, lauric, palmitic, stearic, linoleic, linolenic, olesteric and oleic acids with an aliphatic polyhydric alcohol or its cyclic anhydride. Mixed esters, such as mixed or natural glycerides can be employed. The surfactant can constitute from about 0.1% to about 20% by weight of the composition, preferably from about 0.25% to about 5%. The balance of the composition is ordinarily propellant. A carrier can also be included as desired, e.g., lecithin, for intranasal delivery. These aerosol formulations can be placed into acceptable pressurized propellants, such as dichlorodifluoromethane, propane, nitrogen, and the like. They also can be formulated as pharmaceuticals for non-pressured preparations, such as in a nebulizer or an atomizer. Such spray formulations can be used, e.g., to spray mucosa and may be particularly preferable for interfering with the expression or activity of CUL4A, CUL4B, DDB1, or any combination thereof in the respiratory system or oral cavity or pharynx, and particularly for preventing or treating cancers of the respiratory system or the oral cavity or pharynx.

In some aspects, the formulation is a sunscreen composition comprising a compound of formula (I), formula (II), and/or formula (III) and a cosmetically acceptable carrier. Typically, a sunscreen composition is an oil-in-water or water-in-oil emulsion wherein the oil phase comprises one or more sunscreen compounds, solubilizers, silicone emulsifiers, emollients, and other cosmetically acceptable skin conditioning agents. The aqueous phase is predominantly water, but typically comprises additional ingredients such as humectants (e.g., pentylene glycol and glycerine), preservatives, and thickeners. Additional components such as fragrances, dyes, and extracts may be added to either phase or to the emulsion after it is prepared. Similarly, the compound of formula (I), formula (II), and/or formula (III) may be added to the oil phase or the aqueous phase (or both) prior to or during preparation of the emulsion, or such compound can be added to the emulsion after it is prepared, depending upon the physiochemical characteristics of the compound.

As used herein, the term “sunscreen compound” refers to a compound capable of screening ultraviolet radiation having a wavelength of 280 nm-320 nm (i.e., UV-B) and/or 320 nm-400 nm (i.e., UV-A). The sunscreen compound may be one or more organic chemicals that absorb UV radiation, one or more inorganic chemicals that reflect, scatter, or absorb UV radiation, or any combination thereof. Examples of suitable sunscreen compounds include, without limitation, sulisobenzone, dioxybenzone, methyl anthranilate, 4-aminobenzoic acid (PABA), amyl dimethyl PABA, octyl dimethyl PABA, glyceryl PABA, 2-ethoxyethyl p-methoxycinnamate, diethamolamine p-methoxycinnamate, ethylhexyl p-methoxycinnamate, digalloyl trioleate, ethyl 4-bis(hydroxypropyl) aminobenzoate, 2-ethylhexyl-2-cyano-3,3-diphenylacrylate, 2-ethylhexyl salicylate, homomenthyl salicylate, triethanolamine salicylate, 2-phenylbenzimidazole-5-sulfonic acid, red petrolatum, titanium dioxide, zinc oxide, or any combination thereof.

The sunscreen composition can take the form of a lotion, an oil, a gel, a solid stick, a spray, or a foam. Sunscreen compositions and methods of preparation are well known to one of ordinary skill in the art and are described in, e.g., U.S. Pat. Nos. 5,587,150; 5,770,183; and 6,033,649, each of which is hereby incorporated by reference herein for all purposes.

Disclosed herein is a method of co-administering a compound of formula (I), formula (II), and/or formula (III) that interferes with the expression or activity of CUL4A, CUL4B, DDB1, or any combination thereof with a sunscreen composition to an animal (e.g., human) in need thereof. By “co-administering” is meant administering the sunscreen composition and the compound of formula (I), formula (II), and/or formula (III) sufficiently close in time such that the compound of formula (I), formula (II), and/or formula (III) enhances the effectiveness of the sunscreen composition. In this regard, the compound of formula (I), formula (II), and/or formula (III) can be administered first and the sunscreen composition can be administered second, or vice versa. Alternatively, the compound of formula (I), formula (II), and/or formula (III) and the sunscreen composition can be administered simultaneously.

Also disclosed is a method of co-administering a compound of formula (I), formula (II), and/or formula (III) that interferes with the expression or activity of CUL4A, CUL4B, DDB1, or any combination thereof with a chemotherapeutic agent to an animal (e.g., human) in need thereof. By “co-administering” is meant administering the chemotherapeutic agent and a compound of formula (I), formula (II), and/or formula (III) sufficiently close in time such that the compound of formula (I), formula (II), and/or formula (III) can enhance the effectiveness of the chemotherapeutic agent. In this regard, the compound of formula (I), formula (II), and/or formula (III) can be administered first and the chemotherapeutic agent can be administered second, or vice versa. Alternatively, the compound of formula (I), formula (II), and/or formula (III) and the chemotherapeutic agent can be administered simultaneously.

Any class of chemotherapeutic agent can be co-administered with the compound of formula (I), formula (II), and/or formula (III) including without limitation, an antimicrotubule agent, an antimetabolite, an antimitotic, a DNA damaging agent, a proapoptotic, a differentiation inducing agent, an antibiotic, a hormone, or any combination thereof. Suitable chemotherapeutics include, but are not limited to, tyrosine kinase inhibitors (genistein), biologically active agents (TNF, or tTF), radionuclides (131I, 90Y, 111In, 211At, 32P and other known therapeutic radionuclides), adriamycin, ansamycin antibiotics, asparaginase, bleomycin, busulphan, cisplatin, carboplatin, carmustine, capecitabine, chlorambucil, cytarabine, cyclophosphamide, camptothecin, dacarbazine, dactinomycin, daunorubicin, dexrazoxane, docetaxel, doxorubicin, etoposide, epothilones, floxuridine, fludarabine, fluorouracil, gemcitabine, hydroxyurea, idarubicin, ifosfamide, irinotecan, lomustine, mechlorethamine, mercaptopurine, meplhalan, methotrexate, rapamycin (sirolimus) and derivatives, mitomycin, mitotane, mitoxantrone, nitrosurea, paclitaxel, pamidronate, pentostatin, plicamycin, procarbazine, rituximab, streptozocin, teniposide, thioguanine, thiotepa, taxanes, topotecan, vinblastine, vincristine, vinorelbine, taxol, combretastatins, discodermolides, transplatinum, anti vascular endothelial growth factor compounds (“anti VEGFs”), anti epidermal growth factor receptor compounds (“anti EGFRs”), 5 fluorouracil, or any combination thereof. A dose of one or more chemotherapeutic agents can be administered according to the methods disclosed herein. The type and number of chemotherapeutic agents used in the methods disclosed herein will depend on the standard chemotherapeutic regimen for a particular tumor type. In other words, while a particular cancer may be prevented or treated routinely with a single chemotherapeutic agent, another may be prevented or treated routinely with a combination of chemotherapeutic agents. The chemotherapeutic agent is administered in a dose sufficient to prevent or treat the cancer (e.g., cancer-treatment effective amount of a chemotherapeutic agent). A clinician skilled in the art can determine the therapeutically effective amount of a composition in order to prevent or treat a particular disease condition, or disorder when it is administered.

In some aspects, the compound(s) of formula (I), formula (II), and/or formula (III), or a composition comprising the compound(s) of formula (I), formula (II), and/or formula (III) specifically inhibits CRL4 ubiquitin ligase (e.g., without inhibition of CUL1 and/or CUL3 ubiquitin ligase) and selectively kills CUL4^{high} tumor cells in cell and xenograft mouse models, either alone or synergizing with chemotherapeutic agents to achieve therapeutic efficacy.

Disclosed herein is a method of preventing or treating a disease or condition in an animal or subject, such as a mammal or human, which method comprises administering to the animal or subject (e.g., mammal or human) a compound of formula (I):

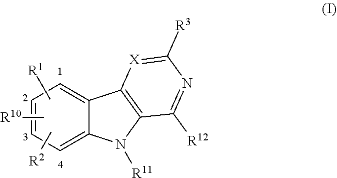

and/or a compound of formula (II):

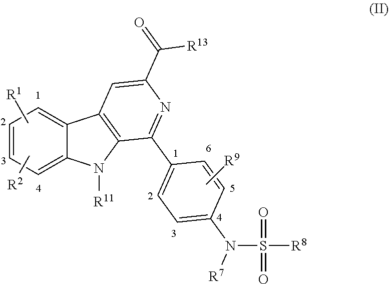

and/or a compound of formula (III):

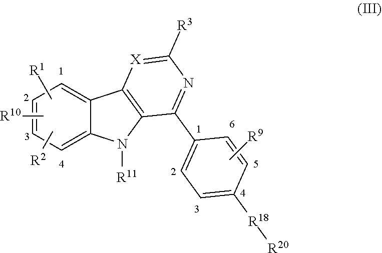

or any salt or ester thereof (including any of the foregoing embodiments provided herein), or composition or formulation comprising same. All aspects and embodiments of the compound of formula (I), formula (II), and/or formula (III) used in the method are as set forth in the description of the compounds themselves. Similarly, all aspects and embodiments of a composition or formulation comprising such compounds are as set forth in the description of such compositions and formulations provided herein.

Without wishing to be bound by any particular theory or mechanism of action, it is believed that compounds of formula (I), formula (II), and/or formula (III) interfere with CRL4A ubiquitin ligase expression or activity, CUL4A expression or activity, CUL4B expression or activity, and/or DDB1 expression or activity (e.g., by disrupting the interaction between CUL4A and/or CUL4B and the beta-propeller B of DDB1), thereby preventing or treating a disease or condition in the animal or subject (e.g., human) that is characterized by abnormal or otherwise pathogenic CRL4A ubiquitin ligase expression or activity, CUL4A expression or activity, CUL4B expression or activity, and/or DDB1 expression or activity. It is also believed that there is a link between inhibition of CUL4A and/or CUL4B expression or activity and DNA damage repair. Accordingly, also provided herein is a method of inhibiting DNA damage (or enhancing DNA repair), and/or inhibiting or slowing aging in an animal or subject (e.g., human), particularly aging associated with DNA damage from external (e.g., UV radiation) or internal DNA damaging sources, which method comprises administering to an animal or subject (e.g., human) an effective amount of a compound of formula (I), formula (II), and/or formula (III), thereby inhibiting or slowing aging in the animal or subject. In some aspects, the compounds of formula (I), formula (II), and/or formula (III) are in the form of a composition further comprising a carrier. In some aspects, the compound of formula (I), formula (II), and/or formula (III), or a composition thereof, is administered to a subject in need thereof (e.g., a subject having a disease or condition, a subject at risk for having a disease or condition, or a subject having an ailment associated with a disease or condition; e.g., in some aspects, such disease or condition is or comprises cancer, aging, DNA damage, or any combination thereof, or any ailment associated therewith). In some aspects, the compound of formula (I), formula (II), and/or formula (III), or a composition thereof, is administered to a subject in an effective amount (e.g., a therapeutically effective amount).

In some aspects, in the method for treating a disease or condition in an animal or subject (e.g., mammal or human) with the compounds of formula (I), formula (II), and/or formula (III), the disease or condition comprises or is cancer, aging, DNA damage, or any combination thereof. In some aspects, the disease or condition comprises or is cancer. Accordingly, in some aspects, disclosed is a method for preventing or treating cancer in a subject comprising administering a compound of formula (I), formula (II), and/or formula (III), or a composition thereof, to the subject. In some aspects, the disease or condition comprises or is aging in a subject. Accordingly, in some aspects, disclosed is a method for inhibiting aging in a subject comprising administering a compound of formula (I), formula (II), and/or formula (III), or a composition thereof, to the subject. In some aspects, the disease or condition comprises or is DNA damage in a subject. Accordingly, in some aspects, disclosed is a method for inhibiting DNA damage (or enhancing or improving DNA repair) in a subject comprising administering a compound of formula (I), formula (II), and/or formula (III), or a composition thereof, to the subject. In some aspects, the subject is an animal, a mammal, or a human. Examples of subjects include humans, cancer patients (e.g., human or otherwise), domestic pets (e.g., dogs, cats, horses), exotic pets (alligators, crocodiles, snakes), zoo animals (e.g., lions, giraffes, hippopotamuses, monkeys, apes, gorillas), marine animals (e.g., whales, sharks, fish), and so forth. In some aspects, the methods comprise administering the compound of formula (I), formula (II), and/or formula (III) to a cell, tissue, or organ, either ex vivo, in vivo, in vitro, or a combination thereof (e.g., as part of a combination therapy).

In some aspects, the disease or condition comprises or is cancer, and the cancer comprises or is breast cancer, colorectal cancer, lung cancer, or brain cancer. In some aspects, the cancer is resistant to one or more topoisomerase I-directed chemotherapy drugs, optionally wherein the one or more drugs comprise camptothecin, irinotecan, or topotecan. As used herein, a cancer that is “resistant to” a particular type of drug would be readily understood in view of standard medical criteria for determining cancer drug resistance. In some aspects, when the disease or condition comprises or is cancer, the cancer is reduced as a result of the method. In some aspects, the method selectively kills tumor cells (e.g., cancer cells) with a high level of expression of CUL4A, CUL4B, or both.

In some aspects, in the method for treating a disease or condition in an animal or subject (e.g., human), the disease or condition is characterized by (1) increased CRL4A ubiquitin ligase expression or activity as compared to that of a normal, non-diseased subject, (2) increased CUL4A or CUL4B expression or activity as compared to that of a normal, non-diseased subject, (3) increased DDB1 expression or activity as compared to that of a normal, non-diseased subject, or (4) any combination thereof. Such combinations include, for example, (1) and (2); (1) and (3); (2) and (3); and (1), (2), and (3).

In some aspects, the method for treating a disease or condition in an animal or subject (e.g., human) disrupts the interaction between CUL4A or CUL4B and the beta-propeller B of DDB1.

In some aspects, the method for treating a disease or condition in an animal or subject (e.g., human) does not inhibit CUL1 and/or CUL 3 ubiquitin ligase (i.e., does not inhibit CUL1 ubiquitin ligase, does not inhibit CUL 3 ubiquitin ligase, or does not inhibit both CUL1 and CUL 3 ubiquitin ligase).

The following examples further illustrate the invention but, of course, should not be construed as in any way limiting its scope.

### Example 1

This example demonstrates the synthesis of compound PA99-1 (methyl 5-fluoro-1-(4-(N-methylphenylsulfonamido)phenyl)-9H-pyrido[3,4-b]indole-3-carboxylate) and compound PA99 (5-fluoro-1-(4-(N-methylphenylsulfonamido)phenyl)-9H-pyrido[3,4-b]indole-3-carboxylic acid). The reaction scheme to produce compound PA99-1 and compound PA99 is shown in FIG. 1, and each individual step in the synthesis is described in detail below.

9[PA99]: To a solution of 8[PA99] (ethyl 4-aminobenzoate) (20.0 g, 121 mmol, 1.0 eq) in THF (200 mL) was added pyridine (28.7 g, 363 mmol, 3.0 eq) and benzenesulfonyl chloride (23.5 g, 133 mmol, 1.1 eq) at 0° C. The mixture was stirred at 25° C. for 12 h. The mixture was diluted with H₂O (100 mL), extracted with EA (150 mL×3). The combined extracts were washed with H₂O (100 mL), brine (100 mL), evaporated in vacuo to give 9[PA99](ethyl 4-(benzenesulfonamido)benzoate) (23.0 g, 55% yield) was brown oil. LCMS: m/z 306.1 [M+H]⁺.

10[PA99]: To a solution of 9[PA99] (ethyl 4-(benzenesulfonamido)benzoate) (23.0 g, 66.6 mmol, 1.0 eq) in THF (200 mL) was added MeI (21.3 g, 150 mmol, 2.3 eq) and K₂CO₃ (27.6 g, 200 mmol, 3.0 eq) at 15° C. The mixture was stirred at 60° C. for 12 h. The mixture was diluted with H₂O (100 mL), extracted with EA (100 mL×3). The combined extracts were washed with H₂O (100 mL), brine (100 mL), evaporated in vacuo to give crude product. Chromatograph column (PE in EA from 0% to 15%) gave 10[PA99] (ethyl 4-[benzenesulfonyl(methyl)amino]benzoate) (20.0 g, 93% yield) as brown oil. LCMS: m/z 320.1 [M+H]⁺.

11[PA99]: To a solution of 10[PA99] (ethyl 4-[benzenesulfonyl(methyl)amino]benzoate) (10.0 g, 30.9 mmol, 1.0 eq) in THF (150 mL) was added LiAlH₄ (2.35 g, 61.9 mmol, 2.0 eq) at 0° C. The mixture was stirred at 0° C. for 1 h. The mixture was quenched with NaOH (15% in H₂O, 2.4 mL) and H₂O (2.4 mL) at 0° C., diluted with EA (100 mL), filtered and evaporated in vacuo to give 11[PA99] (N-[4-(hydroxymethyl)phenyl]-N-methyl-benzenesulfonamide) (7.0 g, 62% yield) as colorless oil. LCMS: m/z 300.1 [M+Na]⁺; 577.2 [2M+Na]⁺.

5[PA99]: To a solution of 11[PA99] (N-[4-(hydroxymethyl)phenyl]-N-methyl-benzenesulfonamide) (7.00 g, 19.1 mmol, 1.0 eq) in DCM (50 mL) was added Dess-Martin (8.10 g, 19.1 mmol, 1.0 eq) at 0° C. The mixture was stirred at 0° C. for 1 h. The mixture was diluted with H₂O (200 mL), extracted with EA (200 mL×3). The combined extracts were washed with H₂O (200 mL), evaporated in vacuo to give crude product. Chromatograph column (EA in PE from 0% to 15%) gave 5[PA99] (N-(4-formylphenyl)-N-methyl-benzenesulfonamide) (3.50 g, 64% yield) as a white solid. LCMS: m/z 276.1 [M+H]⁺.

3[PA99]: To a solution of 1[PA99] (4-fluoro-1H-indole) (10.0 g, 74.0 mmol, 1.0 eq) in AcOH (150 mL) and Ac₂O (54.5 g, 533 mmol, 7.2 eq) was added 2[PA99] (2-amino-3-hydroxy-propanoic acid) (15.5 g, 148 mmol, 2.0 eq). The mixture was stirred at 40° C. for 16 h. The mixture was evaporated in vacuo to get crude product. The residue was dissolved in H₂O (100 mL), extracted with EA (100 mL×3). The combined organic lawyers dried over with Na₂SO₄, filtered and concentrated under reduced pressure to give a residue. The residue was purified by reverse flash (TFA condition) to give 3[PA99] (2-acetamido-3-(4-fluoro-1H-indol-3-yl)propanoic acid) (15.0 g, 63% yield) as brown oil. LCMS: m/z 265.1 [M+H]⁺.

4[PA99]: A mixture of 3[PA99] (2-acetamido-3-(4-fluoro-1H-indol-3-yl)propanoic acid) (10.0 g, 31.4 mmol, 1.0 eq) and HCl (185 g, 1.83 mol, ˜180 mL, 58.3 eq) in H₂O (50 mL) was stirred at 75° C. for 12 h. The mixture was extracted with EA (100 mL×3). The water phase was evaporated in vacuo to give 4[PA99] (2-amino-3-(4-fluoro-1H-indol-3-yl)propanoic acid) (7.5 g, 54% yield) as a brown solid. LCMS: m/z 223.2 [M+H]⁺.

6[PA99]: To a solution of 4[PA99] (2-amino-3-(4-fluoro-1H-indol-3-yl)propanoic acid) (4.00 g, 8.97 mmol, 1.0 eq) in AcOH (60 mL) was added 5[PA99] (N-(4-formylphenyl)-N-methyl-benzenesulfonamide) (2.17 g, 7.63 mmol, 0.85 eq) (5[PA99] was prepared above). The mixture was stirred at 100° C. for 5 h. The mixture was evaporated in vacuo to get crude product 6[PA99] (1-[4-[benzenesulfonyl(methyl)amino]phenyl]-5-fluoro-2,3,4,9-tetrahydro-1H-pyrido[3,4-b]indole-3-carboxylic acid) (6.30 g, crude) as brown oil, which was used directly for next step without further purification. LCMS: m/z 480.2 [M+H]⁺.

7[PA99]: A mixture of 6[PA99] (1-[4-[benzenesulfonyl(methyl)amino]phenyl]-5-fluoro-2,3,4,9-tetrahydro-1H-pyrido[3,4-b]indole-3-carboxylic acid) (6.30 g, 13.1 mmol, 1.0 eq) and HCl/MeOH (4 M HCl in MeOH, 450 mL) was stirred at 25° C. for 12 h. The mixture was evaporated in vacuo to get crude product. The residue was dissolved in H₂O (100 mL), make pH=7 with NaHCO₃ (sat.), extracted with EA (100 mL×3). The combined extracts were washed with H₂O (100 mL), evaporated in vacuo to give crude product. The residue was purified by column chromatography (PE:EA=1:0 to 1:1) to give 7[PA99] (methyl 1-[4-[benzenesulfonyl(methyl)amino]phenyl]-5-fluoro-2,3,4,9-tetrahydro-1H-pyrido[3,4-b]indole-3-carboxylate) (1.00 g, 11% yield) as a brown solid. LCMS: m/z 494.2 [M+H]⁺.

PA99-1: To a solution of 7[PA99] (methyl 1-[4-[benzenesulfonyl(methyl)amino]phenyl]-5-fluoro-2,3,4,9-tetrahydro-1H-pyrido[3,4-b]indole-3-carboxylate) (750 mg, 1.14 mmol, 1.0 eq) in DMF (10 mL) was added PhI(OAc)₂ (1.10 g, 3.41 mmol, 3.0 eq). The mixture was stirred at 25° C. for 12 h. The mixture was evaporated in vacuo to give crude product, extracted with EA (50 mL×3). The combined extracts were washed with H₂O (50 mL), evaporated in vacuo to give crude product. The residue was purified by column chromatography (SiO2, Petroleum ether/Ethyl acetate=5:1 to 2:1) to give crude product, then the crude product was purified by Prep-HPLC (column: Boston pH-lex 150*25 10 um; mobile phase: [water (0.1% TFA)-ACN]; B %: 60%-90%, 10 min) to give PA99-1 (methyl 1-[4-[benzenesulfonyl(methyl)amino]phenyl]-5-fluoro-9H-pyrido[3,4-b]indole-3-carboxylate) (81.0 mg, 14% yield) as yellow solid. LCMS: m/z 490.0 [M+H]⁺. ¹H NMR (DMSO-d₆ 400 MHz) δ 12.30 (S, 1H), 8.71 (s, 1H), 8.01 (d, J=8.4 Hz, 2H), 8.00-7.65 (m, 1H), 7.65-7.64 (m, 5H), 7.57-7.55 (m, 1H), 7.42 (d, J=8.8 Hz, 2H), 7.20-7.15 (m, 1H), 3.95 (s, 3H), 3.27 (s, 3H).

PA99: To a solution of PA99-1 (methyl 1-[4-[benzenesulfonyl(methyl)amino]phenyl]-5-fluoro-9H-pyrido[3,4-b]indole-3-carboxylate) (600 mg, 1.15 mmol, 1.0 eq) in H₂O (5 mL) and MeOH (5 mL) was added NaOH (229 mg, 5.74 mmol, 5.0 eq). The mixture was stirred at 25° C. for 12 h. The residue was dissolved in H₂O (50 mL), make pH=5 with HCl (1 M in H₂O), extracted with EA (50 mL×3), the combined extracts were washed with H₂O (50 mL), evaporated in vacuo to get crude product. The residue was purified by Prep-HPLC (column: Boston pH-lex 150*25 10 um; mobile phase: [water (0.1% TFA)-ACN]; B %: 46%-76%, 10 min) to give product PA99 (1-[4-[benzenesulfonyl(methyl)amino]phenyl]-5-fluoro-9H-pyrido[3,4-b]indole-3-carboxylic acid) (255 mg, 45% yield) as a yellow solid. LCMS: m/z 476.0 [M+H]⁺. H NMR (DMSO-d₆ 400 MHz) (12.27 (s, 1H), 8.70 (s, 1H), 8.06 (d, J=8.4 Hz, 2H), 7.76-7.72 (m, 1H), 7.65-7.63 (m, 5H), 7.57-7.55 (m, 1H), 7.41 (d, J=8.8 Hz, 2H), 7.19-7.17 (m, 1H), 3.27 (s, 3H).

Using similar synthetic procedures as described in this example, various other compounds of the invention, including those set forth in Table 1, can be synthesized.

### Example 2

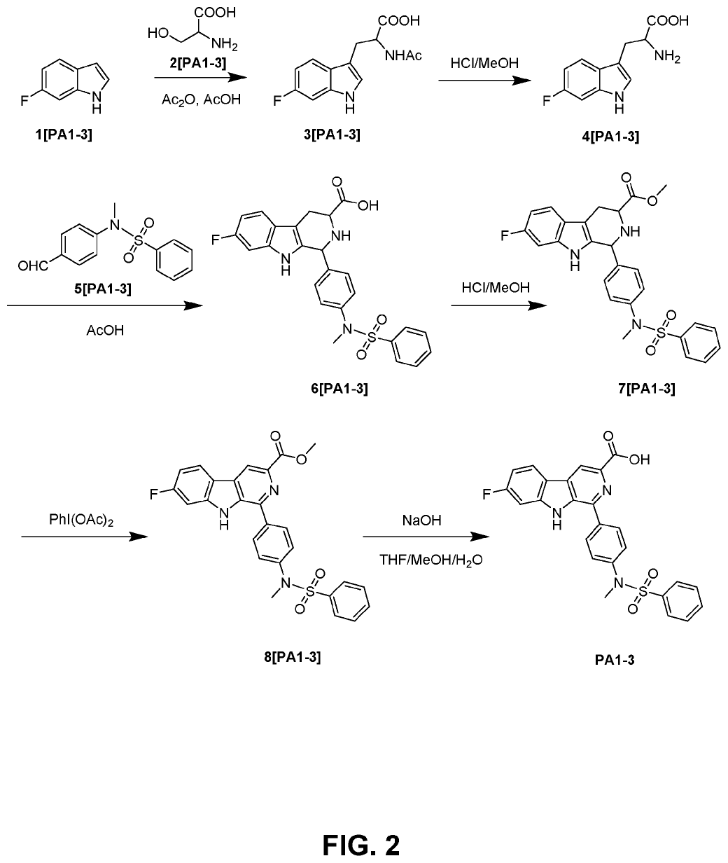

This example demonstrates the synthesis of compound 8[PA1-3] (methyl 7-fluoro-1-(4-(N-methylphenylsulfonamido)phenyl)-9H-pyrido[3,4-b]indole-3-carboxylate) and compound PA1-3 (7-fluoro-1-(4-(N-methylphenylsulfonamido)phenyl)-9H-pyrido[3,4-b]indole-3-carboxylic acid). The reaction scheme to produce compound 8[PA1-3] and compound PA1-3 is shown in FIG. 2, and each individual step in the synthesis is described in detail below.

3[PA1-3]: To the mixture of 1[PA1-3] (6-fluoro-1H-indole) (10.0 g, 74.0 mmol, 1.0 eq) in Ac₂O (50 mL) and CH₃COOH (120 mL) was added 2[PA1-3] (2-amino-3-hydroxypropanoic acid) (15.6 g, 148 mmol, 2 eq) at 25° C. The mixture was stirred at 40° C. for 12 h. The LCMS showed desired MS was detected. The reaction was concentrated in vacuo and then the residue was poured into water (600 mL) and extracted with EA (500 mL×3). The organic layer was combined and washed with brine (1 L), dried over Na₂SO₄, filtered and concentrated in vacuo to give 3[PA1-3] (2-acetamido-3-(6-fluoro-1H-indol-3-yl)propanoic acid) (26.0 g, crude) as red oil. LCMS: m/z 265.0[M+H]⁺.

4[PA1-3]: To a solution of 3[PA1-3] (2-acetamido-3-(6-fluoro-1H-indol-3-yl)propanoic acid) (2.50 g, 9.46 mmol, 1.0 eq) in H₂O (6 mL) was added HCl (18.6 mL, 37% purity) under N₂. The solution was stirred at 75° C. for 12 h. LCMS showed that 3[PA1-3] was consumed completely and desired MS was detected. The reaction was concentrated in vacuo to give 4[PA1-3] (2-amino-3-(6-fluoro-1H-indol-3-yl)propanoic acid) (27.0 g, crude) as black brown oil and carried forward without purification. LCMS: m/z 223.0[M+H]⁺. In a separate batch at 20 g scale of 3[PA1-3], the solution that was stirred for 12 h was poured into water (100 mL) and extracted with EA (150 mL×2). Then the aqueous phase was basified by NH₃/H₂O (con.) to make pH=7-8. The aqueous phase purified by Reversed-flash C-18 column (0.1% TFA condition). 4[PA1-3] (3.90 g, 23% yield, 99.1% purity) was obtained as brown solid, which was confirmed by LCMS: m/z 223.2 [M+H]⁺.

6[PA1-3]: A solution of 4[PA1-3] (2-amino-3-(6-fluoro-1H-indol-3-yl)propanoic acid) (15.0 g, 67.5 mmol, 1.0 eq) and 5[PA1-3] (N-(4-formylphenyl)-N-methylbenzenesulfonamide) (1.94 g, 6.75 mmol, 0.10 eq) in AcOH (100 mL) was stirred at 110° C. for 2 h. Note that 5[PA1-3] is the same as compound 5[PA99] prepared in Example 1. LCMS showed 4[PA1-3] was consumed completely and desired MS was detected. The reaction was concentrated in vacuo. The residue was dried in vacuo to give 6[PA1-3] (7-fluoro-1-(4-(N-methylphenylsulfonamido)phenyl)-2,3,4,9-tetrahydro-1H-pyrido[3,4-b]indole-3-carboxylic acid) (17.0 g, crude) as black brown solid. LCMS: m/z 480.1[M+H]⁺.

7[PA1-3]: Compound [6[PA1-3] (7-fluoro-1-(4-(N-methylphenylsulfonamido)phenyl)-2,3,4,9-tetrahydro-1H-pyrido[3,4-b]indole-3-carboxylic acid) (17.0 g, 35.5 mmol, 1 eq) was dissolved in HCl/MeOH (4 M, 100 mL) and then the mixture was stirred at 25° C. for 12 h. TLC (PE/EA=2:1) showed that the starting material was consumed completely and new spots were formed. The reaction was concentrated in vacuo. Then the residue was poured into water (200 mL) and basified by Na₂CO₃ (solid) to pH=8-9. The mixture was extracted with EA (400 mL×3). The organic layers were combined and washed with brine (500 mL), dried over Na₂SO₄, and concentrated in vacuo. The residue was purified by silica gel chromatography (PE/EA from 2:1 to 1:3) to give 7[PA1-3] (methyl 7-fluoro-1-(4-(N-methylphenylsulfonamido)phenyl)-2,3,4,9-tetrahydro-1H-pyrido[3,4-b]indole-3-carboxylate) (2.00 g, 5.5% yield, 48% purity) as black brown oil, detected by LCMS: m/z 494.1[M+H]⁺.

8[PA1-3]: To a solution of 7[PA1-3] (methyl 7-fluoro-1-(4-(N-methylphenylsulfonamido)phenyl)-2,3,4,9-tetrahydro-1H-pyrido[3,4-b]indole-3-carboxylate) (1.90 g, 1.85 mmol, 1.0 eq) in DMF (20 mL) was added PhI(OAc)₂ (1.79 g, 5.54 mmol, 3.0 eq) at 25° C. and the mixture was stirred at 25° C. for 12 h. The reaction was poured into water (60 mL) and extracted with EA (20 mL×3). The organic layer was combined and washed with brine (50 mL), dried over Na₂SO₄, and concentrated in vacuo. The residue was purified by silica gel chromatography with PE/EA=2:1 to 1:4 to give 8[PA1-3] (methyl 7-fluoro-1-(4-(N-methylphenylsulfonamido)phenyl)-9H-pyrido[3,4-b]indole-3-carboxylate) (0.16 g, 12% yield, 66% purity) as black brown oil, detected by LCMS: m/z 490.1[M+H]⁺.

PA1-3: To a solution of 8[PA1-3] (methyl 7-fluoro-1-(4-(N-methylphenylsulfonamido)phenyl)-9H-pyrido[3,4-b]indole-3-carboxylate) (90.0 mg, 121 umol, 1 eq) in MeOH (3 mL) and THF (3 mL) and H₂O (3 mL) was added NaOH (100 mg, 2.50 mmol, 20.6 eq) at 25° C. and then the mixture was stirred at 60° C. for 12 h. LCMS showed that 8[PA1-3] was consumed completely and desired MS was detected. The reaction was acidified by TFA to pH=3-4, Then the mixture was concentrated in vacuo. The residue was purified by Prep-HPLC (column: Phenomenex Luna C18 150*25 mm*10 um; mobile phase: [water (0.1% TFA)-ACN]; B %: 41%-71%, 10 min) to give PA1-3 (24.0 mg, 40% yield, 97.0% purity) as yellow solid, confirmed by LCMS, ¹H NMR, and HPLC. LCMS: m/z 476.1 [M+H]⁺. ¹H NMR (DMSO-d₆, 400 MHz) δ 12.06 (s, 1H), 8.91 (s, 1H), 8.50-8.48 (m, 1H), 8.05 (d, J=8.4 Hz, 2H), 7.73-7.72 (m, 1H), 7.64-7.63 (m, 4H), 7.41-7.38 (m, 3H), 7.19-7.15 (m, 1H), 3.21 (s, 3H).

Using similar synthetic procedures as described in this example, various other compounds of the invention, including those set forth in Table 1, can be synthesized.

### Example 3

This example demonstrates the synthesis of compound 8[PA1-9] (methyl 7-cyano-1-(4-(N-methylphenylsulfonamido)phenyl)-9H-pyrido[3,4-b]indole-3-carboxylate) and compound PA1-9 (7-cyano-1-(4-(N-methylphenylsulfonamido)phenyl)-9H-pyrido[3,4-b]indole-3-carboxylic acid).

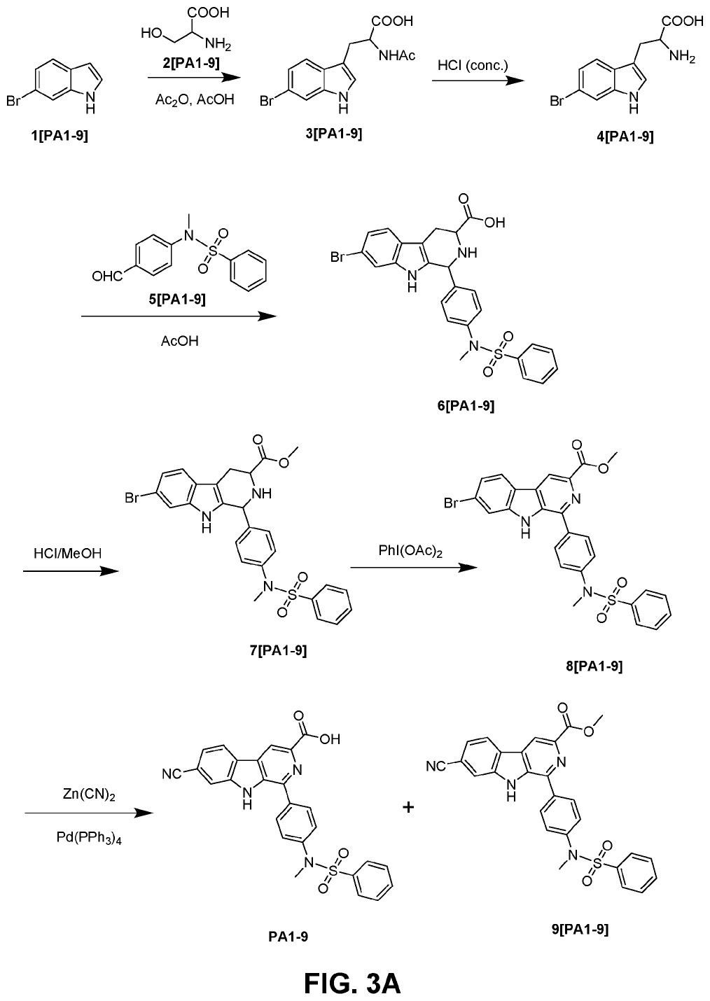

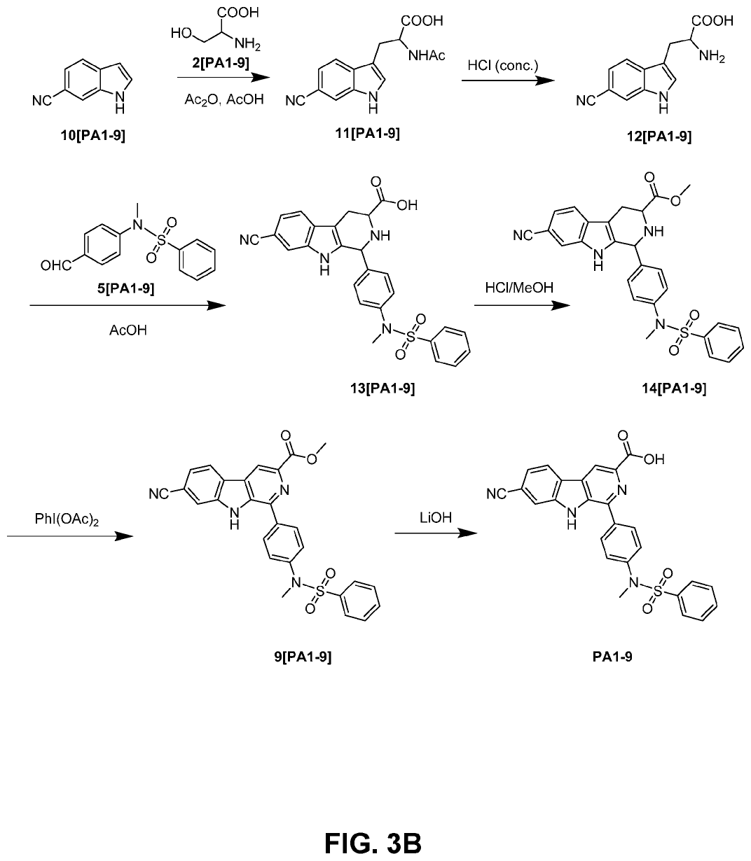

Two possible reaction schemes to produce compound 8[PA1-9] and compound PA1-9 is shown in FIG. 3A and FIG. 3B. The synthetic route shown in FIG. 3A is described in detail below.

3[PA1-9]: To a solution of 2[PA1-9] (2-amino-3-hydroxy-propanoic acid) (10.7 g, 102 mmol, 2.0 eq) and 1[PA1-9] (6-bromo-1H-indole) (10.0 g, 51.0 mmol, 1 eq) in AcOH (120 mL) was added Ac₂O (50 mL) at 25° C. under N₂. The mixture was stirred at 40° C. for 12 h. The LCMS showed there was mainly desired product detected. The reaction was poured into water (400 mL) and extracted with EA (250 mL×3). Then the organic layer were combined and washed with water (300 mL×3). Then the organic layer was dried over Na₂SO₄, filtered and concentrated in vacuo. The residue was dried in vacuo to give 3[PA1-9] (2-acetamido-3-(6-bromo-1H-indol-3-yl)propanoic acid) (25.0 g, crude) as yellow oil. LCMS: m/z 326.8 [M+H]⁺.

4[PA1-9]: To a solution of 3[PA1-9] (2-acetamido-3-(6-bromo-1H-indol-3-yl)propanoic acid) (10.0 g, 30.8 mmol, 1 eq) in H₂O (24 mL) was added HCl (72 mL, 37% purity) at 25° C. and then the mixture was stirred at 75° C. for 12 h. The LCMS showed the starting material 3[PA1-9] was consumed and the desired product was detected. The mixture was poured into water (500 mL) and extracted with EA (500 ml×3). Then the aqueous phase was basified by NH₃/H₂O (con.) to pH=7-8. Then the aqueous phase was purified by reversed-phase C-18 column (0.1% TFA condition) to give 4[PA1-9] (2-amino-3-(6-bromo-1H-indol-3-yl)propanoic acid) (1.90 g, 22% yield) as a yellow solid, which was confirmed by ¹H-NMR. LCMS: m/z 283.0 [M+H]⁺. ¹H NMR (DMSO-d₆, 400 MHz) δ 11.18 (s, 1H), 7.57-7.52 (m, 2H), 7.26-7.24 (m, 1H), 7.15-7.12 (m, 1H), 4.00-4.95 (m, 1H), 3.27-3.16 (m, 2H).

6[PA1-9]: A solution of 4[PA1-9] (2-amino-3-(6-bromo-1H-indol-3-yl)propanoic acid (800 mg, 2.83 mmol, 1 eq)) and 5[PA1-9] (N-(4-formylphenyl)-N-methylbenzenesulfonamide) (778 mg, 2.83 mmol, 1 eq) in AcOH (20 mL) was stirred at 110° C. for 3 h. Note that 5[PA1-9] is the same as compound 5[PA99] prepared in Example 1. LCMS showed that the reactant 4[PA1-9] was consumed completely and desired product was detected. The reaction was concentrated in vacuo to give 6[PA1-9] (7-bromo-1-(4-(N-methylphenylsulfonamido)phenyl)-2,3,4,9-tetrahydro-1H-pyrido[3,4-b]indole-3-carboxylic acid) (2.00 g, crude) as yellow oil, which was used for the next step directly without further purification. LCMS: m/z 542.3 [M+H]⁺.

7[PA1-9]: A solution of 6[PA1-9] (1-[4-[benzenesulfonyl(methyl)amino]phenyl]-7-bromo-2,3,4,9-tetrahydro-1H-pyrido[3,4-b]indole-3-carboxylic acid) (2.00 g, 3.70 mmol, 1 eq) in HCl/MeOH (4 M, 35 mL) was stirred at 25° C. for 12 h. LCMS showed that the reactant 6[PA1-9] was consumed and desired product was detected. The mixture was concentrated in vacuo to afford the residue as black solid. Then the residue was poured into water (20 mL) and basified by K₂CO₃ solid to make pH=10-11. Then the mixture was extracted by EA (10 mL×3), the organic layers were washed by brine (10 mL×3), dried by Na₂SO₄, filtered, concentrated in vacuo. The residue was purified by silica gel chromatography (PE/EA=1:1) to give 7[PA1-9](methyl 1-[4-[benzenesulfonyl(methyl)amino]phenyl]-7-bromo-2,3,4,9-tetrahydro-1H-pyrido[3,4-b]indole-3-carboxylate) (0.85 g, 39% yield, 94.9% purity) as yellow oil. LCMS: m/z 556.0 [M+H]⁺.

8[PA1-9]: To a solution of 7[PA1-9] (methyl 1-[4-[benzenesulfonyl(methyl)amino]phenyl]-7-bromo-2,3,4,9-tetrahydro-1H-pyrido[3,4-b]indole-3-carboxylate) (0.85 g, 1.45 mmol, 94.9% purity, 1 eq) in DMF (15 mL) was added PhI(OAc)₂ (1.41 g, 4.36 mmol, 3.0 eq) and the mixture stirred at 25° C. for 12 h. LCMS showed that the reactant 7[PA1-9] was consumed completely and desired product was obtained. The reaction was poured into water (30 mL) and extracted with EA (30 mL×3). The organic layers were combined and washed with brine (30 mL), dried over Na₂SO₄, and concentrated in vacuo. The residue was purified by silica gel chromatography (PE/EA=3:1 to 1:1) to give 8[PA1-9] (methyl 1-[4-[benzenesulfonyl(methyl)amino]phenyl]-7-bromo-9H-pyrido[3,4-b]indole-3-carboxylate) (0.72 g, 78% yield, 86.7% purity) as yellow oil. LCMS: m/z 551.9[M+H]⁺.

PA1-9 and 9[PA1-9]: To a solution of 8[PA1-9] (methyl 1-[4-[benzenesulfonyl(methyl)amino]phenyl]-7-bromo-9H-pyrido[3,4-b]indole-3-carboxylate) (300 mg, 473 umol, 86.7% purity, 1 eq) in DMF (10 mL) was added Pd(PPh₃)₄ (54.6 mg, 47.25 umol, 0.1 eq) and Zn(CN)₂ (555 mg, 4.73 mmol, 10 eq) at 25° C. under N₂. Then the mixture was stirred at 135° C. for 12 h. LCMS showed 8[PA1-9] was consumed and the desired products were detected. The mixture was poured into water (50 mL) and filtrated, and then the filtrate was extracted with EA (30 mL×3). The organic layer was washed with brine (50 mL), dried over Na₂SO₄, filtered and concentrated in vacuo. The residue was purified by prep-HPLC: column: Phenomenex luna C18 150*40 mm*15 um; mobile phase: [water (0.1% TFA)-ACN]; B %: 47%-77%, 10 min], column: Phenomenex Synergi C18 150*25 mm*10 um; mobile phase: [water (0.1% TFA)-ACN]; B %: 51%-81%, 10 min] to afford PA1-9 (65.5 mg, 27.7% yield, 96.3% purity) and 9[PA1-9] (35.0 mg, 14.8% yield, 99.5% purity). PA1-9 was obtained as a white solid. LCMS: m/z 483.1[M+H]⁺. ¹H NMR (DMSO-d₆, 400 MHz) δ 12.37 (s, 1H), 9.05 (s, 1H), 8.68 (d, J=8.0 Hz, 1H), 8.10-8.04 (m, 3H), 7.76-7.10 (m, 2H), 7.65-7.63 (m, 4H), 7.43-7.40 (m, 2H), 3.26 (s, 3H). 9[PA1-9] was obtained as a white solid. LCMS: m/z 497.1[M+H]⁺. ¹H NMR (DMSO-d₆, 400 MHz) δ 12.42 (s, 1H), 9.09 (s, 1H), 8.71 (d, J=8.4 Hz, 1H), 8.10 (s, 1H), 8.01 (d, J=8.4 Hz, 2H), 7.76-7.73 (m, 2H), 7.66-7.64 (m, 4H), 8.43 (d, J=8.4 Hz, 1H), 3.95 (s, 3H), 3.27 (s, 3H).

Using similar synthetic procedures as described in this example, various other compounds of the invention, including those set forth in Table 1, can be synthesized.

### Example 4

This example demonstrates the synthesis of compound 12[PA1-13A-7] (methyl 7-fluoro-1-(4-((N-methyl-4-(4-methylpiperazin-1-yl)phenyl)sulfonamido)phenyl)-9H-pyrido[3,4-b]indole-3-carboxylate) and compound PA1-13A-7 (7-fluoro-1-(4-((N-methyl-4-(4-methylpiperazin-1-yl)phenyl)sulfonamido)phenyl)-9H-pyrido[3,4-b]indole-3-carboxylic acid).

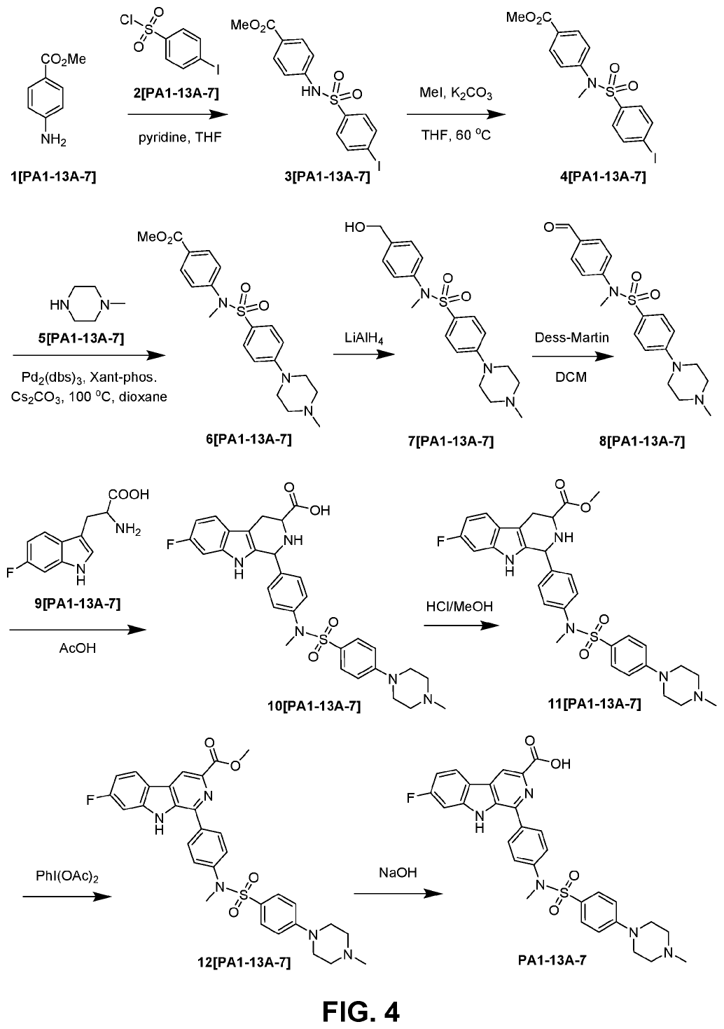

The reaction scheme to produce compound 12[PA1-13A-7] and compound PA1-13A-7 is shown in FIG. 4, and each individual step in the synthesis is described in detail below.

3[PA1-13A-7]: A mixture of 1[PA1-13A-7] (methyl 4-aminobenzoate) (4.00 g, 26.5 mmol, 1 eq), 2[PA1-13A-7] (4-iodobenzenesulfonyl chloride) (8.20 g, 27.1 mmol, 1.02 eq) and pyridine (6.28 g, 79.4 mmol, 3.0 eq) in THF (60 mL) was stirred at 50° C. for 2 h. The LCMS showed 1[PA1-13A-7] was consumed completely and the desired product was detected. The reaction was poured into water (300 mL) and extracted with EA (200 mL×3), the organic layer was combined and washed with brine (300 mL), concentrated in vacuo to give 3[PA1-13A-7](methyl 4-[(4-iodophenyl)sulfonylamino]benzoate) (11.0 g, 99.6% yield) as white solid, which was used for the next step without further purification. LCMS: m/z 417.9 [M+H]⁺.

4[PA1-13A-7]: To a solution of 3[PA1-13A-7] (methyl 4-[(4-iodophenyl)sulfonylamino]benzoate) (14.0 g, 33.6 mmol, 1 eq) and K₂CO₃ (13.9 g, 101 mmol, 3.0 eq) in THF (250 mL) was added MeI (11.9 g, 83.9 mmol, 2.5 eq) at 25° C. under N₂. The mixture was stirred at 60° C. for 12 h. LCMS and TLC (PE/EA=3:1) showed the 3[PA1-13A-7] was consumed and the desired product was detected. After cooling, the reaction was filtrated and the solution was concentrated in vacuo. The residue was purified by silica gel chromatography (PE/EA=1:1 to DCM/MeOH=10:1) to give 4[PA1-13A-7] (methyl 4-[(4-iodophenyl)sulfonyl-methyl-amino]benzoate) (13.0 g, 89.8% yield) as white solid. LCMS: m/z 431.9 [M+H]⁺. ¹H NMR (CDCl₃ 400 MHz) δ 7.99 (d, J=8.8 Hz, 2H), 7.81 (d, J=8.4 Hz, 2H), 7.24-7.20 (m, 4H), 3.93 (s, 2H), 3.21 (s, 3H).

6[PA1-13A-7]: To a solution of 4[PA1-13A-7] (methyl 4-[(4-iodophenyl)sulfonyl-methyl-amino]benzoate) (5.00 g, 11.6 mmol, 1 eq) and 5[PA1-13A-7] (1-methylpiperazine) (1.39 g, 13.9 mmol, 1.2 eq) in dioxane (100 mL) was added Cs₂CO₃ (9.44 g, 29.0 mmol, 2.5 eq), Pd₂(dba)₃ (849 mg, 928 umol, 0.08 eq), and (5-diphenylphosphanyl-9,9-dimethyl-xanthen-4-yl)-diphenyl-phosphane (1.07 g, 1.86 mmol, 0.16 eq) at 25° C. under N₂. The reaction was stirred at 100° C. for 12 h. LCMS and TLC (Ethyl acetate:Methanol=10/1) showed that the reactant 4[PA1-13A-7] was consumed completely and desired product was detected. The mixture was concentrated under reduced pressure affording the crude product as black brown oil. The crude product was purified by silica gel column chromatography (petroleum ether/ethyl acetate from 1/1 to 0/1; then Ethyl acetate/Methanol from 1/0 to 10/1) to give 6[PA1-13A-7] (methyl 4-[methyl-[4-(4-methylpiperazin-1-yl)phenyl]sulfonyl-amino]benzoate) (4.60 g, 98.3% yield) as yellow oil. LCMS: m/z 404.0 [M+H]⁺. ¹H NMR (CDCl₃ 400 MHz) δ 7.97 (d, J=8.8 Hz, 2H), 7.36 (d, J=8.8 Hz, 2H), 7.24 (d, J=8.4 Hz, 2H), 6.81 (d, J=9.2 Hz, 2H), 3.92 (s, 3H), 3.37-3.34 (m, 4H), 3.18 (s, 3H), 2.60-2.56 (m, 2H), 2.05 (s, 3H).

7[PA1-13A-7]: To a solution of 6[PA1-13A-7] (methyl 4-[methyl-[4-(4-methylpiperazin-1-yl)phenyl]sulfonyl-amino]benzoate) (4.60 g, 11.4 mmol, 1 eq) in THF (90 mL) was added LiAlH₄ (1.08 g, 28.5 mmol, 2.5 eq) at 0° C. under N₂. Then the mixture was stirred at 25° C. for 1 h. TLC (EA/MeOH=5:1) showed that the reactant 6[PA1-13A-7] was consumed completely and new spot was formed. The reaction was quenched with NaOH solution (1.1 mL, 15%), followed by water (1.1 mL) at 0° C. Then the mixture was stirred for 15 minutes at room temperature, filtrated, and the organic layer was dried in vacuo to give 7[PA1-13A-7](N-[4-(hydroxymethyl)phenyl]-N-methyl-4-(4-methylpiperazin-1-yl)benzenesulfonamide) (3.88 g, 90.6% yield) was obtained as yellow oil. ¹H NMR (CDCl₃ 400 MHz) δ 7.40 (d, J=8.8 Hz, 2H), 7.31 (d, J=8.8 Hz, 2H), 7.14 (d, J=8.4 Hz, 2H), 6.84 (d, J=9.2 Hz, 2H), 4.70 (s, 2H), 3.36-3.33 (m, 4H), 3.14 (s, 3H), 2.60-2.54 (m, 4H), 2.36 (s, 3H).

8[PA1-13A-7]: To a solution of 7[PA1-13A-7] (N-[4-(hydroxymethyl)phenyl]-N-methyl-4-(4-methylpiperazin-1-yl)benzenesulfonamide) (1.80 g, 4.79 mmol, 1 eq) in DCM (36 mL) was added Dess Martin (2.24 g, 5.27 mmol, 1.1 eq) at 0° C. under N₂. The reaction was stirred at 0° C. for 1 h. Then the solution was stirred at 25° C. for 2 h. TLC (EA/MeOH=5:1) showed that the reactant 7[PA1-13A-7] was consumed completely and new spots were formed. The reaction was filtrated and the solution was concentrated in vacuo. The residue was purified by silica gel chromatography (EA/MeOH=10:1) to give 8[PA1-13A-7] (N-(4-formylphenyl)-N-methyl-4-(4-methylpiperazin-1-yl)benzenesulfonamide) (1.20 g, 51.8% yield, 77.3% purity) as yellow oil. LCMS: m/z 374.0[M+H]⁺. ¹H NMR (CDCl₃ 400 MHz) δ 9.95 (s, 1H), 7.79 (d, J=8.4 Hz, 2H), 7.36-7.31 (m, 4H), 6.81-6.77 (m, 2H), 3.37-3.33 (m, 4H), 3.17 (s, 3H), 2.62-2.59 (m, 4H), 2.37 (s, 3H).

10[PA1-13A-7]: To a solution of 8[PA1-13A-7] (N-(4-formylphenyl)-N-methyl-4-(4-methylpiperazin-1-yl)benzenesulfonamide) (431 mg, 891 umol, 77.3% purity, 1 eq) in AcOH (8 mL) was added 9[PA1-13A-7] (2-amino-3-(6-fluoro-1H-indol-3-yl)propanoic acid) (200 mg, 891 umol, 99% purity, 1 eq). Note that 9[PA1-13A-7] is the same as compound 4[PA1-3] prepared in Example 2. Then the mixture was stirred at 120° C. for 2 h. LCMS showed that the reactant 9[PA1-13A-7] was consumed completely and desired product was detected. After cooling, the reaction was concentrated in vacuo to give 10[PA1-13A-7] (7-fluoro-1-[4-[methyl-[4-(4-methylpiperazin-1-yl)phenyl]sulfonyl-amino]phenyl]-2,3,4,9-tetrahydro-1H-pyrido[3,4-b]indole-3-carboxylic acid) (635 mg, crude) as a yellow oil, which was used directly for the next step without purification. LCMS: m/z 578.4[M+H]⁺.

11[PA1-13A-7]: A solution of 10[PA1-13A-7] (7-fluoro-1-[4-[methyl-[4-(4-methylpiperazin-1-yl)phenyl]sulfonyl-amino]phenyl]-2,3,4,9-tetrahydro-1H-pyrido[3,4-b]indole-3-carboxylic acid) (530 mg, 917 umol, 1 eq) in HCl/MeOH (4 M, 20 mL) was stirred at 25° C. for 12 h. LCMS and TLC (DCM/MeOH=10:1) showed the reactant 10[PA1-13A-7] was consumed completely and desired product was detected. The reaction was concentrated in vacuo and then poured into EA (30 mL) and Na₂CO₃ (sat.) was added to make pH=7-8, then extracted with EA (20 mL×3). The organic layer was combined and washed with brine (30 mL), dried over Na₂SO₄, filtered and concentrated in vacuo. The residue was purified by silica gel chromatography (EA/MeOH=10:1 to 5:1) to give 11[PA1-13A-7] (methyl 7-fluoro-1-[4-[methyl-[4-(4-methylpiperazin-1-yl)phenyl]sulfonyl-amino]phenyl]-2,3,4,9-tetrahydro-1H-pyrido[3,4-b]indole-3-carboxylate) (180 mg, 18.9% yield, 57% purity) as yellow oil. LCMS: m/z 592.1[M+H]⁻.

12[PA1-13A-7]: To a solution of 11[PA1-13A-7] (methyl 7-fluoro-1-[4-[methyl-[4-(4-methylpiperazin-1-yl)phenyl]sulfonyl-amino]phenyl]-2,3,4,9-tetrahydro-1H-pyrido[3,4-b]indole-3-carboxylate) (180 mg, 304 umol, 1 eq) in DMF (4 mL) was added PhI(OAc)₂ (294 mg, 913 umol, 3 eq) at 25° C. and then the mixture was stirred at 25° C. for 3 h. LCMS and TLC (DCM/MeOH=10:1) showed that the reactant 11[PA1-13A-7] was consumed completely and desired product was detected. The reaction was poured into water (20 mL) and extracted with EA (20 mL×2). The organic layers were combined and washed with brine (20 mL), dried over Na₂SO₄, filtered and concentrated in vacuo. The residue was purified by silica gel chromatography (EA/MeOH=10:1 to 3:1) to give 12[PA1-13A-7] (methyl 7-fluoro-1-[4-[methyl-[4-(4-methylpiperazin-1-yl)phenyl]sulfonyl-amino]phenyl]-9H-pyrido[3,4-b]indole-3-carboxylate) (80.0 mg, 28.7% yield, 64.1% purity) as yellow oil. LCMS: m/z 588.1[M+H]⁺.

PA1-13A-7: To a solution of 12[PA1-13A-7] (methyl 7-fluoro-1-[4-[methyl-[4-(4-methylpiperazin-1-yl)phenyl]sulfonyl-amino]phenyl]-9H-pyrido[3,4-b]indole-3-carboxylate) (80.0 mg, 87.3 umol, 64.1% purity, 1 eq) in MeOH (3 mL) and THF (3 mL) and H₂O (3 mL) was added NaOH (80.0 mg, 2.00 mmol, 22.9 eq) and then the mixture was stirred at 45° C. for 12 h. LCMS showed 12[PA1-13A-7] was consumed and the desired product was detected. The mixture was poured into water (10 mL), HCl (1 M) was added to make pH=5-6. Then the solution was extracted with EA (10 mL×3). The organic layers were combined and washed with brine (10 mL×3), dried over Na₂SO₄, filtered, and concentrated in vacuo. The residue was purified by Prep-HPLC 4 times to give PA1-13A-7 (7-fluoro-1-[4-[methyl-[4-(4-methylpiperazin-1-yl)phenyl]sulfonyl-amino]phenyl]-9H-pyrido[3,4-b]indole-3-carboxylic acid) (5.20 mg, 10.4% yield, 100% purity) as a yellow solid, confirmed by ¹H-NMR, LCMS, and HPLC. The Prep-HPLC was performed using the following four methods in sequential order: (1) Prep-HPLC (TFA): column: Phenomenex Luna C18 150*25 mm*10 um; mobile phase: [water (TFA)-ACN]; B %: 26%-46%, 10 min. (2) Prep-HPLC (TFA): column: Phenomenex Luna C18 150*25 mm*10 um; mobile phase: [water (TFA)-ACN]; B %: 28%-48%, 10 min. (3) Prep-HPLC (NH₃/H₂O): column: Phenomenex C18 75*30 mm*3 um; mobile phase: [water (ammonia hydroxide v/v)-ACN]; B %: 1%-31%, 7 min. (4) column: Phenomenex C18 75*30 mm*3 um; mobile phase: [water (ammonia hydroxide v/v)-ACN]; B %: 3%-33%, 7 min. LCMS: m/z 574.2[M+H]⁻. H NMR (DMSO-d₆ 400 MHz) δ 12.0 (s, 1H), 8.87-8.83 (m, 1H), 8.47-8.42 (m, 1H), 8.04-8.01 (m, 2H), 7.42-7.37 (m, 5H), 7.20-7.16 (m, 1H), 7.07-7.04 (m, 2H), 3.76-3.44 (m, 4H), 3.19 (s, 3H), 2.40-2.30 (m, 4H), 2.21 (s, 3H).

Using similar synthetic procedures as described in this example, various other compounds of the invention, including those set forth in Table 1, can be synthesized.

### Example 5

This example demonstrates compounds that were synthesized and tested for various properties.

The various properties tested in this example include solubility, AlphaLISA IC₅₀ (μM) values, fold change (WLC21), inhibition of UV-induced DDB2 degradation, EC₅₀ (μM) values for cell killing in CUL4-high MDA-MB-468 cells, EC₅₀ (μM) values for cell killing in CUL4-low T47D cells, K_{d} (μM) values (SPR), and K_{d} (μM) values (MST). The results are shown in Table 1.

Dynamic light scattering (DLS) (see Table 1, column 3) is a tool for studying the diffusion behavior of macromolecules in solution. To profile compound solubility, DLS assay was developed for measuring aggregation of small molecule compounds. Compound stock solution was prepared in DMSO. Dilution series were made for each compound in DMSO to ensure a final 1× assay concentration range of 200 μM to 0.78 μM. For example, if the final DMSO concentration was 1%, compounds were prepared at 100× concentrations ranging from 10 mM to 78 μM. Dilute compounds in PBS solution to final concentration. Place 60 μl/well in 96 well plates and 3 replicates for each sample. Data were collected using a DynaPro Plate Reader III (WYATT Technologies, Inc., USA). The instrument was adjusted to measure the optimal light scattering intensity according to the manufacturer's instructions. The normalized intensity (Cnt/s) ratio of 25 μM compound vs DMSO was set as the standard for compound solubility in table 1. The ratio over 2 means (−) insoluble. The ratio below 2 means (+) soluble and around 2 means (+/−).

AlphaLISA IC₅₀ (see Table 1, column 4) means the half maximal inhibitory concentration of compound that inhibits CUL4A and DDB1-BPB binding. Our lab established and optimized 384 well-based AlphaLISA in vitro binding assay for testing small molecule inhibitory activity. Small molecule libraries were screened for compounds that disrupt CUL4A-DDB1-BPB interaction using AlphaScreen detection beads (PerkinElmer, #AL112C and #6765300) following the manufacturer's protocol (https://www.perkinelmer.com/PDFs/downloads/GDE-Alphatech.pdf, hereby incorporated by reference in its entirety for all purposes). The 384-well assay plates (Greiner Bio-one, #781075) were read in Synergy Neo2 Hybrid Multi-Mode Reader (BioTek). The IC₅₀ was calculated by Prism 8 XY analysis nonlinear regression (curve fit-Log(inhibitor) vs response).

Cell-based assay for inhibition of UV-induced CUL4-dependent DDB2 degradation by small molecule CUL4 inhibitors (see Table 1, column 5). Briefly, MEF-F-DDB2 cells were seeded in 6-well plates. DMSO or indicated concentrations of compounds were added to MEF-F-DDB2 cells growing in serum-free medium. MEF-F-DDB2 cells were washed once with PBS and subsequently irradiated with a Philips TUV lamp (predominantly 254 nm) at a dose rate of 0.2 W/m². DMSO or compounds were added to MEF-F-DDB2 cells, and then cells were harvested. The cells were lysed, and equal amounts of proteins were resolved on SDS-PAGE gel and Flag-DDB2 levels were detected using anti-DDB2 (D4C4) rabbit antibody (Cell signaling, #5416S). α-tubulin (Proteintech, #66031-1-Ig) was used as an internal loading control. (+) means that the normalized DDB2/α-tubulin ratio is higher in the compound treated sample than DMSO treated sample. (−) means that no change happens between compound treated sample with control sample.

EC₅₀ refers to concentration of compound that induces 50% killing of CUL4A^{high} MDA-MB-468 cells (see Table 1, column 6). CUL4A^{high} MDA-MB-468 breast cancer cells were seeded in 96 well plates. After 24 hours, cells were treated with DMSO or compounds. Change fresh medium and compounds. After 8 days treatment, cell viability was examined by using CellTiter Glo kit (Promega #7570) following the manufacturer's guidelines (https://www.promega.com/-/media/files/resources/protocols/technicalbulletins/0/celltiter-glo-luminescent-cell-viability-assay protocol.pdf?rev=0d95c232094b41daa06d0c110d1d916f&sc_lang=en, hereby incorporated by reference in its entirety for all purposes). EC₅₀ concentration was calculated by Prism 8 XY analysis nonlinear regression (curve fit-Log(inhibitor) vs response). Lower EC₅₀ indicates high cell killing potential in CUL4^{high} MDA-MB-468 cells.

EC₅₀ indicates compound concentration can cause 50% cell killing in CUL4° ^{w} T47D cells (see Table 1, column 7). CUL4A^{low} T47D breast cancer cells were seeded in 96 well plate. After 24 hours, cells were treated with DMSO, or 0 to 20 μM compound respectively. Change fresh medium and compounds every 48 hours. After 8 days treatment, cell viability was examined by using CellTiter Glo kit (Promega #7570) following the manufacturer's guidelines (https://www.promega.com/-/media/files/resources/protocols/technical-bulletins/0/celltiter-glo-luminescent-cell-viability-assay-protocol.pdf?rev=0d95c232094b41daa06d0c110d1d916f&sc_lang=en, hereby incorporated by reference in its entirety for all purposes). EC₅₀ concentration was calculated by Prism 8 XY analysis nonlinear regression (curve fit-Log(inhibitor) vs response). EC₅₀ indicates cell killing potential in CUL4 low T47D cell line and the EC50 difference between CUL4^{high} and CUL4^{low} cells indicates the small molecule cause specific cell killing.

K_{d} represents dissociation constant, which measures the affinity of small molecule binding to the scaffold DDB1-BPB by Surface Plasmon Resonance (SPR) (https://www.bio-rad.com/webroot/web/pdf/lsr/literature/Bulletin_6414.pdf, hereby incorporated by reference in its entirety) (see Table 1, column 8). To assess the interaction between DDB1-BPB and compounds, a multi-parametric SPR instrument (BioRad ProteON XPR360) was used. DDB1-BPB was immobilized onto the GLH sensor surface in a variety of ways as follows: flow channels were activated in parallel with 1/20 diluted EDC/SNHS. DDB1-BPB was diluted to 0.1 mg/mL in sodium acetate pH 4.5 and directly coupled onto channel for 4 min. Excess reactive esters were blocked with a 5-min injection of 1 M ethanolamine. Mean immobilization levels were 17,000 RU with <2% variation along a strip. One channel was left unmodified to provide an additional reference surface. Next, the binding kinetics of compounds on surfaces was determined in a single injection using a “one-shot” kinetic mode. Typically, compound was prepared as a twofold serial dilution (0, 3.125, 6.25, 12.5, 25, 50 μM) and injected for 60 s at 100 L/min. Dissociation was monitored for 800 s. Surfaces were regenerated with multiple 30-s pulses of 60 mM phosphoric acid so that the experiment could be reproduced. All resulting sensor grams were collected and analyzed using the ProteOn manager software.

K_{d} represents dissociation constant, which measures the affinity of small molecule binding to the scaffold DDB1-BPB by Microscale Thermophoresis (MST) (see Table 1, column 9). MST experiments were performed on a Monolith NT 115 system (Nano Temper Technologies, Munich, Germany) using 100% LED and 60% IR-laser power (https://physiology.case.edu/media/eq manuals/eq_manual_nano-temper_mst_starting_guide.pdf, hereby incorporated by reference in its entirety for all purposes). The labeling of DDB1-BPB (10 M) was performed in labeling buffer with NHS-Red reactive dye (30 μM) (Nanotemper), which reacts efficiently with the primary amines of the proteins to form a stable dye protein conjugates. The labeling reaction was carried out at RT. A 16-point serial dilution (1:1) was prepared for compound at the final concentration ranged from 200 M to 3 nM in PBS containing 0.05% tween. The samples were filled into Premium capillaries and measurements were conducted at 25° C. An equation implemented by the software MO-S002 MO Affinity Analysis, provided by the manufacturer, was used for fitting normalized fluorescence values at different concentrations of ligands.

TABLE 1
Compounds corresponding to aspects of the invention.

| | | Solubility | AlphaLisa | Degradation | EC₅₀ (μM) | EC₅₀ (μM) | | |
|---|---|---|---|---|---|---|---|---|
| Ref. | Structure | (DLS Assay) | IC₅₀ (UM) | Inhibition* | CUL4-High Cells† | CUL4-Low Cells‡ | K_{d} (μM) (SPR) | K_{d} (μM) (MST) |
| PA2 | 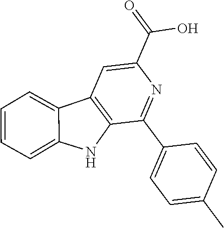 [SMILES: Cc1ccc(-c2nc(C(=O)O)cc3c2[nH]c2ccccc23)cc1] | -(insoluble) | 3.43 ± 1.57 | + | N/A | N/A | N/A | N/A |
| PA3 | 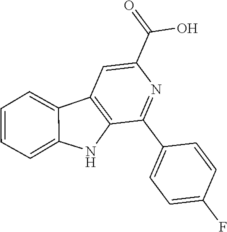 [SMILES: O=C(O)c1cc2c([nH]c3ccccc32)c(-c2ccc(F)cc2)n1] | +/− | 1.41 ± 0.31 | + | N/A | N/A | N/A | N/A |
| PA5 | 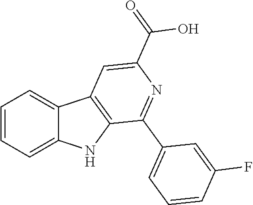 [SMILES: O=C(O)c1cc2c([nH]c3ccccc32)c(-c2cccc(F)c2)n1] | -(insoluble) | 2.34 ± 0.77 | ++ | N/A | N/A | N/A | N/A |
| PA6 | 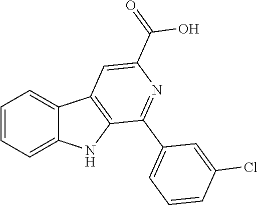 [SMILES: O=C(O)c1cc2c([nH]c3ccccc32)c(-c2cccc(Cl)c2)n1] | + | 3.97 ± 0.79 | ++ | N/A | N/A | N/A | N/A |
| PA7 | 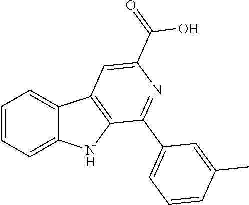 [SMILES: Cc1cccc(-c2nc(C(=O)O)cc3c2[nH]c2ccccc23)c1] | -(insoluble) | 10.18 ± 3.33 | ++ | N/A | N/A | N/A | N/A |
| PA8 | 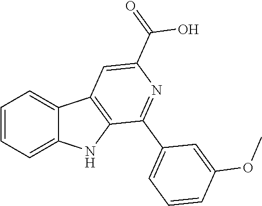 [SMILES: COc1cccc(-c2nc(C(=O)O)cc3c2[nH]c2ccccc23)c1] | -(insoluble) | 7.75 ± 1.82 | ++ | N/A | N/A | N/A | N/A |
| PA14 | 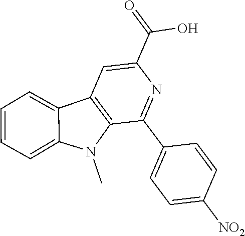 [SMILES: Cn1c2ccccc2c2cc(C(=O)O)nc(-c3ccc([N+](=O)[O-])cc3)c21] | + | 32.2 ± 7.89 | +/− | N/A | N/A | N/A | N/A |
| PA19 | 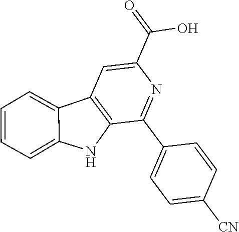 [SMILES: N#Cc1ccc(-c2nc(C(=O)O)cc3c2[nH]c2ccccc23)cc1] | -(insoluble) | 2.01 ± 0.44 | + | N/A | N/A | N/A | N/A |
| PA9 | 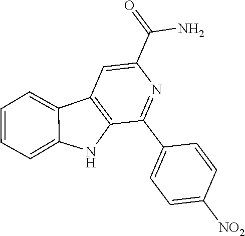 [SMILES: NC(=O)c1cc2c([nH]c3ccccc32)c(-c2ccc([N+](=O)[O-])cc2)n1] | -(insoluble) | Neg | + | N/A | N/A | N/A | N/A |
| PA25 | 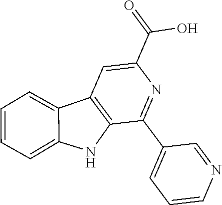 [SMILES: O=C(O)c1cc2c([nH]c3ccccc32)c(-c2cccnc2)n1] | + | 2.52 ± 0.54 | + | N/A | N/A | N/A | N/A |
| PA26 | 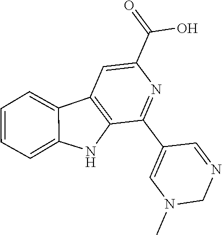 [SMILES: CN1C=C(c2nc(C(=O)O)cc3c2[nH]c2ccccc23)C=NC1] | N/A | 4.81 ± 0.96 | N/A | N/A | N/A | N/A | N/A |
| PA30 | 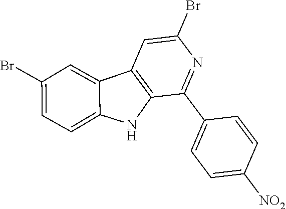 [SMILES: O=[N+]([O-])c1ccc(-c2nc(Br)cc3c2[nH]c2ccc(Br)cc23)cc1] | N/A | Neg | N/A | N/A | N/A | N/A | N/A |
| PA21 | 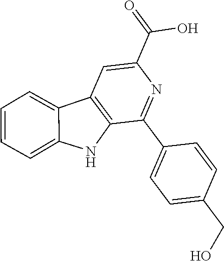 [SMILES: O=C(O)c1cc2c([nH]c3ccccc32)c(-c2ccc(CO)cc2)n1] | N/A | 3.40 ± 0.88 | N/A | N/A | N/A | N/A | N/A |
| PA41 | 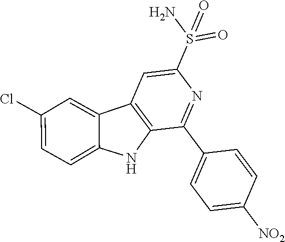 [SMILES: NS(=O)(=O)c1cc2c([nH]c3ccc(Cl)cc32)c(-c2ccc([N+](=O)[O-])cc2)n1] | + | Neg | N/A | N/A | N/A | N/A | N/A |
| PA32 | 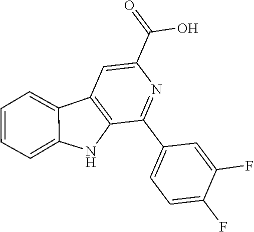 [SMILES: O=C(O)c1cc2c([nH]c3ccccc32)c(-c2ccc(F)c(F)c2)n1] | N/A | 3.54 ± 0.68 | N/A | N/A | N/A | N/A | N/A |
| PA33 | 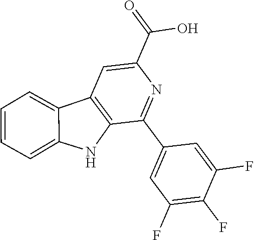 [SMILES: O=C(O)c1cc2c([nH]c3ccccc32)c(-c2cc(F)c(F)c(F)c2)n1] | -(insoluble) | 2.0 ± 0.38 | N/A | N/A | N/A | N/A | N/A |
| PA35 | 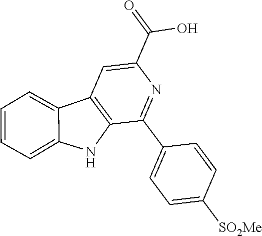 [SMILES: CS(=O)(=O)c1ccc(-c2nc(C(=O)O)cc3c2[nH]c2ccccc23)cc1] | + | 1.06 ± 0.51 | +/− | >100 | >100 | 5.58 | N/A |
| PA36 | 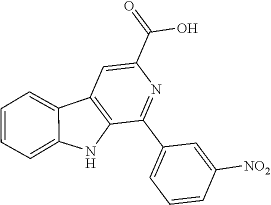 [SMILES: O=C(O)c1cc2c([nH]c3ccccc32)c(-c2cccc([N+](=O)[O-])c2)n1] | N/A | 7.10 ± 0.61 | N/A | N/A | N/A | N/A | N/A |
| PA22 | 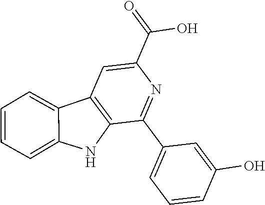 [SMILES: O=C(O)c1cc2c([nH]c3ccccc32)c(-c2cccc(O)c2)n1] | N/A | 14.67 ± 3.25 | N/A | N/A | N/A | N/A | N/A |
| PA42 | 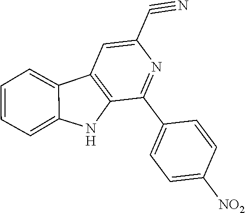 [SMILES: N#Cc1cc2c([nH]c3ccccc32)c(-c2ccc([N+](=O)[O-])cc2)n1] | + | Neg | N/A | 73.12 | >100 | N/A | N/A |
| PA20 | 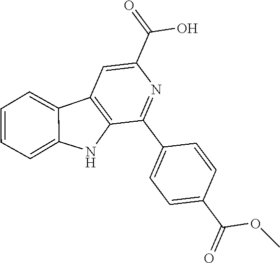 [SMILES: COC(=O)c1ccc(-c2nc(C(=O)O)cc3c2[nH]c2ccccc23)cc1] | + | 2.09 ± 1.26 | + | 91.3 | >100 | N/A | N/A |
| PA38 | 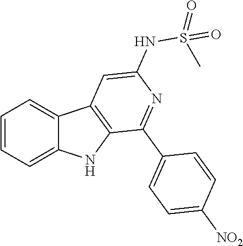 [SMILES: CS(=O)(=O)Nc1cc2c([nH]c3ccccc32)c(-c2ccc([N+](=O)[O-])cc2)n1] | N/A | Neg | N/A | N/A | N/A | N/A | N/A |
| PA23 | 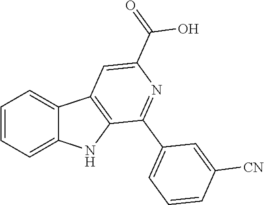 [SMILES: N#Cc1cccc(-c2nc(C(=O)O)cc3c2[nH]c2ccccc23)c1] | N/A | 2.38 ± 0.9 | N/A | N/A | N/A | N/A | N/A |
| PA40 | 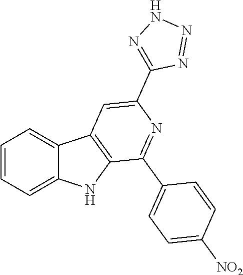 [SMILES: O=[N+]([O-])c1ccc(-c2nc(-c3nn[nH]n3)cc3c2[nH]c2ccccc23)cc1] | -(insoluble) | 12.9 ± 7.44 | N/A | 23.5 | 54.7 | N/A | N/A |
| PA27 | 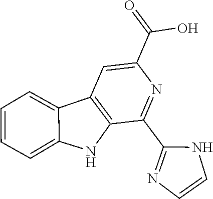 [SMILES: O=C(O)c1cc2c([nH]c3ccccc32)c(-c2ncc[nH]2)n1] | N/A | 23.12 ± 4.02 | N/A | N/A | N/A | N/A | N/A |
| PA29 | 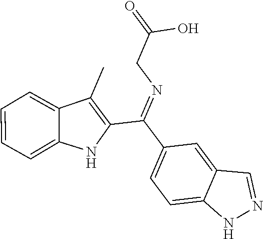 [SMILES: Cc1c(/C(=N\CC(=O)O)c2ccc3[nH]ncc3c2)[nH]c2ccccc12] | N/A | 3.79 ± 1.02 | N/A | N/A | N/A | N/A | N/A |
| PA48 | 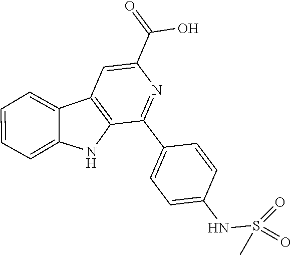 [SMILES: CS(=O)(=O)Nc1ccc(-c2nc(C(=O)O)cc3c2[nH]c2ccccc23)cc1] | N/A | 1.26 ± 0.45 | + | 84.09 | >100 | N/A | N/A |
| PA57 | 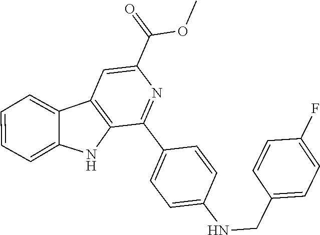 [SMILES: COC(=O)c1cc2c([nH]c3ccccc32)c(-c2ccc(NCc3ccc(F)cc3)cc2)n1] | -(insoluble) | Neg | ++ | 1.08 | 1.6 | N/A | Insoluble |
| PA28 | 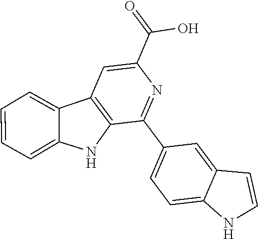 [SMILES: O=C(O)c1cc2c([nH]c3ccccc32)c(-c2ccc3[nH]ccc3c2)n1] | N/A | 34.82 ± 8.75 | N/A | N/A | N/A | N/A | N/A |
| PA59 | 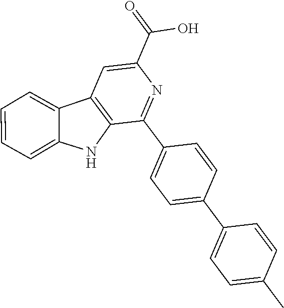 [SMILES: Cc1ccc(-c2ccc(-c3nc(C(=O)O)cc4c3[nH]c3ccccc34)cc2)cc1] | N/A | 25.04 ± 12.47 | ++ | N/A | N/A | N/A | N/A |
| PA60 | 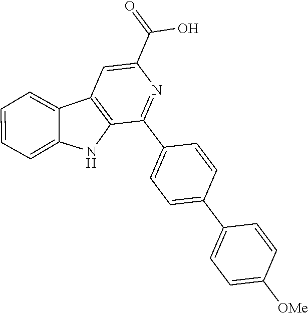 [SMILES: COc1ccc(-c2ccc(-c3nc(C(=O)O)cc4c3[nH]c3ccccc34)cc2)cc1] | N/A | 17.47 ± 6.39 | − | N/A | N/A | N/A | N/A |
| PA61 | 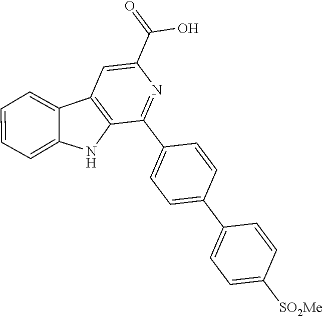 [SMILES: CS(=O)(=O)c1ccc(-c2ccc(-c3nc(C(=O)O)cc4c3[nH]c3ccccc34)cc2)cc1] | N/A | 3.86 ± 0.59 | ++ | N/A | N/A | N/A | N/A |
| PA64 | 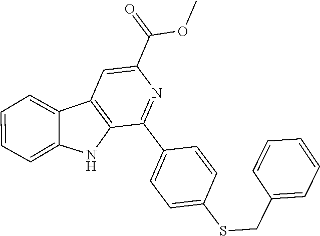 [SMILES: COC(=O)c1cc2c([nH]c3ccccc32)c(-c2ccc(SCc3ccccc3)cc2)n1] | N/A | Neg | N/A | 22.8 | >20 | N/A | Insoluble |
| PA66 | 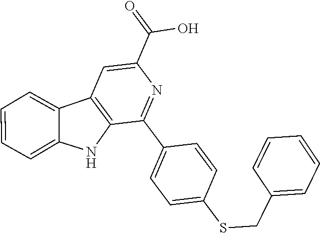 [SMILES: O=C(O)c1cc2c([nH]c3ccccc32)c(-c2ccc(SCc3ccccc3)cc2)n1] | N/A | 5.99 ± 0.43 | N/A | 4.41 | 4 | N/A | 59.6 |
| PA68 | 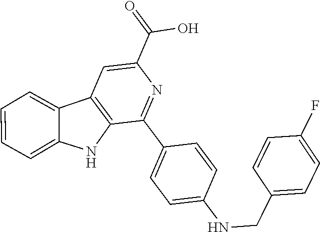 [SMILES: O=C(O)c1cc2c([nH]c3ccccc32)c(-c2ccc(NCc3ccc(F)cc3)cc2)n1] | + | 2.33 ± 0.86 | ++ | 9.12 | 10.95 | 13.2 | Insoluble |
| PA43 | 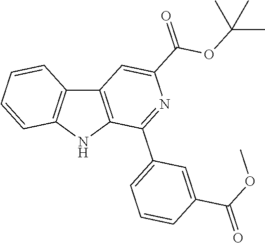 [SMILES: COC(=O)c1cccc(-c2nc(C(=O)OC(C)(C)C)cc3c2[nH]c2ccccc23)c1] | -(insoluble) | Neg | +++ | 28.33 | 48.2 | no binding | N/A |
| PA49 | 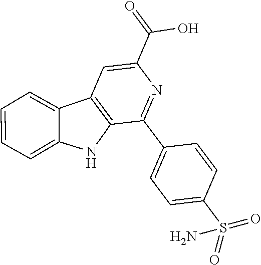 [SMILES: NS(=O)(=O)c1ccc(-c2nc(C(=O)O)cc3c2[nH]c2ccccc23)cc1] | N/A | 0.96 ± 0.096 | ++ | N/A | N/A | N/A | N/A |
| PA54 | 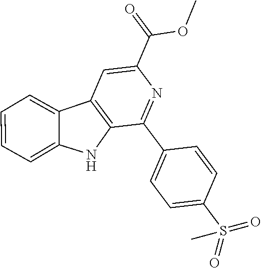 [SMILES: COC(=O)c1cc2c([nH]c3ccccc32)c(-c2ccc(S(C)(=O)=O)cc2)n1] | -(insoluble) | Neg | + | >100 | >100 | N/A | N/A |
| PA58 | 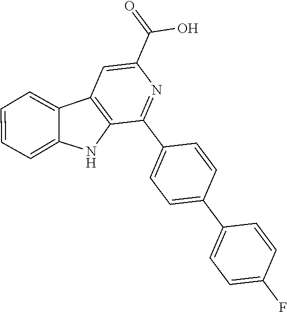 [SMILES: O=C(O)c1cc2c([nH]c3ccccc32)c(-c2ccc(-c3ccc(F)cc3)cc2)n1] | N/A | 5.50 ± 1.89 | +++ | N/A | N/A | N/A | N/A |
| PA65 | 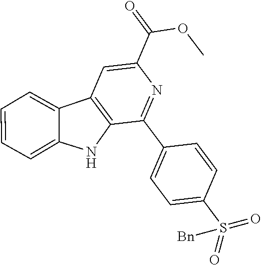 [SMILES: COC(=O)c1cc2c([nH]c3ccccc32)c(-c2ccc(S(=O)(=O)Cc3ccccc3)cc2)n1] | -(insoluble) | Neg | ++ | 18.13 | 44.3 | N/A | N/A |
| PA67 | 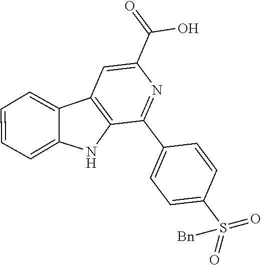 [SMILES: O=C(O)c1cc2c([nH]c3ccccc32)c(-c2ccc(S(=O)(=O)Cc3ccccc3)cc2)n1] | + | 1.22 ± 0.35 | ++ | 53.8 | >100 | N/A | N/A |
| PA24 | 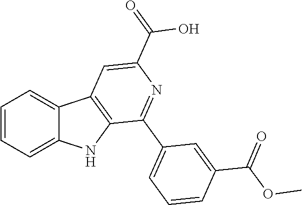 [SMILES: COC(=O)c1cccc(-c2nc(C(=O)O)cc3c2[nH]c2ccccc23)c1] | N/A | 11.97 | + | N/A | N/A | N/A | N/A |
| PA45 | 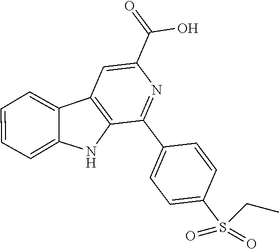 [SMILES: CCS(=O)(=O)c1ccc(-c2nc(C(=O)O)cc3c2[nH]c2ccccc23)cc1] | N/A | 0.94 ± 0.24 | ++ | N/A | N/A | N/A | N/A |
| PA50 | 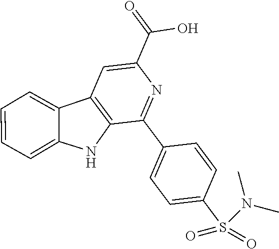 [SMILES: CN(C)S(=O)(=O)c1ccc(-c2nc(C(=O)O)cc3c2[nH]c2ccccc23)cc1] | N/A | 0.81 ± 0.21 | ++ | N/A | N/A | N/A | N/A |
| PA53 | 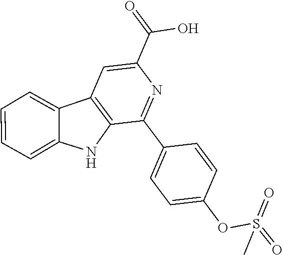 [SMILES: CS(=O)(=O)Oc1ccc(-c2nc(C(=O)O)cc3c2[nH]c2ccccc23)cc1] | N/A | 1.31 ± 0.24 | +/− | 27.51 | 69.5 | N/A | N/A |
| PA62 | 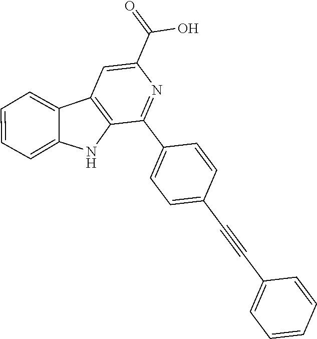 [SMILES: O=C(O)c1cc2c([nH]c3ccccc32)c(-c2ccc(C#Cc3ccccc3)cc2)n1] | N/A | 36.5 ± 9.9 | N/A | N/A | N/A | N/A | N/A |
| PA63 | 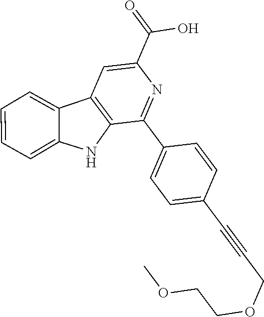 [SMILES: COCCOCC#Cc1ccc(-c2nc(C(=O)O)cc3c2[nH]c2ccccc23)cc1] | N/A | 5.05 ± 0.2 | N/A | N/A | N/A | N/A | N/A |
| PA70 | 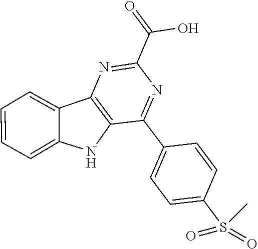 [SMILES: CS(=O)(=O)c1ccc(-c2nc(C(=O)O)nc3c2[nH]c2ccccc23)cc1] | N/A | 9.04 ± 0.89 | N/A | N/A | N/A | N/A | N/A |
| PA72 | 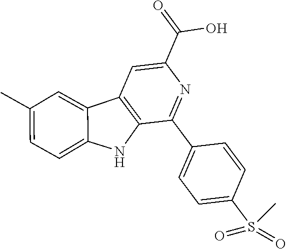 [SMILES: Cc1ccc2[nH]c3c(-c4ccc(S(C)(=O)=O)cc4)nc(C(=O)O)cc3c2c1] | +/− | 0.78 ± 0.02 | − | N/A | N/A | 4.57 | N/A |
| PA71 | 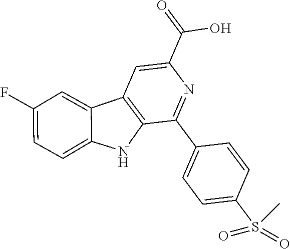 [SMILES: CS(=O)(=O)c1ccc(-c2nc(C(=O)O)cc3c2[nH]c2ccc(F)cc23)cc1] | -(insoluble) | 0.36 ± 0.04 | − | N/A | N/A | N/A | N/A |
| PA77 | 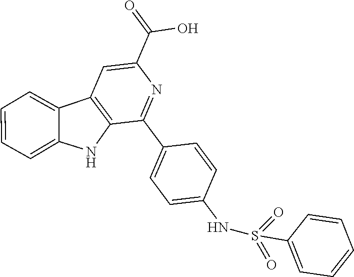 [SMILES: O=C(O)c1cc2c([nH]c3ccccc32)c(-c2ccc(NS(=O)(=O)c3ccccc3)cc2)n1] | N/A | 1.52 ± 0.19 | N/A | N/A | N/A | N/A | N/A |
| PA78 | 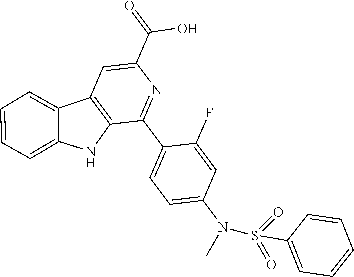 [SMILES: CN(c1ccc(-c2nc(C(=O)O)cc3c2[nH]c2ccccc23)c(F)c1)S(=O)(=O)c1ccccc1] | +/− | 0.84 ± 0.27 | +++ | 46.5 | >100 | 3.44 | N/A |
| PA73 | 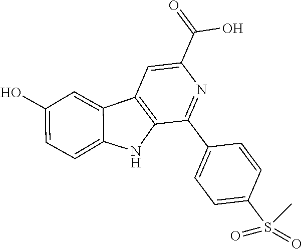 [SMILES: CS(=O)(=O)c1ccc(-c2nc(C(=O)O)cc3c2[nH]c2ccc(O)cc23)cc1] | +/− | 0.73 ± 0.35 | ++ | >100 | >100 | 7.34 | N/A |
| PA76 | 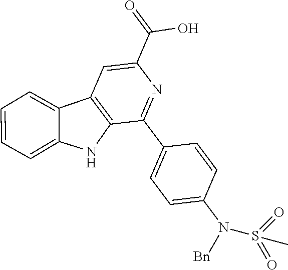 [SMILES: CS(=O)(=O)N(Cc1ccccc1)c1ccc(-c2nc(C(=O)O)cc3c2[nH]c2ccccc23)cc1] | + | 1.52 ± 1.13 | + | 28.95 | >100 | 4.54 | N/A |
| PA74 | 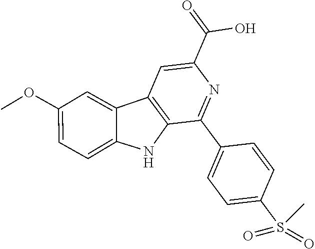 [SMILES: COc1ccc2[nH]c3c(-c4ccc(S(C)(=O)=O)cc4)nc(C(=O)O)cc3c2c1] | -(insoluble) | 1.83 ± 0.09 | + | N/A | N/A | N/A | N/A |
| PA75 | 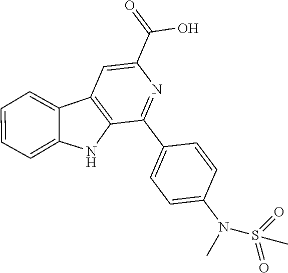 [SMILES: CN(c1ccc(-c2nc(C(=O)O)cc3c2[nH]c2ccccc23)cc1)S(C)(=O)=O] | N/A | 1.39 ± 0.43 | N/A | 38.71 | 64 | N/A | N/A |
| PA55 | 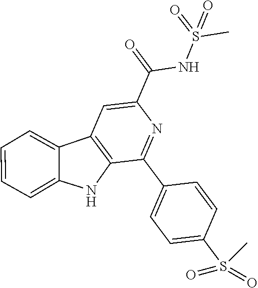 [SMILES: CS(=O)(=O)NC(=O)c1cc2c([nH]c3ccccc32)c(-c2ccc(S(C)(=O)=O)cc2)n1] | + | 11.1 ± 2.71 | − | 30.68 | 53.8 | N/A | N/A |
| PA93 | 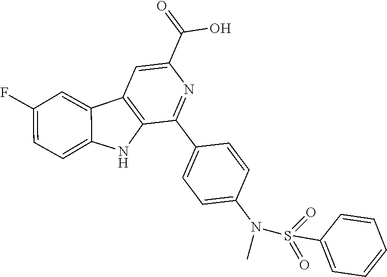 [SMILES: CN(c1ccc(-c2nc(C(=O)O)cc3c2[nH]c2ccc(F)cc23)cc1)S(=O)(=O)c1ccccc1] | -(insoluble) | 0.21 ± 0.1 | ++ | 11.07 | >100 | N/A | Insoluble |
| PA115 | 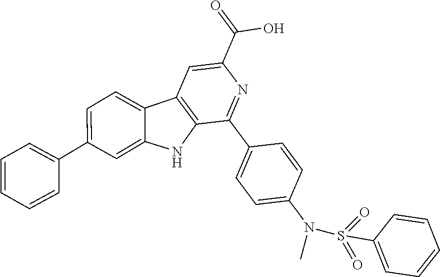 [SMILES: CN(c1ccc(-c2nc(C(=O)O)cc3c2[nH]c2cc(-c4ccccc4)ccc23)cc1)S(=O)(=O)c1ccccc1] | -(insoluble) | 17.4 ± 7.42 | +++ | 2.15 | 3.86 | N/A | 257 |
| PA106 | 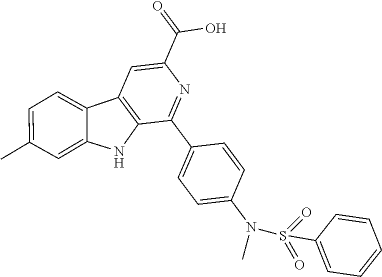 [SMILES: Cc1ccc2c(c1)[nH]c1c(-c3ccc(N(C)S(=O)(=O)c4ccccc4)cc3)nc(C(=O)O)cc12] | +/− | 7.73 ± 0 | +++ | 7.98 | >100 | N/A | 443 |
| PA117 | 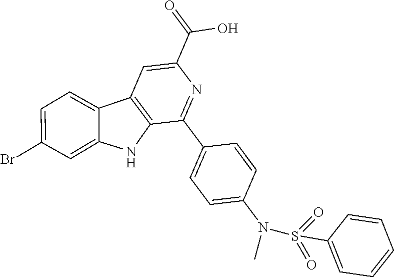 [SMILES: CN(c1ccc(-c2nc(C(=O)O)cc3c2[nH]c2cc(Br)ccc23)cc1)S(=O)(=O)c1ccccc1] | + | 2.5 ± 0 | +++ | 3.42 | 5.48 | N/A | 25.4 |
| PA99 | 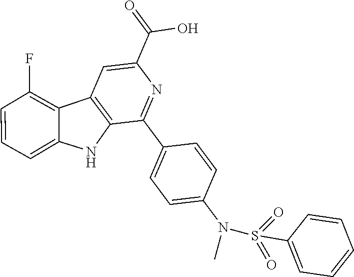 [SMILES: CN(c1ccc(-c2nc(C(=O)O)cc3c2[nH]c2cccc(F)c23)cc1)S(=O)(=O)c1ccccc1] | + | 0.38 | +++ | 1.18 | 8.27 | 2.13 | 2.97 |
| PA1-1 | 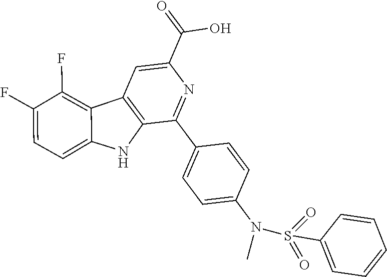 [SMILES: CN(c1ccc(-c2nc(C(=O)O)cc3c2[nH]c2ccc(F)c(F)c23)cc1)S(=O)(=O)c1ccccc1] | -(insoluble) | 0.17 | − | 15.24 | >100 | N/A | N/A |
| PA1-3 | 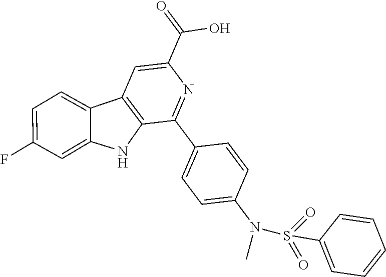 [SMILES: CN(c1ccc(-c2nc(C(=O)O)cc3c2[nH]c2cc(F)ccc23)cc1)S(=O)(=O)c1ccccc1] | + | 0.53 | +/− | 0.48 | 3.42 | N/A | 0.72 |
| PA1-4 | 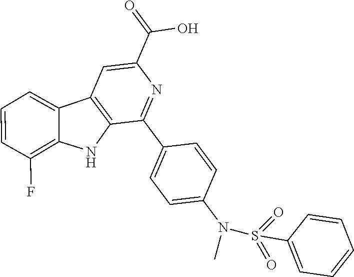 [SMILES: CN(c1ccc(-c2nc(C(=O)O)cc3c2[nH]c2c(F)cccc23)cc1)S(=O)(=O)c1ccccc1] | + | 8.88 | − | 6.9 | 10.93 | N/A | N/A |
| PA2-1 | 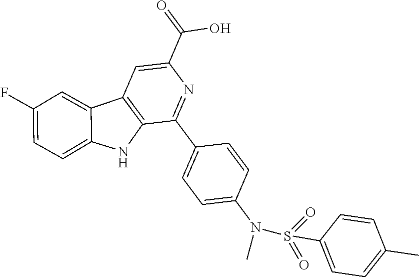 [SMILES: Cc1ccc(S(=O)(=O)N(C)c2ccc(-c3nc(C(=O)O)cc4c3[nH]c3ccc(F)cc34)cc2)cc1] | -(insoluble) | 0.3 | − | 9.32 | >100 | N/A | N/A |
| PA2-2 | 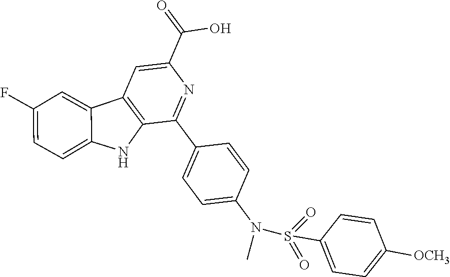 [SMILES: COc1ccc(S(=O)(=O)N(C)c2ccc(-c3nc(C(=O)O)cc4c3[nH]c3ccc(F)cc34)cc2)cc1] | -(insoluble) | 0.89 | − | 8.45 | >100 | N/A | N/A |
| PA2-3 | 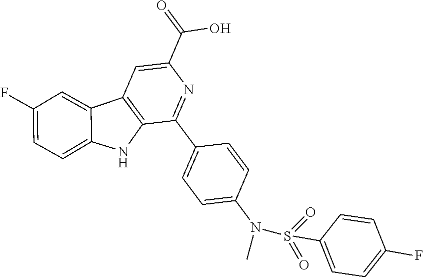 [SMILES: CN(c1ccc(-c2nc(C(=O)O)cc3c2[nH]c2ccc(F)cc23)cc1)S(=O)(=O)c1ccc(F)cc1] | -(insoluble) | 0.48 | − | 13.33 | >100 | N/A | N/A |
| PA2-4 | 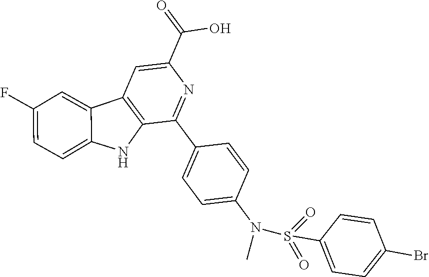 [SMILES: CN(c1ccc(-c2nc(C(=O)O)cc3c2[nH]c2ccc(F)cc23)cc1)S(=O)(=O)c1ccc(Br)cc1] | + | 0.75 | + | 4.49 | >100 | N/A | N/A |
| PA2-5 | 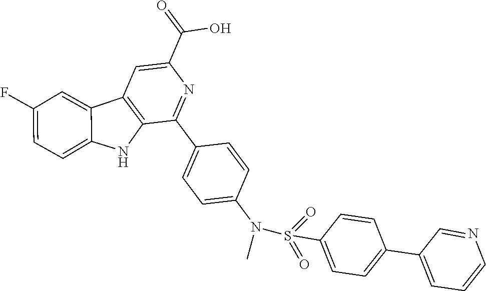 [SMILES: CN(c1ccc(-c2nc(C(=O)O)cc3c2[nH]c2ccc(F)cc23)cc1)S(=O)(=O)c1ccc(-c2cccnc2)cc1] | + | 2.03 | + | 2.23 | 2.96 | N/A | Insoluble |
| PA2-6 | 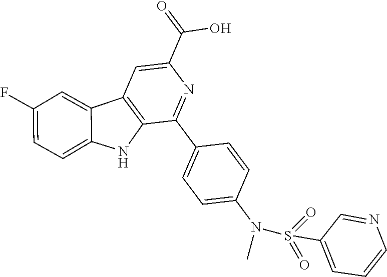 [SMILES: CN(c1ccc(-c2nc(C(=O)O)cc3c2[nH]c2ccc(F)cc23)cc1)S(=O)(=O)c1cccnc1] | -(insoluble) | 0.47 | − | 22.3 | >100 | N/A | 25.5 |
| PA93-1 | 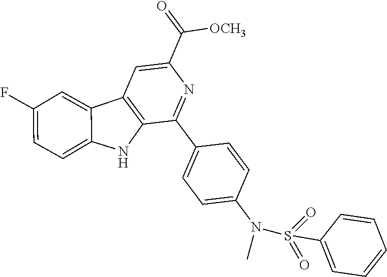 [SMILES: COC(=O)c1cc2c([nH]c3ccc(F)cc32)c(-c2ccc(N(C)S(=O)(=O)c3ccccc3)cc2)n1] | -(insoluble) | Neg | − | 21.8 | >100 | N/A | N/A |
| PA99-1 | 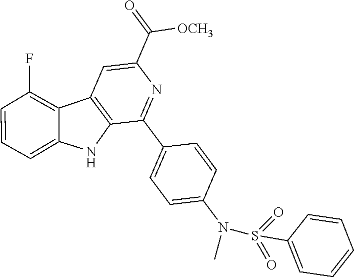 [SMILES: COC(=O)c1cc2c([nH]c3cccc(F)c32)c(-c2ccc(N(C)S(=O)(=O)c3ccccc3)cc2)n1] | -(insoluble) | Neg | + | 3.47 | >100 | N/A | 321 |
| PA1-6 | 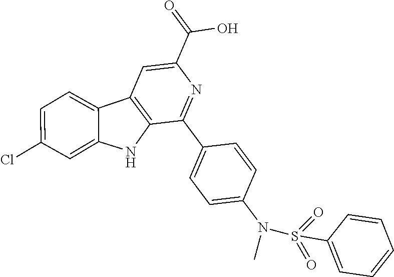 [SMILES: CN(c1ccc(-c2nc(C(=O)O)cc3c2[nH]c2cc(Cl)ccc23)cc1)S(=O)(=O)c1ccccc1] | + | 1.13 | ++ | 7.89 | N/A | N/A | N/A |
| PA1-9 | 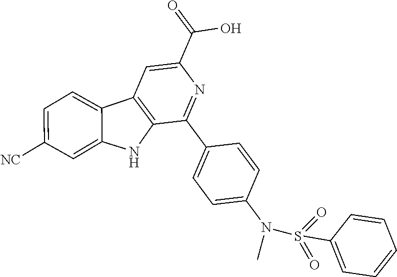 [SMILES: CN(c1ccc(-c2nc(C(=O)O)cc3c2[nH]c2cc(C#N)ccc23)cc1)S(=O)(=O)c1ccccc1] | -(insoluble) | 0.085 | + | 7.87 | N/A | N/A | N/A |
| PA1-5 | 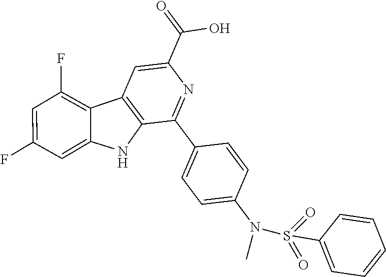 [SMILES: CN(c1ccc(-c2nc(C(=O)O)cc3c2[nH]c2cc(F)cc(F)c23)cc1)S(=O)(=O)c1ccccc1] | + | 0.22 | ++ | 10.65 | N/A | N/A | N/A |
| PA1-7 | 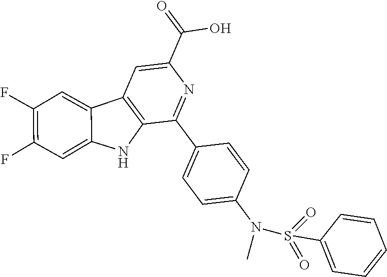 [SMILES: CN(c1ccc(-c2nc(C(=O)O)cc3c2[nH]c2cc(F)c(F)cc23)cc1)S(=O)(=O)c1ccccc1] | -(insoluble) | 0.127 | ++ | 8.65 | N/A | N/A | N/A |
| PA1-14 | 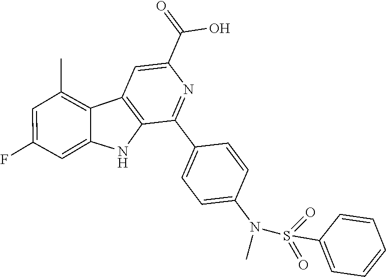 [SMILES: Cc1cc(F)cc2[nH]c3c(-c4ccc(N(C)S(=O)(=O)c5ccccc5)cc4)nc(C(=O)O)cc3c12] | + | 0.139 | +++ | 7.36 | N/A | N/A | N/A |
| PA1-9 ESTER | 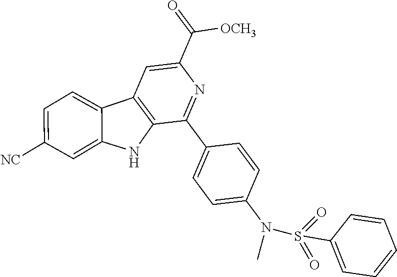 [SMILES: COC(=O)c1cc2c([nH]c3cc(C#N)ccc32)c(-c2ccc(N(C)S(=O)(=O)c3ccccc3)cc2)n1] | -(insoluble) | Neg | ++ | N/A | N/A | N/A | N/A |
| PA1-14 ESTER | 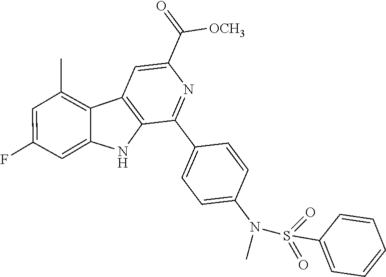 [SMILES: COC(=O)c1cc2c([nH]c3cc(F)cc(C)c32)c(-c2ccc(N(C)S(=O)(=O)c3ccccc3)cc2)n1] | -(insoluble) | Neg | − | N/A | N/A | N/A | N/A |
| PA1-11 | 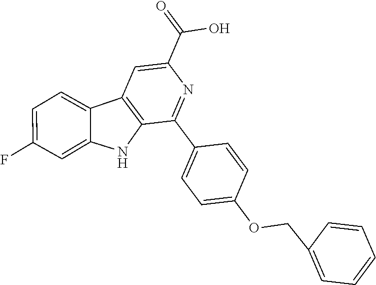 [SMILES: O=C(O)c1cc2c([nH]c3cc(F)ccc32)c(-c2ccc(OCc3ccccc3)cc2)n1] | -(insoluble) | 5.84 | − | 9.9 | N/A | N/A | N/A |
| PA1-11A | 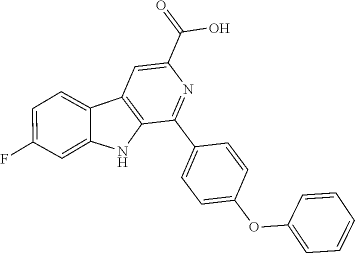 [SMILES: O=C(O)c1cc2c([nH]c3cc(F)ccc32)c(-c2ccc(Oc3ccccc3)cc2)n1] | N/A | N/A | − | 15.71 | N/A | N/A | N/A |
| PA1-11B | 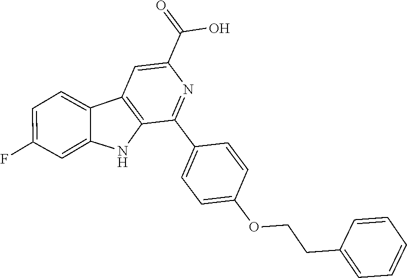 [SMILES: O=C(O)c1cc2c([nH]c3cc(F)ccc32)c(-c2ccc(OCCc3ccccc3)cc2)n1] | N/A | N/A | − | 15.16 | N/A | N/A | N/A |
| PA1-11C | 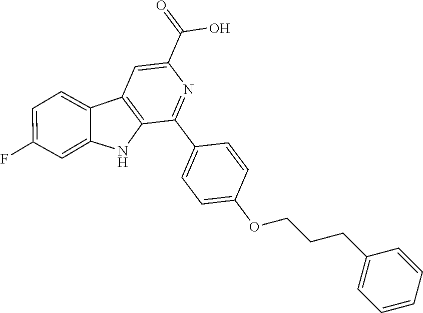 [SMILES: O=C(O)c1cc2c([nH]c3cc(F)ccc32)c(-c2ccc(OCCCc3ccccc3)cc2)n1] | N/A | N/A | − | 7.31 | N/A | N/A | N/A |
| PA1-11D | 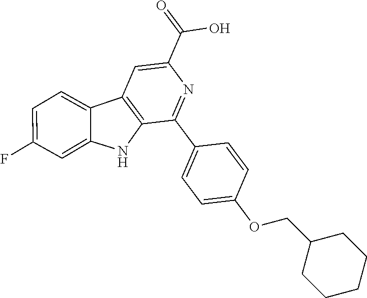 [SMILES: O=C(O)c1cc2c([nH]c3cc(F)ccc32)c(-c2ccc(OCC3CCCCC3)cc2)n1] | N/A | N/A | − | 9.51 | N/A | N/A | N/A |
| PA1-11E | 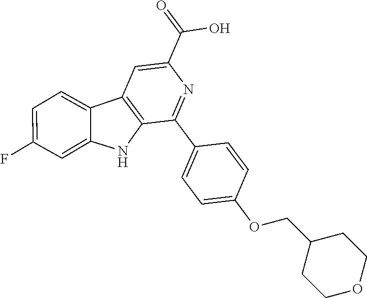 [SMILES: O=C(O)c1cc2c([nH]c3cc(F)ccc32)c(-c2ccc(OCC3CCOCC3)cc2)n1] | N/A | N/A | − | >20 | N/A | N/A | N/A |
| PA1-11F | 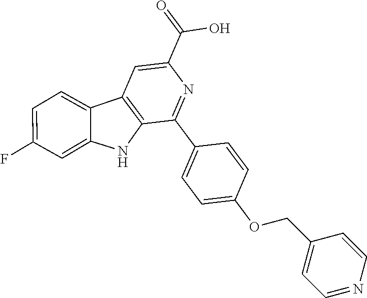 [SMILES: O=C(O)c1cc2c([nH]c3cc(F)ccc32)c(-c2ccc(OCc3ccncc3)cc2)n1] | N/A | N/A | − | >20 | N/A | N/A | N/A |
| PA1-11G | 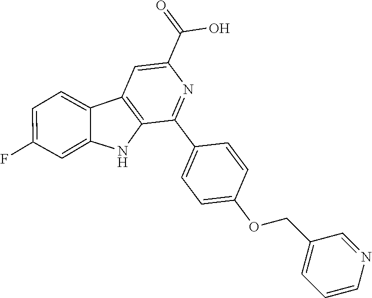 [SMILES: O=C(O)c1cc2c([nH]c3cc(F)ccc32)c(-c2ccc(OCc3cccnc3)cc2)n1] | N/A | N/A | − | >20 | N/A | N/A | N/A |
| PA1-11H | 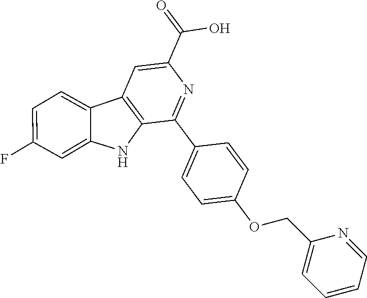 [SMILES: O=C(O)c1cc2c([nH]c3cc(F)ccc32)c(-c2ccc(OCc3ccccn3)cc2)n1] | -(insoluble) | N/A | − | N/A | N/A | N/A | N/A |
| PA1-11I | 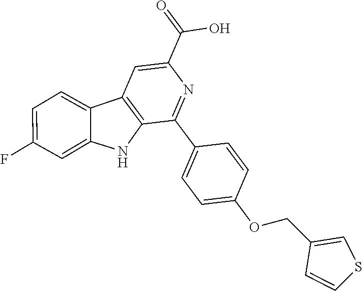 [SMILES: O=C(O)c1cc2c([nH]c3cc(F)ccc32)c(-c2ccc(OCc3ccsc3)cc2)n1] | N/A | N/A | − | 13.95 | N/A | N/A | N/A |
| PA1-11J | 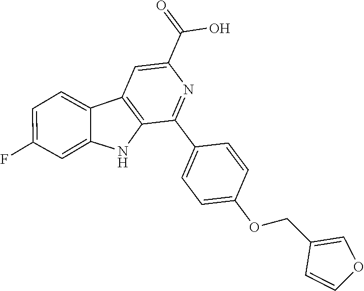 [SMILES: O=C(O)c1cc2c([nH]c3cc(F)ccc32)c(-c2ccc(OCc3ccoc3)cc2)n1] | N/A | N/A | − | N/A | N/A | N/A | N/A |
| PA1-11K | 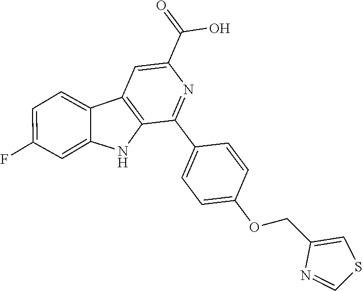 [SMILES: O=C(O)c1cc2c([nH]c3cc(F)ccc32)c(-c2ccc(OCc3cscn3)cc2)n1] | N/A | N/A | − | >20 | N/A | N/A | N/A |
| PA1-11L | 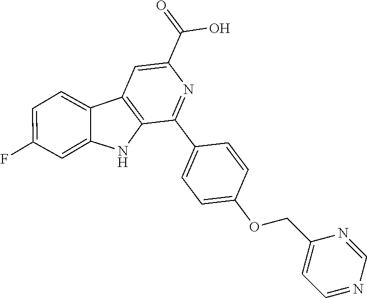 [SMILES: O=C(O)c1cc2c([nH]c3cc(F)ccc32)c(-c2ccc(OCc3ccncn3)cc2)n1] | N/A | N/A | − | N/A | N/A | N/A | N/A |
| PA1-11M | 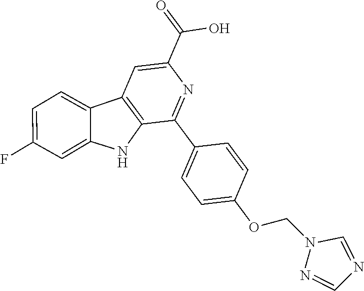 [SMILES: O=C(O)c1cc2c([nH]c3cc(F)ccc32)c(-c2ccc(OCn3cncn3)cc2)n1] | N/A | N/A | − | N/A | N/A | N/A | N/A |
| PA1-12 | 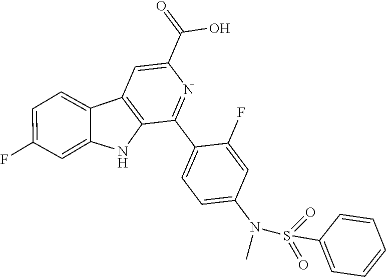 [SMILES: CN(c1ccc(-c2nc(C(=O)O)cc3c2[nH]c2cc(F)ccc23)c(F)c1)S(=O)(=O)c1ccccc1] | -(insoluble) | 0.75 | + | 7.37 | N/A | N/A | N/A |
| PA1-12A | 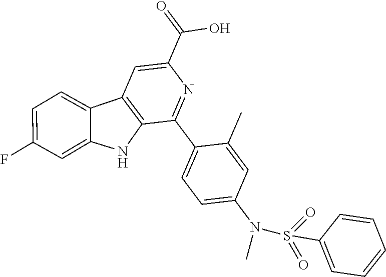 [SMILES: Cc1cc(N(C)S(=O)(=O)c2ccccc2)ccc1-c1nc(C(=O)O)cc2c1[nH]c1cc(F)ccc12] | N/A | 2.4 | − | 9.84 | N/A | N/A | N/A |
| PA1-13A-2 | 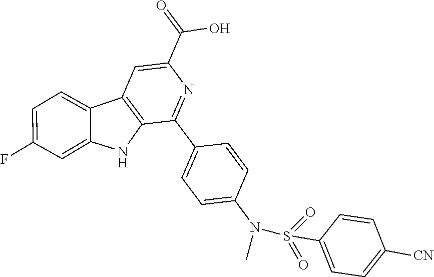 [SMILES: CN(c1ccc(-c2nc(C(=O)O)cc3c2[nH]c2cc(F)ccc23)cc1)S(=O)(=O)c1ccc(C#N)cc1] | N/A | 2.51 | − | 3.54 | N/A | N/A | N/A |
| PA1-13A-2 ESTER | 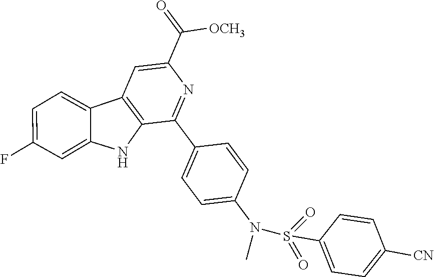 [SMILES: COC(=O)c1cc2c([nH]c3cc(F)ccc32)c(-c2ccc(N(C)S(=O)(=O)c3ccc(C#N)cc3)cc2)n1] | N/A | Neg | + | >20 | N/A | N/A | N/A |
| PA1-13A-3 | 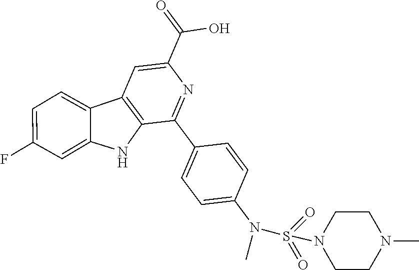 [SMILES: CN1CCN(S(=O)(=O)N(C)c2ccc(-c3nc(C(=O)O)cc4c3[nH]c3cc(F)ccc34)cc2)CC1] | -(insoluble) | 0.45 | − | 7.25 | N/A | N/A | N/A |
| PA1-13A-7 | 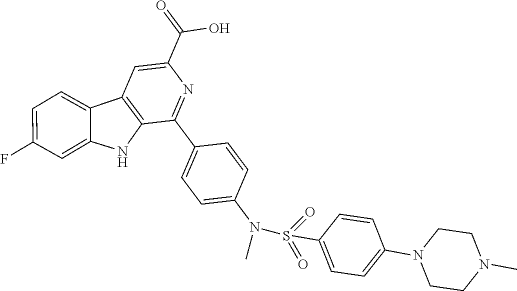 [SMILES: CN1CCN(c2ccc(S(=O)(=O)N(C)c3ccc(-c4nc(C(=O)O)cc5c4[nH]c4cc(F)ccc45)cc3)cc2)CC1] | + | 0.38 | − | 14.45 | N/A | N/A | N/A |

*Cell-based assay inhibition of UV-induced DDB2 degradation
†Cell killing in CUL4-high MDA-MB-468 cells
‡Cell killing in CUL4-low T47D cells

### Example 6

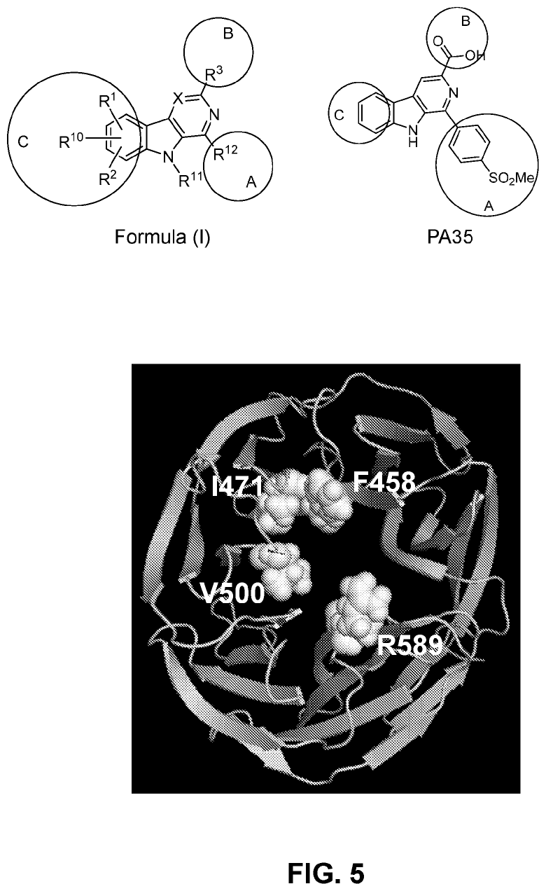

This example demonstrates the ability of compounds disclosed herein to bind to or otherwise interact with certain moieties of the beta-propeller (middle WD40 domain) of DDB1, as depicted in FIG. 5.

In particular, FIG. 5 shows a compound of formula (I) and a specific compound (PA35) falling within the scope of formula (I), both of which have portions of the molecule designated “A,” “B,” and “C.” FIG. 5 also shows the crystal structure of the BPB beta-propeller of DDB1. The “C” portion of the molecule is believed to bind to the hydrophobic pocket formed by F458-1471-V500, and positively charged R589 is believed to interact with the “B” portion of the molecule (e.g., carboxylate), as identified by molecular docking and molecular dynamic simulation. Without wishing to be bound by theory, it is believed that these amino acid residues play a role in PA35˜DDB1-BPB association, as determined using site-directed mutagenesis.

Without wishing to be bound by theory, it is believed that compounds disclosed herein (1) interact at the interface between W561 and CUL4A (L83, Y87) with the “A” portion of the disclosed compounds, (2) have a charge-charge interaction between the “B” portion of the disclosed compounds and R589/R639 of DDB1, (3) have hydrophobic interactions between the “C” portion of the disclosed compounds and the F458-1471-V500 hydrophobic pocket of the BPB beta-propeller of DDB1, or (4) any combination thereof. In addition, without wishing to be bound by theory, it is believed that W561 plays an important role in the interaction between DDB1 and CUL4. In this regard, it is believed that the “A” position of compounds disclosed herein (see, e.g., the phenyl moieties of the PA78 series) interacts with W561 and changes its orientation, thereby disrupting the interaction between DDB1 and CUL4.

Furthermore, substitution at the “A” position (see, e.g., the substitution on the phenyl moieties on compounds PA2-1 through PA2-6) is believed to play a role in solubility. As such, without wishing to be bound by theory, it is believed the solubility of the compounds disclosed herein can be tuned by substitution at this position.

### Example 7

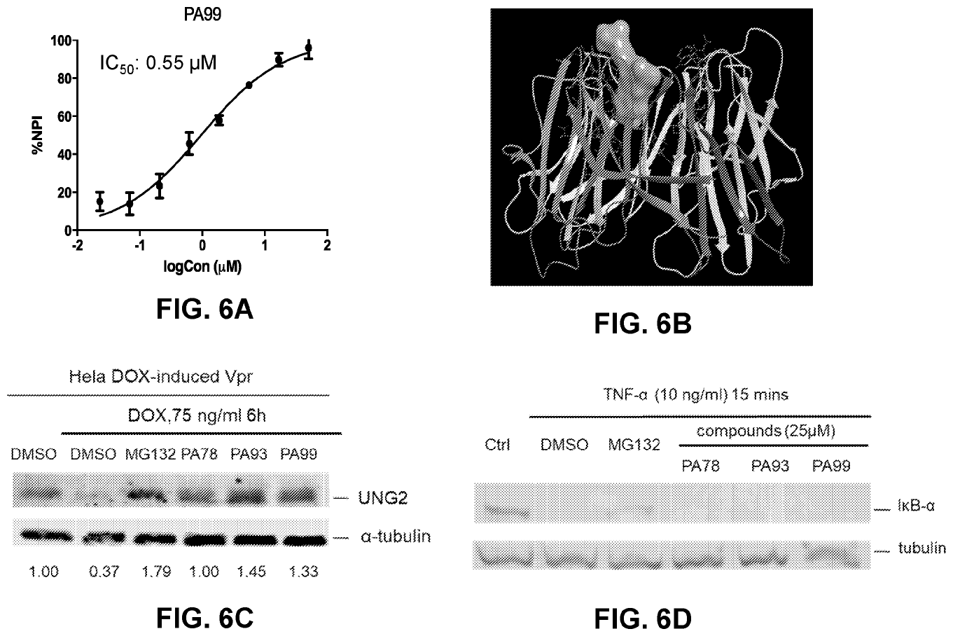

This example demonstrates disruption of CUL4A˜DDB1 binding using compound PA99 (see Table 1). The results are shown in FIGS. 6A-6D.

Measurements were performed according to the following procedure: for FIG. 6A, AlphaLISA (see Table 1, column 4); for FIG. 6B, molecular docking and molecular dynamic simulation; for FIGS. 6C-6D, Western blotting.

To characterize the binding mode of PA99 at DDB1, we carried out a molecular modeling work. We first docked the compound using the induced-fit docking program of Schrodinger¹ in a potential binding pocket on the surface of DDB1 facing CUL4. Based on three selected docking poses, we performed extensive molecular dynamics simulations with the DDB1-PA99 complex immersed in an explicit water box using Desmond², and collected multiple MD trajectories. Our converged simulation results revealed a stable binding mode of PA99 in the pocket in which PA99 is enclosed by residues F458, 1471 and V500.

FIG. 6C. Inhibition of DOX-induced Vpr-mediated CUL4-dependent UNG2 degradation by small molecule CUL4 inhibitors. In brief, Hela DOX-inducible-Vpr cells were seeded in 6-well plates. After 24 hours incubation, medium was changed to growth medium with 75 ng/ml doxycycline (DOX) overnight. On the third day, DMSO or indicated concentrations of compounds in serum-free medium containing DOX were added. The cells were lysed in buffer containing 150 mM NaCl, 1% SDS, 10 mM Tris-HCl (pH 6.8), 1× complete protease inhibitor cocktail and 10 U Benzo nuclease. Equal amounts of proteins were resolved on 9% SDS-PAGE gel and UNG2 levels were detected using anti-UNG2 (2C12) antibody (Origene #TA503563). α-tubulin (Proteintech, #66031-1-Ig) was used as an internal control. Normalized UNG2/α-tubulin ratio is higher in the compound treated sample than DMSO treated sample. (−) means that no change happens between compound treated sample with control sample.

FIG. 6D Western blotting of TNFα-induced CUL1/SCFPTCP-dependent IκBα degradation assay. Hela cells were seeded in 6-well plates. After 24 hours incubation, DMSO or indicated concentration of compounds were added with serum-free medium for 2 hours incubation. TNFα was added and cells were harvested, lysed in buffer containing 150 mM NaCl, 1% SDS, 10 mM Tris-HCl (pH 6.8), 1× complete protease inhibitor cocktail and 10 U Benzo nuclease. Equal amounts of proteins were resolved on 9% SDS-PAGE gel and IκBα levels were detected using anti-IκBα rabbit polyclonal antibody (Proteintech, 10268-1-AP). α-tubulin (Proteintech, #66031-1-Ig) was used as an internal loading control. TNFα-induced IκBα degradation was mediated by CUL1/SCF^{βTrCP} E3 ligase. This assay indicates that small molecule CUL4 inhibitors have no inhibition of CUL1/SCF^{βTrCP} E3 ligase when TNFα-induced IκBα degradation also happens in the compound treated sample.

References for Example 1, both of which are hereby incorporated by reference in their entireties for all purposes: (1) Sherman, W.; Day, T.; Jacobson, M. P.; Friesner, R. A.; Farid, R., Novel Procedure for Modeling Ligand/Receptor Induced Fit Effects. J Med Chem 2006, 49, 534-53; (2) Bowers, K. J.; Chow, E.; Xu, H.; Dror, R. O.; Eastwood, M. P.; Gregersen, B. A.; Klepeis, J. L.; Kolossvary, I.; Moraes, M. A.; Sacerdoti, F. D.; Salmon, J. K.; Shan, Y.; Shaw, D. E. Scalable Algorithms for Molecular Dynamics Simulations on Commodity Clusters. In ACM/IEEE SC 2006 Conference (SC'06), 2006; 2006; p 43.

### Example 8

This example demonstrates the identification of a compound disclosed herein (compound PA99) in xenograft tumors.

Measurements were performed via a pharmokinetic study demonstrating that PA99 was delivered/found in the breast cancer MDA-MB-468 xenograft tumors. Female athymic Nu/J nude mice, 6 weeks old were purchased from the Jackson Laboratory. Mice were housed in laminar air-flow cabinet under specific pathogen-free conditions. 5×10⁶ MDA-MB-468 cells in 100 μl diluted BD Matrigel™ Basement Membrane Matrix (BD Bioscience, Matrix:PBS is 1:1) were injected subcutaneously in each flank of mouse. We detected tumors once every four days by fine calipers measurement. When tumor size reached 100 mm³, mice were randomly grouped and treated with DMSO or 50 mg/kg PA99 compound. The stock solution of compound was 10 mg/ml, which formulated with 10% DMSO, 10% Tween-80 and 80% H₂O. PA99 was injected into mouse by intraperitoneal injection (5 l/g) three times per week. Mouse weights and tumor volumes were measured twice/week. For the pharmacokinetic study, samples were collected after compound injection (30 mins, 1 h, 3 h, 6 h). Four tumor samples were collected at each time point for mass spectrometry analysis to determine the compound concentrations in tumor tissues.

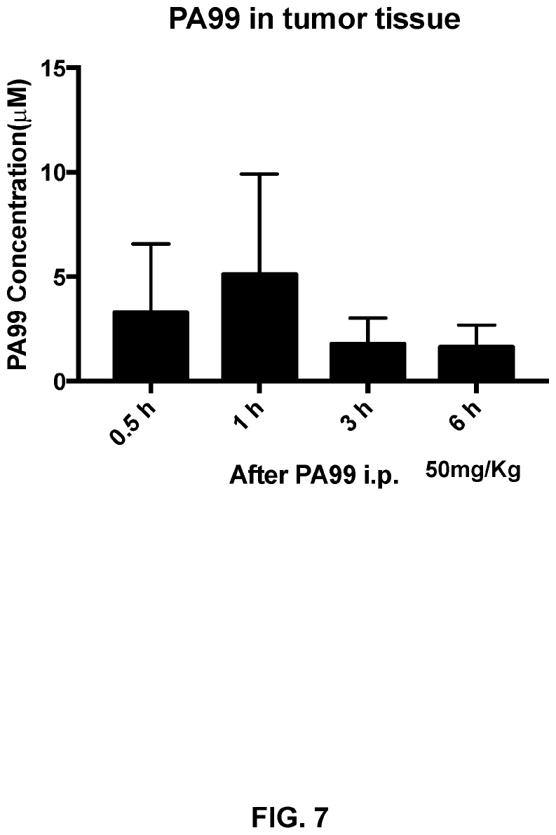

The results are set forth in Table 2 and also shown in FIG. 7. The Vial ID in Table 2 indicates the time period.

TABLE 2

| | | Tissue | |
|---|---|---|---|
| Vial ID | | ng/mL | μM |
| Tumor #1L control | | <10 | <0.02 |
| Tumor #2L control | | 18 | 0.04 |
| Tumor #3L 30 min | | 666 | 1.4 |
| Tumor #3R 30 min | | 670 | 1.4 |
| Tumor #4L30 min | | 1010 | 2.1 |
| Tumor #4R 30 min | | 3910 | 8.2 |
| | Average | 1564 | 3.3 |
| Tumor #5L 1 hr | | 5780 | 12.2 |
| Tumor #5R 1 hr | | 1790 | 3.8 |
| Tumor #11L 1 hr | | 1230 | 2.6 |
| Tumor #11R 1 hr | | 870 | 1.8 |
| | Average | 2418 | 5.1 |
| Tumor #12L 3 hr | | 1400 | 2.9 |
| Tumor #12R 3 hr | | 1340 | 2.8 |
| Tumor #13L 3 hr | | 392 | 0.8 |
| Tumor #13R 3 hr | | 290 | 0.6 |
| | Average | 856 | 1.8 |
| Tumor #14L 6 hr | | 419 | 0.88 |
| Tumor #14R 6 hr | | 342 | 0.72 |
| Tumor #15L 6 hr | | 1440 | 3.0 |
| Tumor #15R 6 hr | | 913 | 1.9 |
| | Average | 779 | 1.6 |

### Example 9

This example demonstrates the suppression of CUL4A^{high} xenograft and PDX tumor growth by a compound disclosed herein (compound PA99).

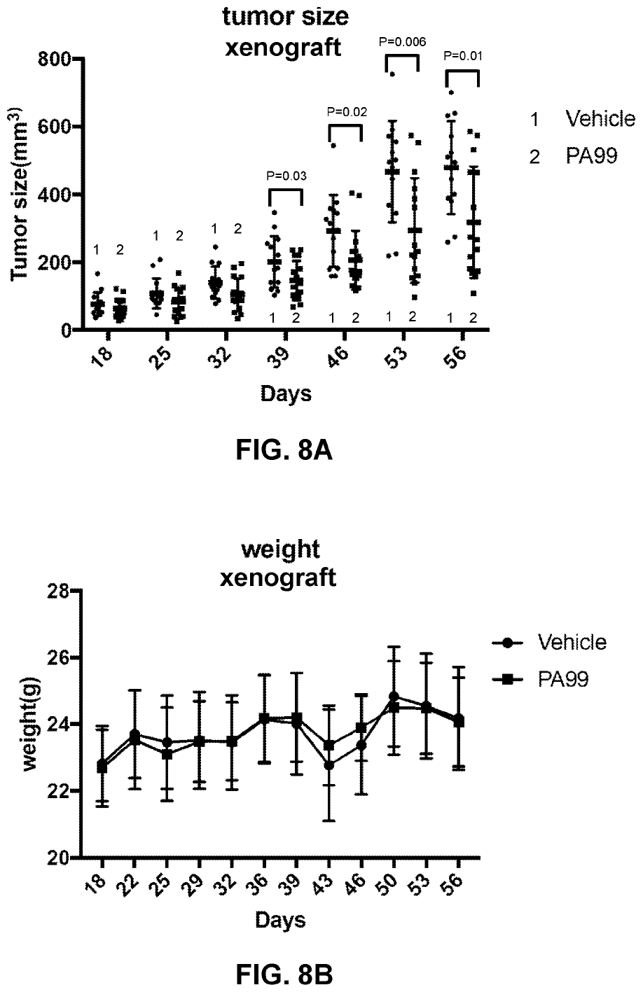

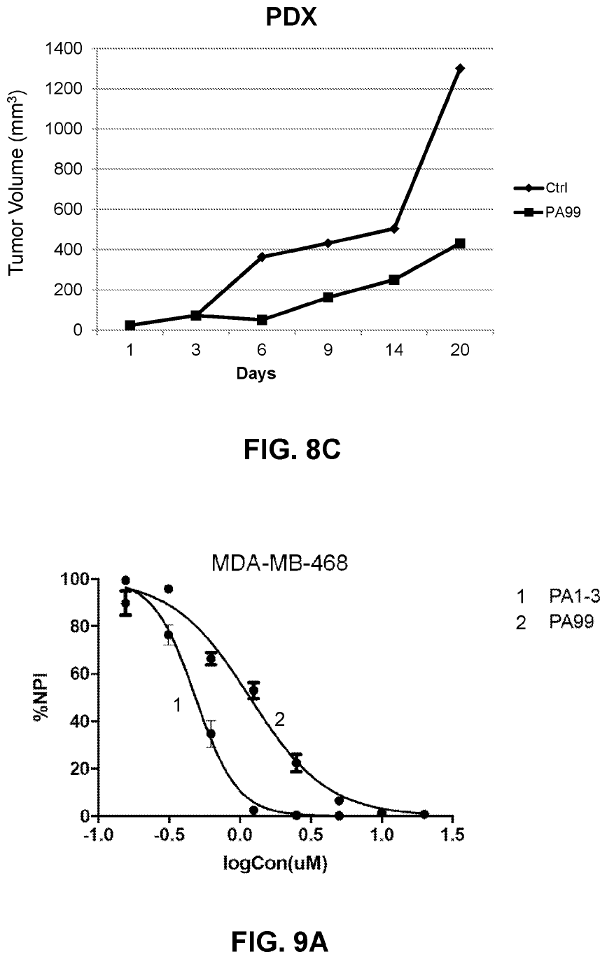

Measurements were performed according to the following procedure. Mouse body weights were monitored using a scale. Tumors were assessed and volumes measured with fine calipers twice a week. The tumor volumes were calculated with the formula: Volume=(Length×Width²)/2. Compound PA99 was administered via intraperitoneal doses in an amount of 50 mg/kg every X days, where X is the number of days shown in FIGS. 8A-8C.

The results are shown in FIGS. 8A-8C. FIG. 8A is a breast cancer xenograft tumor model. FIG. 8B is a cancer patient-derived xenograft tumor model. FIG. 8C relates to PDX tumors.

### Example 10

This example demonstrates the EC₅₀ (μM) of cell killing in CUL4^{high} (MDA-MB-468), CUL4^{low} (T47D), and MCF7 breast cancer cells.

Measurements were performed via a Cell Titer Glo assay that measured selective killing of CUL4^{high} (MDA-MB-468), CUL4^{low} (T47D), and MCF7 cells. CUL4A^{high} MDA-MB-468, CUL4^{low} (T47D), and MCF7 breast cancer cells were seeded in 96 well plates separately. Cells were treated with DMSO, or from 0 to 20 μM compound respectively. Replace with fresh medium and compounds every 48 hours. After 8 days treatment, cell viability was examined by CellTiter Glo kit (Promega #7570). First, transfer the appropriate volume (10 ml for Cat. #G7570) of CellTiter-Glo® Buffer into the amber bottle containing CellTiter-Glo® Substrate to reconstitute the lyophilized enzyme/substrate mixture. This forms the CellTiter-Glo® Reagent. Second, add CellTiter-Glo® Reagent in an equal volume (100 μl per well for 96-well plates) to all wells. Mix contents for 2 minutes on an orbital shaker to induce cell lysis. Incubate at room temperature for 15 minutes to stabilize luminescent signal. Then record luminescence. EC₅₀ concentration was calculated by Prism 8 XY analysis nonlinear regression (curve fit-Log(inhibitor) vs response).

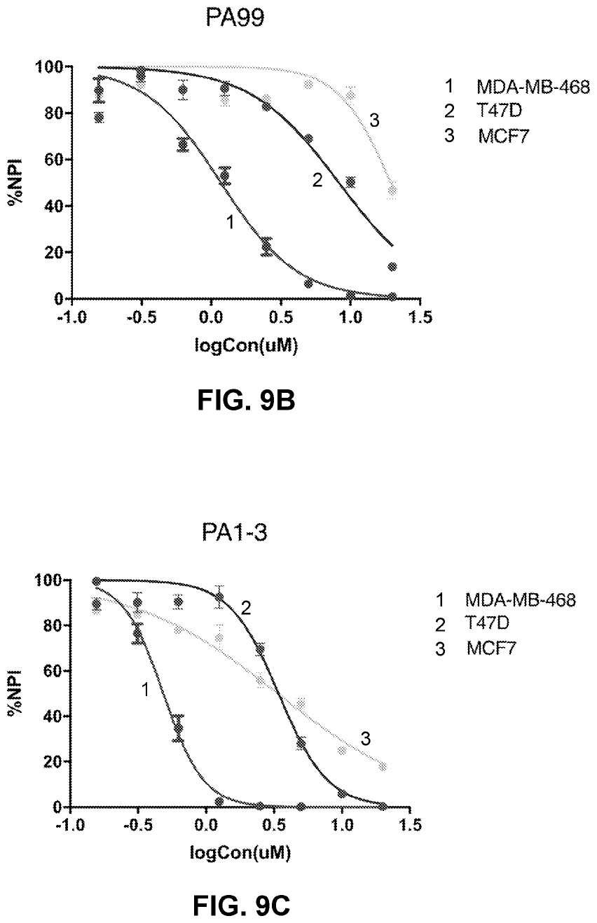

The results are set forth in Table 3 and also shown in FIGS. 9A-9C.

TABLE 3

| EC₅₀ OF cell | | | |
|---|---|---|---|
| killing(μM) | MDA-MB-468 | T47D | MCF7 |
| PA1-3 | 0.49 | 3.42 | 3.36 |
| PA99 | 1.18 | 8.27 | 19.51 |

### Example 11

This example demonstrates the selective killing of CUL4A^{high} tumors by compound PA99 in a colony formation assay.

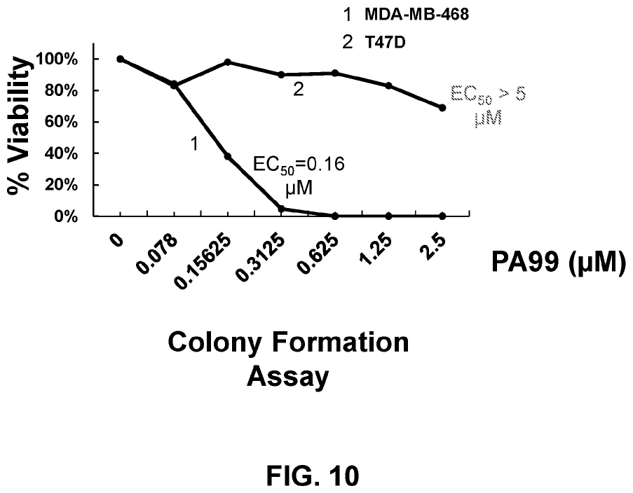

Measurements were performed via a Cell Titer Glo assay that measured selective killing of CUL4^{high} tumors by PA99. CUL4A^{high} MDA-MB-468 breast cancer cells and CUL4^{low} (T47D) cells were seeded in 96 well plates. After 24 hours, cells were treated with DMSO or compound PA99. After 8 days treatment, cell viability was examined by using CellTiter Glo kit (Promega #7570) following the manufacturer's guidelines. EC₅₀ concentration was calculated by Prism 8 XY analysis nonlinear regression (curve fit-Log(inhibitor) vs response). The results are shown in FIG. 10. Lower EC₅₀ indicates high cell killing potential in CUL4^{high} MDA-MB-468 cells.

### Example 12

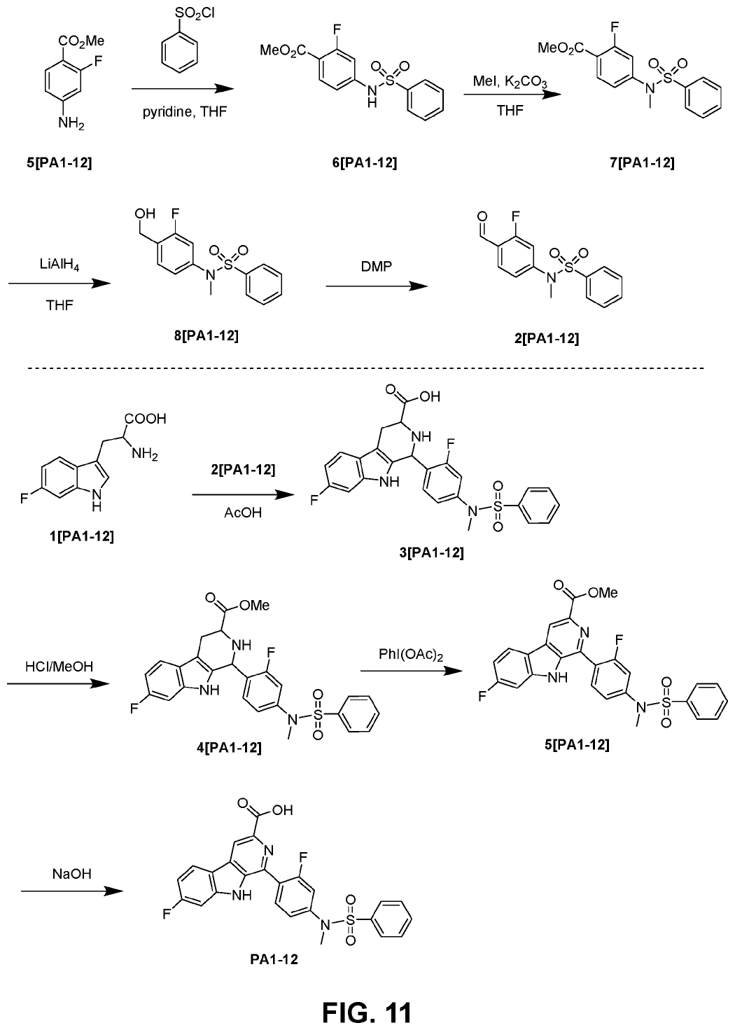

This example demonstrates the synthesis of compound 5[PA1-12] (methyl 1-[4-[benzenesulfonyl(methyl)amino]-2-fluoro-phenyl]-7-fluoro-9H-pyrido[3,4-b]indole-3-carboxylate) and PA1-12 (1-[4-[benzenesulfonyl(methyl)amino]-2-fluoro-phenyl]-7-fluoro-9H-pyrido[3,4-b]indole-3-carboxylic acid). The reaction scheme to produce compound 5[PA1-12] and compound PA1-12 is shown in FIG. 11, and each individual step in the synthesis is described in detail below.

6[PA1-12]: To a 250 mL round-bottom flask equipped with a magnetic stir bar was added 5[PA1-12] (methyl 4-amino-2-fluoro-benzoate) (4.50 g, 26.6 mmol, 1 eq), followed by the addition of THF (100 mL). Then pyridine (Py) (6.31 g, 79.8 mmol, 3 eq) and benzenesulfonyl chloride (5.17 g, 29.3 mmol, 1.1 eq) was added into the mixture at 25° C. The mixture was stirred at 25° C. for 12 h. The LCMS showed the starting material 5[PA1-12] was consumed and the desired product was detected. The mixture was concentrated under reduced pressure affording the crude product as yellow oil. The crude product was purified by silica gel column chromatography (petroleum ether/ethyl acetate, from 10/1 to 1/1) to give 6[PA1-12] (methyl 2-fluoro-4-(phenylsulfonamido)benzoate) (8.85 g, 97.9% yield, 91% purity) as a yellow solid. LCMS: m/z 309.9[M+H]⁺. 1H NMR (CDCl₃ 400 MHz) δ 7.90-7.80 (m, 3H), 7.70-7.60 (m, 1H), 7.60-7.50 (m, 2H), 7.00-6.96 (m, 1H), 6.90-6.86 (m, 1H), 3.89 (s, 3H).

7[PA1-12]: To a solution of 6[PA1-12] (methyl 4-(benzenesulfonamido)-2-fluoro-benzoate) (8.80 g, 25.9 mmol, 91% purity, 1 eq) in THF (100 mL) was added K₂CO₃ (10.7 g, 77.7 mmol, 3.0 eq) and MeI (9.19 g, 64.7 mmol, 2.5 eq) at 25° C. under N₂. Then the mixture was stirred at 60° C. for 12 h. The LCMS showed the starting material 6[PA1-12] was consumed and the desired product was detected. The TLC (petroleum ether/ethyl acetate=2/1) showed the starting material 6[PA1-12] was consumed and one new spot was observed. The reaction was poured into water (400 mL) and extracted with EA (180 mL×3). The combined organic phase was washed with brine (100 mL×2), dried with anhydrous Na₂SO₄, filtered and concentrated in vacuo. The mixture was concentrated under reduced pressure affording the crude product as yellow oil. The crude product was purified by silica gel column chromatography (petroleum ether/ethyl acetate, from 10/1 to 1/1) to give 7[PA1-12] (methyl 4-[benzenesulfonyl(methyl)amino]-2-fluoro-benzoate) (8.30 g, 99% yield) as a light yellow oil. LCMS: m/z 324.1[M+H]⁺. 1H NMR (CDCl₃ 400 MHz) δ 7.90-7.85 (m, 1H), 7.61-7.55 (m, 3H), 7.50-7.47 (m, 2H), 7.06-6.97 (in, 2H), 3.93 (s, 3H), 3.21 (s, 3H).

8[PA1-12]: To a solution of 7[PA1-12] (methyl 4-[benzenesulfonyl(methyl)amino]-2-fluoro-benzoate) (8.30 g, 25.67 mmol, 1 eq) in THF (120 mL) was added LiAlH₄ (2.44 g, 64.2 mmol, 2.5 eq) at 0° C. under N₂. Then the mixture was stirred at 25° C. for 1 h. The TLC (petroleum ether/ethyl acetate=3/1) showed the starting material 7[PA1-12] was consumed and one new spot was observed. The reaction was quenched with NaOH (2.5 mL, 15% in water), water (2.5 mL), then the mixture was stirred for 15 mins and then filtered. The filtrate was concentrated in vacuo to give 8[PA1-12] (N-[3-fluoro-4-(hydroxymethyl)phenyl]-N-methyl-benzenesulfonamide) (7.40 g, 92.54% yield, 94.8% purity), obtained as a light yellow oil and used for the next step directly without further purification. LCMS: m/z 317.9[M+Na]⁺.

2[PA1-12]: To a solution of 8[PA1-12] (N-[3-fluoro-4-(hydroxymethyl)phenyl]-N-methyl-benzenesulfonamide) (3.60 g, 11.6 mmol, 94.8% purity, 1 eq) in DCM (100 mL) was added Dess-Martin (5.15 g, 12.1 mmol, 1.05 eq) at 0° C. under N₂. The reaction was stirred at 0° C. for 2 h. Then the solution was stirred at 25° C. for 12 hour. The TLC (petroleum ether/ethyl acetate: 2/1) showed the starting material 8[PA1-12] was consumed and one new spot was observed. The mixture was poured into water (70 mL) and extracted with EA (100 mL×3). The combined organic phase was washed with brine (70 mL×2), dried with anhydrous Na₂SO₄, filtered and concentrated in vacuo. The mixture was concentrated under reduced pressure affording the crude product as black brown oil. The crude product was purified by silica gel column chromatography (petroleum ether/ethyl acetate, from 5/1 to 0/1) to give 2[PA1-12] (N-(3-fluoro-4-formyl-phenyl)-N-methyl-benzenesulfonamide) (3.46 g, 89.83% yield, 88% purity) was obtained as a brown solid. LCMS: m/z 294.2[M+H]⁺. ¹H NMR (CDCl₃ 400 MHz) δ 10.31 (s, 1H), 7.83-7.80 (m, 1H), 7.63-7.57 (m, 3H), 7.52-7.45 (m, 2H), 7.13-7.05 (in, 1H), 3.23 (s, 3H).

3[PA1-12]: To a solution of 1[PA1-12] (2-amino-3-(6-fluoro-1H-indol-3-yl)propanoic acid) (200 mg, 900 umol, 1 eq) in AcOH (8 mL) was added 2[PA1-12] (N-(3-fluoro-4-formyl-phenyl)-N-methyl-benzenesulfonamide) (300 mg, 900 umol, 88% purity, 1 eq.). Note that 1[PA1-12] is the same as compound 4[PA1-3] prepared in Example 2. Then the mixture was stirred at 120° C. for 2 h. LCMS showed that 1[PA1-12] was consumed completely and the desired product was detected. The reaction was concentrated in vacuo to give 3[PA1-12](1-[4-[benzenesulfonyl(methyl)amino]-2-fluoro-phenyl]-7-fluoro-2,3,4,9-tetrahydro-1H-pyrido[3,4-b]indole-3-carboxylic acid) (500 mg, crude) as yellow oil, which was used directly for the next step without further purification. LCMS: m/z 498.3 [M+H]⁺.

4[PA1-12]: A mixture of 3[PA1-12] (1-[4-[benzenesulfonyl(methyl)amino]-2-fluoro-phenyl]-7-fluoro-2,3,4,9-tetrahydro-1H-pyrido[3,4-b]indole-3-carboxylic acid) (470 mg, 945 umol, 1 eq.) in HCl/MeOH (4 M, 20 mL) was stirred at 25° C. for 12 h. LCMS and TLC (PE/EA=1:1) showed that the reactant 3[PA1-12] was consumed completely and desired product was detected. The reaction was concentrated in vacuo and then poured into EA (30 mL) and Na₂CO₃ (sat.) was added to make pH=7-8, then extracted with EA (20 mL×3). The organic layers were combined and washed with brine (30 mL), dried over Na₂SO₄, filtered, and concentrated in vacuo. The residue was purified by silica gel chromatography (PE/EA=3:1 to 1:1) to give 4[PA1-12] (methyl 1-[4-[benzenesulfonyl(methyl)amino]-2-fluoro-phenyl]-7-fluoro-2,3,4,9-tetrahydro-1H-pyrido[3,4-b]indole-3-carboxylate) (200 mg, 30.5% yield, 73.8% purity) as yellow oil. LCMS: m/z 512.0 [M+H]⁺.

5[PA1-12]: To a solution of 4[PA1-12] (methyl 1-[4-[benzenesulfonyl(methyl)amino]-2-fluoro-phenyl]-7-fluoro-2,3,4,9-tetrahydro-1H-pyrido[3,4-b]indole-3-carboxylate) (200 mg, 289 umol, 73.8% purity, 1 eq) in DMF (5 mL) was added PhI(OAc)₂ (279 mg, 866 umol, 3.0 eq) at 25° C. and the mixture was stirred at 25° C. for 2 h. LCMS and TLC (PE/EA=1:1) showed that the reactant 4[PA1-12] was consumed completely and desired product was detected. The reaction was poured into water (20 mL) and extracted with EA (20 mL×2). The organic layer was combined and washed with brine (20 mL), dried over Na₂SO₄, filtered, and concentrated in vacuo. The residue was purified by silica gel chromatography (PE/EA=3:1 to 1:1) to give 5[PA1-12] (methyl 1-[4-[benzenesulfonyl(methyl)amino]-2-fluoro-phenyl]-7-fluoro-9H-pyrido[3,4-b]indole-3-carboxylate) (0.16 g, 83.2% yield, 76.1% purity) was obtained as brown oil. LCMS: m/z 508.0 [M+H]⁺.

PA1-12: To a solution of 5[PA1-12] (methyl 1-[4-[benzenesulfonyl(methyl)amino]-2-fluoro-phenyl]-7-fluoro-9H-pyrido[3,4-b]indole-3-carboxylate) (160 mg, 240 umol, 76.1% purity, 1 eq) in MeOH (3 mL) and THF (3 mL) and H₂O (3 mL) was added NaOH (160 mg, 4.00 mmol, 16.7 eq) and the mixture was stirred at 45° C. for 12 h. LCMS showed 5[PA1-12] was consumed and the desired product was detected. The reaction was acidified by TFA to pH=3-4. Then the solution was concentrated in vacuo. The residue was purified by Prep-HPLC (column: Phenomenex Luna C18 150*25 mm*10 um; mobile phase: [water (0.1% TFA)-ACN]; B %: 46%-76%, 10 min) to give PA1-12 (1-[4-[benzenesulfonyl(methyl)amino]-2-fluoro-phenyl]-7-fluoro-9H-pyrido[3,4-b]indole-3-carboxylic acid) (46.9 mg, 38.9% yield, 98.2% purity) as a yellow solid, confirmed by LCMS, ¹H NMR, and HPLC. LCMS: m/z 494.1[M+H]⁺. ¹H NMR (DMSO-d₆ 400 MHz) δ 11.97 (S, 1H), 8.98 (s, 1H), 8.52-8.48 (m, 1H), 7.78-7.74 (m, 2H), 7.68-7.66 (m, 4H), 7.35-7.33 (m, 2H), 7.32-7.25 (m, 2H), 3.27 (s, 3H).

### Example 13

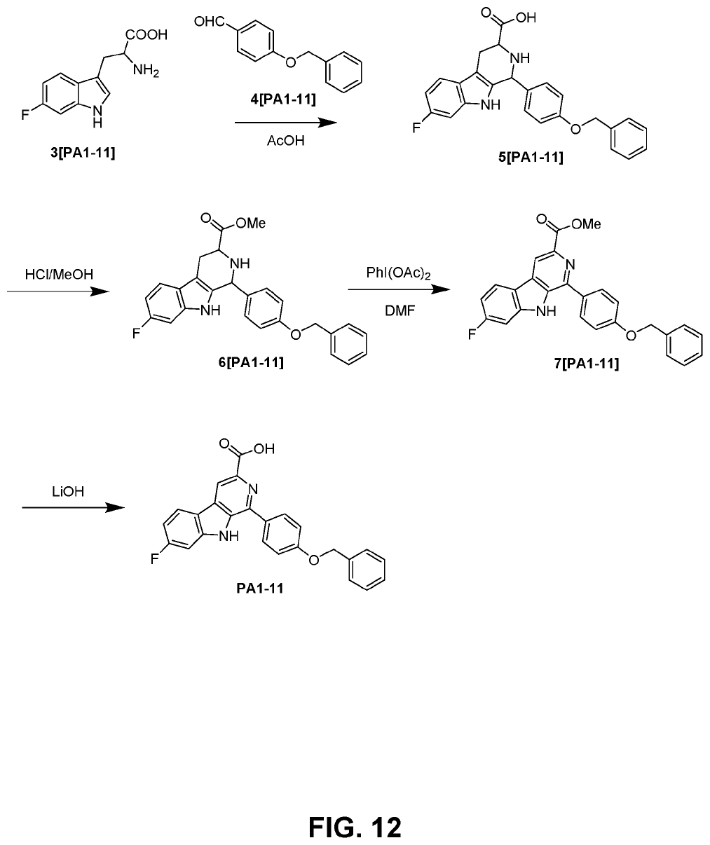

This example demonstrates the synthesis of compound 7[PA1-11] (methyl 1-(4-benzyloxyphenyl)-7-fluoro-9H-pyrido[3,4-b]indole-3-carboxylate) and PA1-11 (1-(4-benzyloxyphenyl)-7-fluoro-9H-pyrido[3,4-b]indole-3-carboxylic acid). The reaction scheme to produce compound 7[PA1-11] and compound PA1-11 is shown in FIG. 12, and each individual step in the synthesis is described in detail below.

5[PA1-11]: To a solution of 3[PA1-11] (2-amino-3-(6-fluoro-1H-indol-3-yl)propanoic acid) (220 mg, 990 umol, 1 eq) in AcOH (12 mL) was added 4-benzyloxybenzaldehyde (210 mg, 990 umol, 1 eq) and then the mixture was stirred at 120° C. for 2 h. Note that 3[PA1-11] is the same as compound 4[PA1-3] prepared in Example 2. The LCMS showed the starting material 3[PA1-11] was consumed completely and the desired product was obtained. The mixture concentrated in vacuo to give 5[PA1-11] (1-(4-(benzyloxy)phenyl)-7-fluoro-2,3,4,9-tetrahydro-1H-pyrido[3,4-b]indole-3-carboxylic acid) (500 mg, crude) as black brown oil. LCMS: m/z 417.4 [M+H]⁺.

6[PA1-11]: To a solution of 5[PA1-11] (1-(4-benzyloxyphenyl)-7-fluoro-2,3,4,9-tetrahydro-1H-pyrido[3,4-b]indole-3-carboxylic acid) (500 mg, 1.20 mmol, 1 eq) was added HCl/MeOH (4 M, 10 mL) and then the mixture was stirred at 25° C. for 12 h. and TLC (Petroleum ether:Ethyl acetate=3:1) showed that the starting material 5[PA1-11] was consumed and desired product was detected. The mixture was concentrated in vacuo to afford the residue as black solid. Then the residue was poured into water (20 mL) and basified with K₂CO₃ solid to pH=10-11. Then the mixture was extracted by EA (10 mL×3). Then the organic layers were washed by brine (10 mL×3), dried by Na₂SO₄, filtered, and concentrated in vacuo. The crude product was purified by silica gel column chromatography (petroleum ether/ethyl acetate, from 10/1 to 1/1) to give 6[PA1-11] (methyl 1-(4-benzyloxyphenyl)-7-fluoro-2,3,4,9-tetrahydro-1H-pyrido[3,4-b]indole-3-carboxylate) (310 mg, 56.6% yield, 94.4% purity) as a yellow oil. LCMS: m/z 430.9 [M+H]f.

7[PA1-11]: To a solution of 6[PA1-11] (methyl 1-(4-benzyloxyphenyl)-7-fluoro-2,3,4,9-tetrahydro-1H-pyrido[3,4-b]indole-3-carboxylate) (280 mg, 614 umol, 94.4% purity, 1 eq) in DMF (15 mL) was added PhI(OAc)₂ (593 mg, 1.84 mmol, 3.0 eq) at 25° C. and then the mixture was stirred for 5 h. LCMS and TLC (PE/EA=3:1) showed that the reactant 6[PA1-11] was consumed completely and desired product was detected. The reaction was poured into water (30 mL) and extracted with EA (20 mL×3). Then the organic layer was combined and washed with brine (30 mL), dried over Na₂SO₄, filtered and concentrated in vacuo. The residue was purified by silica gel chromatography (PE/EA=1:1) to give 7[PA1-11] (methyl 1-(4-benzyloxyphenyl)-7-fluoro-9H-pyrido[3,4-b]indole-3-carboxylate) (0.16 g, 40.3% yield, 65.9% purity) as a yellow oil. LCMS: m/z 427.1[M+H]⁺.

PA1-11: To a mixture of 7[PA1-11] (methyl 1-(4-benzyloxyphenyl)-7-fluoro-9H-pyrido[3,4-b]indole-3-carboxylate) (70.0 mg, 108 umol, 65.9% purity, 1 eq) in H₂O (3 mL), THF (3 mL) and MeOH (3 mL) was added NaOH (64.9 mg, 1.62 mmol, 15 eq). Then the mixture was stirred at 60° C. for 2 h. LCMS showed that the reactant 7[PA1-11] was consumed completely and desired product was detected. The reaction was acidified by TFA to pH=3-4. Then the solution was concentrated in vacuo. The residue was purified by prep-HPLC (column: Phenomenex Luna C18 150*25 mm*10 um; mobile phase: [water (0.1% TFA)-ACN]; B %: 48%-68%, 10 min) twice to give PA1-11 (1-(4-benzyloxyphenyl)-7-fluoro-9H-pyrido[3,4-b]indole-3-carboxylic acid) (10.6 mg, 23.2% yield, 97.8% purity) as white solid, confirmed by LCMS, ¹H NMR, and HPLC. LCMS: m/z 413.3[M+H]⁺. ¹H NMR (DMSO-d₆ 400 MHz) δ 11.96 (S, 1H), 8.86 (s, 1H), 8.49-8.40 (m, 1H), 8.03 (d, J=8.8 Hz, 2H), 7.54-7.51 (m, 2H), 7.46-7.37 (m, 4H), 7.29-7.26 (in, 2H), 7.20-7.15 (in, 1H), 5.26 (s, 2H).

PA1-11A: PA1-11A (7-fluoro-1-(4-phenoxyphenyl)-9H-pyrido[3,4-b]indole-3-carboxylic acid) was prepared using similar methodologies to PA1-11 except for 4-phenoxybenzaldehyde being used in place of 4-benzyloxybenzaldehyde. PA1-11A was confirmed by LCMS, ¹H NMR, and HPLC. LCMS: m/z 398.0 [M+H]⁺. ¹H NMR (DMSO-d₆ 400 MHz) δ=13.32-12.21 (m, 1H), 12.01 (s, 1H), 8.89 (s, 1H), 8.48 (dd, J=5.6, 8.8 Hz, 1H), 8.08 (d, J=8.4 Hz, 2H), 7.51-7.43 (m, 2H), 7.38 (dd, J=2.0, 10 Hz, 1H), 7.27-7.18 (m, 4H), 7.15 (d, J=8.0 Hz, 2H).

PA1-11B: To a mixture of 7[PA1-11] (methyl 1-(4-benzyloxyphenyl)-7-fluoro-9H-pyrido[3,4-b]indole-3-carboxylate) (0.7 g, 1.64 mmol, 1 eq) in THF (15 mL) was added Pd/C (0.3 g, 10% purity, 1.00 eq, stirred at 20° C. for 2 hr under H₂ (50 Psi). The LC-MS showed that 7[PA1-11] was consumed and the desired product was detected. The reaction was filtered and washed by MeOH (20 mL*3), concentrated in vacuo to afford 8[PA1-11] (methyl 7-fluoro-1-(4-hydroxyphenyl)-9H-pyrido[3,4-b]indole-3-carboxylate) (500 mg, crude) as yellow solid, confirmed by LCMS: m/z 337.1[M+H]⁺. To a mixture of 8[PA1-11] (methyl 7-fluoro-1-(4-hydroxyphenyl)-9H-pyrido[3,4-b]indole-3-carboxylate) (80 mg, 237.87 umol, 1 eq) in DMF (2 mL) was added 2-iodoethylbenzene (66.24 mg, 285.44 umol, 41.40 uL, 1.2 eq), K₂CO₃ (98.63 mg, 713.61 umol, 3 eq), stirred at 80° C. for 1 hr. The LC-MS showed that 8[PA1-11] was consumed and the desired product was detected. The mixture was poured to water (20 mL) was extracted by ethyl acetate (40 mL*3). Then the organic layers were combined and washed by brine (40 mL), dried by Na₂SO₄, concentrated in vacuo to afford the residue as brown oil. The crude product was purified by prep-HPLC (column: Phenomenex luna C18 150*25 mm*10 um; mobile phase: [water (FA)-ACN]; B %: 51%-81%, 10 min) to give 9[PA1-11] (methyl 7-fluoro-1-[4-(2-phenylethoxy)phenyl]-9H-pyrido[3,4-b]indole-3-carboxylate) (40 mg, crude) as yellow solid, which was confirmed by LCMS and ¹H NMR. LCMS: m/z 494.1[M+H]⁺. ¹H NMR (DMSO-d₆ 400 MHz) δ=12.11-11.79 (m, 1H), 8.88 (s, 1H), 8.60-8.45 (m, 1H), 7.96 (d, J=8.8 Hz, 2H), 7.40-7.33 (m, 5H), 7.28-7.18 (m, 4H), 4.34 (t, J=6.8 Hz, 2H), 3.93 (s, 3H), 3.12 (t, J=6.8 Hz, 2H). To a mixture of 9[PA1-11] (methyl 7-fluoro-1-[4-(2-phenylethoxy)phenyl]-9H-pyrido[3,4-b]indole-3-carboxylate) (40 mg, 90.81 umol, 1 eq) and NaOH (3.63 mg, 90.81 umol, 1 eq) in MeOH (1 mL) and THF (1 mL), H₂O (1 mL) was stirred at 80° C. for 1 hr. The LC-MS showed that 9[PA1-11] was consumed and the desired product was detected. The mixture was poured to water (20 mL) was extracted by ethyl acetate (40 mL*3). Then the organic layers were combined and washed by brine (40 mL*3), dried by Na₂SO₄, concentrated in vacuo to afford the residue as brown oil. The crude product was purified by prep-HPLC (column: Phenomenex luna C18 150*25 mm*10 um; mobile phase: [water (FA)-ACN]; B %: 51%-81% O10 min) and PA1-11B (7-fluoro-1-(4-phenethoxyphenyl)-9H-pyrido[3,4-b]indole-3-carboxylic acid) (7.5 mg, 17.18 umol, 18.91% yield, 97.66% purity) was obtained as yellow solid, which was confirmed by LCMS, ¹H NMR, and HPLC. LCMS: m/z 427.1[M+H]⁺. ¹H NMR (DMSO-d₆ 400 MHz) δ=11.95 (br d, J=2.0 Hz, 1H), 8.85 (s, 1H), 8.46 (dd, J=5.6, 8.8 Hz, 1H), 8.01 (d, J=8.4 Hz, 2H), 7.40-7.32 (m, 5H), 7.27-7.23 (m, 1H), 7.22-7.15 (m, 3H), 4.34 (t, J=6.8 Hz, 2H), 3.12 (br t, J=6.4 Hz, 2H).

PA1-11C: PA1-11C (7-fluoro-1-(4-(3-phenylpropoxy)phenyl)-9H-pyrido[3,4-b]indole-3-carboxylic acid) was prepared using similar methodologies to PA1-11B except for 3-iodopropylbenzene being used in place of 2-iodoethylbenzene. PA1-11C was confirmed by LCMS, ¹H NMR, and HPLC. LCMS: m/z 441.2 [M+H]⁺. ¹H NMR (DMSO-d₆ 400 MHz) δ=11.96 (br s, 1H), 8.85 (s, 1H), 8.46 (dd, J=5.6, 8.6 Hz, 1H), 8.01 (d, J=8.8 Hz, 2H), 7.39 (dd, J=2.0, 10.0 Hz, 1H), 7.34-7.26 (m, 4H), 7.23-7.15 (m, 4H), 4.10 (t, J=6.0 Hz, 2H), 2.80 (t, J=7.6 Hz, 3H), 2.14-2.06 (m, 2H).

PA1-11D: PA1-11D (1-(4-(cyclohexylmethoxy)phenyl)-7-fluoro-9H-pyrido[3,4-b]indole-3-carboxylic acid) was prepared using similar methodologies to PA1-11B except for bromomethylcyclohexane being used in place of 2-iodoethylbenzene. PA1-11D was confirmed by LCMS, ¹H NMR, and HPLC. LCMS: m/z 433.1 [M+H]⁺. ¹H NMR (DMSO-d₆ 400 MHz) S=11.97 (s, 1H), 8.87 (s, 1H), 8.47 (dd, J=5.6, 8.8 Hz, 1H), 7.94 (d, J=8.8 Hz, 2H), 7.38 (dd, J=2.0, 10.0 Hz, 1H), 7.20-7.14 (m, 3H), 3.93-3.89 (m, 5H), 1.91-1.64 (m, 9H), 1.49-1.34 (m, 2H).

PA1-11E: PA1-11E (7-fluoro-1-(4-((tetrahydro-2H-pyran-4-yl)methoxy)phenyl)-9H-pyrido[3,4-b]indole-3-carboxylic acid) was prepared using similar methodologies to PA1-11B except for 4-(bromomethyl)tetrahydropyran being used in place of 2-iodoethylbenzene. PA1-11E was confirmed by LCMS, ¹H NMR, and HPLC. LCMS: m/z 421.1 [M+H]⁺. ¹H NMR (DMSO-d₆ 400 MHz) δ=11.96 (br s, 1H), 8.86 (s, 1H), 8.47 (dd, J=5.6, 8.8 Hz, 1H), 8.02 (d, J=8.4 Hz, 2H), 7.39 (dd, J=2.0, 10.0 Hz, 1H), 7.25-7.11 (m, 3H), 3.98 (d, J=6.4 Hz, 2H), 3.94-3.89 (m, 2H), 3.45-3.35 (m, 2H), 2.13-2.05 (m, 1H), 1.81-1.66 (m, 2H), 1.45-1.36 (m, 2H).

PA1-11F: PA1-11F (7-fluoro-1-(4-(pyridin-4-ylmethoxy)phenyl)-9H-pyrido[3,4-b]indole-3-carboxylic acid) was prepared using similar methodologies to PA1-11B except for 4-(chloromethyl)pyridine being used in place of 2-iodoethylbenzene. PA1-11F was confirmed by LCMS, ¹H NMR, and HPLC. LCMS: m/z 414.1 [M+H]⁺. ¹H NMR (DMSO-d₆ 400 MHz) δ=11.98 (br s, 1H), 8.87 (s, 1H), 8.62 (d, J=5.6 Hz, 2H), 8.47 (dd, J=5.6, 8.8 Hz, 1H), 8.04 (d, J=8.4 Hz, 2H), 7.51 (d, J=5.2 Hz, 2H), 7.38 (dd, J=2.0, 9.6 Hz, 1H), 7.28 (d, J=8.4 Hz, 2H), 7.22-7.14 (m, 1H), 5.35 (s, 2H).

PA1-11G: PA1-11G (7-fluoro-1-(4-(pyridin-3-ylmethoxy)phenyl)-9H-pyrido[3,4-b]indole-3-carboxylic acid) was prepared using similar methodologies to PA1-11B except for 3-(chloromethyl)pyridine being used in place of 2-iodoethylbenzene. PA1-11G was confirmed by LCMS, ¹H NMR, and HPLC. LCMS: m/z 414.0 [M+H]⁺. ¹H NMR (DMSO-d₆ 400 MHz) δ=12.09-11.91 (m, 1H), 8.86 (s, 1H), 8.75 (s, 1H), 8.58 (d, J=4.0 Hz, 1H), 8.46 (dd, J=5.6, 8.8 Hz, 1H), 8.04 (d, J=8.4 Hz, 2H), 7.95 (d, J=7.6 Hz, 1H), 7.50-7.45 (m, 1H), 7.39 (dd, J=2.0, 9.6 Hz, 1H), 7.29 (d, J=8.4 Hz, 2H), 7.20-7.15 (m, 1H), 5.32 (s, 2H).

PA1-11H: PA1-11H (7-fluoro-1-(4-(pyridin-2-ylmethoxy)phenyl)-9H-pyrido[3,4-b]indole-3-carboxylic acid) was prepared using similar methodologies to PA1-11B except for 2-(chloromethyl)pyridine being used in place of 2-iodoethylbenzene. PA1-11H was confirmed by LCMS, ¹H NMR, and HPLC. LCMS: m/z 414.1 [M+H]⁺. ¹H NMR (DMSO-d₆ 400 MHz) δ=12.15-11.70 (m, 1H), 8.79 (s, 1H), 8.61 (d, J=4.4 Hz, 1H), 8.45-8.40 (m, 1H), 8.05-8.00 (m, 2H), 7.88-7.85 (m, 1H), 7.60-7.57 (m, 1H), 7.38-7.35 (m, 2H), 7.28-7.24 (m, 2H), 7.20-7.10 (m, 1H), 5.31 (s, 3H).

PA1-11I: PA1-11I (7-fluoro-1-(4-(thiophen-3-ylmethoxy)phenyl)-9H-pyrido[3,4-b]indole-3-carboxylic acid) was prepared using similar methodologies to PA1-11B except for 3-(bromomethyl)thiophene being used in place of 2-iodoethylbenzene. PA1-11I was confirmed by LCMS, ¹H NMR, and HPLC. LCMS: m/z 476.1 [M+H]⁺. ¹H NMR (DMSO-d₆ 400 MHz) δ=12.00-11.89 (m, 1H), 8.85 (s, 1H), 8.46 (dd, J=5.6, 8.8 Hz, 1H), 8.03 (d, J=8.8 Hz, 2H), 7.66 (d, J=2.0 Hz, 1H), 7.60 (dd, J=2.8, 4.8 Hz, 1H), 7.39 (dd, J=2.0, 10.0 Hz, 1H), 7.30-7.24 (m, 3H), 7.20-7.10 (m, 1H), 5.25 (s, 2H).

PA1-11J: PA1-11J (7-fluoro-1-(4-(furan-3-ylmethoxy)phenyl)-9H-pyrido[3,4-b]indole-3-carboxylic acid) was prepared using similar methodologies to PA1-11 except for 4-(3-furylmethoxy)benzaldehyde being used in place of 4-benzyloxybenzaldehyde. PA1-11J was confirmed by LCMS, ¹H NMR, and HPLC. LCMS: m/z 403.1 [M+H]⁺. ¹H NMR (DMSO-d₆ 400 MHz) δ=12.19-11.89 (m, 1H), 8.86 (s, 1H), 8.47 (dd, J=5.2, 8.8 Hz, 1H), 8.24-8.01 (m, 2H), 7.87 (s, 1H), 7.71 (s, 1H), 7.47-7.38 (m, 1H), 7.27 (d, J=8.8 Hz, 2H), 7.22-7.07 (m, 1H), 6.64 (s, 1H), 5.11 (s, 2H).

PA1-11K: PA1-11K (7-fluoro-1-(4-(thiazol-4-ylmethoxy)phenyl)-9H-pyrido[3,4-b]indole-3-carboxylic acid) was prepared using similar methodologies to PA1-11B except for 4-(chloromethyl)thiazole being used in place of 2-iodoethylbenzene. PA1-11K was confirmed by LCMS, ¹H NMR, and HPLC. LCMS: m/z 420.1 [M+H]⁺. ¹H NMR (DMSO-d₆ 400 MHz) δ=12.04-11.75 (m, 1H), 9.17 (d, J=1.6 Hz, 1H), 8.85 (s, 1H), 8.46 (dd, J=5.6, 8.4 Hz, 1H), 8.03 (d, J=8.8 Hz, 2H), 7.88 (s, 1H), 7.42-7.36 (m, 1H), 7.30 (d, J=8.8 Hz, 2H), 7.21-7.12 (m, 1H), 5.36 (s, 2H).

PA1-11L: PA1-11L (7-fluoro-1-(4-(pyrimidin-4-ylmethoxy)phenyl)-9H-pyrido[3,4-b]indole-3-carboxylic acid) was prepared using similar methodologies to PA1-11B except for 4-(chloromethyl)pyrimidine being used in place of 2-iodoethylbenzene. PA1-11L was confirmed by LCMS, ¹H NMR, and HPLC. LCMS: m/z 415.0 [M+H]⁺. ¹H NMR (DMSO-d₆ 400 MHz) 5=11.97-11.88 (m, 1H), 9.23 (s, 1H), 8.87 (d, J=4.8 Hz, 1H), 8.85-8.80 (m, 1H), 8.49-8.41 (m, 1H), 8.09-8.01 (m, 2H), 7.70 (d, J=5.2 Hz, 1H), 7.41-7.34 (m, 1H), 7.30 (d, J=8.8 Hz, 2H), 7.21-7.12 (m, 1H), 5.38 (s, 2H).

PA1-11M: PA1-11M (1-(4-((1H-1,2,4-triazol-1-yl)methoxy)phenyl)-7-fluoro-9H-pyrido[3,4-b]indole-3-carboxylic acid) was prepared using similar methodologies to PA1-11B except for 1-(chloromethyl)-1,2,4-triazole being used in place of 2-iodoethylbenzene. PA1-11M was confirmed by LCMS, ¹H NMR, and HPLC. LCMS: n/z 404.0 [M+H]⁺. ¹H NMR (DMSO-d₆ 400 MHz) δ=11.98 (br s, 1H), 8.89 (d, J=17.2 Hz, 2H), 8.46 (d, J=3.2 Hz, 1H), 8.13 (s, 1H), 8.04 (br d, J=8.4 Hz, 2H), 7.46-7.35 (m, 3H), 7.20-7.10 (m, 1H), 6.35 (s, 2H).

### Example 14

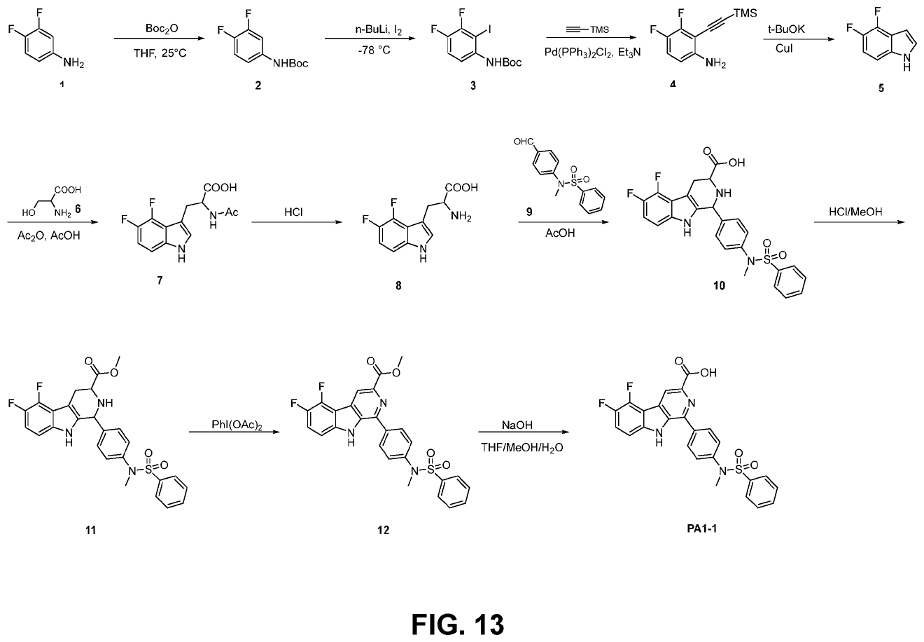

This example demonstrates the synthesis of PA1-1 (1-[4-[benzenesulfonyl(methyl) amino]phenyl]-5,6-difluoro-9H-pyrido[3,4-b]indole-3-carboxylic acid) and its ester. The reaction scheme to produce compound PA1-11 and its ester is shown in FIG. 13, and each individual step in the synthesis is described in detail below. Note that compound 9 in FIG. 13 is the same as compound 5[PA99] described in Example 1.

To a solution of compound 1 (25.0 g, 194 mmol, 1.0 eq) in THF (250 mL). Then Boc₂O (50.7 g, 232 mmol, 1.2 eq) was added into the mixture and the mixture was stirred at 70° C. for 2 h. The LC-MS showed that compound 1 was consumed and the desired mass was detected. The reaction was poured into water (500 mL) and extracted with EA (200 mL×3). The organic layer was combined and washed with brine (500 mL), dried over Na₂SO₄, and concentrated in vacuo. The residue was purified by silica gel chromatography with PE/EA=5:1 to 1:1 to give compound 2 (43.0 g, 97% yield) as white solid. LCMS: m/z 174.2 [M-tBu]⁺.

To a solution of compound 2 (43.0 g, 188 mmol, 1.0 eq) in THF (800 mL) was added n-BuLi (2.5 M, 165 mL, 2.2 eq) at −78° C. under N₂. The mixture was stirred at −78° C. for 1 h. Then I₂ (143 g, 563 mmol, 3.0 eq) was added to the mixture and then the solution was stirred at −78° C. for 1 h. The LCMS showed that compound 2 was consumed completely and desired mass was detected. TLC (PE/EA=3:1) showed that compound 2 was consumed completely and new spots were formed. The reaction was quenched with NH₄Cl (400 mL, sat.) and extracted with EA (500 mL×3). The organic layers were combined and washed with brine (1.0 L), dried over Na₂SO₄, and concentrated in vacuo. The residue was purified by silica gel chromatography with PE/EA=5:1 to 3:1 to give compound 3 (60.0 g, 82% yield, 91.3% purity) as brown solid, confirmed by LCMS, ¹H NMR, and 2D-NMR. LCMS: m/z 299.9 [M+H-56]⁺. ¹H NMR (CDCl₃, 400 MHz) δ 7.85-7.82 (m, 1H), 7.16 (q, J=8.4 Hz, 1H), 6.77 (br s, 1H), 1.53 (s, 9H).

To a solution of compound 3 (20.0 g, 56.3 mmol, 1.0 eq) and ethynyl(trimethyl)silane (8.30 g, 84.5 mmol, 1.5 eq) in TEA (200 mL) was added CuI (10.7 g, 56.3 mmol, 1.0 eq) and Pd(PPh₃)₂Cl₂ (1.98 g, 2.82 mmol, 0.05 eq) at 25° C. under N₂. Then the mixture was stirred at 80° C. for 12 h. TLC (PE/EA=10:1) showed that compound 3 was consumed completely and new spots were detected. The reaction was poured into water (400 mL) and extracted with EA (200 mL×3). The organic layer was combined and washed with brine (300 mL), dried over Na₂SO₄, and concentrated in vacuo. The residue was purified by silica gel chromatography with PE/EA=1:0 to 100:1 to give compound 4 (17 g, 92.8% yield) as black brown oil, confirmed by H NMR. ¹H NMR (CDCl₃, 400 MHz) δ 7.86-7.84 (m, 1H), 7.20-7.10 (m, 1H), 6.77 (br s, 1H), 1.52 (s, 9H), 0.31 (s, 9H).

To a solution of compound 4 (15.0 g, 46.1 mmol, 1 eq) in DMF (150 mL) was added CuI (4.39 g, 23.1 mmol, 0.5 eq) and t-BuOK (1 M in THF, 71 mL, 1.5 eq) at 25° C. under N₂. Then the mixture was stirred at 80° C. for 12 h. TLC (PE/EA=3:1) showed that compound 4 was consumed and new spots were detected. The reaction was poured into water (50 mL) and extracted with EA (150 mL×2). The organic layers were combined and washed with brine (100 mL), dried over Na₂SO₄, filtered and concentrated in vacuo. The residue was purified by silica gel chromatography with PE/EA=20:1 to 10:1 to give compound 5 (2.40 g, 34% yield) as yellow oil, confirmed by ¹H NMR. ¹H NMR (CDCl₃, 400 MHz) δ 8.31 (br s, 1H), 7.23-7.22 (m, 1H), 7.06-7.02 (m, 2H), 6.67-6.66 (m, 1H).

To a solution of compound 5 (2.40 g, 15.7 mmol, 1 eq) and compound 6 (3.29 g, 31.4 mmol, 2.0 eq) in AcOH (48 mL) was added Ac₂O (10.5 mL) at 25° C. under N₂. Then the mixture was stirred at 40° C. for 12 h. LCMS showed that compound 5 was consumed completely and desired mass was detected. The reaction was concentrated in vacuo to give a crude of compound 7 (7.5 g, crude) as yellow oil. LCMS: m/z 283.1[M+H]⁺.

To a solution of compound 7 (500 mg, 1.77 mmol, 1.0 eq) in H₂O (5 mL) was added conc.HCl (15 mL, 37% purity) at 25° C. The reaction solution was stirred at 75° C. for 12 h. LCMS showed that compound 7 was consumed completely and desired mass was detected. The reaction was basified by NH₃/H₂O (con., ˜28% purity) to make pH=8-9. Then the solution was poured into water (100 mL), extracted with EA (100 mL×3) and the organic layers were discarded. The water layer was combined and then purified by reversed-Phase to get a crude product. The crude product was purified by reversed-phase HPLC (0.1% TFA condition) to give compound 8 (500 mg, 7.1% yield, 84.8% purity) as yellow solid which was confirmed by LCMS: m/z 241.1[M+H]⁺.

To a solution of compound 8 (117 mg, 413 umol, 1.0 eq) and compound 9 (119 mg, 413 umol, 1.0 eq) in AcOH (8 mL). The solution was stirred at 120° C. for 2 h. LCMS showed compound 8 was consumed completely and desired mass was detected. The reaction was concentrated in vacuo to give a crude of compound 10 (500 mg, crude) as yellow oil, which was used directly for next step. LCMS: m/z 498.2[M+H]⁺.

To a solution of compound 10 (500 mg, 1.00 mmol, 1.0 eq) was dissolved in HCl/MeOH (4 M, 25 mL) at 25° C. under N₂. Then the mixture was stirred at 25° C. for 12 h. The LCMS showed that compound 10 was consumed and the desired mass was detected. The reaction was concentrated in vacuo. The crude product was purified by Perp-HPLC (column: Waters Xbridge 150*25 mm*5 um; mobile phase: [water (0.05% ammonia hydroxide v/v)-ACN]; B %: 48%-68%, 10 min) to give compound 11 (30.0 mg, 5.8% yield) as brown solid. LCMS: m/z 512.2[M+H]⁺.

To a solution of compound 11 (5.00 mg×12, 9.77 umol×12, 1.0 eq) in DMF (0.5 mL×12) was added PhI(OAc)₂ (9.44 mg×12, 29.3 umol×12, 3.0 eq) at 25° C. under N₂. Then the mixture was stirred at 25° C. for 12 h. The LCMS showed that compound 11 was consumed and the desired mass was detected and TLC (PE/EA=1:1) showed that compound 11 was consumed completely and new spots were formed. The reactions were poured into water (20 mL) and extracted with EA (10 mL×3). The organic layer was combined and washed with brine (20 mL), dried over Na₂SO₄, and concentrated in vacuo. The residue was purified by silica gel chromatography with PE/EA=3:1 to 1:1 to give compound 12 (0.03 g, 50% yield, 100% purity) as yellow oil, confirmed by LCMS: m/z 508.1[M+H]⁺.

To a solution of compound 12 (30.0 mg, 59.1 umol, 1.0 eq) in THF (3 mL) and H₂O (3 mL) was added NaOH (60.0 mg, 1.50 mmol, 25 eq) at 25° C. Then the mixture was stirred at 60° C. for 12 h. After cooling, the reaction was acidified by pure TFA (purity: 95%) to pH=2-3. Then the mixture was concentrated in vacuo. The residue was purified by Prep-HPLC (column: Phenomenex Luna C18 150*25 mm*10 um; mobile phase: [water (0.10% TFA)-ACN]; B %: 48%-78%, 10 min) to give PA1-1 (5.0 mg, 17% yield, 98.3% purity) as yellow solid. LCMS, HPLC, and ¹H NMR confirmed the product. LCMS: m/z 494.1 [M+H]⁺. ¹H NMR (DMSO-d₆, 400 MHz) δ 12.29 (s, 1H), 8.70 (s, 1H), 8.06-8.03 (m, 2H), 7.75-7.72 (m, 6H), 7.64-7.63 (m, 1H), 7.41-7.39 (m, 2H), 3.22 (s, 3H).

### Example 15

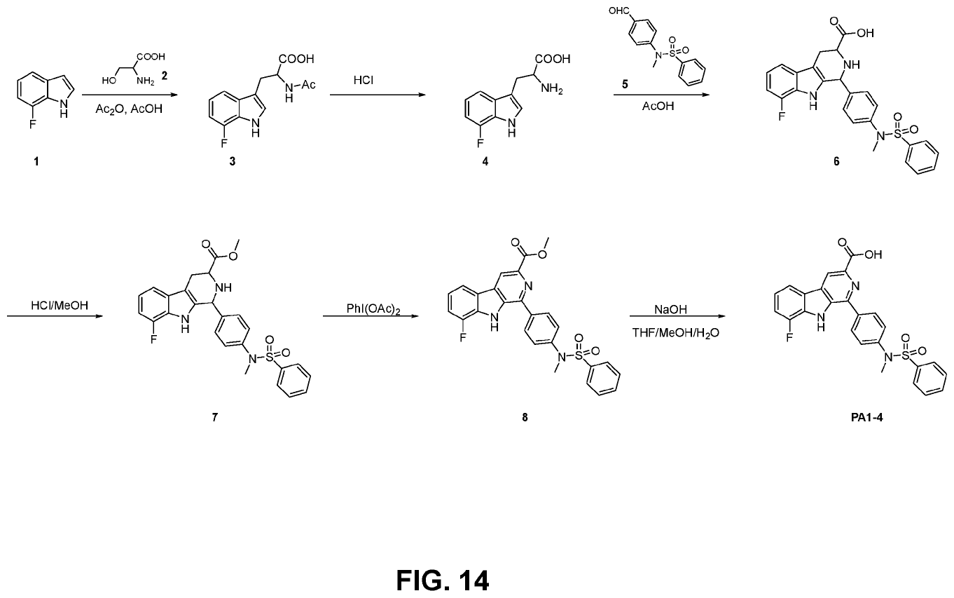

This example demonstrates the synthesis of PA1-4 (8-fluoro-1-(4-(N-methylphenylsulfonamido)phenyl)-9H-pyrido[3,4-b]indole-3-carboxylic acid) and its ester. The reaction scheme to produce compound PA1-4 and its ester is shown in FIG. 14, and each individual step in the synthesis is described in detail below. Note that compound 5 in FIG. 14 is the same as compound 5[PA99] described in Example 1.

To the mixture of compound 1 (10.0 g, 74.0 mmol, 1.0 eq) in Ac₂O (50 mL) and CH₃COOH (120 mL) was added compound 2 (15.6 g, 148 mmol, 2.0 eq) at 25° C. The mixture was stirred at 40° C. for 12 h. The LCMS showed that compound 1 was consumed completely and desired mass was detected. The reaction was concentrated in vacuo and then the residue was poured into water (600 mL) and extracted with EA (500 mL×3). The organic layers were combined and washed with brine (1 L), dried over Na₂SO₄, and concentrated in vacuo. The residue was dried in vacuo to give compound 3 (27.0 g, crude) as black brown oil. LCMS: m/z 265.0 [M+H]⁺.

To a solution of compound 3 (2.50 g, 9.46 mmol, 1.0 eq) in H₂O (6 mL) was added HCl (16 mL, 37% purity) under N₂. The solution was stirred at 75° C. for 12 h. The LCMS showed compound 3 was consumed completely and desired mass was detected. The reaction was concentrated in vacuo to give compound 4 (20.0 g, crude) as black brown oil. LCMS: m/z 223.0 [M+H]⁺. Note: 10 parallel reactions (2.5 g each).

To a solution of compound 4 (10.8 g, 48.6 mmol, 1.0 eq) and compound 5 (2.80 g, 9.72 mmol, 0.20 eq) in AcOH (80 mL). The solution was stirred at 110° C. for 2 h. LCMS showed compound 4 was consumed completely and desired mass was detected. The reaction was concentrated in vacuo. The residue was dried in vacuo to give compound 6 (14.0 g, crude) as black brown solid. LCMS: m/z 480.1 [M+H]⁺.

The compound 6 (13.0 g, 27.1 mmol, 1.0 eq) was dissolved in HCl/MeOH (4 M, 100 mL) and then the mixture was stirred at 25° C. for 12 h. LCMS showed that compound 6 was consumed completely and desired mass was detected. TLC (PE/EA=2:1) showed that compound 6 was consumed completely and new spots were formed. The reaction was concentrated in vacuo. Then the residue was poured into water (200 mL) and basified by Na₂CO₃ (solid) to pH=8-9, Then the mixture was extracted with EA (400 mL×3). The organic layer was combined and washed with brine (500 mL), dried over Na₂SO₄, and concentrated in vacuo. The residue was purified by silica gel chromatography with PE/EA=2:1 to 1:3 to give compound 7 (2.40 g, 12% yield, 66% purity) as black brown oil, detected by LCMS: m/z 494.1 [M+H]⁺.

To a solution of compound 7 (800 mg, 1.07 mmol, 1.0 eq) in DMF (10 mL) was added PhI(OAc)₂ (1.03 g, 3.21 mmol, 3.0 eq) at 25° C. and the mixture was stirred at 25° C. for 12 h. LCMS and TLC (PE/EA=2:1) showed that compound 7 was consumed completely and desired product was detected. The reaction was poured into water (30 mL) and extracted with EA (10 mL×3). The organic layer was combined and washed with brine (50 mL), dried over Na₂SO₄, and concentrated in vacuo. The residue was purified by silica gel chromatography with PE/EA=2:1 to 1:4 to give compound 8 (0.23 g, 41% yield, 93.6% purity) as yellow solid, detected by LCMS: m/z 490.1 [M+H]⁺.

To a solution of compound 8 (85.5 mg, 163 umol, 1.0 eq) in MeOH (3 mL) and THF (3 mL) and H₂O (3 mL) was added NaOH (100 mg, 2.50 mmol, 15.3 eq) at 25° C. and then the mixture was stirred at 60° C. for 12 h. LCMS showed that compound 8 was consumed completely and desired mass was detected. The reaction was acidified by TFA (95% purity) to pH=3-4, Then the mixture was concentrated in vacuo. The residue was purified by Prep-HPLC (column: Phenomenex Luna C18 150*25 mm*10 um; mobile phase: [water (0.1% TFA)-ACN]; B %: 43%-73%, 10 min) to give PA1-4 (45.0 mg, 57% yield, 98.9% purity) as yellow solid, confirmed by ¹H NMR, HPLC, and LCMS. LCMS: m/z 476.1 [M+H]⁺. ¹H NMR (DMSO-d₆, 400 MHz) δ 12.30 (s, 1H), 8.94 (s, 1H), 8.29 (d, J=8.0 Hz, 2H), 8.05 (d, J=8.4 Hz, 1H), 7.73-7.72 (m, 1H), 7.65-7.64 (m, 4H), 7.48-7.45 (m, 1H), 7.37-7.31 (m, 1H), 3.25 (s, 3H).

### Example 16

This example demonstrates the synthesis of PA1-5 (5,7-difluoro-1-(4-(N-methylphenylsulfonamido)phenyl)-9H-pyrido[3,4-b]indole-3-carboxylic acid), PA1-6 (1-[4-[benzenesulfonyl(methyl)amino]phenyl]-7-chloro-9H-pyrido[3,4-b]indole-3-carboxylic acid), PA1-7 (6,7-difluoro-1-(4-(N-methylphenylsulfonamido)phenyl)-9H-pyrido[3,4-b]indole-3-carboxylic acid) and each of their esters.

PA1-5, PA1-6, PA1-7, and each of their esters were prepared using the same synthetic route as PA1-4 in Example 15, except the starting material or other reactant(s) was varied to achieve the appropriate ring substitutions. See, for example, FIG. 14 for the illustrative synthetic route.

PA1-5: LCMS: m/z 494.0[M+H]⁺. ¹H NMR (DMSO-d₆ 400 MHz) δ 12.38 (s, 1H), 8.65 (s, 1H), 8.03 (d, J=8.4 Hz, 2H), 7.80-7.73 (in, 1H), 7.65-7.63 (in, 4H), 7.41 (d, J=8.4 Hz, 2H), 7.31-7.28 (m, 2H), 3.26 (s, 3H). Methyl ester of PA1-5: LCMS: m/z 508.0[M+H]⁻.

PA1-6: LCMS: m/z 492.3[M+H]⁺. ¹H NMR (DMSO-d₆ 400 MHz) δ 12.07 (s, 1H), 8.94 (s, 1H), 8.48 (d, J=8.4 Hz, 1H), 8.04 (d, J=8.8 Hz, 2H), 7.74-7.63 (m, 6H), 7.41-7.37 (m, 3H), 3.26 (s, 3H). Methyl ester of PA1-6: LCMS: m/z 506.0[M+H]⁺.

PA1-7: LCMS: m/z 493.9[M+H]⁺. ¹H NMR (DMSO-d₆ 400 MHz) δ 12.11 (s, 1H), 8.95 (s, 1H), 8.63-8.57 (m, 1H), 8.05-8.02 (m, 2H), 7.76-7.72 (m, 1H), 7.65-7.60 (m, 5H), 8.39 (d, J=8.4 Hz, 2H), 3.26 (s, 3H). Methyl ester of PA1-7: LCMS: m/z 283.1[M+H]⁺.

### Example 17

This example demonstrates the synthesis of PA1-12A (7-fluoro-1-(2-methyl-4-(N-methylphenylsulfonamido)phenyl)-9H-pyrido[3,4-b]indole-3-carboxylic acid) and its ester. PA1-12A and its ester were prepared using the same synthetic route as PA1-12 in Example 12, except the starting material or other reactant(s) was varied to achieve the appropriate ring substitutions. See, for example, FIG. 11 for the illustrative synthetic route.

PA1-12A: LCMS: m/z 490.0 [M+H]⁺. ¹H NMR (DMSO-d₆ 400 MHz) δ 11.81 (s, 1H), 8.96 (s, 1H), 8.52-8.50 (m, 1H), 7.76-7.66 (m, 5H), 7.51-7.48 (m, 1H), 7.32-7.30 (m, 1H), 7.25-7.15 (m, 3H), 3.24 (s, 3H), 2.15 (s, 3H). Methyl ester of PA1-12A: LCMS: m/z 504.1 [M+H]⁺.

### Example 18

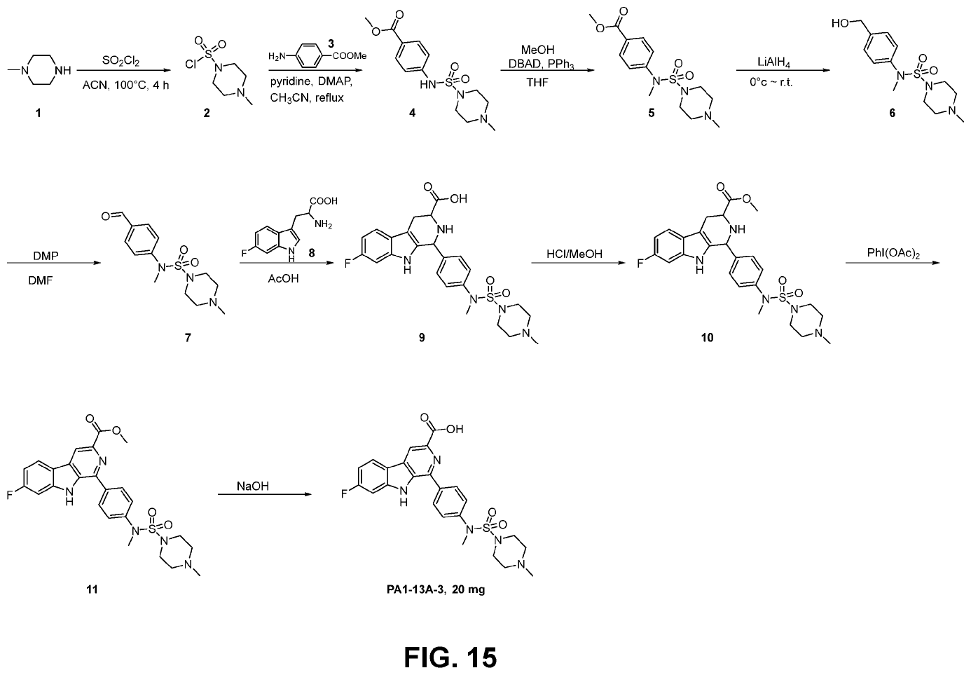

This example demonstrates the synthesis of PA1-13A-3 (1-(4-(N,4-dimethylpiperazine-1-sulfonamido)phenyl)-7-fluoro-9H-pyrido[3,4-b]indole-3-carboxylic acid) and its ester. The reaction scheme to produce compound PA1-13A-3 and its ester is shown in FIG. 15, and each individual step in the synthesis is described in detail below. Note that compound 8 in FIG. 15 is the same as compound 4[PA1-3] described in Example 2.

A mixture of 1-methylpiperazine (10.0 g, 99.84 mmol, 11.07 mL, 1 eq) in CH₃CN (180 mL) was added sulfuryl chloride (26.95 g, 199.68 mmol, 19.96 mL, 2.0 eq) slowly at 0° C. and then the mixture was stirred at 100° C. for 4 h. The mixture was concentrated in vacuo to afford 4-methylpiperazine-1-sulfonyl chloride (20 g, crude, HCl salt) as brown solid, which was used for the next step directly without further purification.

To a solution of 4-methylpiperazine-1-sulfonyl chloride (8.00 g, crude, HCl salt) in THF (180 mL) was added methyl 4-aminobenzoate (5.14 g, 34.02 mmol, 1 eq), pyridine (13.46 g, 170.12 mmol, 5 eq), DMAP (8.31 g, 68.05 mmol, 2 eq) at 0° C. Then the mixture was stirred at 100° C. for 2 h. LCMS and TLC (DCM:MeOH=10:1) showed the desired product was detected. The mixture was concentrated in vacuo to afford the residue as yellow solid. The residue was purified by flash column (EA:PE=1:10 to EA:PE=1:0) to afford methyl 4-(4-methylpiperazine-1-sulfonamido)benzoate (2 g, crude) as yellow solid, which was confirmed by ¹H NMR. LCMS: m/z 313.9 [M+H]⁺. ¹H NMR (CDCl₃, 400 MHz) δ 8.02-7.97 (m, 2H), 7.21-7.17 (m, 2H), 3.91 (s, 3H), 3.34-3.30 (m, 4H), 2.41-2.38 (m, 4H), 2.26 (s, 3H).

To a solution of methyl 4-(4-methylpiperazine-1-sulfonamido)benzoate (1.60 g, 5.11 mmol, 1 eq) in THF (20 mL) was added to PPh₃ (6.72 g, 25.62 mmol, 5.02 eq), DBAD (5.88 g, 25.53 mmol, 5 eq), MeOH (490.79 mg, 15.32 mmol, 619.84 uL, 3 eq). Then the mixture was stirred at 25° C. for 1 h under N₂. The LC-MS showed the methyl 4-(4-methylpiperazine-1-sulfonamido)benzoate was consumed and the desired product was detected. The mixture was added HCl (1M, 20 mL) to make pH=1. Then it was extracted by EA (10 mL×3), which was discarded. The water phase was basified by K₂CO₃ solid to make pH=8 to 9, extracted by EA (10 mL×3) and the organic layers were combined and washed by brine (10 mL×3), dried by Na₂SO₄, concentrated in vacuo to afford methyl 4-(N,4-dimethylpiperazine-1-sulfonamido)benzoate (1.5 g, crude) as yellow solid, which was confirmed by H NMR. The crude product was used into the next step without further purification. LCMS: m/z 327.9 [M+H]⁺. ¹H NMR (CDCl₃, 400 MHz) δ 8.03 (d, J=8.8 Hz, 2H), 3.93 (s, 3H), 3.30-3.25 (m, 4H), 2.41-2.35 (m, 4H), 2.26 (s, 3H).

To a solution of methyl 4-[methyl-(4-methylpiperazin-1-yl)sulfonyl-amino]benzoate (0.8 g, 2.44 mmol, 1 eq) in CHCl₃ (20 mL) was slowly added LiAlH₄ (300 mg, 7.91 mmol, 3.24 eq) at 0° C. under N₂. Then the mixture was added THF (10 mL) and stirred at 0° C. for 0.5 h. LCMS showed the methyl 4-[methyl-(4-methylpiperazin-1-yl)sulfonyl-amino]benzoate was remained and desired product was detected. To the mixture was added NaOH (15% purity, 0.3 mL) and 0.3 mL H₂O at 0° C. Then the mixture was filtered, concentrated in vacuo to afford N-[4-(hydroxymethyl)phenyl]-N,4-dimethyl-piperazine-1-sulfonamide (0.7 g, crude) as yellow solid. The crude product was used into the next step without further purification. LCMS: m/z 300.2 [M+H]⁺.

To a solution of N-[4-(hydroxymethyl)phenyl]-N,4-dimethyl-piperazine-1-sulfonamide (0.7 g, 2.34 mmol, 1 eq) in DMF (20 mL) was added DMP (1.49 g, 3.51 mmol, 1.5 eq) under N₂ at 0° C. for 1 h. LCMS showed the N-[4-(hydroxymethyl)phenyl]-N,4-dimethyl-piperazine-1-sulfonamide was consumed and the desired product was detected. The mixture was poured into water (200 mL), extracted by EA (100 mL×3). Then the organic layers were combined and washed by brine (100 mL×3), dried by Na₂SO₄, concentrated in vacuo to afford N-(4-formylphenyl)-N,4-dimethyl-piperazine-1-sulfonamide (0.4 g, crude) as brown oil. The crude product was used into the next step without further purification. LCMS: m/z 298.1[M+H]⁺.

To a solution of N-(4-formylphenyl)-N,4-dimethylpiperazine-1-sulfonamide (40 mg, 134.51 umol, 1 eq) in AcOH (2 mL) was added 2-amino-3-(6-fluoro-1H-indol-3-yl)propanoic acid (29.89 mg, 133.30 umol, 99.1% purity, 1 eq). Then the mixture was stirred at 120° C. for 1 h. LCMS showed the 2-amino-3-(6-fluoro-1H-indol-3-yl)propanoic acid was consumed and the desired product was detected. The mixture was concentrated in vacuo to afford 1-(4-(N,4-dimethylpiperazine-1-sulfonamido)phenyl)-7-fluoro-2,3,4,9-tetrahydro-1H-pyrido[3,4-b]indole-3-carboxylic acid (70 mg, crude) as brown solid. It's used to next step without purification. LCMS: m/z 502.3[M+H]⁺.

To a solution of 1-(4-(N,4-dimethylpiperazine-1-sulfonamido)phenyl)-7-fluoro-2,3,4,9-tetrahydro-1H-pyrido[3,4-b]indole-3-carboxylic acid (70 mg, 139.56 umol, 1 eq) in HCl/MeOH (15 mL, 4 M in MeOH). Then the mixture was stirred at 25° C. for 12 h. LCMS showed the 1-[4-[benzenesulfonyl(methyl)amino]phenyl]-5,7-difluoro-2,3,4,9-tetrahydro-1H-pyrido[3,4-b]indole-3-carboxylic acid was consumed and the desired product was detected. The mixture was concentrated in vacuo to afford the residue as black solid.

Then the residue was poured into water (15 mL) and it was basified by K₂CO₃ solid to make pH=10 to 11. Then the mixture was extracted by EA (10 mL×3). The organic layers were washed by brine (20 mL×2), dried by Na₂SO₄, concentrated in vacuo to get methyl 7-fluoro-1-[4-[methyl-(4-methylpiperazin-1-yl)sulfonyl-amino]phenyl]-2,3,4,9-tetrahydro-1H-pyrido[3,4-b]indole-3-carboxylate (40 mg, crude) as yellow solid. LCMS: m/z 516.4[M+H]1.

To a solution of methyl 7-fluoro-1-[4-[methyl-(4-methylpiperazin-1-yl)sulfonyl-amino]phenyl]-2,3,4,9-tetrahydro-1H-pyrido[3,4-b]indole-3-carboxylate (30 mg, 58.18 umol, 1 eq) in DMF (3 mL) was added PhI(OAc)₂ (74.97 mg, 232.74 umol, 4 eq). Then the mixture was stirred at 25° C. for 2 h. LCMS and TLC (PE:EA=1:1) showed the methyl 7-fluoro-1-[4-[methyl-(4-methylpiperazin-1-yl)sulfonyl-amino]phenyl]-2,3,4,9-tetrahydro-1H-pyrido[3,4-b]indole-3-carboxylate was consumed and the desired product was detected. To the solution was added water (30 mL), extracted by EA (10 mL×3). Then the organic layers were combined and washed water (10 mL×3), dried by Na₂SO₄, concentrated in vacuo. The crude product was purified by silica gel column chromatography (PE:EA=10:1 to PE:EA=1:1) to afford methyl 7-fluoro-1-[4-[methyl-(4-methylpiperazin-1-yl)sulfonyl-amino]phenyl]-9H-pyrido[3,4-b]indole-3-carboxylate (30 mg, crude) as yellow solid. LCMS: m/z 512.1[M+H]r.

To a solution of methyl 7-fluoro-1-[4-[methyl-(4-methylpiperazin-1-yl)sulfonyl-amino]phenyl]-9H-pyrido[3,4-b]indole-3-carboxylate (30 mg, 58.64 umol, N/A purity, 1 eq) in H₂O (2 mL), THF (2 mL), MeOH (2 mL) was added NaOH (23.46 mg, 586.43 umol, 10 eq). Then the mixture was stirred at 60° C. for 2 h. LCMS showed the methyl 7-fluoro-1-[4-[methyl-(4-methylpiperazin-1-yl)sulfonyl-amino]phenyl]-9H-pyrido[3,4-b]indole-3-carboxylate was consumed and the desired product was detected. The mixture was poured into water (40 mL), extracted with EA (20 mL×3). The organic layer was washed with brine (20 mL), dried over Na₂SO₄, concentrated in vacuo. The residue was purified by prep-HPLC: column: Phenomenex luna C18 150*40 mm*15 um; mobile phase: [water (0.1% TFA)-ACN]; B %: 16%-46%, 10 min], column: Phenomenex Synergi C18 150*25 mm*10 um; mobile phase: [water (0.1% TFA)-ACN]; B %: 51%-81%, 10 min] twice to give 7-fluoro-1-[4-[methyl-(4-methylpiperazin-1-yl)sulfonyl-amino]phenyl]-9H-pyrido[3,4-b]indole-3-carboxylic acid (1 mg, 1.95 umol, 3.33% yield, 97.2% purity) as a light yellow solid, which was confirmed by ¹H-NMR, LC-MS and HPLC. LCMS: m/z 498.2[M+H]⁺. ¹H NMR (DMSO-d₆ 400 MHz) δ 12.09 (s, 1H), 8.93 (s, 1H), 8.51-8.48 (m, 1H), 8.12-8.09 (m, 2H), 7.72-7.69 (m, 2H), 7.42-7.38 (m, 1H), 7.23-7.17 (m, 1H), 3.85-3.75 (m, 4H), 3.20-3.15 (m, 7H).

### Example 19

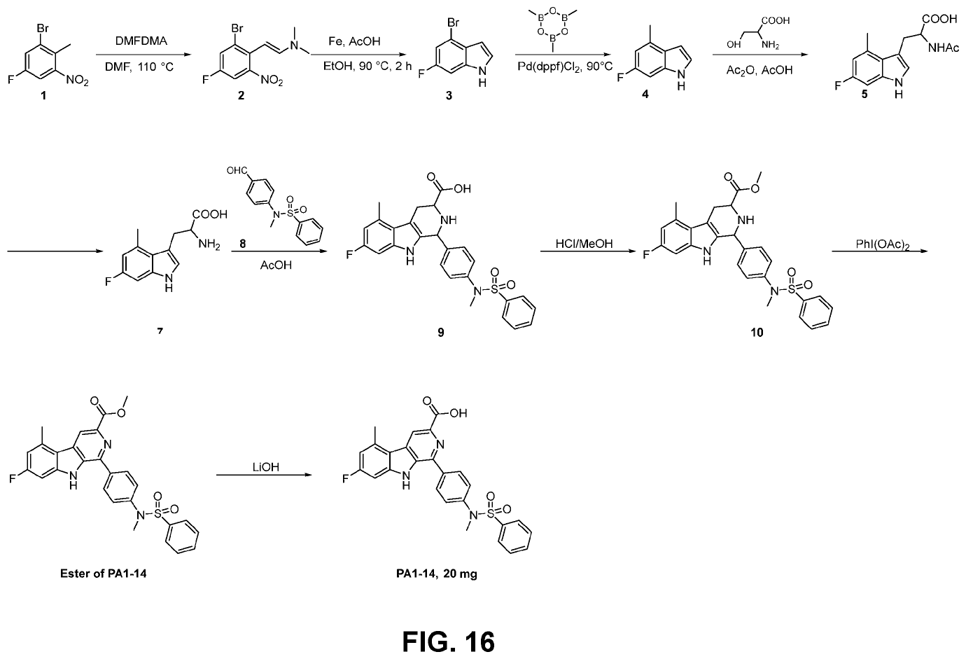

This example demonstrates the synthesis of PA1-14 (7-fluoro-5-methyl-1-(4-(N-methylphenylsulfonamido)phenyl)-9H-pyrido[3,4-b]indole-3-carboxylic acid) and its ester. The reaction scheme to produce compound PA1-14 and its ester is shown in FIG. 16, and each individual step in the synthesis is described in detail below. Note that compound 8 in FIG. 16 is the same as compound 5[PA99] described in Example 1.

The mixture of 1-bromo-5-fluoro-2-methyl-3-nitro-benzene (10 g, 42.73 mmol, 1 eq) and DMFDMA (17.94 g, 150.55 mmol, 20 mL, 3.52 eq) and pyrrolidine (3.41 g, 47.92 mmol, 4 mL, 1.12 eq) in DMF (100 mL) was stirred at 120° C. for 3 h. The mixture was poured into water (200 mL) and extracted with EA (150 mL×3). The combined organic phase was washed with brine (100 mL×2), dried with anhydrous Na₂SO₄, filtered and concentrated in vacuum. The mixture was concentrated under reduced pressure affording (E)-2-(2-bromo-4-fluoro-6-nitro-phenyl)-N,N-dimethyl-ethenamine (14 g, crude) as brown oil, which was used directly for next step without further purification.

To a solution of (E)-2-(2-bromo-4-fluoro-6-nitro-phenyl)-N,N-dimethyl-ethenamine (13 g, 44.97 mmol, 1 eq) in AcOH (100 mL) and EtOH (100 mL) was added Fe (25.11 g, 449.67 mmol, 10 eq) at 25° C. and then the mixture was stirred at 90° C. for 2 h. LCMS showed the 2(E)-2-(2-bromo-4-fluoro-6-nitro-phenyl)-N,N-dimethyl-ethenamine was consumed and the desired product was detected. The TLC (petroleum ether/ethyl acetate: 5/1) showed the starting material (E)-2-(2-bromo-4-fluoro-6-nitro-phenyl)-N,N-dimethyl-ethenamine was consumed and 3 new spots were observed. The mixture was poured into water (100 mL) and extracted with EA (100 mL×3). The organic layer was combined and washed with brine (80 mL×3), dried over Na₂SO₄, and concentrated in vacuo. The mixture was concentrated under reduced pressure affording the crude product as brown oil. The crude product was purified by silica gel column chromatography (petroleum ether/ethyl acetate, from 1/to 10/1) to give 4-bromo-6-fluoro-1H-indole (6 g, 25.12 mmol, 55.86% yield, 89.6% purity) as black brown oil, which was confirmed by ¹H-NMR and LCMS. LCMS: m/z 214.0 [M+H]⁺.

To a solution of 4-bromo-6-fluoro-1H-indole (5 g, 20.93 mmol, 89.6% purity, 1 eq) and 2,4,6-trimethyl-1,3,5,2,4,6-trioxatriborinane (52.55 g, 209.31 mmol, 58.52 mL, 50% purity, 10 eq) in dioxane (10 mL) and H₂O (0.5 mL) was added K₂CO₃ (8.68 g, 62.79 mmol, 3 eq) and Pd(dppf)Cl₂ (1.53 g, 2.09 mmol, 0.1 eq) at 25° C. and then the mixture was stirred at 90° C. for 12 h. The LC-MS and TLC (PE:PA=5:1) showed the starting material 4-bromo-6-fluoro-1H-indole was consumed and the desired product was detected. The mixture was concentrated under reduced pressure affording the crude product as brown oil. The crude product was purified by silica gel column chromatography (petroleum ether/ethyl acetate, from 1/0 to 1/1) to give the desired product as a brown oil. 6-fluoro-4-methyl-1H-indole (4 g, crude) was obtained as a brown oil, which was confirmed by ¹H-NMR. LCMS: m/z 150.0 [M+H]⁺. ¹H NMR (CDCl₃, 400 MHz) δ 8.16 (br S, 1H), 7.19-7.17 (m, 1H), 6.94-6.91 (m, 1H), 6.74-6.71 (m, 1H), 6.57-6.53 (m, 1H), 2.55 (s, 3H).

To a solution of 6-fluoro-4-methyl-1H-indole (4 g, 23.87 mmol, 89% purity, 1 eq) and 2-amino-3-hydroxy-propanoic acid (5.02 g, 47.73 mmol, 2.0 eq) in AcOH (50 mL) was added Ac₂O (19.92 g, 195.10 mmol, 18.27 mL, 8.17 eq) at 25° C. and then the mixture was stirred at 40° C. for 12 h. The LC-MS showed the starting material 6-fluoro-4-methyl-1H-indole was consumed and the desired product was detected. The mixture was diluted with H₂O (200 mL), extracted with EA (150 mL×4). The combined extracts were washed with H₂O (300 mL×3), brine (400 mL), evaporated in vacuo to get 2-acetamido-3-(6-fluoro-4-methyl-1H-indol-3-yl)propanoic acid (20 g, crude) as a black brown oil. The crude product was used into the next step without further purification. LCMS: m/z 279.0 [M+H]⁺.

The mixture of 2-acetamido-3-(6-fluoro-4-methyl-1H-indol-3-yl)propanoic acid (8.0 g, 28.75 mmol, 1 eq) in H₂O (85 mL) was added NaOH (15 g, 0.375 mol, 34.78 eq) at 25° C. and then the mixture was stirred at 75° C. for 12 h. LCMS showed the 2-acetamido-3-(6-fluoro-4-methyl-1H-indol-3-yl)propanoic acid was consumed and the desired product was detected. After cooling, the mixture was diluted with H₂O (50 mL), adjusted to make pH=7 with HCl (1 M in H₂O). The water phase was purified by Reversed-phase HPLC (0.1% TFA condition) to give 2-amino-3-(6-fluoro-4-methyl-1H-indol-3-yl)propanoic acid (650 mg, 2.58 mmol, 8.98% yield, 93.8% purity) as a brown solid, which was confirmed by LC-MS. LCMS: m/z 236.9 [M+H]⁺.

To a solution of 2-amino-3-(6-fluoro-4-methyl-1H-indol-3-yl)propanoic acid (300 mg, 1.19 mmol, 93.8% purity, 1 eq) in AcOH (15 mL) was added N-(4-formylphenyl)-N-methyl-benzenesulfonamide (339.14 mg, 1.19 mmol, 96.7% purity, 1 eq). Then the mixture was stirred at 120° C. for 1 h. LCMS showed the 2-amino-3-(6-fluoro-4-methyl-1H-indol-3-yl)propanoic acid was consumed and the desired product was detected. The mixture was concentrated under reduced pressure affording t1-[4-[benzenesulfonyl(methyl)amino]phenyl]-7-fluoro-5-methyl-2,3,4,9-tetrahydro-1H-pyrido[3,4-b]indole-3-carboxylic acid (650 mg, crude) as brown oil. The crude product was used into the next step without further purification. LCMS: m/z 494.2[M+H]⁺.

To a solution of 1-[4-[benzenesulfonyl(methyl)amino]phenyl]-7-fluoro-5-methyl-2,3,4,9-tetrahydro-1H-pyrido[3,4-b]indole-3-carboxylic acid (650 mg, 1.32 mmol, 1 eq) in HCl/MeOH (4 M, 188.73 mL, 573.23 eq). Then the mixture was stirred at 25° C. for 4 h. LCMS and TLC (Petroleum ether:Ethyl acetate=3:1) showed the 11-[4-[benzenesulfonyl(methyl)amino]phenyl]-7-fluoro-5-methyl-2,3,4,9-tetrahydro-1H-pyrido[3,4-b]indole-3-carboxylic acid was consumed and the desired product was detected. The mixture was concentrated in vacuo to afford the residue as black solid. Then the residue was poured into water (15 mL) and basified by K₂CO₃ solid to make pH=10-11. Then the mixture was extracted by EA (10 mL×3). Then the organic layers were washed by brine (20 mL×2), dried by Na₂SO₄, concentrated in vacuo. The crude product was purified by silica gel column chromatography (petroleum ether/ethyl acetate, from 10/1 to 0/1) to give the desired product as a yellow solid. methyl 1-[4-[benzenesulfonyl(methyl)amino]phenyl]-7-fluoro-5-methyl-2,3,4,9-tetrahydro-1H-pyrido[3,4-b]indole-3-carboxylate (240 mg, 472.84 umol, 35.90% yield) was obtained as yellow solid. LCMS: m/z 508.1 [M+H]⁻.

To a solution of methyl 1-[4-[benzenesulfonyl(methyl)amino]phenyl]-7-fluoro-5-methyl-2,3,4,9-tetrahydro-1H-pyrido[3,4-b]indole-3-carboxylate (120 mg, 219.87 umol, 93% purity, 1 eq) in DMF (5 mL) was added PhI(OAc)₂ (212.46 mg, 659.61 umol, 3 eq). Then the mixture was stirred at 25° C. for 1 h. LCMS and TLC (Petroleum ether:Ethyl acetate=4:1) showed the methyl 1-[4-[benzenesulfonyl(methyl)amino]phenyl]-7-fluoro-5-methyl-2,3,4,9-tetrahydro-1H-pyrido[3,4-b]indole-3-carboxylate was consumed and the desired product was detected. To the solution was added water (30 mL), extracted by EA (10 mL×3). Then the organic layers were combined and washed by brine (10 mL×3), dried by Na₂SO₄, concentrated in vacuo. There were 2 batches of Ester of PA1-14 obtained. Batch 1: The pure Ester of PA1-14 (11 mg, 21.85 umol, 9.94% yield, 100% purity) was purified by prep-HPLC: column: Phenomenex luna C18 150*40 mm*15 um; mobile phase: [water (0.1% TFA)-ACN]; B %: 54%-84%, 10 min] to be obtained as yellow solid, confirmed by ¹H-NMR and LC-MS and HPLC. It was delivered. Batch 2: The crude Ester of PA1-14 was purified by silica gel column chromatography (petroleum ether/ethyl acetate, from 10/1 to 0/1) to give Ester of PA1-14 (60 mg, 119.16 umol, 54.19% yield) as a yellow solid. It was used for next step. LCMS: m/z 504.2 [M+H]⁺. ¹H NMR (DMSO-d₆ 400 MHz) δ 8.74 (s, 1H), 7.98-7.95 (m, 2H), 7.75-7.73 (m, 1H), 7.65-7.63 (m, 4H), 7.42-7.39 (m, 2H), 7.28-7.25 (m, 1H), 7.06-7.04 (m, 2H), 3.93 (s, 3H), 3.26 (s, 3H), 2.90 (s, 3H).

To a solution of methyl 1-[4-[benzenesulfonyl(methyl)amino]phenyl]-7-fluoro-5-methyl-9H-pyrido[3,4-b]indole-3-carboxylate (60 mg, 119.16 umol, 1 eq) in H₂O (2 mL), THF (2 mL) and MeOH (2 mL) was added NaOH (47.66 mg, 1.19 mmol, 10 eq). Then the mixture was stirred at 60° C. for 12 h. LCMS showed the methyl 1-[4-[benzenesulfonyl(methyl)amino]phenyl]-7-fluoro-5-methyl-9H-pyrido[3,4-b]indole-3-carboxylate was consumed and the desired product was detected. The mixture was poured into water (10 mL) and HCl (1 M) to make pH=5-6. Then the solution was extracted with EA (20 mL×3). The organic layers were combined and washed by water (15 mL×2), dried over Na₂SO₄, concentrated in vacuo. The residue was purified by prep-HPLC (column: Phenomenex Luna C18 150*25 mm*10 um; mobile phase: [water (0.1% TFA)-ACN]; B %: 45%-75%, 10 min) twice. 1-[4-[benzenesulfonyl(methyl)amino]phenyl]-7-fluoro-5-methyl-9H-pyrido[3,4-b]indole-3-carboxylic acid (8.6 mg, 17.21 umol, 14.44% yield, 97.95% purity) was obtained as yellow solid. ¹HNMR, HPLC and LCMS confirmed the product. LCMS: m/z 490.1 [M+H]⁺. ¹H NMR (DMSO-d₆ 400 MHz) δ 12.08 (s, 1H), 8.73 (s, 1H), 8.03 (d, J=8.4 Hz, 2H), 7.74-7.71 (m, 1H), 7.65-7.62 (m, 4H), 7.42-7.38 (m, 2H), 7.28-7.25 (m, 1H), 7.06-7.04 (m, 1H), 3.26 (s, 3H), 2.89 (s, 3H).

### Example 20

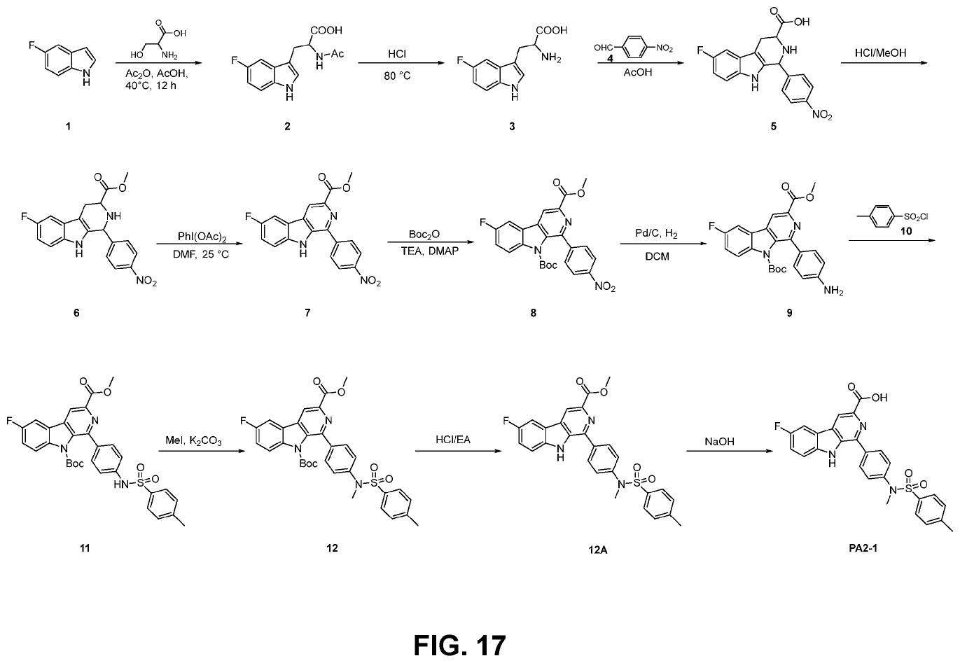

This example demonstrates the synthesis of PA2-1 (6-fluoro-1-[4-[methyl-(p-tolylsulfonyl)amino]phenyl]-9H-pyrido[3,4-b]indole-3-carboxylic acid) and its ester. The reaction scheme to produce compound PA2-1 and its ester is shown in FIG. 17, and each individual step in the synthesis is described in detail below. Note that compound 3 in FIG. 17 is the same as compound 4[PA1-3] described in Example 2, and this compound was prepared in the same way.

To a solution of compound 3 (18.0 g, 81.0 mmol, 1.0 eq) and compound 4 (7.65 g, 50.6 mmol, 0.63 eq) in AcOH (150 mL). The solution was stirred at 100° C. for 12 h. LCMS showed the compound 3 was consumed completely and desired mass was detected. There were 4 parallel reactions (18 g×4 of compound 3) The reactions were concentrated in vacuo to give a crude compound 5 (105 g, crude) as black brown oil. LCMS: m/z 356.0 [M+H]⁺.

To a solution of compound 5 (52.0 g, 146 mmol, 1.0 eq) was added HCl/MeOH (4 M, 500 mL) at 25° C. under N₂. The mixture was stirred at 25° C. for 12 h. The reaction was poured into water (1.5 L) and the solution was adjust by NaHCO₃ (sat.) to pH=7-8, then was extracted with EA (1 L×4). The organic layers were combined and washed with brine (2 L), dried over Na₂SO₄, and concentrated in vacuo. The residue was purified by silica gel chromatography with PE/EA=1:1 to 1:3 to give compound 6 (13.0 g, 5.3% yield, 56.3% purity) as black brown solid, detected by LCMS. LCMS: m/z 370.3 [M+H]⁺.

To a solution of compound 6 (12.0 g, 18.3 mmol, 1.0 eq) in DMF (120 mL) was added PhI(OAc)₂ (23.6 g, 73.2 mmol, 4.0 eq) at 25° C. under N₂. Then the mixture was stirred at 25° C. for 12 h. The reaction was poured into water (100 mL), extracted with EA (100 mL×2). The organic layer was combined and washed with brine (200 mL), dried over Na₂SO₄, and concentrated vacuo. The crude was purified by silica gel chromatography with PE/EA=3:1 to 1:2 to give a crude. The crude product was purified by reversed-phase HPLC (0.1% TFA condition) to give compound 7 (5.20 g, 35% yield, 45.7% purity) as black brown solid, detected by LCMS. LCMS: n/z 366.3 [M+H]⁺.

To a solution of compound 7 (2.60 g, 3.25 mmol, 1.0 eq) in DCM (50 mL) was added DMAP (119 mg, 976 umol, 0.3 eq) and TEA (658 mg, 6.51 mmol, 2.0 eq) at 25° C. under N₂. Then Boc₂O (1.42 g, 6.51 mmol, 2.0 eq) was added to the solution and then the mixture was stirred at 25° C. for 4 h. The LCMS and HPLC showed the compound 7 was consumed and the desired mass was detected. The reaction was poured into water (100 mL), extracted with DCM (100 mL×2). Then the organic layers were combined and washed with brine (100 mL×3), dried over Na₂SO₄, and concentrated in vacuo. The residue was purified by prep-HPLC (column: Phenomenex Luna C18 250×50 mm*15 um; mobile phase: [water (0.1% TFA)-ACN]; B %: 40%-70%, 20 min) to give compound 8 (1.00 g, 27% yield, 81% purity) as yellow solid, detected by LCMS: m/z 466.2 [M+H]⁺.

To a solution of compound 8 (600 mg, 1.04 mmol, 1.0 eq) in DCM (30 mL) was added Pd/C (500 mg, 10% purity) at 25° C. Then the mixture was stirred at 25° C. under H₂ (15 psi) for 2 h. LCMS showed the compound 8 was consumed and the desired mass was detected. The reaction was filtrated and the solution was concentrated in vacuo. The residue was dried in vacuo to give compound 9 (550 mg, crude) as yellow solid. LCMS: m/z 436.2 [M+H]⁻.

To a solution of compound 9 (130 mg, 298 umol, 1.0 eq) and pyridine (188 mg, 2.39 mmol, 8.0 eq) in THF (8 mL) was added compound 10 (142 mg, 746 umol, 2.5 eq) at 25° C. under N₂. Then the mixture was stirred at 25° C. for 12 h. The LCMS and TLC (PE/EA=3:1) showed the compound 9 was consumed and the desired mass was detected. The reaction was poured into water (10 mL) and extracted with EA (10 mL×3). The organic layers were combined and washed with brine (30 mL), dried over Na₂SO₄, concentrated in vacuo. The residue was purified by silica gel chromatography with PE/EA=3:1 to 2:1 to give compound 11 (160 mg, 91% yield) as yellow solid. LCMS: m/z 590.3 [M+H]⁺.

To a solution of compound 11 (0.160 g, 271 umol, 1.0 eq) in THF (8 mL) was added K₂CO₃ (113 mg, 814 umol, 3.0 eq) and MeI (116 mg, 814 umol, 3.0 eq) at 25° C. under N₂. Then the mixture was stirred at 60° C. for 4 h. LCMS showed that the compound 11 was consumed completely and desired mass was detected. The reaction was poured into water (20 mL), extracted with EA (20 mL×3). The organic layer was combined and washed with brine (30 mL), dried over Na₂SO₄, concentrated in vacuo to give a crude compound 12 (0.160 g, 98% yield) as yellow solid. ¹H NMR confirmed the product. LCMS: n/z 604.1 [M+H]⁺; ¹H NMR (CDCl₃, 400 MHz) δ 8.67 (s, 1H), 8.20-8.18 (m, 1H), 7.90-7.88 (m, 2H), 7.80-7.75 (m, 1H), 7.56-7.54 (m, 2H), 7.40-7.35 (m, 1H), 7.29-7.26 (m, 4H), 4.07 (s, 3H), 3.20 (s, 3H), 2.43 (s, 3H), 1.33 (s, 9H).

The compound 12 (160 mg, 265 umol, 1.0 eq) was dissolved in HCl/EtOAc (4 M, 20 mL) at 25° C. Then the mixture was stirred at 60° C. for 1 h. LCMS showed the compound 12 was consumed and the desired mass was detected. The reaction was concentrated in vacuo to give a crude compound 12A (130 mg, 97% yield) as yellow solid. LCMS: m/z 504.2 [M+H]⁺.

To a solution of compound 12A (130 mg, 258 umol, 1.0 eq) in MeOH (5 mL) and H₂O (5 mL) was added NaOH (260 mg, 6.50 mmol, 25.2 eq) at 25° C. Then the mixture was stirred at 60° C. for 2 h. LCMS showed the compound 12A was consumed and the desired mass was detected. The reaction was acidified by TFA (purity: 95%) to pH=3-4. Then the mixture was concentrated in vacuo. The residue was purified by Prep-HPLC (column: Phenomenex Luna C18 150*25 mm*10 um; mobile phase: [water (0.1% TFA)-ACN]; B %: 46%-76%, 10 min) to give PA2-1 (44.0 mg, 35% yield, 99.4% purity) as yellow solid, confirmed by HPLC, LCMS, and ¹H NMR. LCMS: m/z 490.1 [M+H]⁺; ¹H NMR (DMSO-d₆, 400 MHz) δ 12.00 (s, 1H), 8.95 (s, 1H), 8.33-8.30 (m, 1H), 8.06 (d, J=8.8 Hz, 2H), 7.70-7.69 (m, 1H), 7.52-7.50 (m, 3H), 7.48-7.38 (m, 4H), 3.23 (s, 3H), 2.38 (s, 3H).

### Example 21

This example demonstrates the synthesis of PA2-2 (6-fluoro-1-[4-[(4-methoxyphenyl)-sulfonyl-methyl-amino]phenyl]-9H-pyrido[3,4-b]indole-3-carboxylic acid), PA2-3 (6-fluoro-1-[4-[(4-fluorophenyl)sulfonyl-methyl-amino]phenyl]-9H-pyrido[3,4-b]indole-3-carboxylic acid), PA2-4 (1-[4-[(4-bromophenyl)sulfonyl-methyl-amino]phenyl]-6-fluoro-9H-pyrido[3,4-b]indole-3-carboxylic acid), PA2-6 (6-fluoro-1-(4-(N-methylpyridine-3-sulfonamido)phenyl)-9H-pyrido[3,4-b]indole-3-carboxylic acid), PA1-13A-2 (1-(4-((4-cyano-N-methylphenyl)sulfonamido)phenyl)-7-fluoro-9H-pyrido[3,4-b]indole-3-carboxylic acid) and each of their esters.

PA2-2, PA2-3, PA2-4, PA2-6, and PA1-13A-2 and each of their esters were prepared using the same synthetic route as PA2-1 in Example 20, starting from compound 9 (see FIG. 17), except that the appropriate sulfonyl chloride was employed to achieve the appropriate ring substitutions. See, for example, FIG. 14 for the illustrative synthetic route.

PA2-2: LCMS: m/z 506.1 [M+H]⁺. ¹H NMR (CDCl₃, 400 MHz) δ 8.92-8.88 (m, 2H), 7.95-7.88 (m, 3H), 7.59-7.57 (m, 3H), 7.45-7.42 (m, 3H), 6.98-6.96 (m, 2H), 3.88 (s, 3H), 3.26 (s, 3H). Methyl ester of PA2-2: LCMS: m/z 520.2 [M+H]⁺.

PA2-3: LCMS: m/z 494.1 [M+H]⁺. ¹H NMR (CDCl₃, 400 MHz) δ 8.91 (s, 1H), 8.86 (s, 1H), 7.96-7.89 (m, 3H), 7.68-7.66 (m, 2H), 7.61-7.55 (m, 1H), 7.44-7.42 (m, 3H), 7.23-7.19 (m, 2H), 3.29 (s, 3H). Methyl ester of PA2-3: LCMS: m/z 508.1 [M+H]⁺.

PA2-4: LCMS: m/z 556.0 [M+H]⁺. ¹H NMR (DMSO-d₆, 400 MHz) δ 12.00 (s, 1H), 8.96 (s, 1H), 8.31 (d, J=2.8 Hz, 1H), 8.06 (d, J=8.4 Hz, 2H), 7.87-7.84 (m, 2H), 7.56 (d, J=2.0 Hz, 1H), 7.53-7.41 (m, 5H), 3.31 (s, 3H). Methyl ester of PA2-4: LCMS: m/z 570.0 [M+H]⁺.

PA2-6: LCMS: m/z 476.9 [M+H]⁺. ¹H NMR (400 MHz, DMSO-d₆, δ 12.02 (s, 1H), 8.96 (s, 1H), 8.93-8.89 (m, 1H), 8.78 (s, 1H), 8.35-8.30 (m, 1H), 8.08 (d, J=8.4 Hz, 2H), 8.05-7.99 (m, 1H), 7.73-7.68 (m, 2H), 7.52-7.45 (m, 1H), 7.43 (d, J=8.4 Hz, 2H), 3.31 (s, 3H). Methyl ester of PA2-6: LCMS: m/z 491.2 [M+H]⁺.

PA1-13A-2: LCMS: m/z 501.1 [M+H]⁺. ¹H NMR (400 MHz, DMSO-d₆, δ 12.07 (s, 1H), 8.93 (s, 1H), 8.51-8.49 (m, 1H), 8.13 (d, J=8.4 Hz, 2H), 8.07 (d, J=8.4 Hz, 2H), 7.81 (d, J=8.4 Hz, 2H), 7.44-7.40 (m, 3H), 7.23-7.18 (m, 1H), 3.30 (s, 3H). Methyl ester of PA1-13A-2: LCMS: m/z 515.2 [M+H]⁺. ¹H NMR (400 MHz, DMSO-d₆, δ 12.09 (s, 1H), 8.95 (s, 1H), 8.51-8.49 (m, 1H), 8.13 (d, J=8.8 Hz, 2H), 8.02-7.99 (m, 2H), 7.80 (d, J=8.4 Hz, 2H), 7.44-7.41 (m, 3H), 7.23-7.18 (m, 1H), 3.94 (s, 3H), 3.29 (s, 3H).

### Example 22

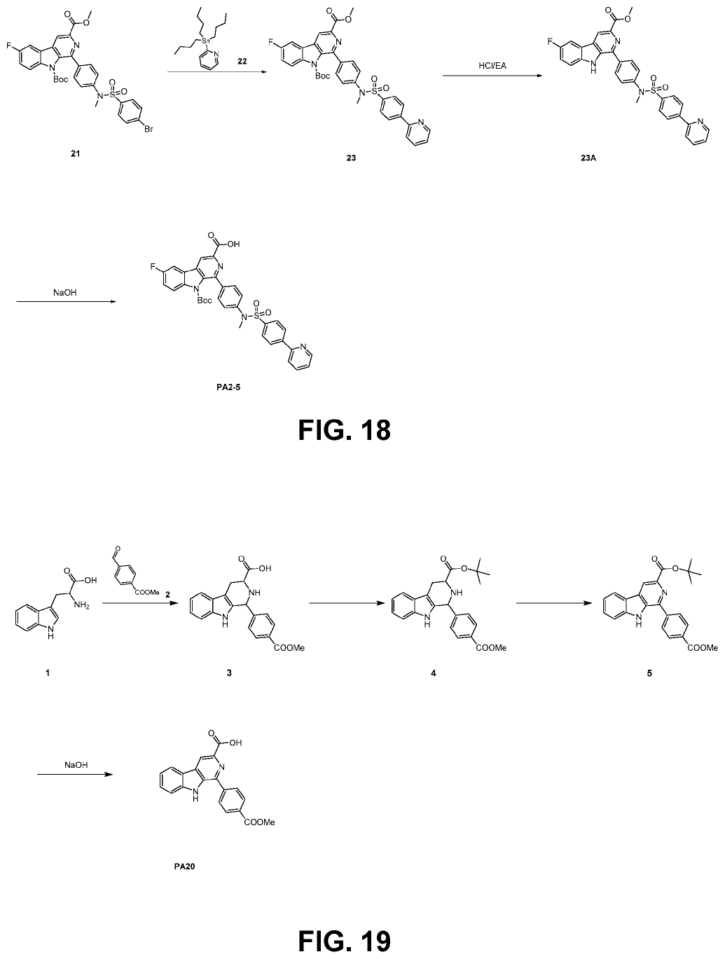

This example demonstrates the synthesis of PA2-5 (6-fluoro-1-[4-[methyl-[4-(2-pyridyl)phenyl]sulfonyl-amino]phenyl]-9H-pyrido[3,4-b]indole-3-carboxylic acid) and its ester. The reaction scheme to produce compound PA2-5 and its ester is shown in FIG. 18, and each individual step in the synthesis is described in detail below. This synthesis starts from the methyl ester of PA2-4.

To a solution of the methyl ester of PA2-4 (50.0 mg, 74.8 umol, 1.0 eq) in dioxane (10 mL) was added compound 22 (41.3 mg, 112 umol, 1.5 eq) and Pd(PPh₃)₄ (8.64 mg, 7.48 umol, 0.1 eq) at 25° C. under N₂. Then the mixture was purged with N₂ for three times and stirred at 100° C. for 4 h. The LC-MS showed the methyl ester of PA2-4 was consumed and the desired mass was detected. TLC (EA/MeOH=20:1) showed the methyl ester of PA2-4 was consumed and new spots were formed. The reaction was concentrated in vacuo. The residue was purified by silica gel chromatography with EA/MeOH=20:1 to give compound 23 (40.0 mg, 80% yield) as yellow oil. LCMS: m/z 667.2 [M+H]⁺.

The compound 23 (50.0 mg, 74.9 umol, 1.0 eq) was dissolved in HCl/EtOAc (4 M, 15 mL). Then the mixture was stirred at 60° C. for 0.5 h. LCMS showed that the compound 23 was consumed completely and desired mass was detected. The reaction was concentrated in vacuo to give a crude of compound 23A (50 mg, crude) as yellow solid. LCMS: m/z 567.2 [M+H]⁺.

To a solution of compound 23A (50.0 mg, 88.3 umol, 1.0 eq) in MeOH (3 mL) and H₂O (3 mL) was added NaOH (80.0 mg, 2.00 mmol, 22.6 eq). Then the mixture was stirred at 60° C. for 12 h. LCMS showed that compound 23A was consumed completely and desired mass was detected. The reaction was concentrated in vacuo. The residue was purified by Prep-HPLC (column: Phenomenex luna C18 150*25 mm*10 um; mobile phase: [water (0.1% TFA)-ACN]; B %: 33%-63%, 10 min) to give PA2-5 (18.0 mg, 31% yield, 99.8% purity, TFA salt) as yellow solid, confirmed by ¹H NMR and LCMS and HPLC. LCMS: m/z 553.1 [M+H]⁺. ¹H NMR (DMSO-d₆, 400 MHz) δ 12.01 (s, 1H), 8.95 (s, 1H), 8.75 (d, J=8.4 Hz, 1H), 8.35-8.32 (m, 3H), 8.10-8.06 (m, 3H), 7.96-7.94 (m, 1H), 7.75-7.73 (m, 3H), 7.45-7.42 (m, 4H), 3.30 (s, 3H).

### Example 23

This example demonstrates the synthesis of PA20 (1-(4-methoxycarbonylphenyl)-9H-pyrido[3,4-b]indole-3-carboxylic acid) and its t-butoxy ester. The reaction scheme to produce compound PA20 and its ester is shown in FIG. 19, and each individual step in the synthesis is described in detail below.

A suspension of compound 1 (1.0 g, 4.90 mmol, 1.0 eq) and compound 2 (804 mg, 4.90 mmol, 1.0 eq) in AcOH (20 mL) was stirred at 120° C. for 1 h. The reaction mixture was concentrated under reduced pressure to give compound 3 (1.40 g, 3.60 mmol, 73% yield, 90% purity) as light brown solid. LCMS: m/z 350.7 [M+H]⁺.

To a suspension of compound 3 (700 mg, 2.00 mmol, 1 eq) in dioxane (30 mL) was added H₂SO₄ (3.68 g, 37.5 mmol, 2 mL, 18.8 eq). Then 2-methylprop-1-ene (1.0 eq) was bubbled into the suspension and the reaction was stirred at 25° C. for 37 h. The reaction solution was poured onto ice (˜100 g) in a separating funnel. DCM (100 mL) was added and the mixture was basified by solid NaOH to pH=9. The organic layer was separated and the aqueous layer was extracted with DCM (40 mL). The combined organic layer was concentrated under reduced pressure to afford a residue. The residue was purified by column (PE/EA=3/1˜2/1) to afford the crude product which was purified by prep-HPLC (column: Luna C18 150*25 5u; mobile phase: [water (0.225% FA)-ACN]; B %: 28%-58%, 10 min) to give the product compound 4 (250 mg, 442 umol, 22.1% yield, 71.9% purity) as white solid. LCMS: m/z 406.9 [M+H]⁺.

To a solution of compound 4 (250 mg, 615 umol, 1.0 eq) in DMF (1 mL) was added PhI(OAc)₂ (374 mg, 1.16 mmol, 1.89 eq). The reaction was stirred at 25° C. for 2 hours to afford brown solution. The reaction solution was directly purified by prep-HPLC (FA) to afford the product compound 5 (120 mg, 295 umol, 48% yield, 99.1% purity) as white solid. LCMS: m/z 403.0 [M+H]⁺.

A solution of compound 5 (110 mg, 273 umol, 1.0 eq) in DCM (1 mL) and TFA (3 mL) was stirred at 25° C. for 2 hours. The solution was concentrated under reduced pressure and the residue was dissolved in DCM/MeOH=5/1 (20 mL). The solution was neutralized with ammonia to pH=9. Then the solution was concentrated and the residue was washed with water (30 mL) to give PA20 (59.0 mg, 169 umol, 62% yield, 99.0% purity) as light yellow solid. LCMS: m/z 347.0 [M+H]⁺; ¹H NMR (DMSO-d₆, 400 MHz) δ ppm 11.87 (s, 1H), 8.89 (s, 1H), 8.39-8.36 (m, 1H), 8.30-8.10 (m, 4H), 7.69-7.66 (m, 1H), 7.62-7.57 (m, 1H), 7.34-7.29 (m, 1H), 3.91 (s, 3H).

### Example 24

This example demonstrates the synthesis of PA24 (1-(3-methoxycarbonylphenyl)-9HPage pyrido[3,4-b]indole-3-carboxylic acid), its t-butoxy ester PA43, PA28 (1-(1H-indol-5-yl)-9H-pyrido[3,4-b]indole-3-carboxylic acid), and its t-butoxy ester, each of which were prepared using the same synthetic route as PA20 in Example 23, except compound 2 was substituted from the appropriate aldehyde to achieve the appropriate ring structure/substitutions. See, for example, FIG. 19 for the illustrative synthetic route.

PA24: LCMS: m/z 347.0 [M+H]⁺; ¹H NMR (DMSO-d₆, 400 MHz) δ ppm 12.02 (s, 1H), 8.95 (s, 1H), 8.59 (d, J=1.6 Hz, 1H), 8.44 (d, J=7.6 Hz, 1H), 8.32-8.31 (m, 1H), 8.15 (d, J=8.0 Hz, 1H), 7.83-7.80 (m, 1H), 7.71-7.68 (m, 1H), 7.68-7.62 (m, 1H), 7.40-7.35 (m, 1H), 3.93 (s, 3H).

PA43: LCMS: m/z 403.2 [M+H]⁺; ¹H NMR (CDCl₃ 400 MHz) δ ppm 8.92 (s, 1H), 8.77 (s, 1H), 8.64 (d, J=1.6 Hz, 1H), 8.28-8.25 (m, 2H), 8.14 (d, J=7.6 Hz, 1H), 7.66-7.61 (m, 3H), 7.45-7.40 (m, 1H), 3.95 (s, 3H), 1.72 (s, 9H).

PA28: LCMS: m/z 328.0 [M+H]⁺; ¹H NMR (DMSO-d₆, 400 MHz) δ ppm 11.81 (s, 1H), 11.32 (s, 1H), 8.82 (s, 1H), 8.40-8.34 (m, 2H), 8.26 (s, 1H), 7.85-7.80 (m, 1H), 7.71-7.68 (m, 1H), 7.64-7.57 (m, 2H), 7.47 (s, 1H), 7.31-7.29 (m, 1H), 6.62 (s, 1H). T-butoxy ester of PA28: LCMS: m/z 384.1 [M+H]⁺.

### Example 25

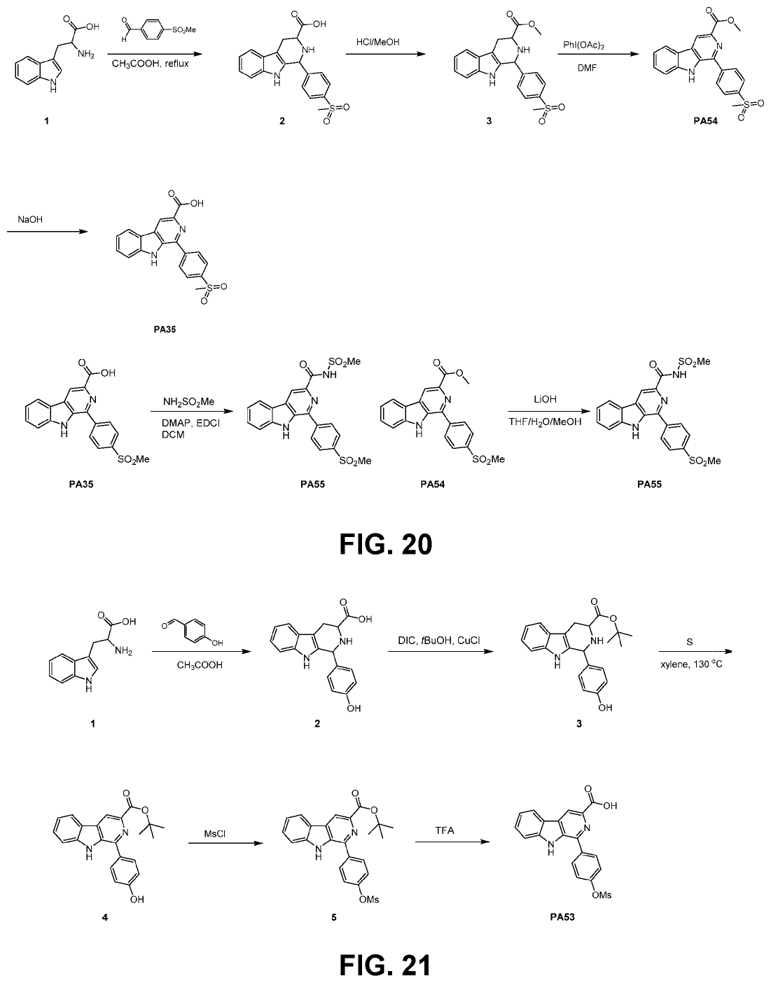

This example demonstrates the synthesis of PA35 (1-(4-methylsulfonylphenyl)-9H-pyrido[3,4-b]indole-3-carboxylic acid), PA54 (which is the methyl ester of PA35), and PA55. The reaction scheme to produce PA35, PA54, and PA55 is shown in FIG. 20, and each individual step in the synthesis is described in detail below.

A suspension of compound 1 (500 mg, 2.45 mmol, 1.0 eq) and 4-methylsulfonylbenzaldehyde (451 mg, 2.45 mmol, 1.0 eq) in HOAc (20 mL) was heated at 120° C. for 2 h under nitrogen to give a yellow solution. The solution was concentrated under reduced pressure to give the crude product which was dried by azeotropy with EA (30 mL×3) to give compound 2 (900.00 mg, crude) as yellow solid. LCMS: m/z 371.0 [M+H]⁺.

A solution of compound 2 (900 mg, 2.43 mmol, 1.0 eq) in MeOH (30 mL) was added H₂SO₄ (3.68 g, 37.5 mmol, 2 mL, 15.4 eq), then the reaction was stirred at 65° C. for 16 h. The solution was concentrated under reduced pressure to get a residue, diluted with water (20 mL) and adjusted to pH=9 by NaOH, extracted with DCM (30 mL×2), combined organic layer was dried over Na₂SO₄, concentrated to give compound 3 (1.60 g, crude) as light yellow solid. LCMS: m/z 384.8 [M+H]⁺.

A solution of compound 3 (1.60 g, 4.16 mmol, 1.0 eq) in DMF (8 mL) was added PhI(OAc)₂ (2.33 g, 7.23 mmol, 1.74 eq), then stirred at 25° C. for 2 h. The solution was added water (100 mL) and extracted with EA (50 mL), the aqueous phase was filtered to give PA54 (500 mg, 959 umol, 23.1% yield, 73% purity) as light yellow solid. LCMS: m/z 380.8 [M+H]⁺. Alternatively, to a suspension of compound 3 (3.75 g, 9.75 mmol, 1.00 eq) in DMF (8.00 mL) was added PhI(OAc)₂ (6.28 g, 19.50 mmol, 2.00 eq). The solution was stirred at 20° C. for 2 h. The suspension was poured into water (20 mL), extracted with DCM (30 mL×3). The combined organic phase was washed with brine (50 mL), dried with anhydrous Na₂SO₄, filtered and concentrated to give residue. The residue was diluted with DMSO (50 mL) and stirred at 20° C. for 1 h. The suspension was filtered and washed with MeOH (10 mL×3) to afford PA54 (1.80 g, 4.57 mmol, 46.87% yield, 96.5% purity) as a white solid, confirmed by LCMS, HPLC, and H NMR. LCMS: m/z 381.2 [M+H]⁺; ¹H NMR (DMSO-d₆, 400 MHz) δ ppm 12.11 (s, 1H), 9.01 (s, 1H), 8.47 (d, J=7.6 Hz, 1H), 8.27 (d, J=8.4 Hz, 2H), 8.18 (d, J=8.4 Hz, 2H), 7.71-7.68 (m, 1H), 7.66-7.63 (m, 1H), 7.38-7.34 (m, 1H), 3.95 (s, 3H), 3.34 (s, 3H).

A solution of PA54 (500 mg, 1.31 mmol, 1.0 eq) in THF (50 mL) and MeOH (10 mL) and H₂O (10 mL) was added LiOH·H₂O (550 mg, 13.1 mmol, 10.0 eq), then stirred at 25° C. for 16 hours. The solution was concentrated under reduced pressure and the residue was diluted with water (4 mL). The mixture was acidified by HCl (12 M) to pH=2. The precipitate was collected by filtration as the crude product. The crude product was pulped with DCM/MeOH=10:1, filtered to give PA35 (134 mg, 351 umol, 26.8% yield, 96% purity) as white solid. LCMS: m/z 366.7 [M+H]⁺; ¹H NMR (DMSO-d₆, 400 MHz) δ ppm 11.95 (s, 1H), 8.91 (s, 1H), 8.41 (d, J=8.0 Hz, 1H), 8.30-8.27 (m, 2H), 8.14-8.12 (m, 2H), 7.68-7.66 (m, 1H), 7.66-7.62 (m, 1H), 7.35-7.32 (m, 1H), 3.35 (s, 3H).

To the mixture of PA35 (120 mg, 328 umol, 1.0 eq), methanesulfonamide (37.4 mg, 393 umol, 1.2 eq) and 4-dimethylaminopyridine (120 mg, 983 umol, 3.0 eq) in DCM (5 mL) was added EDCI (126 mg, 655 umol, 2.0 eq) and the mixture was stirred at 15° C. for 12 h. The mixture was diluted with H₂O (30 mL), extracted with DCM (30 mL×5). The combined extracts were washed with H₂O (30 mL), brine (30 mL), evaporated in vacuum to get the crude product, which is a mixture of PA54 and PA55, both giving the same LCMS: n/z 444.0 [M+H]⁺. Both PA55 and PA54 are in one peak in LCMS (have the same retention time in LCMS). So the crude product was hydrolyzed to get the pure PA55.

The crude mixture of PA54 and PA55 (150 mg, crude) in MeOH (2 mL), THF (2 mL) and H₂O (1 mL) was added LiOH (40.0 mg, 1.67 mmol). The mixture was stirred at 23° C. for 48 h. The reaction mixture was concentrated under reduced pressure to remove THF and MeOH. Then the residue was acidized by HCl aqueous (1 M in H₂O) to pH=4˜5. The residue was purified by Prep-HPLC (HCl conditions) to give the desired product PA55. LCMS: m/z 444.2 [M+H]⁺; ¹H NMR (DMSO-d₆, 400 MHz) δ ppm 12.22 (s, 1H), 9.02 (s, 1H), 8.51-8.47 (m, 3H), 8.19 (d, J=8.4 Hz, 2H), 7.74-7.71 (m, 1H), 7.68-7.65 (m, 1H), 7.40-7.36 (m, 1H), 3.44 (s, 3H), 3.40 (s, 3H).

### Example 26

This example demonstrates the synthesis of PA53 (1-(4-methylsulfonyloxyphenyl)-9H-pyrido[3,4-b]indole-3-carboxylic acid) and its t-butoxy ester. The reaction scheme to produce compound PA53 and its ester is shown in FIG. 21, and each individual step in the synthesis is described in detail below.

A mixture of compound 1 (500 mg, 2.45 mmol, 1.0 eq.) and 4-hydroxybenzaldehyde (299 mg, 2.45 mmol, 1.0 eq.) in CH₃COOH (10 mL) was heated at 120° C. for 1 hour. The mixture was concentrated under reduced pressure to give a residue. The residue was triturated with EA (40 mL) to afford compound 2 (789 mg, 81% yield) as yellow solid. LCMS: m/z 309.1 [M+H]⁺.

A mixture of t-BuOH (9.48 g, 128 mmol, 50 eq.), DIC (12.9 g, 102 mmol, 40 eq.) and CuCl (507 mg, 5.12 mmol, 2.0 eq.) was stirred at 25° C. for 3 days. Then a solution of compound 2 (789 mg, 2.56 mmol, 1.0 eq.) in DCM (3 mL) was added and the mixture was stirred at 25° C. for 16 h. The reaction solution was poured into water (10 mL), extracted with DCM (10 mL×3). The combined organic phase was washed with brine (20 mL) and concentrated in vacuum to give residue. The residue was purified by column chromatography (DCM:MeOH=20:1) to afford compound 3 (400 mg, 32% yield) as brown solid. LCMS: m/z 365.1 [M+H]⁺.

To a solution of compound 3 (400 mg, 1.10 mmol, 1.0 eq.) in xylene (10 mL) was added S (352 mg, 11.0 mmol, 10 eq.), it was stirred at 130° C. for 10 h. The mixture was concentrated in vacuum. It was purified by column chromatography (PE/EA=10:1 to 1:1) to afford compound 4 (300 mg, 38% yield) as black brown solid. LCMS: m/z 361.1 [M+H]⁺.

To a solution of compound 4 (290 mg, 805 umol, 1.0 eq.) in Ethyl acetate (5 mL) was added Et₃N (163 mg, 1.61 mmol, 2.0 eq.) and MsCl (111 mg, 966 umol, 1.2 eq.) at 0° C., then it was stirred at 20° C. for 30 minutes. The reaction mixture was diluted with water (50 mL) and extracted with EA (50 mL×2). The combined organic layers were washed with brine (50 mL), dried over Na₂SO₄, filtered and concentrated under reduced pressure to give compound 5, which is the t-butoxy ester of PA53, (295 mg, crude) as black brown solid. LCMS: m/z 439.1 [M+H]⁺.

To a solution of compound 5 (290 mg, 661 umol, 1.0 eq.) in DCM (60 mL) was added TFA (44.7 g, 392 mmol, 590 eq.). The mixture was stirred at 20° C. for 10 h. It was purified by Prep-HPLC to afford PA53 (68.10 mg, 27% yield) as a yellow solid. LCMS: m/z 383.0 [M+H]⁺. ¹H NMR (DMSO-d₆, 400 MHz) δ 12.06 (s, 1H), 8.96 (s, 1H), 8.46 (d, J=8.0 Hz, 1H), 8.23-8.11 (m, 2H), 7.75-7.69 (m, 1H), 7.66-7.61 (m, 3H), 7.40-7.32 (m, 1H), 3.51 (s, 3H).

### Example 27

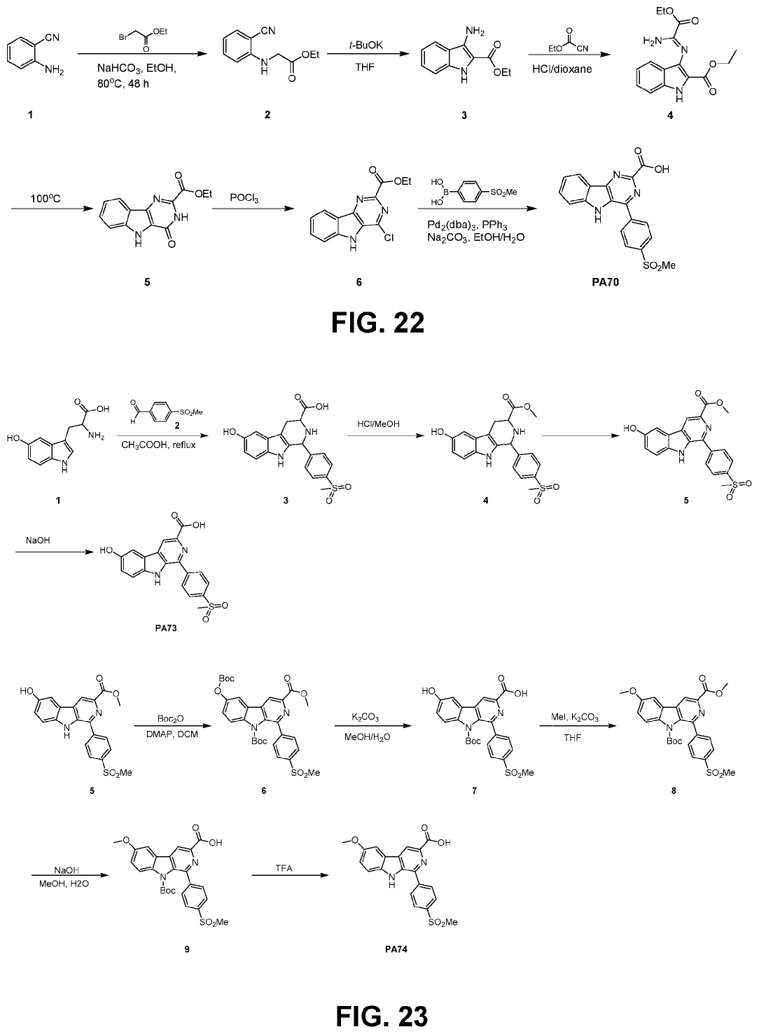

This example demonstrates the synthesis of PA70 (4-(4-methylsulfonylphenyl)-5H-pyrimido[5,4-b]indole-2-carboxylic acid) and its ethyl ester. The reaction scheme to produce compound PA70 and its ester is shown in FIG. 22, and each individual step in the synthesis is described in detail below.

To a mixture of 2-aminobenzonitrile (9.73 g, 82.4 mmol, 1.0 eq.) and ethyl 2-bromoacetate (14.3 g, 85.7 mmol, 1.0 eq.) in EtOH (50 mL) was added NaHCO₃ (8.23 g, 98.0 mmol, 1.2 eq.) in one portion. The mixture was stirred at 80° C. for 48 h. The mixture was cooled to 25° C. and concentrated in reduced pressure at 45° C. The residue was poured into water (200 mL). The aqueous phase was extracted with ethyl acetate (200 mL×3). The combined organic phase was washed with brine (200 mL×3), dried with anhydrous Na₂SO₄, filtered and concentrated in vacuum. The residue was purified by reversed-phase preparative chromatography to give compound 2 (7.00 g, 41% yield) was obtained as a yellow solid. LCMS: m/z 205.1 [M+H]⁺.

To a mixture of compound 2 (7.00 g, 36.8 mmol, 1.0 eq.) in THF (200 mL) was added t-BuOK (4.13 g, 36.8 mmol, 1.0 eq.) in THF (100 mL) dropwise at 20° C. under N₂. The mixture was stirred at 40° C. for 12 h. The mixture was cooled to 25° C. and concentrated in reduced pressure. The residue was poured into water (500 mL) and the aqueous phase was extracted with ethyl acetate (200 mL×3). The combined organic phase was washed with brine (25 mL), dried with anhydrous Na₂SO₄, filtered and concentrated in vacuum to give compound 3 (1.85 g, 14% yield) as a yellow solid. LCMS: m/z 205.0 [M+H]⁺.

Compound 3 (1.00 g, 4.90 mmol, 1.0 eq.) and ethyl cyanoformate (1.16 g, 11.8 mmol, 2.4 eq.) was added to HCl/dioxane (4 M, 50 mL) in one portion at 20° C. under N₂. The mixture was stirred at 20° C. for 12 h. The mixture was concentrated in reduced pressure to afford the crude product compound 4 (1.40 g, crude) as a yellow solid. LCMS: m/z 304.1 [M+H]1.

Compound 4 (1.40 g, 4.62 mmol, 1.0 eq.) was dissolved in dioxane (20 mL) under N₂. The mixture was stirred at 100° C. for 1.5 h. The mixture was cooled to 20° C. and concentrated in reduced pressure to give compound 5 (1.20 g, crude) as a yellow solid which was used crude in the next step. LCMS: m/z 258.0 [M+H]⁺.

To a solution of compound 5 (499 mg, 1.94 mmol, 1.0 eq.) in POCl₃ (37.8 g, 247 mmol, 130 eq.) was added DMF (142 mg, 1.94 mmol, 1.0 eq.). The mixture was stirred at 80° C. for 5 h. It was poured into ice-water (50 mL), extracted with DCM (50 mL×3), washed with brine (50 mL), dried over Na₂SO₄, filtered and concentrated in vacuum. compound 6 (600 mg, crude) was obtained as black brown oil which was used crude in the next step. LCMS: m/z 276.0 [M+H]⁺.

To a solution of (4-methylsulfonylphenyl)boronic acid (203 mg, 1.02 mmol, 1.4 eq.) and compound 6 (200 mg, 725 umol, 1.0 eq.) in H₂O (10 mL) and EtOH (20 mL) was added Na₂CO₃ (231 mg, 2.18 mmol, 3.0 eq.), Pd₂(dba)₃ (33.2 mg, 36.3 umol, 0.05 eq.) and PPh₃ (38.06 mg, 145.09 umol, 0.20 eq.). The mixture was stirred at 90° C. for 10 h under N₂. It was concentrated in vacuum. The residue was purified by Prep-HPLC to afford PA70 (12.9 mg, 4.8% yield) as yellow solid. LCMS: m/z 368.0 [M+H]⁺. ¹H NMR (DMSO-d₆, 400 MHz) δ 13.24 (s, 1H), 12.42 (s, 1H), 8.47 (d, J=8.4 Hz, 2H), 8.39 (d, J=7.6 Hz, 1H), 8.23 (d, J=8.4 Hz, 2H), 7.87-7.66 (m, 2H), 7.45-7.40 (m, 1H), 3.36 (s, 3H).

### Example 28

This example demonstrates the synthesis of PA73 and PA74 and the methyl ester of PA73. The methyl ester of PA74 could also be readily prepared if desired. The reaction scheme to produce such compounds is shown in FIG. 23, and each individual step in the synthesis is described in detail below.

To a solution of compound 1 (1.0 g, 4.54 mmol, 1.0 eq) in HOAc (50 mL) was added compound 2 (836 mg, 4.54 mmol, 1.0 eq), it was stirred at 120° C. for 2 h. The reaction mixture was concentrated in vacuum to give compound 3 (1.70 g, crude) as yellow solid. LCMS: m/z 387.0 [M+H]1.

Compound 3 (1.20 g, 3.11 mmol, 1.0 eq) was dissolved in HCl/MeOH (4 M, 50 mL). The mixture was stirred at 20° C. for 10 h. The mixture was concentrated in vacuum to give compound 4 (1.20 g, crude) as a yellow solid. LCMS: m/z 401.3 [M+H]⁺;

To a solution of compound 4 (100 mg, 249 umol, 1.0 eq) in xylene (5 mL) was added S (80.1 mg, 2.50 mmol, 10.0 eq). The mixture was stirred at 130° C. for 5 h under N₂. It was concentrated in vacuum, diluted in DCM/MeOH (v/v=1:1, 50 mL), filtered and concentrated in vacuum to give compound 5 (the methyl ester of PA73) (100 mg, crude) as gray solid. LCMS: m/z 397.0 [M+H]⁺.

To a solution of compound 5 (200 mg, 504 umol, 1.0 eq) in THF (5 mL), MeOH (5 mL) and H₂O (2.5 mL) was added LiOH·H₂O (169 mg, 4.04 mmol, 8.0 eq). The mixture was stirred at 50° C. for 10 h. The reaction mixture was concentrated under reduced pressure to remove solvent. The residue was diluted with HCl (1 N) to make pH to ˜3 and a solid came out, filtered to get a yellow solid. The residue was purified by prep-HPLC (HCl conditions) to give PA73 (15.2 mg, 39.4 umol, 7.8% yield, 99% purity) as a yellow solid. LCMS: m/z 382.9 [M+H]⁺; ¹H NMR (DMSO-d₆, 400 MHz) δ ppm 11.84 (s, 1H), 8.85 (s, 1H), 8.32 (d, J=8.4 Hz, 2H), 8.16 (d, J=8.4 Hz, 2H), 7.68 (d, J=2.4 Hz, 1H), 7.52 (d, J=8.8 Hz, 1H), 7.18-7.14 (m, 1H), 3.3 (s, 3H).

To a solution of compound 5 (150 mg, 378 umol, 1.0 eq) in DCM (20 mL) was added Et₃N (153 mg, 1.51 mmol, 4.0 eq) and Boc₂O (99.1 mg, 454 umol, 1.20 eq) and 4-dimethylaminopyridine (92.5 mg, 757 umol, 2.0 eq). The mixture was stirred at 20° C. for 10 h. The reaction mixture was diluted with H₂O (20 mL) and extracted with DCM (50 mL×3). The combined organic layers were washed with brine (50 mL), dried over Na₂SO₄, filtered and concentrated under reduced pressure to give the crude product, compound 6. LCMS: m/z 597.2 [M+H]⁺.

To a solution of compound 6 (130 mg, 218 umol, 1.0 eq) in MeOH (10 mL) was added K₂CO₃ (1 M in H₂O, 20 mL, 92 eq). The mixture was stirred at 20° C. for 10 h. The reaction mixture was concentrated under reduced pressure to remove solvent. The residue was diluted with toluene (20 mL) and concentrated under reduced pressure for three times to give the crude product, compound 7. LCMS: m/z 483.1 [M+H]⁺.

To a solution of compound 7 (300 mg, 622 umol, 1.0 eq) and K₂CO₃ (172 mg, 1.24 mmol, 2.0 eq) in THF (10 mL) was added MeI (265 mg, 1.87 mmol, 3.0 eq). The mixture was stirred at 40° C. for 3 h. The reaction mixture was concentrated under reduced pressure to give the crude product, compound 8, which was directly used for next step. LCMS: m/z 511.2 [M+H]⁺.

To a solution of compound 8 (280 mg, 548 umol, 1.0 eq.) in NaOH (1 M in H₂O, 30 mL, 55 eq) and MeOH (30 mL). The mixture was stirred at 20° C. for 4 h. The reaction mixture was diluted with water 100 mL and then acidized by HCl aqueous (1 M in water) to pH=3˜4, extracted with EA (100 mL×2). The combined organic layers were washed with brine 100 mL, dried over Na₂SO₄, filtered and concentrated under reduced pressure to give the crude product, compound 9. LCMS: m/z 497.0 [M+H]⁺.

To a solution of compound 9 (150 mg, 302 umol, 1.0 eq) in DCM (3 mL) was added TFA (34.5 mg, 302 umol, 1.0 eq). The mixture was stirred at 20° C. for 4 h. The reaction mixture was concentrated under reduced pressure to give a residue. The residue was purified by Prep-HPLC (HCl conditions) to give PA74 (17.2 mg, 14% yield) as a yellow solid. LCMS: m/z 397.1 [M+H]⁺; ¹H NMR (DMSO-d₆, 400 MHz) δ ppm 11.93 (s, 1H), 9.01 (s, 1H), 8.33 (d, J=8.0 Hz, 2H), 8.17 (d, J=8.0 Hz, 2H), 8.06 (s, 1H), 7.59 (d, J=8.8 Hz, 1H), 7.26 (d, J=9.2 Hz, 1H), 3.89 (s, 3H), 3.30 (s, 3H).

### Example 29

This example demonstrates the synthesis of PA106 (1-[4-[benzenesulfonyl(methyl) amino]phenyl]-7-methyl-9H-pyrido[3,4-b]indole-3-carboxylic acid), PA115 (1-[4-[benzenesulfonyl(methyl)amino]phenyl]-7-phenyl-9H-pyrido[3,4-b]indole-3-carboxylic acid), and PA117 (1-[4-[benzenesulfonyl(methyl)amino]phenyl]-7-bromo-9H-pyrido[3,4-b]indole-3-carboxylic acid). Various esters of these compounds can be readily synthesized if desired. The synthesis of each of PA106, PA115, and PA1 17 begins with compound 8[PA1-9] produced as described in Example 3 (see FIG. 3A).

PA106: a mixture of 8[PA1-9] (80.0 mg, 141 umol, 1.0 eq.), 2,4,6-trimethyl-1,3,5,2,4,6-trioxatriborinane (106 mg, 423 umol, 50% in THF, 3.0 eq.), Na₂CO₃ (44.8 mg, 423 umol, 3.0 eq.) and Pd(dppf)Cl₂ (10.3 mg, 14.1 umol, 0.1 eq.) in dioxane (4 mL) and H₂O (1 mL) was stirred at 100° C. for 12 h. The mixture was filtered by celite, washed with MeOH (40 mL), evaporated in vacuo to get crude product. Prep-HPLC gave PA106 (14.0 mg, 21% yield) as a yellow solid. LCMS: m/z 472.1 [M+H]⁺; ¹H NMR (DMSO-d₆, 400 MHz) (11.86 (s, 1H), 8.86 (s, 1H), 8.30 (d, J=8.0 Hz, 1H), 8.06 (d, J=8.8 Hz, 2H), 7.74 (d, J=8.4 Hz, 1H), 7.66-7.64 (m, 4H), 7.49 (s, 3H), 7.39 (d, J=8.8 Hz, 1H), 7.20-7.10 (m, 1H), 3.27 (s, 3H), 2.53 (s, 3H).

PA115: a mixture of 8[PA1-9] (80.0 mg, 141 umol, 1.0 eq.) phenylboronic acid (20.7 mg, 170 umol, 1.2 eq.), K₂CO₃ (45.0 mg, 325 umol, 2.3 eq.) and Pd(dppf)Cl₂ (10.4 mg, 14.1 umol, 0.1 eq.) in dioxane (4 mL) and H₂O (1 mL) was stirred at 80° C. for 12 h. The mixture was filtered by celite, washed with MeOH (40 mL), evaporated in vacuo to get crude product. Prep-HPLC gave PA115 (26.0 mg, 34% yield) as a gray solid. LCMS: m/z 534.0 [M+H]⁺; ¹H NMR (DMSO-d₆, 400 MHz) δ 12.06 (s, 1H), 8.95 (s, 1H), 8.55-8.50 (m, 1H), 8.09 (d, J=8.8 Hz, 2H), 7.90 (s, 1H), 7.80-7.70 (m, 3H), 7.66-7.64 (m, 5H), 7.55-7.50 (m, 2H), 7.45-7.40 (m, 3H), 3.27 (s, 3H).

### Example 30

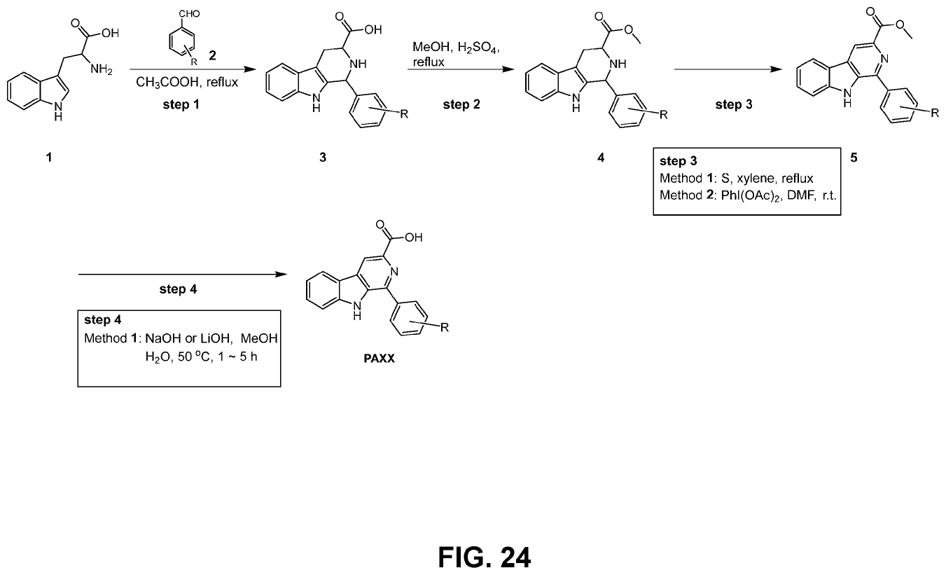

This example demonstrates the synthesis of PA2, PA3, PA5, PA6, PA7, PA8, PA19, PA21, PA22, PA23, PA25, PA26, PA27, PA29, PA32, PA33, PA36, PA71, and PA72, and the methyl esters of each such compound, though other esters could be readily prepared if desired. The general reaction scheme for each of these compounds is shown in FIG. 24.

Compound 3: A suspension of amino acid compound 1 (1.0 eq.) and aldehyde compound 2 (1.1 eq.) in CH₃COOH (4 mL) was heated at 120° C. for 1˜3 hours at nitrogen atmosphere to give a solution. The solution was concentrated under reduced pressure and dried by azeotropy with EA (30 mL×3) to give the crude product, acid compound 3, which was directly used for next step without further purification. Certain products were confirmed by LCMS.

Compound 4: To a solution of acid compound 3 (1.0 eq.) in MeOH (10 mL) was added H₂SO₄ (98%, 8.2 eq.). Then the solution was stirred at 65° C. for 18 h. The solution was concentrated under reduced pressure and the residue was diluted with EA (50 mL) and water (20 mL). Then the mixture was neutralized by solid NaOH to pH=10. The organic layer was separated and concentrated to afford ester compound 4, which was used directly for next step without purification. Certain products were confirmed by LCMS.

Compound 5: Generally, either of two methods can be used to produce compound 5 from compound 4, as shown in FIG. 24. Namely, compound 5 can be prepared either by using method (1) S in xylene or method (2) PhI(OAc)₂ in DMF. Method (1): To a solution of ester compound 4 (1.0 eq.) in xylene (3˜10 mL) was added S (8.0 eq.). The reaction was stirred at 140° C. for 5 hours. After cooling, the mixture was concentrated in vacuum to afford residue. The residue was purified by silica gel chromatography (PE/EA=10/1 to 1/1, then pure DCM until the product came out) to afford the desired product 5. Method (2): To a solution of ester compound 4 (1.0 eq.) in DMF (3 mL) was added PhI(OAc)₂ (2.0 eq.). The solution was stirred at 25° C. for 1˜3 hours. The solution was diluted with water (40 mL) and extracted with EA (40 mL×3). The combined organic layer was washed with brine (50 mL). The resulting organic layer was concentrated under reduced pressure to afford the crude product, which was directly used for next step or purified by Prep-HPLC (TFA or FA) or chromatograph column. Certain products were confirmed by LCMS.

PAXX compounds: A solution of ester compound 5 (1.0 eq.), and NaOH or LiOH H₂O (2.0 eq.) in H₂O (3 mL) and MeOH (6 mL) was stirred at 50° C. for 1˜5 hours. TLC (DCM/MeOH=20/1) indicated the reaction completed. The solution was acidified by the addition of concentrated HCl (1 M in H₂O) to pH=3˜5. The resulting mixture was concentrated under reduced pressure. The residue was washed with DMF (40 mL), filtered and evaporated in vacuo to get crude product. Prep-HPLC (TFA or FA) gave the desired product.

PA2: 215 mg, 67% yield, yellow solid, LCMS: m/z 303.0 [M+H]⁺; ¹H NMR (DMSO-d₆, 400 MHz) δ ppm 11.90 (s, 1H), 8.90 (s, 1H), 8.42 (d, J=8.0 Hz, 1H), 7.98 (d, J=8.0 Hz, 2H), 7.70 (d, J=8.0 Hz, 1H), 7.63-7.58 (m, 1H), 7.45 (d, J=8.0 Hz, 2H), 7.35-7.32 (m, 1H), 2.46 (s, 3H).)

PA3: 45 mg, 12% yield, light yellow solid; LCMS: m/z 307.0 [M+H]⁺; ¹H NMR (DMSO-d₆, 400 MHz) δ ppm 11.77 (s, 1H), 8.84 (s, 1H), 8.40-8.30 (m, 1H), 8.20-8.00 (m, 2H), 7.67-7.56 (m, 2H), 7.40-7.29 (m, 3H).

PA5: 41.0 mg, 11% yield, pale solid; LCMS: m/z 307.0 [M+H]⁺; ¹H NMR (DMSO-d₆, 400 MHz) δ ppm 11.97 (s, 1H), 8.93 (s, 1H), 8.43 (d, J=8.0 Hz, 1H), 7.94-7.92 (m, 2H), 7.71-7.62 (m, 3H), 7.40-7.32 (m, 2H).

PA6: 180 mg, 30% yield, light yellow solid; LCMS: m/z 323.0 [M+H]⁺; ¹H NMR (DMSO-d₆, 400 MHz) δ ppm 11.98 (s, 1H), 8.92 (s, 1H), 8.42 (d, J=7.6 Hz, 1H), 8.15-8.05 (m, 2H), 7.69-7.61 (m, 4H), 7.36-7.33 (m, 1H).

PA7: 108 mg, 55% yield, yellow solid; LCMS: m/z 303.1 [M+H]⁺; ¹H NMR (DMSO-d₆, 400 MHz) δ ppm 11.93 (s, 1H), 8.92 (s, 1H), 8.30 (d, J=8.0 Hz, 1H), 7.87-7.84 (m, 2H), 7.72-7.69 (m, 1H), 7.65-7.55 (m, 1H), 7.53-7.51 (m, 1H), 7.40-7.33 (m, 2H), 2.49 (s, 3H).

PA8: 38.0 mg, 31% yield, light yellow solid; LCMS: m/z 319.0 [M+H]⁺; ¹H NMR (DMSO-d₆, 400 MHz) δ ppm 11.88 (s, 1H), 8.90 (s, 1H), 8.41 (d, J=8.0 Hz, 1H), 7.71-7.68 (m, 1H), 7.62-7.54 (m, 4H), 7.35-7.32 (m, 1H), 7.15-7.13 (m, 1H), 3.89 (s, 3H).

PA19: 45.0 mg, 23% yield, off-white solid; LCMS: m/z 314.0 [M+H]⁺; ¹H NMR (DMSO-d₆, 400 MHz) δ ppm 12.00 (s, 1H), 8.95 (s, 1H), 8.43 (d, J=8.0 Hz, 1H), 8.29-8.27 (m, 2H), 8.10-8.07 (m, 2H), 7.71-7.68 (m, 1H), 7.64-7.62 (m, 1H), 7.37-7.32 (m, 1H).

PA21: 232 mg, 74% yield, yellow solid; LCMS: m/z 319.0 [M+H]⁺; ¹H NMR (DMSO-d₆, 400 MHz) δ ppm 11.90 (s, 1H), 8.90 (s, 1H), 8.41 (d, J=8.0 Hz, 1H), 8.05 (d, J=8.0 Hz, 2H), 7.71-7.69 (m, 1H), 7.62-7.57 (m, 3H), 7.35-7.32 (m, 1H), 5.37 (s, 1H), 4.66 (s, 2H).

PA22: 73.0 mg, 73% yield, yellow solid; LCMS: m/z 305.0 [M+H]⁻; ¹H NMR (DMSO-d₆, 400 MHz) δ ppm 11.88 (s, 1H), 9.78 (s, 1H), 8.89 (s, 1H), 8.40 (d, J=7.6 Hz, 1H), 7.72-7.69 (m, 1H), 7.65-7.55 (m, 1H), 7.47-7.40 (m, 3H), 7.34-7.32 (m, 1H), 6.99-6.96 (m, 1H).

PA23: 18.0 mg, 26% yield, white floccus; LCMS: m/z 313.6 [M+H]⁺; ¹H NMR (DMSO-d₆, 400 MHz) δ ppm 12.05 (s, 1H), 8.95 (s, 1H), 8.52-8.42 (m, 3H), 8.03-8.00 (m, 1H), 7.85-7.80 (m, 1H), 7.72-7.69 (m, 1H), 7.66-7.61 (m, 1H), 7.37-7.34 (m, 1H).

PA25: 284 mg, 59% yield, yellow solid; LCMS: m/z 290.0 [M+H]⁺; ¹H NMR (DMSO-d₆, 400 MHz) δ ppm 12.25 (s, 1H), 9.35 (d, J=1.6 Hz, 1H), 8.99 (s, 1H), 8.84 (d, J=3.6 Hz, 1H), 8.65 (d, J=8.0 Hz, 1H), 8.46 (d, J=8.0 Hz, 1H), 7.83-7.80 (m, 1H), 7.73-7.70 (m, 1H), 7.66-7.63 (m, 1H), 7.38-7.35 (m, 1H).

PA26: 210 mg, 98% yield, yellow solid; LCMS: m/z 293.0 [M+H]⁺; ¹H NMR (DMSO-d₆, 400 MHz) δ ppm 11.64 (s, 1H), 8.79 (s, 1H), 8.72 (s, 1H), 8.44 (s, 1H), 8.39 (d, J=7.6 Hz, 1H), 7.75 (d, J=8.4 Hz, 1H), 7.64-7.59 (m, 1H), 7.35-7.31 (m, 1H), 4.01 (s, 3H).

PA27: 47.0 mg, 40% yield, light yellow solid; LCMS: m/z 279.0 [M+H]⁺; ¹H NMR (DMSO-d₆, 400 MHz) δ 11.95 (s, 1H), 8.91 (s, 1H), 8.43 (d, J=7.6 Hz, 1H), 7.96 (d, J=8.4 Hz, 1H), 7.64-7.56 (m, 2H), 7.36-7.31 (m, 2H).

PA29: 124 mg, 98% yield, yellow solid; LCMS: m/z 329.0 [M+H]; ¹H NMR (DMSO-d₆, 400 MHz) δ ppm 13.28 (s, 1H), 11.96 (s, 1H), 8.88 (s, 1H), 8.48 (s, 1H), 8.42 (d, J=8.0 Hz, 1H), 8.27 (s, 1H), 8.10 (d, J=8.8 Hz, 1H), 7.77 (d, J=8.8 Hz, 1H), 7.75-7.70 (m, 1H), 7.70-7.60 (m, 1H), 7.35-7.30 (m, 1H).

PA32: 85.0 mg, 33% yield, light yellow solid; LCMS: m/z 324.6 [M+H]⁺; ¹H NMR (DMSO-d₆, 400 MHz) δ ppm 11.97 (s, 1H), 8.91 (s, 1H), 8.42 (d, J=8.0 Hz, 1H), 8.20-8.10 (m, 1H), 8.00-7.90 (m, 1H), 7.71-7.60 (m, 3H), 7.36-7.32 (m, 1H).

PA33: 340 mg, 41% yield, white solid; LCMS: m/z 342.7 [M+H]⁺; ¹H NMR (DMSO-d₆, 400 MHz) δ ppm 12.07 (s, 1H), 8.96 (s, 1H), 8.44 (d, J=8.0 Hz, 1H), 8.08-8.03 (m, 2H), 7.73-7.70 (m, 1H), 7.66-7.64 (m, 1H), 7.38-7.33 (m, 1H).

PA36: 64.0 mg, 41% yield, light yellow solid; LCMS: n/z 333.6 [M+H]⁺; ¹H NMR (DMSO-d₆, 400 MHz) δ ppm 12.10 (s, 1H), 8.95 (s, 1H), 8.85-8.75 (m, 1H), 8.51-8.37 (m, 3H), 7.95-7.90 (m, 1H), 7.70-7.62 (m, 2H), 7.37-7.33 (m, 1H).

PA71: 21.0 mg, 50% yield, white solid; LCMS: m/z 385.0 [M+H]⁺; ¹H NMR (DMSO-d₆, 400 MHz) δ ppm 11.76 (s, 1H), 8.84 (s, 1H), 8.23-8.06 (m, 5H), 7.65-7.60 (m, 1H), 7.48-7.42 (m, 1H), 3.30 (m, 3H).

PA72: 64.1 mg, 25% yield, yellow solid; LCMS: m/z 381.1 [M+H]⁻; ¹H NMR (DMSO-d₆, 400 MHz) δ ppm 12.01 (s, 1H), 8.93 (s, 1H), 8.35-8.32 (m, 2H), 8.25 (s, 1H), 8.19-8.16 (m, 2H), 7.61-7.58 (m, 1H), 7.49-7.43 (m, 1H), 3.44 (s, 3H), 2.55 (s, 3H).

### Example 31

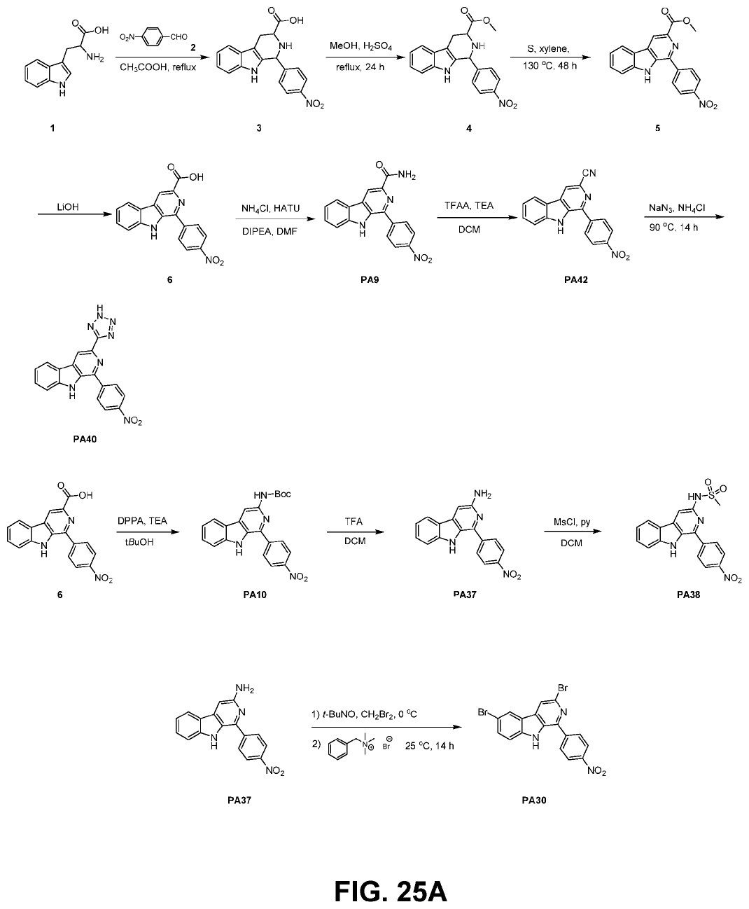

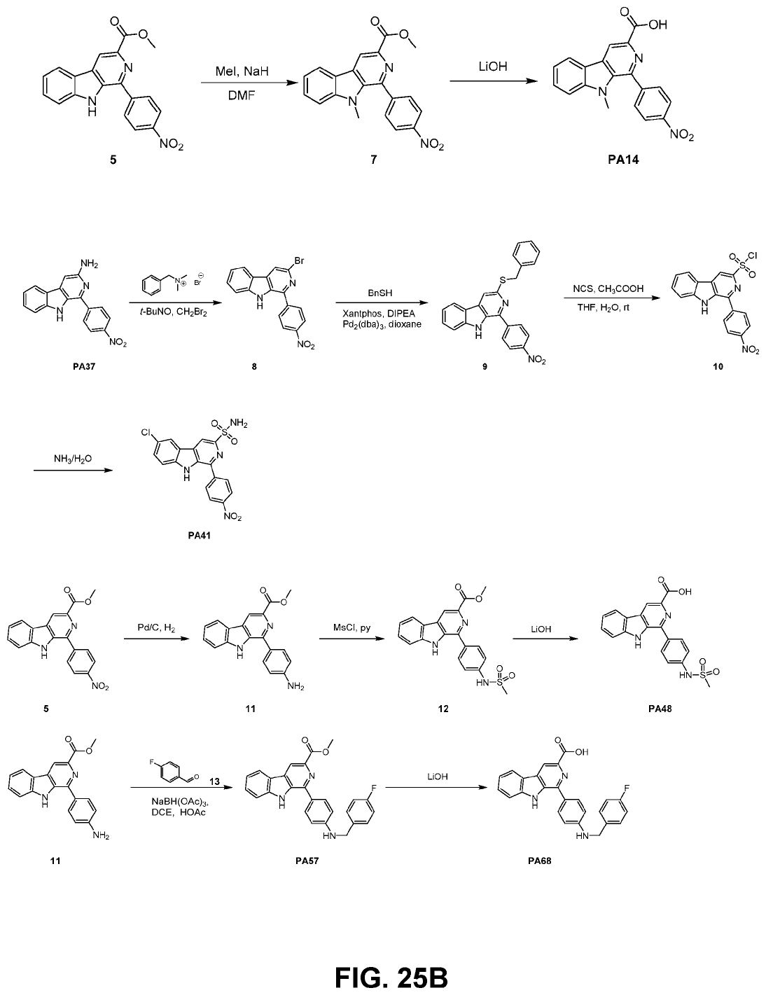

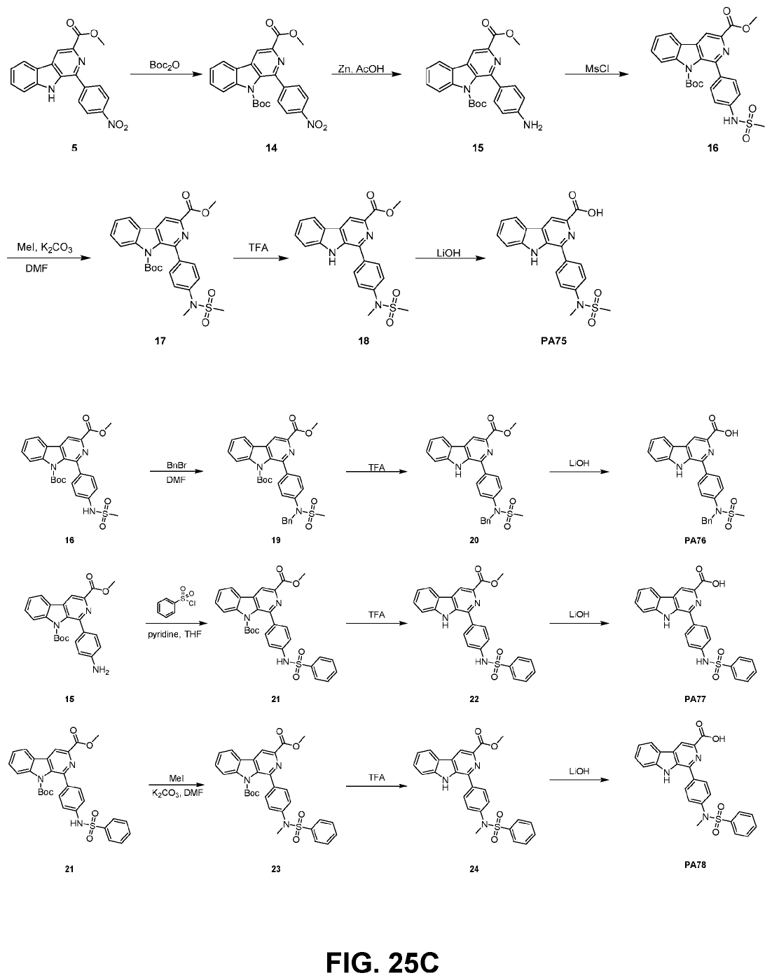

This example demonstrates the synthesis of PA9, PA14, PA30, PA38, PA40, PA41, PA42, PA48, PA57, PA68, PA75, PA76, PA77, and PA78, and for some of these compounds the esters thereof, though other esters could be readily prepared if desired. The synthetic schemes for each of these compounds is shown in FIGS. 25A, 25B, and 25C.

Compound 3: A suspension of compound 1 (5.00 g, 24.5 mmol, 1.0 eq.) and compound 2 (4.44 g, 29.4 mmol, 1.2 eq.) in CH₃COOH (50 mL) was stirred at 120° C. for 12 hours to give a solution. The solution was concentrated under reduced pressure to afford compound 3 (9.00 g, crude) as yellow gum, which was used directly for next step. LCMS: m/z 338.1 [M+H]⁺.

Compound 4: To a solution of crude compound 3 (10.0 g, 29.6 mmol, 1.0 eq.) in MeOH (150 mL) was added H₂SO₄ (con., 7.36 g, 75.0 mmol, 4 mL, 2.5 eq.). And then the solution was stirred at 65° C. for 20 hours. TLC (DCM/MeOH=10/1) indicated the reaction completed. The solution was concentrated under reduced pressure and the residue was diluted with DCM (200 mL) and water (50 mL). The mixture was neutralized by the addition of solid NaOH to pH=9. The organic layer was separated, dried over Na₂SO₄, concentrated. The crude product was purified by column chromatography (pure DCM) to give compound 4 (5.20 g, 50% yield) as yellow solid. LCMS: m/z 352.0 [M+H]⁺.

Compound 5: A mixture of compound 4 (5.00 g, 14.2 mmol, 1.0 eq.) and S (2.00 g, 62.3 mmol, 4.4 eq.) in xylene (50 mL) was stirred at 130° C. for 48 hours. The mixture was allowed to cool to room temperature (25° C.) and then poured onto silica gel in a column. It was eluted with pure PE to 5% of MeOH in DCM to afford compound 5 (3.30 g, 61% yield) as a yellow solid. LCMS: m/z 348.0 [M+H]⁺.

Compound 6: To a yellow suspension of compound 5 (3.30 g, 9.50 mmol, 1.0 eq.) in THF (40 mL), MeOH (10 mL) and H₂O (2 mL) was added LiOH H₂O (1.00 g, 23.9 mmol, 2.5 eq.). Then the resulting red suspension was stirred at 50° C. for 4 hours. TLC (PE/EA=5/2) indicated the starting material was consumed completely. The mixture was concentrated under reduced pressure. The residue was diluted with water (200 mL) and then acidified by HCl (12 M) to pH=2. The resulting precipitate was collected by filtration as the wet product, which was dried by lyophilized to get compound 6 (3.00 g, 84% yield) as a yellow solid. LCMS: m/z 334.0 [M+H]⁺.

PA9: To a suspension of compound 6 (150 mg, 450 umol, 1.0 eq.) and NH₄Cl (96.3 mg, 1.80 mmol, 4.0 eq.) in DMF (2 mL) was added DIPEA (349 mg, 2.70 mmol, 6.0 eq.). After stirred for 15 minutes, HATU (171 mg, 450 umol, 1.0 eq.) was added. And then the mixture was stirred at 25° C. for 2 hours. The solution was diluted with water (40 mL) and the precipitate was collected by filtration. The precipitate was purified by Prep-HPLC (FA) to give PA9 (1-(4-nitrophenyl)-9H-pyrido[3,4-b]indole-3-carboxamide) (36.0 mg, 24% yield) as a yellow solid. LCMS: m/z 333.0 [M+H]⁺; ¹H NMR (DMSO-d₆, 400 MHz) δ 12.09 (s, 1H), 8.93 (s, 1H), 8.52-8.43 (m, 5H), 8.22 (s, 1H), 7.72-7.69 (m, 1H), 7.65-7.61 (m, 2H), 7.37-7.32 (m, 1H).

PA42: To a solution of PA9 (700 mg, 2.11 mmol, 1.0 eq.) in THF (10 mL) was added TFAA (886 mg, 4.22 mmol, 2.0 eq.). Then TEA (854 mg, 8.44 mmol, 4.0 eq.) was added at 0° C. The reaction was stirred at 0° C.-25° C. for 20 hours. TLC (DCM/MeOH=10/1) indicated the starting material was consumed mostly and one new spot was detected. The solution combined was concentrated under reduced pressure to give a residue. The residue was purified by column chromatography (DCM/MeOH=50/1 to 10/1) to give PA42 (1-(4-nitrophenyl)-9H-pyrido[3,4-b]indole-3-carbonitrile) (220 mg, 32% yield) as a yellow solid. LCMS: m/z 314.6 [M+H]⁺; ¹H NMR (DMSO-d₆, 400 MHz) δ 12.35 (s, 1H), 9.01 (s, 1H), 8.49-8.46 (m, 2H), 8.41 (d, J=8.0 Hz, 1H), 8.32-8.28 (m, 2H), 7.75-7.68 (m, 2H), 7.42 (t, J=7.2 Hz, 1H).

PA40: A mixture of PA42 (170 mg, 541 umol, 1.0 eq.), NaN₃ (141 mg, 2.16 mmol, 4.0 eq.) and NH₄Cl (116 mg, 2.16 mmol, 4.0 eq.) in DMAC (3 mL) was heated to 90° C. for 14 hours. The mixture was filtered through a silica gel (eluted with pure DCM) to give the crude product (contained DMAc). The crude product was diluted with EtOH (5 mL) to give a suspension. The precipitate was collected by filtration and the filtrate was purified by Prep-HPLC (TFA) to afford PA40 (1-(4-nitrophenyl)-3-(1H-tetrazol-5-yl)-9H-pyrido[3,4-b]indole) (4.00 mg, 2% yield) as an orange solid. LCMS: m/z 358.0 [M+H]⁺; ¹H NMR (DMSO-d₆, 400 MHz) δ 12.15 (s, 1H), 9.10 (s, 1H), 8.52-8.49 (m, 5H), 7.74-7.71 (m, 1H), 7.68-7.63 (m, 1H), 7.40-7.35 (m, 1H).

Compound 310: To a suspension of compound 6 (3.00 g, 9.00 mmol, 1.0 eq.) in t-BuOH (78.0 g, 1.05 mol, 120 eq.) was added TEA (1.82 g, 18.0 mmol, 2.0 eq.) and DPPA (4.95 g, 18.0 mmol, 2.0 eq.). The mixture was stirred at 120° C. for 20 hours at nitrogen atmosphere. The mixture was concentrated under reduced pressure to give a residue. The residue was purified by column chromatography on silica gel to afford the product compound 310 (tert-butyl N-[1-(4-nitrophenyl)-9H-pyrido[3,4-b]indol-3-yl]carbamate) (1.50 g, 38% yield) as a red solid. Partial product was further purified by Prep-TLC (100% of DCM) to afford the pure product 28.0 mg for delivery. LCMS: m/z 405.0 [M+H]⁺; ¹H NMR (DMSO-d₆, 400 MHz) (11.55 (s, 1H), 9.67 (s, 1H), 8.48 (s, 1H), 8.44 (d, J=8.8 Hz, 2H), 8.33 (d, J=8.8 Hz, 2H), 8.25 (d, J=8.0 Hz, 1H), 7.62-7.56 (m, 2H), 7.27-7.24 (m, 1H), 1.53 (s, 9H).

Compound 337: To a solution of compound 310 (270 mg, 668 umol, 1.0 eq.) in DCM (4 mL) was added TFA (4 mL) and the reaction was stirred at 25° C. for 1 hour. The reaction solution was concentrated under reduced pressure to give the crude product. The crude product was triturated with DMSO (3 mL) and NH₃H₂O (1 mL) to afford compound 337 (1-(4-nitrophenyl)-9H-pyrido[3,4-b]indol-3-amine) (130 mg, 62% yield) as a black brown solid. LCMS: m/z 305.1 [M+H]⁺; ¹H NMR (DMSO-d₆, 400 MHz) δ 11.02 (s, 1H), 8.40 (d, J=8.8 Hz, 2H), 8.27 (d, J=8.8 Hz, 2H), 8.07 (d, J=8.0 Hz, 1H), 7.49-7.47 (m, 2H), 7.22 (s, 1H), 7.16-7.13 (m, 1H), 5.60 (brs, 2H).

PA38: To a suspension of compound 337 (100 mg, 329 umol, 1.0 eq.) and pyridine (260 mg, 3.29 mmol, 10 eq.) in DCM (2 mL) was added MsCl (376 mg, 3.29 mmol, 10 eq.). The reaction was stirred at 25° C. for 45 hours. The red suspension was concentrated under reduced pressure to afford the crude, which was purified by Prep-HPLC to afford PA38 (N-[1-(4-nitrophenyl)-9H-pyrido[3,4-b]indol-3-yl]methanesulfonamide) (38.0 mg, 30% yield) as a yellow solid. LCMS: m/z 383.0 [M+H]⁺; ¹H NMR (DMSO-d₆, 400 MHz) (11.63 (s, 1H), 10.36 (s, 1H), 8.46 (d, J=8.8 Hz, 2H), 8.31 (d, J=9.2 Hz, 2H), 8.25 (d, J=8.0 Hz, 1H), 7.81 (s, 1H), 7.62-7.58 (m, 2H), 7.29-7.26 (m, 1H), 3.35 (s, 3H).

PA30: To a suspension of compound 337 (100 mg, 329 umol, 1.0 eq.) in dibromomethane (1 mL) was added tert-butyl nitrite (33.9 mg, 329 umol, 1.0 eq.) at 0° C. After 30 minutes, benzyl(trimethyl)ammonium;bromide (75.6 mg, 329 umol, 1.0 eq.) was added and the reaction was stirred at 25° C. for 12 hours at nitrogen atmosphere to give a red solution. TLC (PE/EA=3/1) indicated starting material was consumed completely. The mixture was concentrated under reduced pressure to give a red residue. The residue was purified by column chromatography (PE/DCM=1/1) to afford the crude product, which was purified again by Prep-HPLC (FA) to afford PA30 (3,6-dibromo-1-(4-nitrophenyl)-9H-pyrido[3,4-b]indole) (21.0 mg, 14% yield) as an orange solid. LCMS: m/z 447.8 [M+H]⁺; ¹H NMR (DMSO-d₆, 400 MHz) δ 8.63 (s, 1H), 8.58 (s, 1H), 8.45 (d, J=8.8 Hz, 2H), 8.27 (d, J=8.8 Hz, 2H), 7.74 (dd, J₁=2.0 Hz, J₂=8.8 Hz, 1H), 7.61 (d, J=8.8 Hz, 1H).

Compound 7: To a suspension of compound 5 (200 mg, 576 umol, 1.0 eq.) in dry DMF (2 mL) was added NaH (46.1 mg, 1.15 mmol, 60% purity, 2.0 eq.) at 0° C. under nitrogen. The mixture turned purple immediately. After 10 minutes, MeI (1.83 g, 12.9 mmol, 22 eq.) was added and the reaction was stirred at 25° C. for 3 hours to give a light yellow solution. The solution was diluted with water (80 mL) and the resulting precipitate was collected by filtration to get compound 7 (200 mg, crude) as a yellow solid. LCMS: m/z 362.0 [M+H]⁺.

PA14: A mixture of compound 7 (200 mg, 553 umol, 1.0 eq.) and LiOH·H₂O (69.7 mg, 1.66 mmol, 3.0 eq.) in MeOH (5 mL), THF (5 mL) and H₂O (2 mL) was stirred at 25° C. for 14 hours to give a solution. The solution was concentrated under reduced pressure. The residue was diluted with water (30 mL) and then acidified by HCl (12 M) to pH=2. The precipitate was collected by filtration as the crude product. The crude product was triturated with EtOH (15 mL) to afford PA14 (9-methyl-1-(4-nitrophenyl)pyrido[3,4-b]indole-3-carboxylic acid) (150 mg, 75% yield) as a yellow solid. LCMS: m/z 348.0 [M+H]⁺; ¹H NMR (DMSO-d₆, 400 MHz) δ 12.83 (s, 1H), 9.01 (s, 1H), 8.50 (d, J=8.0 Hz, 1H), 8.42 (d, J=8.4 Hz, 2H), 7.99 (d, J=8.4 Hz, 2H), 7.76-7.69 (m, 2H), 7.45-7.35 (m, 1H), 3.52 (s, 3H).

Compound 8: To a suspension of compound 337 (300 mg, 986 umol, 1.0 eq.) in dibromomethane (4 mL) was added tert-butyl nitrite (203 mg, 1.97 mmol, 2.0 eq.) at 0° C. After 30 minutes, benzyl(trimethyl)ammonium;bromide (454 mg, 1.97 mmol, 2.0 eq.) was added and the reaction was stirred at 25° C. for 12 hours at nitrogen atmosphere to give a red solution. TLC (PE/EA=3/1) indicated starting material consumed completely and there was a new spot generated. The solution was poured onto silica gel in a column. It was eluted with pure DCM, then 0.5% of MeOH in DCM to give compound 8 (150 mg, crude) as a red solid. LCMS: m/z 368.0 [M+H]⁺.

Compound 9: To a solution of compound 8 (110 mg, 299 umol, 1.0 eq.) dissolved in dioxane (3 mL) was added BnSH (371 mg, 2.99 mmol, 10 eq.), Xantphos (34.6 mg, 59.8 umol, 0.2 eq.), DIPEA (77.2 mg, 598 umol, 2.0 eq.) and Pd₂(dba)₃ (27.4 mg, 29.9 umol, 0.1 eq.) under nitrogen, the mixture was heated at 80° C.-100° C. for 18 hours. The suspension was concentrated under reduced pressure. The residue was purified by column (PE/DCM=1/1 to pure DCM) to get compound 9 (120 mg, crude) as a red solid. LCMS: m/z 411.9 [M+H]⁺.

Compound 10: To a solution of compound 9 (120 mg, 292 umol, 1.0 eq.) in THF (3 mL), HOAc (1 mL) and H₂O (0.5 mL) was added NCS (79.8 mg, 598 umol, 2.1 eq.) at 0° C. The orange solution was stirred at 0° C. for 0.5 hour and 25° C. for further 1 hour to give a yellow solution. Another batch of NCS (156 mg, 1.17 mmol, 4.0 eq.) was added and the reaction was stirred for further 30 minutes. The solution was concentrated under reduced pressure to get the product, compound 10 (113 mg, crude), which was used directly and immediately for next step.

PA41: To a solution of compound 10 (113 mg, 291 umol, 1.0 eq.) in MeCN (3 mL) was added NH₃H₂O (10.2 mg, 291 umol, 1.0 eq.). The suspension was stirred at 25° C. for 1 hour to give a yellow suspension. The suspension was diluted with water (20 mL) and the resulting precipitate was collected by filtration. The crude product was purified by Prep-HPLC (FA) to get PA41 (6-chloro-1-(4-nitrophenyl)-9H-pyrido[3,4-b]indole-3-sulfonamide) (40.0 mg) as a yellow solid. LCMS: m/z 402.9 [M+H]⁺; ¹H NMR (DMSO-d₆, 400 MHz) δ 8.92 (s, 1H), 8.67 (s, 1H), 8.50-8.47 (m, 2H), 8.44-8.37 (m, 3H), 7.72 (d, J=8.8 Hz, 1H), 7.67-7.64 (m, 1H), 7.39 (s, 2H).

Compound 11: A mixture of compound 5 (200 mg, 576 umol, 1.0 eq.) and Pd/C (100 mg, 10% purity) in MeOH (8 mL) was stirred at 25° C. at hydrogen atmosphere for 14 hours. TLC (PE/EA=3/1) indicated the reaction completed. The mixture was filtered and the filtrate was concentrated under reduced pressure to give compound 11 (200 mg, crude) as a yellow solid. LCMS: m/z 318.2 [M+H]⁺.

Compound 12: To a solution of compound 11 (180 mg, 567 umol, 1.0 eq.) and Pyridine (179 mg, 2.27 mmol, 4.0 eq.) in DCM (4 mL) was added methanesulfonyl chloride (65.0 mg, 567 umol, 1.0 eq.). The mixture was stirred at 25° C. for 3 hours. The solution was poured into water (10 mL), adjusted pH=7 with 1 M HCl, extracted with DCM (10 mL×3). The combined organic phase was washed with brine (30 mL), dried with anhydrous Na₂SO₄, filtered and concentrated to afford residue. The residue was purified by silica gel chromatography (DCM/MEOH=50/1 to 5/1) to afford compound 12 (245 mg, 98% yield) as red gum. LCMS: m/z 396.1 [M+H]⁺.

PA48: To a solution of crude compound 12 (245 mg, 620 umol, 1.0 eq.) in MeOH (2 mL)/H₂O (2 mL) was added LiOH H₂O (52.0 mg, 1.24 mmol, 2.0 eq.). The mixture was stirred at 25° C. for 4 hours. The mixture was acidified by HCl (1 M) to pH=2. And then the mixture was concentrated under reduced pressure to afford a residue. The residue was filtered through a shot layer of silica gel and eluted with DCM/MeOH=10/1 to afford the crude product, which was further purified by Prep-HPLC to get PA48 (1-[4-(methanesulfonamido)phenyl]-9H-pyrido[3,4-b]indole-3-carboxylic acid) (46.0 mg, 19% yield) as a yellow solid. LCMS: m/z 382.0 [M+H]⁺; ¹H NMR (DMSO-d₆, 400 MHz) δ 11.88 (s, 1H), 8.87 (s, 1H), 8.40 (s, 1H), 8.06 (d, J=8.8 Hz, 2H), 7.70-7.68 (m, 1H), 7.62-7.57 (m, 1H), 7.46 (d, J=8.8 Hz, 2H), 7.34-7.32 (m, 1H), 3.10 (s, 3H).

PA57: To a solution of compound 11 (60.0 mg, 189 umol, 1.0 eq.) and 4-fluorobenzaldehyde (23.5 mg, 189 umol, 1.0 eq.) in DCE (2 mL) was added HOAc (11.4 mg, 189 umol, 1.0 eq.) and NaBH(OAc)₃ (160 mg, 756 umol, 4.0 eq.). The mixture was stirred at 20° C. for 14 hours. The mixture was diluted with DCM (30 mL) and washed with water (20 mL×2). The resulting organic layer was concentrated under reduced pressure to afford a residue. The residue was purified by Prep-TLC (PE/EA=1/1) to afford PA57 (methyl 1-[4-[(4-fluorophenyl)methylamino]phenyl]-9H-pyrido[3,4-b]indole-3-carboxylate) (42.0 mg, 52% yield) as a light yellow solid. LCMS: n/z 426.0 [M+H]⁺; ¹H NMR (CDCl₃, 400 MHz) δ 8.81 (s, 1H), 8.77 (s, 1H), 8.21 (d, J=8.0 Hz, 1H), 7.81 (d, J=8.4 Hz, 2H), 7.60-7.50 (m, 2H), 7.37-7.34 (m, 3H), 7.09-7.04 (m, 2H), 6.77 (d, J=8.4 Hz, 2H), 4.39 (s, 2H), 4.33 (brs, 1H), 4.05 (s, 3H).

PA68: A mixture of PA57 (26.0 mg, 61.1 umol, 1.0 eq.) and LiOH H₂O (25.6 mg, 611 umol, 10 eq.) in MeOH (1.5 mL) and H₂O (0.1 mL) was stirred at 50° C. for 1 hour. The mixture was acidified by HCl (1 M) to pH=4. The mixture was poured onto silica gel in a column and eluted with DCM/MeOH=10/1 to afford PA68 (1-[4-[(4-fluorophenyl) methylamino]phenyl]-9H-pyrido[3,4-b]indole-3-carboxylic acid) (24.0 mg, 92% yield) as a yellow solid. LCMS: m/z 412.0 [M+H]⁺; ¹H NMR (DMSO-d₆, 400 MHz) δ 11.72 (s, 1H), 8.76 (s, 1H), 8.40-8.30 (m, 1H), 7.86 (d, J=8.4 Hz, 2H), 7.70-7.65 (m, 1H), 7.60-7.55 (m, 1H), 7.46-7.43 (m, 2H), 7.30-7.25 (m, 1H), 7.21-7.15 (m, 2H), 6.81-6.78 (m, 3H), 4.38 (d, J=4.8 Hz, 2H), 3.17 (s, 1H).

Compound 14: To a solution of compound 5 (1.90 g, 5.47 mmol, 1.0 eq.) in DCM (50 mL) was added Et₃N (1.11 g, 10.9 mmol, 2.0 eq.), DMAP (66.8 mg, 547 umol, 0.1 eq.) and Boc₂O (1.79 g, 8.21 mmol, 1.5 eq.) at 5° C. After addition, the mixture was stirred at 20° C. for 12 hours. The reaction mixture was poured into water (50 mL), the separated organic layer was washed with brine (10 mL), dried over Na₂SO₄, filtered and concentrated to give a yellow solid. The yellow solid was purified by column chromatography on silica gel (PE/EA=10/1 to 6/1) to give compound 14 (500 mg, 18% yield) as a off-white solid. LCMS: m/z 448.1 [M+H]⁺.

Compound 15: To a solution of compound 14 (500 mg, 934 umol, 1.0 eq.) in AcOH (50 mL) was added Zn (305 mg, 4.67 mmol, 5.0 eq.) powder. The mixture was stirred under N₂ protection at 50° C. for 2 hours. The reaction mixture was filtered and the filter was concentrated to get compound 15 (380 mg, crude) as a yellow solid. LCMS: m/z 418.1 [M+H]⁺.

Compound 16: To a solution of compound 15 (350 mg, 838 umol, 1.0 eq.) and pyridine (380 mg, 4.80 mmol, 4.0 eq.) in THF (20 mL) was added MsCl (125 mg, 1.09 mmol, 1.3 eq.) at 0° C. The mixture was stirred at 10° C. for 3 hours. The reaction mixture was quenched by addition water (30 mL), and then extracted with EA (100 mL×2). The combined organic layers were washed with brine (100 mL), dried over Na₂SO₄, filtered and concentrated under reduced pressure to give compound 16 (430 mg, crude) as a yellow solid. LCMS: m/z 496.1 [M+H]⁺.

Compound 17: To a solution of compound 16 (200 mg, 404 umol, 1.0 eq.) in DMF (3 mL) was added MeI (115 mg, 807 umol, 2.0 eq.) and K₂CO₃ (112 mg, 807 umol, 2.0 eq.). The mixture was stirred at 20° C. for 5 hours. The reaction mixture was quenched by addition water (100 mL), extracted with EA (100 mL×2). The combined organic layers were washed with brine (100 mL), dried over Na₂SO₄, filtered and concentrated under reduced pressure to give a residue. The residue was purified by Prep-TLC (PE/EA=3/1) to get compound 17 (98.0 mg, 41% yield) as a white solid. LCMS: m/z 510.0 [M+H]⁺.

Compound 18: To a solution of compound 17 (115 mg, 192 umol, 1.0 eq.) in DCM (2 mL) was added TFA (7.55 g, 66.2 mmol, 340 eq.). The mixture was stirred at 20° C. for 2 hours. The reaction mixture was concentrated under reduced pressure to give compound 17 (80.0 mg, crude) as a yellow oil. LCMS: m/z 410.1 [M+H]⁺.

PA75: To a solution of compound 18 (80.0 mg, 195 umol, 1.0 eq.) in MeOH (2 mL), THF (2 mL), H₂O (1 mL) was added LiOH (23.4 mg, 977 umol, 5.0 eq.). The mixture was stirred at 20° C. for 4 hours. The reaction mixture was concentrated under reduced pressure to removed THF and MeOH and then acidized by HCl aqueous (1 N) to pH=3˜4 and diluted with DMSO (3 mL). The residue was purified by Prep-HPLC (HCl) to get PA75 (1-[4-[methyl(methylsulfonyl)amino]phenyl]-9H-pyrido[3,4-b]indole-3-carboxylic acid) (15.1 mg, 19% yield) as a white solid. LCMS: m/z 396.1 [M+H]⁺; ¹H NMR (DMSO-d₆, 400 MHz) δ 11.99 (s, 1H), 8.92 (s, 1H), 8.43 (d, J=8.0 Hz, 1H), 8.12 (d, J=8.8 Hz, 2H), 7.72-7.61 (m, 4H), 7.40-7.30 (m, 1H), 3.36 (s, 3H), 3.05 (s, 3H).

Compound 19: To a solution of compound 16 (200 mg, 404 umol, 1.0 eq.) and bromomethylbenzene (345 mg, 2.02 mmol, 5.0 eq.) in DMF (3 mL) was added K₂CO₃ (112 mg, 807 umol, 2.0 eq.). The mixture was stirred at 20° C. for 5 hours. The reaction mixture was quenched by addition water 100 mL, and then extracted with EA (100 mL×2). The combined organic layers were washed with brine (50 mL), dried over Na₂SO₄, filtered and concentrated under reduced pressure to give a residue. The residue was purified by column chromatography (PE/EA=20/1 to 1/1). Compound 19 (120 mg, 42% yield) was obtained as a yellow solid. LCMS: m/z 586.0 [M+H]⁺.

Compound 20: To a solution of compound 19 (120 mg, 168 umol, 1.0 eq.) in DCM (3 mL) was added TFA (4.62 g, 40.5 mmol, 240 eq.). The mixture was stirred at 20° C. for 4 hours. Then the reaction mixture was concentrated under reduced pressure to give compound 20 (100 mg, crude) as yellow oil. LCMS: m/z 486.1 [M+H]⁻

PA76: To a solution of compound 20 (100 mg, 206 umol, 1.0 eq.) in MeOH (2 mL), THF (2 mL) and H₂O (1 mL) was added LiOH (24.66 mg, 1.03 mmol, 5.0 eq.). The mixture was stirred at 20° C. for 6 hours. The reaction mixture was concentrated under reduced pressure to removed THF and MeOH and the residue was acidized by HCl aqueous (1 N) to pH=3˜4, diluted with DMSO (3 mL). The residue was purified by Prep-HPLC (HCl) to get PA76 (1-[4-[benzyl(methylsulfonyl)amino]phenyl]-9H-pyrido[3,4-b]indole-3-carboxylic acid) (22.8 mg, 23% yield) as a yellow solid. LCMS: m/z 472.1 [M+H]⁺; ¹H NMR (DMSO-d₆, 400 MHz) δ 11.98 (s, 1H), 8.90 (s, 1H), 8.43-8.38 (m, 1H), 8.05 (d, J=8.4 Hz, 2H), 7.68-7.60 (m, 4H), 7.38-7.31 (m, 6H), 5.00 (s, 2H), 3.19 (s, 3H).

Compound 21: To a solution of compound 15 (250 mg, 599 umol, 1.0 eq.) in THF (2 mL) was added benzenesulfonyl chloride (116 mg, 659 umol, 1.1 eq.) and pyridine (189 mg, 2.40 mmol, 4.0 eq.). The mixture was stirred at 20° C. for 10 hours. It was diluted with DCM (20 mL), washed with saturated NaHCO₃ (50 mL), dried over Na₂SO₄, filtered and concentrated in vacuum to get compound 21 (300 mg, crude) as a yellow solid. LCMS: m/z 558.2 [M+H]⁻.

Compound 22: To a solution of compound 21 (100 mg, 179 umol, 1.0 eq.) in DCM (6 mL) was added TFA (3.08 g, 27.01 mmol, 150 eq.) at 20° C. The resulting mixture was stirred at 20° C. for 10 hours. It was concentrated in vacuum to get compound 22 (80.0 mg, crude) as a yellow solid. LCMS: m/z 458.1 [M+H]⁺.

PA77: To a solution of compound 22 (80.0 mg, 175 umol, 1.0 eq.) in H₂O (2 mL), THF (4 mL) and MeOH (4 mL) was added LiOH H₂O (58.7 mg, 1.40 mmol, 8.0 eq.). The mixture was stirred at 50° C. for 10 hours. It was concentrated in vacuum, adjusted with 1M HCl to pH=5, filtered and the yellow solid was obtained. The yellow solid was purified by Prep-HPLC to afford PA77 (1-[4-(benzenesulfonamido)phenyl]-9H-pyrido[3,4-b]indole-3-carboxylic acid) (15.0 mg, 19% yield) as a yellow solid. LCMS: m/z 444.1 [M+H]⁻; ¹H NMR (DMSO-d₆, 400 MHz) δ 12.02-11.93 (m, 1H), 10.71 (s, 1H), 8.98-8.90 (m, 1H), 8.42 (d, J=8.0 Hz, 1H), 7.96 (d, J=8.8 Hz, 2H), 7.89 (d, J=6.8 Hz, 2H), 7.71-7.54 (m, 5H), 7.43-7.28 (m, 3H).

Compound 23: To a solution of compound 21 (150 mg, 269 umol, 1.0 eq.) in DMF (2 mL) was added K₂CO₃ (74.4 mg, 538 umol, 2.0 eq.) and MeI (260 mg, 1.83 mmol, 6.8 eq.). The resulting mixture was stirred at 20° C. for 5 hours. It was filtered and concentrated in vacuum to get compound 23 (300 mg, crude) as a yellow solid. LCMS: m/z 572.2 [M+H]⁺.

Compound 24: To a solution of compound 23 (300 mg, 525 umol, 1.0 eq.) in DCM (9 mL) was added TFA (4.62 g, 40.5 mmol, 77 eq.) at 20° C. The resulting mixture was stirred at 20° C. for 10 hours. It was concentrated in vacuum. The residue was purified by Prep-HPLC to get compound 24 (70.0 mg, 28% yield) as a yellow solid. LCMS: m/z 472.1 [M+H]⁺.

PA78: To a solution of compound 24 (70.0 mg, 148 umol, 1.0 eq.) in H₂O (3 mL), THF (6 mL), MeOH (6 mL) was added LiOH·H₂O (49.8 mg, 1.19 mmol, 8.0 eq.). The mixture was stirred at 50° C. for 10 hours. TLC (PE/EA=1/1) showed the starting material was consumed. It was concentrated in vacuum, adjusted with HCl (1 M in H₂O) to pH=5, filtered to get PA78 (1-[4-[benzenesulfonyl(methyl)amino]phenyl]-9H-pyrido[3,4-b]indole-3-carboxylic acid) (42.0 mg, 61% yield) as a yellow solid. LCMS: m/z 458.0 [M+H]⁺; ¹H NMR (DMSO-d₆, 400 MHz) δ 8.69 (s, 1H), 8.14 (d, J=8.0 Hz, 1H), 7.80 (d, J=8.0 Hz, 2H), 7.54-7.42 (m, 7H), 7.24-7.14 (m, 3H), 3.09 (s, 3H).

### Example 32

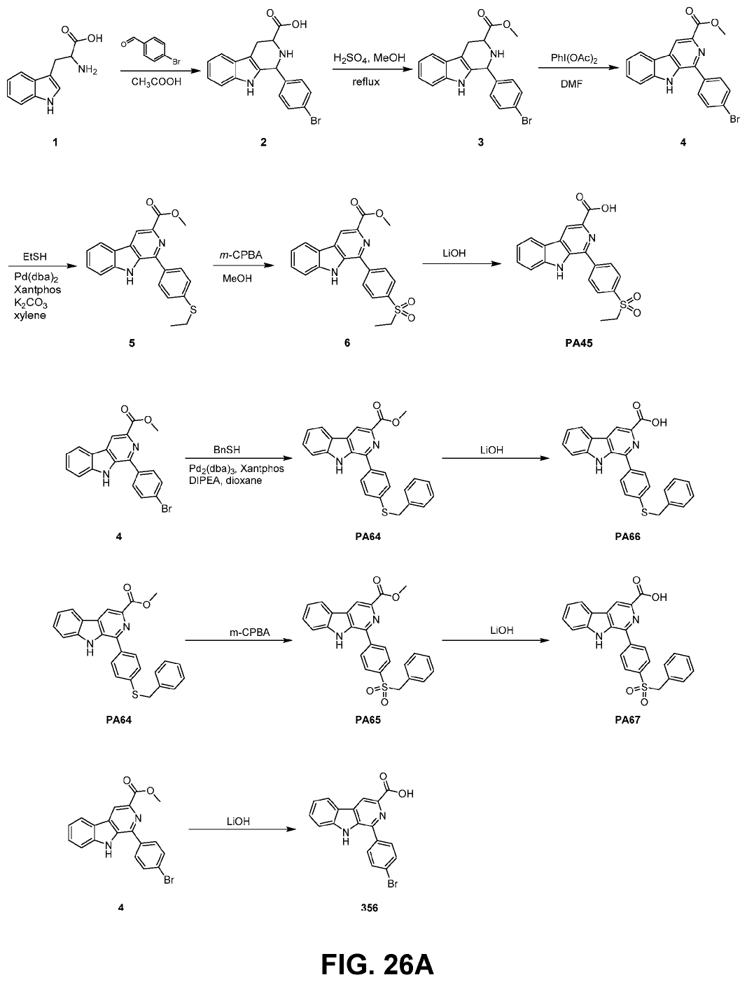

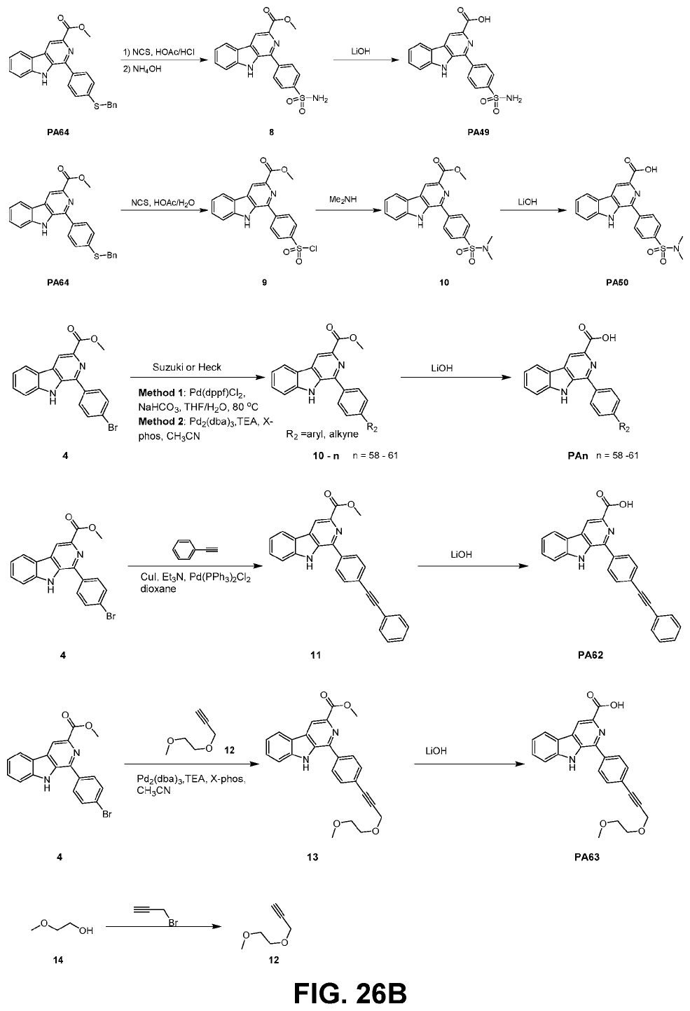

This example demonstrates the synthesis of PA45, PA49, PA50, PA58, PA59, PA60, PA61, PA62, PA63, PA64, PA65, PA66, and PA67, and for some of these compounds the esters or carboxylic acids thereof, though other esters or carboxylic acids could be readily prepared if desired. The synthetic schemes for each of these compounds is shown in FIGS. 26A and 26B.

Compound 4 was synthesized using the procedure shown in Example 30 and FIG. 24 using 4-bromobenzaldehyde.

Compound 5: In a tube were charged successively K₂CO₃ (45.7 mg, 330 umol, 0.63 eq.) and xylene (1 mL). After purging with N₂ using 3 evacuate-fill cycles, the slurry was cooled to 0° C. and the ethanethiol (40.7 mg, 656 umol, 1.3 eq.) was added dropwise. The resulting mixture was then allowed to warm to 25° C. and stirred for 1 hour. To a tube were placed successively compound 4 (200 mg, 525 umol, 1.0 eq.), Pd(dba)₂ (30.2 mg, 52.5 umol, 0.10 eq.), Xantphos (36.4 mg, 63.0 umol, 0.12 eq.) and xylene (5 mL). After purging with N₂ using 3 evacuate-fill cycles, the mixture was stirred at 25° C. for 30 minutes and transferred via a cannula to the previously formed potassium thiolate. The dark solution was then purged with N₂ and heated to 140° C. for 16 hours. The reaction solution was purified directly by silica gel chromatography (PE:EA=10:1 to 6:1) to afford compound 5 (50.0 mg, 24% yield) as a brown solid. LCMS: m/z 363.3 [M+H]⁺.

Compound 6: A solution of compound 5 (50.0 mg, 138 umol, 1.0 eq.) and m-CPBA (95.2 mg, 552 umol, 4.0 eq.) in MeOH (10 mL) was stirred at 15° C. for 12 hours to give a suspension. The suspension was concentrated and the residue was triturated with saturated NaHCO₃ (40 mL). The resulting suspension was filtered and the filter-cake was dried in vacuum to afford compound 6 (50.0 mg, 84% yield) as white solid. LCMS: m/z 395.0 [M+H]⁺.

PA45: A solution of compound 6 (50.0 mg, 127 umol, 1.0 eq.) and LiOH·H₂O (53.2 mg, 1.27 mmol, 10 eq.) in THF (2 mL), MeOH (2 mL) and H₂O (1 mL) was stirred at 50° C. for 24 hours. The solution was concentrated and the residue was diluted with water (20 mL). It was acidified by HCl (1 M in H₂O) to pH=2. The resulting precipitate was collected by filtration as the crude product. The crude product was purified by Prep-HPLC to afford PA45 (1-(4-ethylsulfonylphenyl)-9Hpyrido[3,4-b]indole-3-carboxylic acid) (17.0 mg, 34% yield) as white solid. LCMS: m/z 381.0 [M+H]⁺; ¹H NMR (DMSO-d₆, 400 MHz) δ 12.00 (s, 1H), 8.93 (s, 1H), 8.42 (d, J=8.0 Hz, 1H), 8.40-8.30 (m, 2H), 8.11-8.08 (m, 2H), 7.69-7.66 (m, 1H), 7.63-7.61 (m, 1H), 7.36-7.31 (m, 1H), 1.19 (t, J=7.2 Hz, 3H).

PA64: To a solution of compound 4 (1.60 g, 4.20 mmol, 1.0 eq.) in dioxane (10 mL) was added DIPEA (1.63 g, 12.6 mmol, 3.0 eq.), Xantphos (243 mg, 420 umol, 0.10 eq.) and Pd₂(dba)₃ (384 mg, 420 umol, 0.10 eq.), BnSH (1.04 g, 840 umol, 2.0 eq.) under N₂. The reaction was stirred at 80° C. for 16 hours under N₂. The solution was concentrated to afford residue. The residue was purified by column chromatography (PE:EA=10:1) to give PA64 (methyl 1-(4-benzylsulfanylphenyl)-9H-pyrido[3,4-b]indole-3-carboxylate) (1.80 g, 100% yield) as a white solid. LCMS: m/z 425.3 [M+H]⁺; ¹H NMR (DMSO-d₆, 400 MHz) δ 11.93 (s, 1H), 8.91 (s, 1H), 8.50-8.40 (m, 1H), 7.96 (d, J=8.4 Hz, 2H), 7.68-7.65 (m, 1H), 7.61-7.57 (m, 3H), 7.48-7.45 (m, 2H), 7.35-7.32 (m, 4H), 4.38 (s, 2H), 3.93 (s, 3H).

PA66: To a suspension of PA64 (200 mg, 1.0 eq.) in MeOH (4 mL)/H₂O (1 mL) was added LiOH·H₂O (158 mg, 3.77 mmol, 8.0 eq.). The suspension solution was stirred at 50° C. for 16 hours. The solution was concentrated in vacuum to remove solvent and diluted with water (5 mL) and adjusted pH=2 with 1N HCl, filtered. The crude product was washed with water (5 mL×3) to afford PA66 (1-(4-benzylsulfanylphenyl)-9H-pyrido[3,4-b]indole-3-carboxylic acid) (161 mg, 80% yield) as a white solid. LCMS: m/z 411.1 [M+H]⁺; ¹H NMR (DMSO-d₆, 400 MHz) d 11.70 (s, 1H), 8.80 (s, 1H), 8.35 (d, J=8.0 Hz, 1H), 7.92 (d, J=8.0 Hz, 2H), 7.63-7.48 (m, 2H), 7.43-7.41 (m, 4H), 7.33-7.26 (m, 4H), 4.31 (s, 2H).

PA65: To a solution of PA64 (400 mg, 1.0 eq.) in MeOH (5 mL) was added m-CPBA (650 mg, 3.77 mmol, 4.0 eq.) at 10° C., then the solution was stirred at 20° C. for 3 hours. The reaction solution was purified by Prep-HPLC (FA) to afford PA65 (methyl 1-(4-benzylsulfonylphenyl)-9H-pyrido[3,4-b]indole-3-carboxylate) (300 mg, 69% yield) as a white solid. LCMS: m/z 457.2 [M+H]⁺; ¹H NMR (DMSO-d₆, 400 MHz) δ 12.09 (s, 1H), 9.01 (s, 1H), 8.47 (d, J=8.0 Hz, 1H), 8.23 (d, J=8.0 Hz, 2H), 7.99 (d, J=8.4 Hz, 2H), 7.71-7.69 (m, 1H), 7.68-7.60 (m, 1H), 7.36-7.33 (m, 4H), 7.28-7.26 (m, 2H), 4.81 (s, 2H), 3.95 (s, 3H).

PA67: To a solution of PA65 (200 mg, 438 umol, 1.0 eq.) in MeOH (3 mL)/H₂O (1 mL) was added LiOH·H₂O (147 mg, 3.50 mmol, 8.0 eq.), the solution was stirred at 50° C. for 16 hours. The solution was concentrated in vacuum to remove solvent and diluted with H₂O (3 mL). The suspension was adjusted pH=2 with 1N HCl, filtered and washed with water (3 mL×3) to afford residue. The residue was purified by Prep-HPLC to afford PA67 (1-(4-benzylsulfonylphenyl)-9H-pyrido[3,4-b]indole-3-carboxylic acid) (49.5 mg, 25% yield) as a light yellow solid. LCMS: m/z 443.1 [M+H]⁺; ¹H NMR (DMSO-d₆, 400 MHz) δ 12.01 (s, 1H), 8.94 (s, 1H), 8.42 (d, J=8.0 Hz, 1H), 8.28-8.24 (m, 2H), 7.96-7.94 (m, 2H), 7.66-7.60 (m, 2H), 7.33-7.25 (m, 6H), 4.79 (s, 2H).

Compound 356: To a solution of compound 4 (200 mg, 525 umol, 1.0 eq.) in MeOH (4 mL) was added LiOH·H₂O (176 mg, 4.20 mmol, 8.0 eq.). The reaction was stirred at 50° C. for 16 hours. The suspension was concentrated and the residue was diluted with water (3 mL). It was acidified by HCl (1 M in H₂O) to pH=3. The insoluble was collected by filtration and wash with water (2 mL×3) to afford compound 356 (80.0 mg, 42% yield) as a yellow solid. LCMS: m/z 369.2 [M+H]⁺; ¹H NMR (DMSO-d₆, 400 MHz) δ 11.97 (s, 1H), 8.93 (s, 1H), 8.43 (d, J=8.0 Hz, 1H), 8.04 (d, J=8.4 Hz, 2H), 7.84 (d, J=8.4 Hz, 2H), 7.70-7.61 (m, 2H), 7.35-7.30 (m, 1H).

Compound 8: To a suspension of PA64 (500 mg, 1.18 mmol, 1.0 eq.) in CH₃COOH (33.3 mL) was added HCl (con., 1 mL, 36% in H₂O), the solution was cooled to 0° C. and NCS (236 mg, 1.77 mmol, 1.5 eq.) was added, then the solution was stirred at 20° C. for 3 hours. The solution was concentrated in vacuum to remove solvent and diluted with water (3 mL). The suspension was added into NH₃·H₂O (5 mL) dropwise at 0° C., filtered and washed with water (3 mL×3) to afford compound 8 (110 mg, 20% yield) as a white solid. LCMS: m/z 382.3 [M+H]⁺.

PA49: To a suspension of compound 8 (100 mg, 262 umol, 1.0 eq.) in MeOH (5 mL)/H₂O (1 mL) was added LiOH·H₂O (88.0 mg, 2.10 mmol, 8.0 eq.), the solution was stirred at 50° C. for 16 hours. The solution was concentrated in vacuum to remove solvent and diluted with H₂O (3 mL). The suspension was adjusted pH=2 with 1N HCl, filtered and washed with water (3 mL×3) to afford residue. The residue was purified by Prep-HPLC to afford PA49 (1-(4-sulfamoylphenyl)-9H-pyrido[3,4-b]indole-3-carboxylic acid) (40.5 mg, 42% yield) as a light yellow solid. LCMS: m/z 368.3 [M+H]⁺; ¹H NMR (DMSO-d₆, 400 MHz) δ 12.02 (s, 1H), 8.94 (s, 1H), 8.43 (d, J=7.6 Hz, 1H), 8.27-8.24 (m, 2H), 8.07-8.04 (m, 2H), 7.70-7.56 (m, 4H), 7.37-7.34 (m, 1H).

Compound 9: To a mixture of PA64 (65.0 mg, 153 umol, 1.0 eq.) in CH₃COOH (3 mL) and water (0.5 mL) was added NCS (61.3 mg, 459 umol, 3.0 eq.). The reaction was stirred at 15° C. for 20 hours to afford a yellow suspension. The reaction mixture was poured into ice-water and the precipitate was collected by filtration as the crude product compound 9 (40.0 mg, 65% yield) as yellow solid, which was used directly for next step.

Compound 10: To a stirring solution of Me₂NH (407 mg, 4.99 mmol, 50 eq., HCl salt) in NaHCO₃ (sat., 10 mL) was added a suspension of compound 9 (40.0 mg, 99.8 umol, 1.0 eq.) in THF (20 mL). The mixture was stirred at 10° C. for 1 hour. The reaction mixture was concentrated under reduced pressure and the residue was diluted with water (20 mL). It was extracted with DCM (20 mL×3) and the combined organic layer was concentrated under reduced pressure to give the crude product. The crude product was purified by Prep-HPLC to give compound 10 (25.0 mg, 61% yield) as white solid. LCMS: m/z 410.1 [M+H]⁺.

PA50: To a solution of compound 10 (25.0 mg, 61.1 umol, 1.0 eq.) in THF (1 mL), MeOH (1 mL) and H₂O (0.5 mL)) was added LiOH·H₂O (20.5 mg, 488 umol, 8.0 eq.). The mixture was heated to 50° C. and stirred for 24 hours. The mixture was concentrated in vacuo, the residue was adjusted with HCl (1 M in H₂O) to pH=4 and precipitate formed, the solid was collected and washed with H₂O (3 mL×3), dried in vacuo to get PA50 (1-[4-(dimethylsulfamoyl)phenyl]-9H-pyrido[3,4-b]indole-3-carboxylic acid) (8.10 mg, 33% yield) was obtained as yellow solid. LCMS: m/z 396.0 [M+H]⁺; ¹H NMR (DMSO-d₆, 400 MHz) δ 12.00 (s, 1H), 8.94 (s, 1H), 8.44 (d, J=8.0 Hz, 1H), 8.32-8.29 (m, 2H), 7.96 (d, J=8.0 Hz, 2H), 7.71-7.69 (m, 1H), 7.65-7.60 (m, 1H), 7.37-7.32 (m, 1H), 2.71 (s, 6H).

Compound 12: To a suspension of NaH (3.63 g, 90.7 mmol, 60% purity, 1.2 eq.) in THF (100 mL) was added compound 14 (6.00 g, 78.9 mmol, 1.0 eq.) dropwise over 30 minutes at 0° C. The mixture was stirred at 0° C. for 1 hour, and then 3-bromoprop-1-yne (9.38 g, 78.9 mmol, 1.0 eq.) was added. The mixture was stirred at 25° C. for 17 hours, filtered and concentrated in vacuum. The residue was poured into water (100 mL) and the aqueous phase was extracted with DCM (100 mL×3). The combined organic phase was washed with brine (100 mL), dried with anhydrous Na₂SO₄, filtered and concentrated in vacuum. The residue was purified by silica gel chromatography (PE/EA=1/0 to 10/1) to afford compound 12 (3.20 g, 36% yield) as a light yellow liquid. ¹H NMR (CDCl₃, 400 MHz) δ 4.27 (s, 1H), 3.71-3.68 (m, 2H), 3.59-3.56 (m, 2H), 3.39 (s, 3H), 2.46-2.42 (m, 1H).

Compounds 10-58, 10-59, 10-60, and 10-61: To a solution of compound 4 (1.0 eq.), boronic acid (1.1 eq.) and NaHCO₃ (2.0 eq.) in THF (1˜3 mL)/H₂O (0.3˜1 mL) was added Pd(dppf)Cl₂ (0.10 eq.). The suspension was degassed under vacuum and purged with N₂ three times and stirred at 80° C. for 16 hours under N₂. The suspension was poured into water (5 mL) and extracted with DCM (10 mL×3). The combined organic phase was washed with brine (20 mL), dried with anhydrous Na₂SO₄, concentrated to give residue. The residue was purified by Prep-TLC (PE:EA=2:1) to afford the desired product. Compound 10-58 (4-fluorophenyl): 150 mg, 44% yield, white solid; LCMS: m/z 397.1 [M+H]⁺. Compound 10-59 (4-methylphenyl): 100 mg, 32% yield, white solid; LCMS: m/z 393.3 [M+H]⁺. Compound 10-60 (4-methoxyphenyl): 260 mg, 77% yield, white solid; LCMS: m/z 409.2 [M+H]⁺. Compound 10-61 (4-methylsulfonylphenyl): 93.0 mg, 36% yield, white solid; LCMS: m/z 457.2 [M+H]⁺.

Compound 11: To a solution of compound 4 (200 mg, 525 umol, 1.0 eq.), ethynylbenzene (268 mg, 2.62 mmol, 5.0 eq.), TEA (26.5 mg, 262 umol, 5.0 eq.) and CuI (5.00 mg, 26.2 umol, 0.05 eq.) in dioxane (5 mL) was added Pd(PPh₃)₂Cl₂ (29.5 mg, 42.0 umol, 0.08 eq.). The suspension was degassed under vacuum and purged with N₂ three times. The reaction was stirred at 80° C. for 16 hours under N₂. The mixture was concentrated in vacuum to give compound 11 (300 mg, crude) as black gum. LCMS: m/z 403.1 [M+H]⁺.

Compound 13: To a mixture of compound 4 (200 mg, 525 umol, 1.0 eq.) and 3-(2-methoxyethoxy)prop-1-yne (299 mg, 2.62 mmol, 5.0 eq.) in CH₃CN (10 mL) was added Pd₂(dba)₃ (48.0 mg, 52.5 umol, 0.10 eq.), XPhos (50.0 mg, 105.0 umol, 0.20 eq.), CuI (50.0 mg, 262 umol, 0.50 eq.), TEA (159 mg, 1.57 mmol, 3.0 eq.) in one portion under N₂. The mixture was stirred at 80° C. stirred for 12 hours. The mixture was concentrated in reduced pressure. The residue was poured into water (50 mL). The aqueous phase was extracted with ethyl acetate (20 mL×3). The combined organic phase was washed with brine (20 mL), dried with anhydrous Na₂SO₄, filtered and concentrated in vacuum. The residue was purified by Prep-TLC (DCM:MeOH=20:1) to give the product compound 13 (50.0 mg, 23% yield) as a yellow solid.

PA58, PA59, PA60, PA61, PA62, and PA63: To a solution of ester compound 10-n (1.0 eq.) in MeOH (2 mL)/H₂O (2 mL) was added LiOH·H₂O (2.0 eq.). The mixture was stirred at 25° C.˜50° C. for 4˜24 hours. The mixture was acidified by HCl (1 M) to pH=2. And then the mixture was concentrated under reduced pressure to afford a residue. The residue was filtered through a shot layer of silica gel and eluted with DCM/MeOH=10/1 to afford the crude product, which was further purified by Prep-HPLC to get the desired product.

PA58: 52.0 mg, 35% yield, yellow solid; LCMS: m/z 383.1 [M+H]⁻; ¹H NMR (DMSO-d₆, 400 MHz) δ 11.95 (s, 1H), 8.90 (s, 1H), 8.50-8.40 (m, 1H), 8.20 (s, 2H), 7.92-7.84 (m, 4H), 7.72-7.69 (m, 1H), 7.63-7.60 (m, 1H), 7.36-7.30 (m, 3H).

PA59: 32.0 mg, 32% yield, yellow solid; LCMS: m/z 379.1 [M+H]⁻; ¹H NMR (DMSO-d₆, 400 MHz) δ 11.99 (s, 1H), 8.92 (s, 1H), 8.43 (d, J=7.6 Hz, 1H), 8.17 (d, J=8.0 Hz, 2H), 7.92 (d, J=8.0 Hz, 2H), 7.73-7.70 (m, 3H), 7.64-7.59 (m, 1H), 7.40-7.30 (m, 3H), 2.38 (s, 3H).

PA60: 90.0 mg, 34% yield, yellow solid; LCMS: m/z 395.1 [M+H]⁻; ¹H NMR (DMSO-d₆, 400 MHz) δ 11.97 (s, 1H), 8.91 (s, 1H), 8.42 (d, J=8.0 Hz, 1H), 8.16 (d, J=8.0 Hz, 2H), 7.90 (d, J=8.4 Hz, 2H), 7.78-7.70 (m, 3H), 7.65-7.55 (m, 1H), 7.40-7.30 (m, 1H), 7.10 (d, J=8.8 Hz, 2H), 3.83 (s, 3H).

PA61: 93.0 mg, 36% yield, white solid; LCMS: nm/z 443.0 [M+H]⁺; ¹H NMR (DMSO-d₆, 400 MHz) δ 12.03 (s, 1H), 8.94 (s, 1H), 8.44 (d, J=8.4 Hz, 1H), 8.25 (d, J=8.4 Hz, 2H), 8.10-8.04 (m, 6H), 7.73-7.70 (m, 1H), 7.65-7.55 (m, 1H), 7.35-7.33 (m, 1H), 3.30 (s, 3H).

PA62: 81.0 mg, 32% yield, yellow solid; LCMS: m/z 389.1 [M+H]⁻; ¹H NMR (DMSO-d₆, 400 MHz) δ 12.00 (s, 1H), 8.93 (s, 1H), 8.44 (d, J=8.4 Hz, 1H), 8.18 (d, J=8.4 Hz, 2H), 7.82 (d, J=8.4 Hz, 2H), 7.75-7.70 (m, 1H), 7.64-7.62 (m, 3H), 7.48-7.45 (m, 3H), 7.40-7.30 (m, 1H).

PA63: 81.0 mg, 32% yield, yellow solid; LCMS: m/z 401.1 [M+H]⁻; ¹H NMR (MeOD-d₄, 400 MHz) δ 8.99 (s, 1H), 8.35 (d, J=8.4 Hz, 1H), 8.04-8.00 (m, 2H), 7.73-7.64 (m, 4H), 7.43-7.38 (m, 1H), 4.48 (s, 2H), 3.80-3.77 (m, 2H), 3.65-3.62 (m, 2H), 3.41 (s, 3H).

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A compound of formula (I): 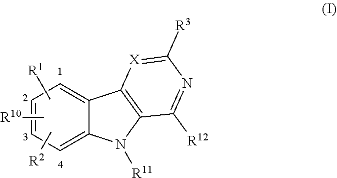
wherein:
X is C—H or N;
R¹, R², and R¹⁰ independently are —H, —OH, —CN, -haloalkyl, —O—(C₁-C₆ alkyl), halogen, —C₁-C₆ alkyl, or phenyl;
R³ is —COOH, —COO—(C₁-C₆ alkyl), —CONH₂, —CON(C₁-C₆ alkyl)₂, —CONH(C₁-C₆ alkyl), —CONHSO₂(C₁-C₆ alkyl), —CON(C₁-C₆ alkyl)SO₂(C₁-C₆ alkyl), halogen, —SO₂NH₂, —NHSO₂(C₁-C₆ alkyl), —CN, or tetrazole;
R¹¹ is —H or —C₁-C₆ alkyl;
wherein any alkyl, phenyl, or tetrazole moiety of an R¹, R², R³, R¹⁰, or R¹¹ group can be further substituted with —C₁-C₆ alkyl, —O—(C₁-C₆ alkyl), —SO₂(C₁-C₆ alkyl), halogen, —CN, haloalkyl, —OH, pyridinyl, —NO₂, —N-piperazinyl-N—(C₁-C₆ alkyl), phenyl, Bn, or any combination thereof; and
R¹² is phenyl, a 5-membered heterocycle, or a 6-membered heterocycle, each of which is substituted with R⁴, R⁵, and R⁶ on a carbon atom and/or heteroatom, wherein:
R⁴, R⁵, and R⁶ independently are —H, —C₁-C₆ alkyl, halogen, —OH, —O(C₁-C₆ alkyl), —CN, —NO₂, —(C₁-C₆ alkyl)-OH, SO₂(C₁-C₆ alkyl), -haloalkyl, —CO₂(C₁-C₆ alkyl), —NHSO₂(C₁-C₆ alkyl), —NHBn, phenyl, —SBn, —OBn, —SO₂NH₂, —SO₂(C₁-C₆ alkyl), —SO₂Bn, SO₂N(C₁-C₆ alkyl)₂, —OSO₂(C₁-C₆ alkyl), —C≡C-phenyl, —C≡CCH₂(OCH₂CH₂)₁₋₄₀H, —C≡CCH₂(OCH₂CH₂)₁₋₄OMe, —NHSO₂-pyridinyl, —NBnSO₂-pyridinyl, —NHSO₂-phenyl, —NHSO₂(N-piperazinyl-N—(C₁-C₆ alkyl)), —N(C₁-C₆ alkyl)SO₂-pyridinyl, —N(C₁-C₆ alkyl)SO₂-phenyl, —N(C₁-C₆ alkyl)SO₂(N-piperazinyl-N—(C₁-C₆ alkyl)), —NBnSO₂(C₁-C₆ alkyl), —NBnSO₂(N-piperazinyl-N—(C₁-C₆ alkyl)), —NBnSO₂-phenyl, or —N(C₁-C₆ alkyl)SO₂(C₁-C₆ alkyl); or
R⁴ and R⁵ taken together form a fused pyrazole ring, a fused pyrrole ring, or a fused phenyl ring, and R⁶ is as defined above;
wherein any alkyl, phenyl, Bn, pyridinyl, fused pyrazole ring, fused pyrrole ring, or fused phenyl ring moiety of an R⁴, R⁵, or R⁶ group can be further substituted with —C₁-C₆ alkyl, —O—(C₁-C₆ alkyl), —SO₂(C₁-C₆ alkyl), halogen, —CN, haloalkyl, —OH, pyridinyl, furanyl, tetrahydrofuranyl, tetrahydropyranyl, thiophenyl, thiazolyl, pyrrolyl, pyrrolidinyl, piperidinyl, morpholinyl, pyrimidinyl, triazolyl, imidazolyl, —NO₂, —N-piperazinyl-N—(C₁-C₆ alkyl), phenyl, -Bn, or any combination thereof;
provided that:
when R¹² is phenyl; R¹, R², R⁴, R⁵, and R¹⁰ are —H; and R³ is tetrazole or tetrazole with its N—H group substituted with N-Bn; then R⁶ is not —H, 4-methoxy, or 4-fluoro;
when R¹² is phenyl; R¹, R², R⁴, R⁵, and R¹⁰ are —H; and R³ is —COOH; then R⁶ is not —H, 4-nitro, 4-chloro, 4-methoxyl, 4-CF₃, 4-bromo, 4-isopropyl, or 4-hydroxyl;
when R¹² is phenyl; R¹, R², R⁴, and R¹⁰ are —H; R³ is —COOH; and R⁵ is 4-hydroxyl; then R⁶ is not CO₂Me or methoxyl;
when R¹² is phenyl; R¹ R², and R¹⁰ are —H; and R³ is —COOH, —COOMe, or —COOEt; then R⁴, R⁵, and R⁶ are not 3-methoxyl, 4-methoxyl, and 5-methoxyl, respectively;
when R¹² is phenyl; R¹, R², R⁴, R⁵, and R¹⁰ are —H; and R³ is —COOMe; then R⁶ is not —H, 4-methoxyl, 2-chloro, 4-chloro, 4-hydroxyl, 3-nitro, 4-nitro, 4-bromo, 4-methoxyl, or 4-CF₃; and
when R¹² is phenyl; R¹, R⁴, R⁵, and R¹⁰ are —H; R² is methoxyl or methyl; and R³ is —COOEt; then R⁶ is not —H, 4-methoxyl, 2-chloro, 4-hydroxyl, or 3-nitro;
or a salt or ester thereof;
or
a compound of formula (II): 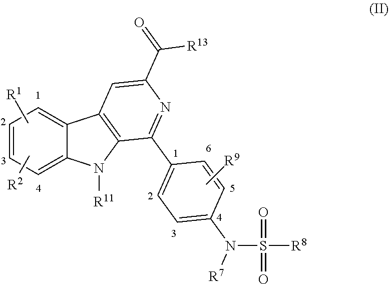
wherein:
R¹ and R² independently are —H, —OH, —C₁-C₆ alkyl, —O—(C₁-C₆ alkyl), halogen, —CN, haloalkyl, or phenyl;
R¹¹ is —H or —C₁-C₆ alkyl;
R¹³ is —OH, —O—(C₁-C₆ alkyl), —NH₂, —NHSO₂—(C₁-C₆ alkyl), or —N(C₁-C₆ alkyl)SO₂(C₁-C₆ alkyl);
R⁷ is —H, —C₁-C₆ alkyl, or -Bn;
R⁸ is —C₁-C₆ alkyl, —N-piperazinyl-N—(C₁-C₆ alkyl), pyridinyl, or phenyl; and
R⁹ is —H, —OH, —C₁-C₆ alkyl, —O—(C₁-C₆ alkyl), haloalkyl, or a halogen:
wherein any alkyl, phenyl, Bn, or pyridinyl moiety on an R¹, R², R⁷, R⁸, R⁹, R¹¹, or R¹³ group can be further substituted with —C₁-C₆ alkyl, —O—(C₁-C₆ alkyl), —SO₂(C₁-C₆ alkyl), halogen, —CN, haloalkyl, —OH, pyridinyl, furanyl, tetrahydrofuranyl, tetrahydropyranyl, thiophenyl, thiazolyl, pyrrolyl, pyrrolidinyl, piperidinyl, morpholinyl, pyrimidinyl, triazolyl, imidazolyl, —NO₂, —N-piperazinyl-N—(C₁-C₆ alkyl), phenyl, -Bn, or any combination thereof;
or a salt or ester thereof
or
a compound of formula (III): 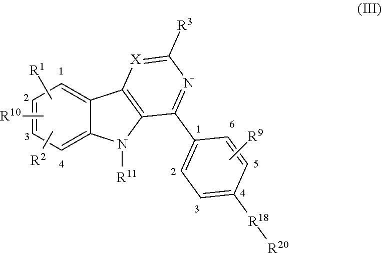
wherein:
X is C—H or N,
R¹, R², and R¹⁰ independently are —H, —OH, —CN, haloalkyl, —O—(C₁-C₆ alkyl), halogen, —C₁-C₆ alkyl, or phenyl;
R³ is —COOH, —COO—(C₁-C₆ alkyl), —CONH₂, —CON(C₁-C₆ alkyl)₂, —CONH(C₁-C₆ alkyl), —CONHSO₂(C₁-C₆ alkyl), —CON(C₁-C₆ alkyl)SO₂(C₁-C₆ alkyl), halogen, —SO₂NH₂, —NHSO₂(C₁-C₆ alkyl), —CN, or tetrazole:
R¹¹ is —H or C₁-C₆ alkyl;
R¹⁸ is —O—, —S—, —NR¹⁹—, —(C₁-C₆ alkyl)-, —(O—(C₁-C₆ alkyl))-, —(O—(C₁-C₆ alkyl)-O)—, —(OCH₂CH₂)ₙ—O—, —(NR¹⁹—(C₁-C₆ alkyl))-, —(NR¹⁹—(C₁-C₆ alkyl)-O)—, —(NR¹⁹—(C₁-C₆ alkyl)-NR¹⁹)—, —(OCH₂CH₂)ₙ—NR¹⁹—, —(S—(C₁-C₆ alkyl))-, —(S—(C₁-C₆ alkyl)-S)—, —(OCH₂CH₂)ₙ—S—, —(NR¹⁹—(C₁-C₆ alkyl)-S)—, —(S—(C₁-C₆ alkyl)-O)—, or —(CONR¹⁹)ₙ—;
wherein each n independently is an integer from 1 to 6:
wherein each R¹⁹ independently is H or C₁-C₆ alkyl; and
R⁹ is —H, —OH, —C₁-C₆ alkyl, —O—(C₁-C₆ alkyl), phenyl, haloalkyl, or halogen:
R²⁰ is C₃-C₆ cycloalkyl, phenyl, a 5-membered heterocycle, or a 6-membered heterocycle, each of which is substituted with R¹⁴, R¹⁵, and R¹⁶ on a carbon atom and/or heteroatom,
wherein:
R¹⁴, R¹⁵ and R¹⁶ independently are —H, —C₁-C₆ alkyl, halogen, —OH, —O(C₁-C₆ alkyl), —CN, —NO₂, —(C₁-C₆ alkyl)-OH, SO₂(C₁-C₆ alkyl), haloalkyl, —CO₂(C₁-C₆ alkyl), —NHSO₂(C₁-C₆ alkyl), —NHBn, phenyl, —SBn, —OBn, —SO₂NH₂, —SO₂(C₁-C₆ alkyl), —SO₂Bn, SO₂N(C₁-C₆ alkyl)₂, —OSO₂(C₁-C₆ alkyl), —C≡C-phenyl, —C≡CCH₂(OCH₂CH₂)₁₋₄₀H, —C≡CCH₂(OCH₂CH₂)₁₋₄OMe, —NHSO₂-pyridinyl, —NBnSO₂-pyridinyl, —NHSO₂-phenyl, —NHSO₂(N-piperazinyl-N—(C₁-C₆ alkyl)), —N(C₁-C₆ alkyl)SO₂-pyridinyl, —N(C₁-C₆ alkyl)SO₂-phenyl, —N(C₁-C₆ alkyl)SO₂(N-piperazinyl-N—(C₁-C₆ alkyl)), —NBnSO₂(C₁-C₆ alkyl), —NBnSO₂(N-piperazinyl-N—(C₁-C₆ alkyl)), —NBnSO₂-phenyl, or —N(C₁-C₆ alkyl)SO₂(C₁-C₆ alkyl); or
R¹⁴ and R¹⁵ taken together form a fused pyrazole ring, a fused pyrrole ring, or a fused phenyl ring, and R¹⁶ is as defined above; and
wherein any -Bn, pyridinyl, alkyl, tetrazole, fused pyrazole ring, fused pyrrole ring, fused phenyl ring, or phenyl moiety of the foregoing groups can be substituted with C₁-C₆ alkyl, —O—(C₁-C₆ alkyl), —SO₂(C₁-C₆ alkyl), halogen, —CN, haloalkyl, —OH, pyridinyl, furanyl, tetrahydrofuranyl, tetrahydropyranyl, thiophenyl, thiazolyl, pyrrolyl, pyrrolidinyl, piperidinyl, morpholinyl, pyrimidinyl, triazolyl, imidazolyl, nitro, —N-piperazinyl-N—(C₁-C₆ alkyl), phenyl, Bn, or any combination thereof;
or a salt or ester thereof.

2. (canceled)

3. The compound of claim 1, wherein, in formula (II):
R¹ and R² independently are —H, —OH, methyl, methoxyl, fluoro, chloro, bromo, phenyl, —CF₃, —CH₂F, —CHF₂, —CCl₃, or —CN;
R¹³ is —OH, —NH₂, —O—(C₁-C₆ alkyl), or —NHSO₂Me;
R⁷ is —H, methyl, or -Bn;
R⁸ is methyl, N-piperazinyl-N-methyl, pyridinyl, phenyl, or phenyl substituted with at least one of methyl, methoxyl, pyridinyl, fluoro, chloro, bromo, or N-piperazinyl-N-methyl; and
R⁹ is H, methyl, fluoro, chloro, bromo, haloalkyl, —O—(C₁-C₆ alkyl), or —OH;
or.

4. The compound of claim 1, wherein, in formula (II):
R¹ is —H, fluoro, chloro, bromo, cyano, phenyl, methoxyl, hydroxyl, or methyl;
R² is fluoro, chloro, bromo, cyano, phenyl, methoxyl, hydroxyl, or methyl;
R¹³ is —OH or —O—(C₁-C₆ alkyl);
R⁷ is —H, methyl, or -Bn;
R⁸ is methyl, N-piperazinyl-N-methyl, pyridinyl, phenyl, or phenyl substituted with at least one of methyl, methoxyl, pyridinyl, fluoro, chloro, bromo, or N-piperazinyl-N-methyl; and
R⁹ is —H, fluoro, or methyl.

5. The compound of claim 1, wherein, in formula (II):
R¹ is —H, fluoro, or methyl;
R² is fluoro, chloro, bromo, or cyano;
R¹³ is —OH or —O—(C₁-C₆ alkyl);
R⁷ is —H or methyl;
R⁸ is methyl, N-piperazinyl-N-methyl, pyridinyl, phenyl, or phenyl substituted with at least one of methyl, methoxyl, pyridinyl, fluoro, chloro, bromo, or N-piperazinyl-N-methyl;
R⁹ is —H or fluoro.

6. The compound of claim 1, wherein, in formula (II):
R¹ is —H;
R² is fluoro or cyano;
R¹³ is —OH or —O—(C₁-C₆ alkyl);
R⁷ is —H or methyl;
R⁸ is methyl, N-piperazinyl-N-methyl, pyridinyl, phenyl, or phenyl substituted with at least one of methyl, methoxyl, pyridinyl, fluoro, chloro, bromo, or N-piperazinyl-N-methyl;
R⁹ is —H.

7. The compound of claim 1, wherein R¹³ is —OH, methoxyl, ethoxyl, isopropoxyl, n-propoxyl, isobutoxyl, sec-butoxyl, n-butoxyl, or t-butoxyl.

8. The compound of claim 1, wherein the compound is: 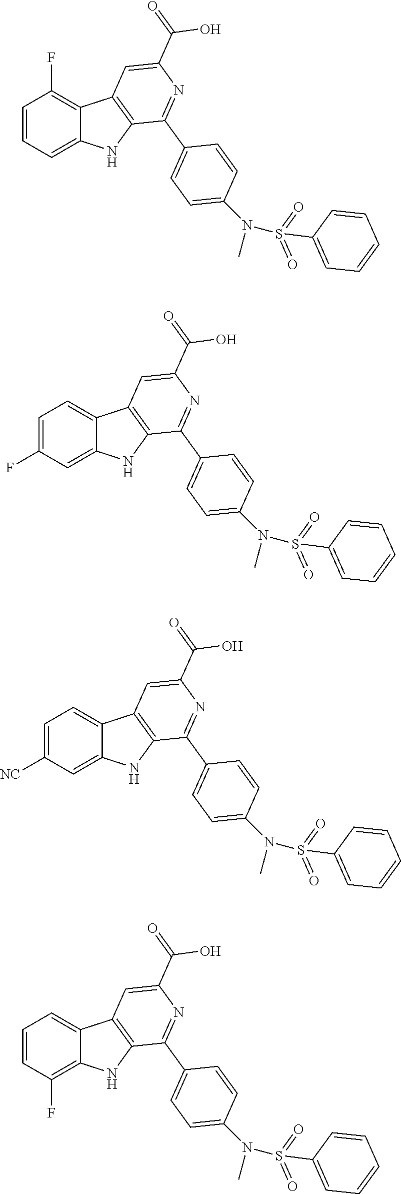 [SMILES: CN(c1ccc(-c2nc(C(=O)O)cc3c2[nH]c2cccc(F)c23)cc1)S(=O)(=O)c1ccccc1 ; CN(c1ccc(-c2nc(C(=O)O)cc3c2[nH]c2cc(F)ccc23)cc1)S(=O)(=O)c1ccccc1 ; CN(c1ccc(-c2nc(C(=O)O)cc3c2[nH]c2cc(C#N)ccc23)cc1)S(=O)(=O)c1ccccc1 ; CN(c1ccc(-c2nc(C(=O)O)cc3c2[nH]c2c(F)cccc23)cc1)S(=O)(=O)c1ccccc1] 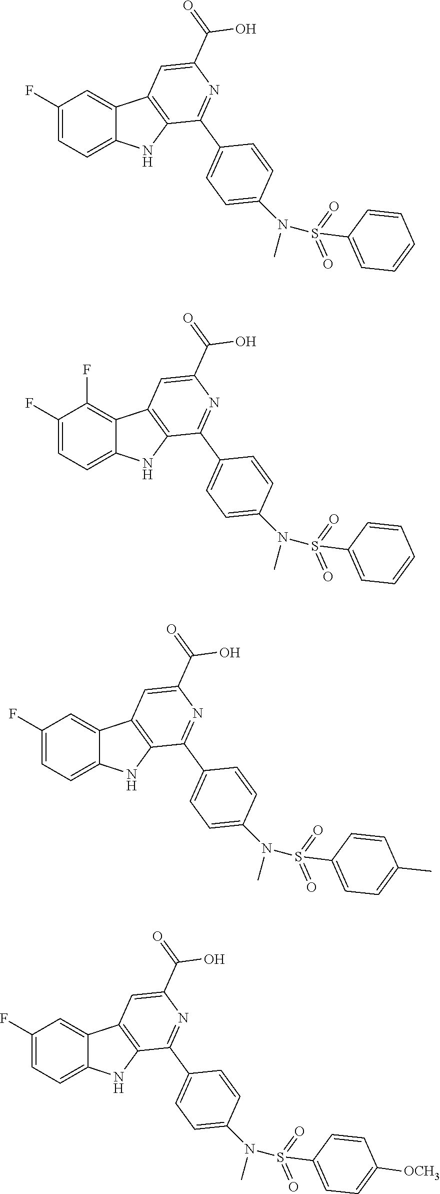 [SMILES: CN(c1ccc(-c2nc(C(=O)O)cc3c2[nH]c2ccc(F)cc23)cc1)S(=O)(=O)c1ccccc1 ; CN(c1ccc(-c2nc(C(=O)O)cc3c2[nH]c2ccc(F)c(F)c23)cc1)S(=O)(=O)c1ccccc1 ; Cc1ccc(S(=O)(=O)N(C)c2ccc(-c3nc(C(=O)O)cc4c3[nH]c3ccc(F)cc34)cc2)cc1 ; COc1ccc(S(=O)(=O)N(C)c2ccc(-c3nc(C(=O)O)cc4c3[nH]c3ccc(F)cc34)cc2)cc1] 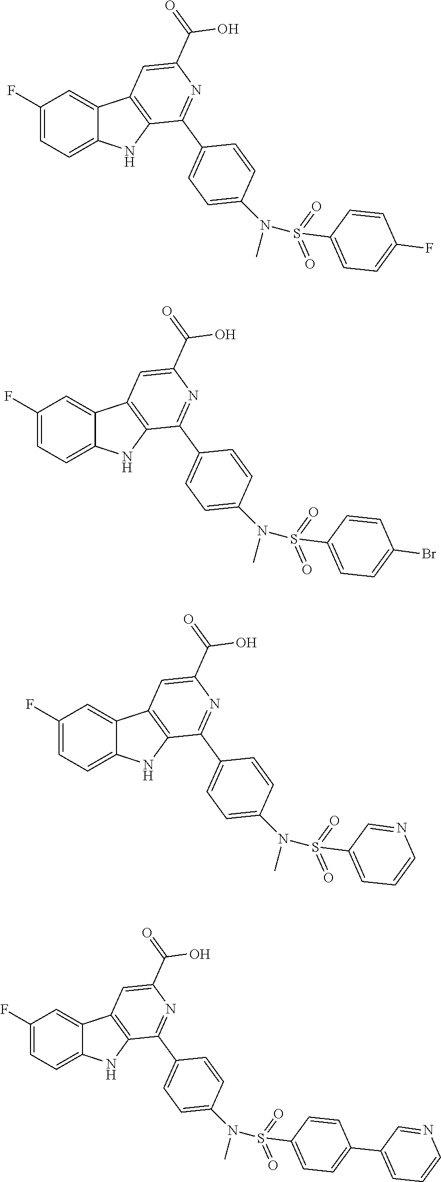 [SMILES: CN(c1ccc(-c2nc(C(=O)O)cc3c2[nH]c2ccc(F)cc23)cc1)S(=O)(=O)c1ccc(F)cc1 ; CN(c1ccc(-c2nc(C(=O)O)cc3c2[nH]c2ccc(F)cc23)cc1)S(=O)(=O)c1ccc(Br)cc1 ; CN(c1ccc(-c2nc(C(=O)O)cc3c2[nH]c2ccc(F)cc23)cc1)S(=O)(=O)c1cccnc1 ; CN(c1ccc(-c2nc(C(=O)O)cc3c2[nH]c2ccc(F)cc23)cc1)S(=O)(=O)c1ccc(-c2cccnc2)cc1] 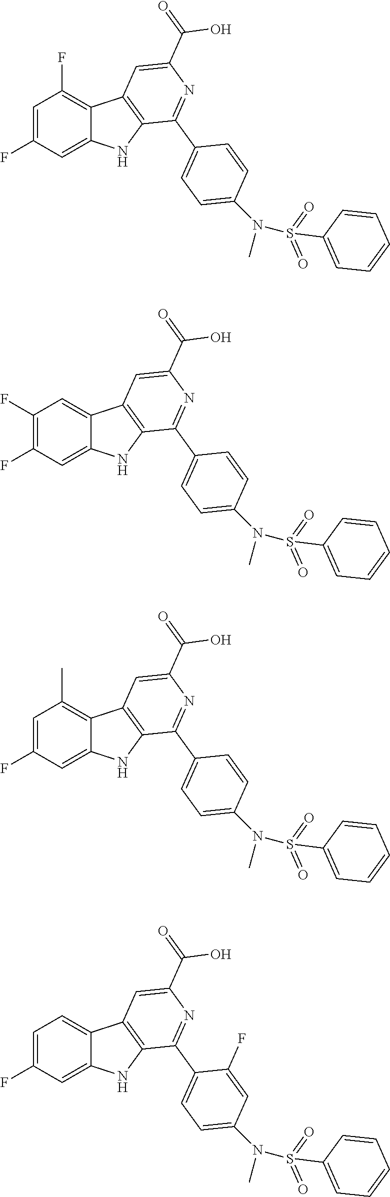 [SMILES: CN(c1ccc(-c2nc(C(=O)O)cc3c2[nH]c2cc(F)cc(F)c23)cc1)S(=O)(=O)c1ccccc1 ; CN(c1ccc(-c2nc(C(=O)O)cc3c2[nH]c2cc(F)c(F)cc23)cc1)S(=O)(=O)c1ccccc1 ; Cc1cc(F)cc2[nH]c3c(-c4ccc(N(C)S(=O)(=O)c5ccccc5)cc4)nc(C(=O)O)cc3c12 ; CN(c1ccc(-c2nc(C(=O)O)cc3c2[nH]c2cc(F)ccc23)c(F)c1)S(=O)(=O)c1ccccc1] 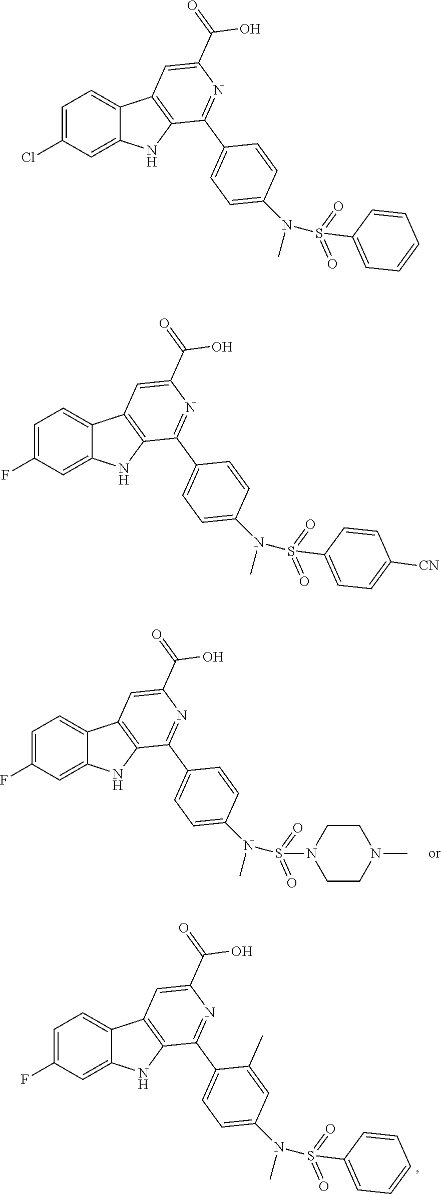 [SMILES: CN(c1ccc(-c2nc(C(=O)O)cc3c2[nH]c2cc(Cl)ccc23)cc1)S(=O)(=O)c1ccccc1 ; CN(c1ccc(-c2nc(C(=O)O)cc3c2[nH]c2cc(F)ccc23)cc1)S(=O)(=O)c1ccc(C#N)cc1 ; CN1CCN(S(=O)(=O)N(C)c2ccc(-c3nc(C(=O)O)cc4c3[nH]c3cc(F)ccc34)cc2)CC1 ; Cc1cc(N(C)S(=O)(=O)c2ccccc2)ccc1-c1nc(C(=O)O)cc2c1[nH]c1cc(F)ccc12]
or a salt or ester thereof, optionally 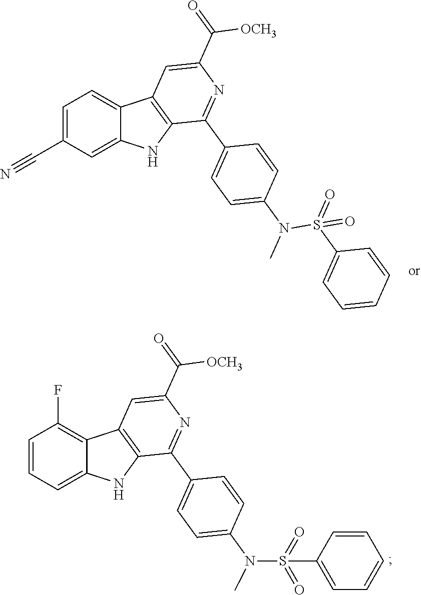 [SMILES: COC(=O)c1cc2c([nH]c3cc(C#N)ccc32)c(-c2ccc(N(C)S(=O)(=O)c3ccccc3)cc2)n1 ; COC(=O)c1cc2c([nH]c3cccc(F)c32)c(-c2ccc(N(C)S(=O)(=O)c3ccccc3)cc2)n1]
or a compound of Table 1 or a salt or ester thereof.

9. The compound of claim 1, wherein the compound is: 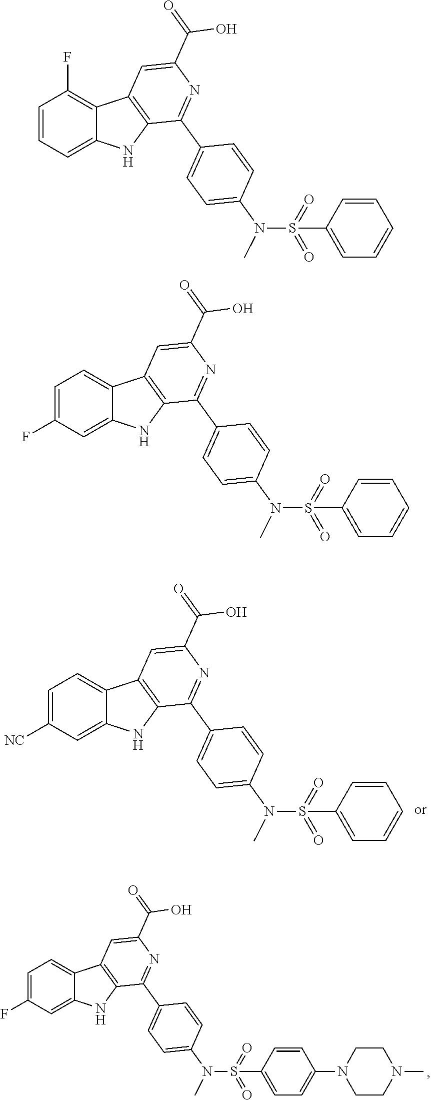 [SMILES: CN(c1ccc(-c2nc(C(=O)O)cc3c2[nH]c2cccc(F)c23)cc1)S(=O)(=O)c1ccccc1 ; CN(c1ccc(-c2nc(C(=O)O)cc3c2[nH]c2cc(F)ccc23)cc1)S(=O)(=O)c1ccccc1 ; CN(c1ccc(-c2nc(C(=O)O)cc3c2[nH]c2cc(C#N)ccc23)cc1)S(=O)(=O)c1ccccc1 ; CN1CCN(c2ccc(S(=O)(=O)N(C)c3ccc(-c4nc(C(=O)O)cc5c4[nH]c4cc(F)ccc45)cc3)cc2)CC1]
or a salt or ester thereof.

10. The compound of claim 1, wherein, in formula (I):
R¹, R², and R¹⁰ independently are H, hydroxyl, methoxyl, halogen, methyl, —CN, —CF₃, —CH₂F, —CHF₂, —CCl₃, or phenyl;
R³ is —COOH, —COO—(C₁-C₆ alkyl), —CONH₂, —CONHSO₂(C₁-C₆ alkyl), or tetrazole;
R¹¹ is H or —C¹-C⁶ alkyl;
wherein any alkyl, phenyl, or tetrazole moiety of an R¹, R², R³, R¹⁰, or R¹¹ group can be further substituted with —C₁-C₆ alkyl, —O—(C₁-C₆ alkyl), —SO₂(C₁-C₆ alkyl), halogen, —CN, haloalkyl, —OH, pyridinyl, —NO₂, —N-piperazinyl-N—(C₁-C₆ alkyl), phenyl, -Bn, or any combination thereof; and
R¹² is phenyl, imidazole, pyridine, or dihydropyrimidine, each of which is substituted with R⁴, R⁵, and R⁶ on a carbon atom and/or heteroatom;
wherein:
R⁴, R⁵, and R⁶ independently are —H, methyl, fluoro, chloro, bromo, —OH, methoxyl, —CN, —NO₂, —CH₂OH, —SO₂Me, —CF₃, —CH₂F, —CHF₂, —CCl₃, —COOMe, —COOEt, —NHSO₂Me, —NHBn, phenyl, —SBn, —OBn, —SO₂NH₂, —SO₂Me, —SO₂Et, —SO₂Bn, —SO₂NMe₂, —OSO₂Me, —C≡C-phenyl, —C≡CCH₂(OCH₂CH₂)OH, or —C≡CCH₂(OCH₂CH₂)OMe, or
R⁴ and R⁵ taken together form a fused pyrazole ring, a fused pyrrole ring, or a fused phenyl ring, and R⁶ is as defined above; and
wherein any alkyl, phenyl, -Bn, pyridinyl, fused pyrazole ring, fused pyrrole ring, or fused phenyl ring moiety of an R⁴, R⁵, or R⁶ group can be further substituted with —C₁-C₆ alkyl, —O—(C₁-C₆ alkyl), —SO₂(C₁-C₆ alkyl), halogen, —CN, haloalkyl, —OH, pyridinyl, furanyl, tetrahydrofuranyl, tetrahydropyranyl, thiophenyl, thiazolyl, pyrrolyl, pyrrolidinyl, piperidinyl, morpholinyl, pyrimidinyl, triazolyl, imidazolyl, —NO₂, —N-piperazinyl-N—(C₁-C₆ alkyl), phenyl, Bn, or any combination thereof;
provided that:
when R¹² is phenyl; R¹, R², R⁴, R⁵, and R¹⁰ are —H; and R³ is tetrazole or tetrazole with its N—H group substituted with N-Bn; then R⁶ is not phenyl, 4-methoxy, or 4-fluoro;
when R¹² is phenyl; R¹, R², R⁴, R⁵, and R¹⁰ are —H; and R³ is —COOH; then R⁶ is not —H, 4-nitro, 4-chloro, 4-methoxyl, 4-CF₃, 4-bromo, 4-isopropyl, or 4-hydroxyl;
when R¹² is phenyl; R¹, R², R⁴, and R¹⁰ are —H; R³ is —COOH; and R⁵ is 4-hydroxyl; then R⁶ is not CO₂Me or methoxyl;
when R¹² is phenyl; R¹, R², and R¹⁰ are —H; and R³ is —COOH, —COOMe, or —COOEt; then R⁴, R⁵, and R⁶ are not 3-methoxyl, 4-methoxyl, and 5-methoxyl, respectively;
when R¹² is phenyl; R¹, R², R⁴, R⁵, and R¹⁰ are —H; and R³ is —COOMe; then R⁶ is not —H, 4-methoxyl, 2-chloro, 4-chloro, 4-hydroxyl, 3-nitro, 4-nitro, 4-bromo, 4-methoxyl, or 4-CF₃; and
when R¹² is phenyl; R¹, R⁴, R⁵, and R¹⁰ are —H; R² is methoxyl or methyl; and
R³ is —COOEt; then R⁶ is not —H, 4-methoxyl, 2-chloro, 4-hydroxyl, or 3-nitro.

11. The compound of claim 1, wherein R⁴, R⁵, and R⁶ independently are —H; methyl; fluoro; chloro; bromo; hydroxyl; methoxyl; —CN; —NO₂; —CH₂OH; —SO₂Me; —CF₃; —CH₂F; —CHF₂; —CCl₃; —CO₂Me; —CO₂Et; —NHSO₂Me; —NHBn; phenyl; —SBn; —OBn; —SO₂NH₂; —SO₂Me; —SO₂Et; —SO₂Bn; —SO₂NMe₂; —OSO₂Me; —C≡C-phenyl; —C≡CCH₂(OCH₂CH₂)OH; —C≡CCH₂(OCH₂CH₂)OMe; phenyl substituted with at least one of methyl, methoxyl, —SO₂Me, or halogen; or —NHBn wherein -Bn is substituted with at least one of —C₁-C₆ alkyl, —O—(C₁-C₆ alkyl), —SO₂(C₁-C₆ alkyl), halogen, —CN, haloalkyl, —OH, pyridinyl, —NO₂, —N-piperazinyl-N—(C₁-C₆ alkyl), or phenyl.

12. The compound of claim 1, wherein
R¹² is phenyl, imidazole, pyridine, or dihydropyrimidine, each of which is substituted with R⁴, R⁵, and R⁶;
R¹, R², R⁴, R⁵, and R¹⁰ are H;
R³ is —COOH or —COO—(C₁-C₆ alkyl);
R⁶ is methyl; fluoro; chloro; bromo; hydroxyl; methoxyl; —CN; —NO₂; —CH₂OH; —SO₂Me; —CF₃; —CH₂F; —CHF₂; —CCl₃; —CO₂Me; —NHSO₂Me; —SBn; —OBn; —SO₂NH₂; —SO₂Me; —SO₂Bn; —SO₂NMe₂; —OSO₂Me; —C≡C-phenyl; —C≡CCH₂(OCH₂CH₂)OH; —C≡CCH₂(OCH₂CH₂)OMe; —NHBn; —NHBn wherein -Bn is substituted with at least one of —C₁-C₆ alkyl, —O—(C₁-C₆ alkyl), —SO₂(C₁-C₆ alkyl), halogen, —CN, —CF₃, —OH, pyridinyl, —NO₂, —N-piperazinyl-N—(C₁-C₆ alkyl), or phenyl;
phenyl; or phenyl substituted with at least one of methyl, methoxyl, —SO₂Me, or halogen; and
R¹¹ is H or —C¹-C⁶ alkyl;
provided that:
when R¹² is phenyl, then R⁶ is not 4-nitro, 4-chloro, 4-methoxyl, 4-CF₃, 4-bromo, 4-isopropyl, 2-chloro, 4-chloro, 4-hydroxyl, 3-nitro, or 4-hydroxyl.

13. The compound of claim 1, wherein:
R¹² is phenyl, imidazole, pyridine, or dihydropyrimidine, each of which is substituted with R⁴, R⁵, and R⁶;
R¹, R², R⁴, R⁵, and R¹⁰ are H;
R³ is —COOH or —COO—(C₁-C₆ alkyl);
R⁶ is 4-methyl, 4-fluoro, 3-fluoro, 3-chloro, 3-methyl, 3-methoxy, 4-cyano, 4-(CH₂OH), 4-(SO₂Me), 3-nitro, 3-hydroxyl, 4-(CO₂Me), 3-cyano, 4-(NHSO₂Me), 4-(4-methylphenyl), 4-(4-methoxyphenyl), 4-(4-(SO₂Me)phenyl), 4-(SBn), 4-(NHCH₂(4-fluorophenyl)), 4-(SO₂NH₂), 4-(4-fluorophenyl), 4-(SO₂Bn), 3-(CO₂Me), 4-(SO₂Et), 4-(SO₂NMe₂), 4-(OSO₂Me), 4-(C≡C-phenyl), 4-(C≡CCH₂OCH₂CH₂OCH₃), 4-(NHSO₂-phenyl), 4-(NBnSO₂Me), or 4-(NMeSO₂Me); and
R¹¹ is H or —C¹-C⁶ alkyl.

14. The compound of claim 1, wherein R³ is —COOH, —COOMe, —COOEt, —COO(n-propyl), —COO(i-propyl), —COO(n-butyl), —COO(i-butyl), —COO(s-butyl), —COO(t-butyl), or tetrazole.

15. The compound of claim 1, wherein R¹² is:

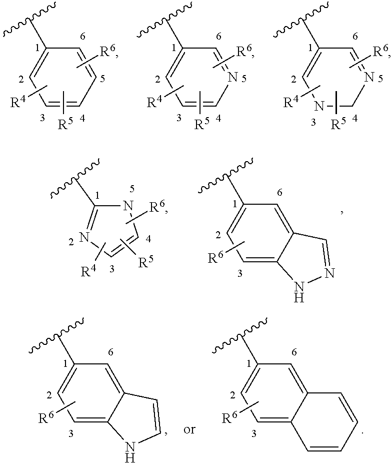

16. The compound of claim 1, wherein R³ is tetrazole having the following structure: 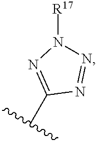
wherein R¹⁷ is —H, C₁-C₆ alkyl, —O—(C₁-C₆ alkyl), —SO₂(C₁-C₆ alkyl), halogen, —CN, —OH, phenyl, or Bn, and
wherein any phenyl, -Bn, or alkyl moiety of an R¹⁷ group can be further substituted with C₁-C₆ alkyl, —O—(C₁-C₆ alkyl), —SO₂(C₁-C₆ alkyl), halogen, —CN, haloalkyl, —OH, pyridinyl, nitro, —N-piperazinyl-N—(C₁-C₆ alkyl), phenyl, -Bn, or any combination thereof.

17. The compound of claim 1, wherein R¹¹ is —H.

18. The compound of claim 1, wherein X is C—H.

19. (canceled)

20. The compound of claim 1, wherein the compound is of formula (IIIa): 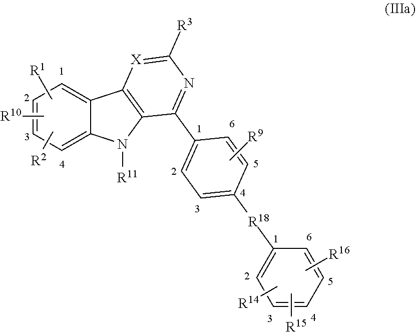
wherein:
X is C—H or N,
R¹, R², and R¹⁰ independently are —H, —OH, —CN, haloalkyl, —O—(C₁-C₆ alkyl), halogen, —C₁-C₆ alkyl, or phenyl;
R³ is —COOH, —COO—(C₁-C₆ alkyl), —CONH₂, —CON(C₁-C₆ alkyl)₂, —CONH(C₁-C₆ alkyl), —CONHSO₂(C₁-C₆ alkyl), —CON(C₁-C₆ alkyl)SO₂(C₁-C₆ alkyl), halogen, —SO₂NH₂, —NHSO₂(C₁-C₆ alkyl), —CN, or tetrazole;
R¹¹ is —H or C₁-C₆ alkyl;
R¹⁸ is —O—, —S—, —NR¹⁹—, —(C₁-C₆ alkyl)-, —(O—(C₁-C₆ alkyl))-, —(O—(C₁-C₆ alkyl)-O)—, —(OCH₂CH₂)ₙ—O—, —(NR¹⁹—(C₁-C₆ alkyl))-, —(NR¹⁹—(C₁-C₆ alkyl)-O)—, —(NR¹⁹—(C₁-C₆ alkyl)-NR¹⁹)—, —(OCH₂CH₂)ₙ—NR¹⁹—, —(S—(C₁-C₆ alkyl))-, —(S—(C₁-C₆ alkyl)-S)—, —(OCH₂CH₂)ₙ—S—, —(NR¹⁹—(C₁-C₆ alkyl)-S)—, —(S—(C₁-C₆ alkyl)-O)—, or —(CONR¹⁹)ₙ—;
wherein each n independently is an integer from 1 to 6;
wherein each R¹⁹ independently is H or C₁-C₆ alkyl; and
R⁹ is —H, —OH, —C₁-C₆ alkyl, —O—(C₁-C₆ alkyl), phenyl, haloalkyl, or halogen;
wherein:
R¹⁴, R¹⁵, and R¹⁶ independently are —H, —C₁-C₆ alkyl, halogen, —OH, —O(C₁-C₆ alkyl), —CN, —NO₂, —(C₁-C₆ alkyl)-OH, SO₂(C₁-C₆ alkyl), haloalkyl, —CO₂(C₁-C₆ alkyl), —NHSO₂(C₁-C₆ alkyl), —NHBn, phenyl, —SBn, —OBn, —SO₂NH₂, —SO₂(C₁-C₆ alkyl), —SO₂Bn, SO₂N(C₁-C₆ alkyl)₂, —OSO₂(C₁-C₆ alkyl), —C≡C-phenyl, —C≡CCH₂(OCH₂CH₂)₁₋₄₀H, —C≡CCH₂(OCH₂CH₂)₁₋₄OMe, —NHSO₂-pyridinyl, —NBnSO₂-pyridinyl, —NHSO₂-phenyl, —NHSO₂(N-piperazinyl-N—(C₁-C₆ alkyl)), —N(C₁—C₆ alkyl)SO₂-pyridinyl, —N(C₁-C₆ alkyl)SO₂-phenyl, —N(C₁-C₆ alkyl)SO₂(N-piperazinyl-N—(C₁-C₆ alkyl)), —NBnSO₂(C₁-C₆ alkyl), —NBnSO₂(N-piperazinyl-N—(C₁-C₆ alkyl)), —NBnSO₂-phenyl, or —N(C₁-C₆ alkyl)SO₂(C₁-C₆ alkyl); or
R¹⁴ and R¹⁵ taken together form a fused pyrazole ring, a fused pyrrole ring, or a fused phenyl ring, and R¹⁶ is as defined above; and
wherein any -Bn, pyridinyl, alkyl, tetrazole, fused pyrazole ring, fused pyrrole ring, fused phenyl ring, or phenyl moiety of the foregoing groups can be substituted with C₁-C₆ alkyl, —O—(C₁-C₆ alkyl), —SO₂(C₁-C₆ alkyl), halogen, —CN, haloalkyl, —OH, pyridinyl, furanyl, tetrahydrofuranyl, tetrahydropyranyl, thiophenyl, thiazolyl, pyrrolyl, pyrrolidinyl, piperidinyl, morpholinyl, pyrimidinyl, triazolyl, imidazolyl, nitro, —N-piperazinyl-N—(C₁-C₆ alkyl), phenyl, Bn, or any combination thereof;
or a salt or ester thereof.

21. The compound of claim 1, wherein the compound of formula (III) is: 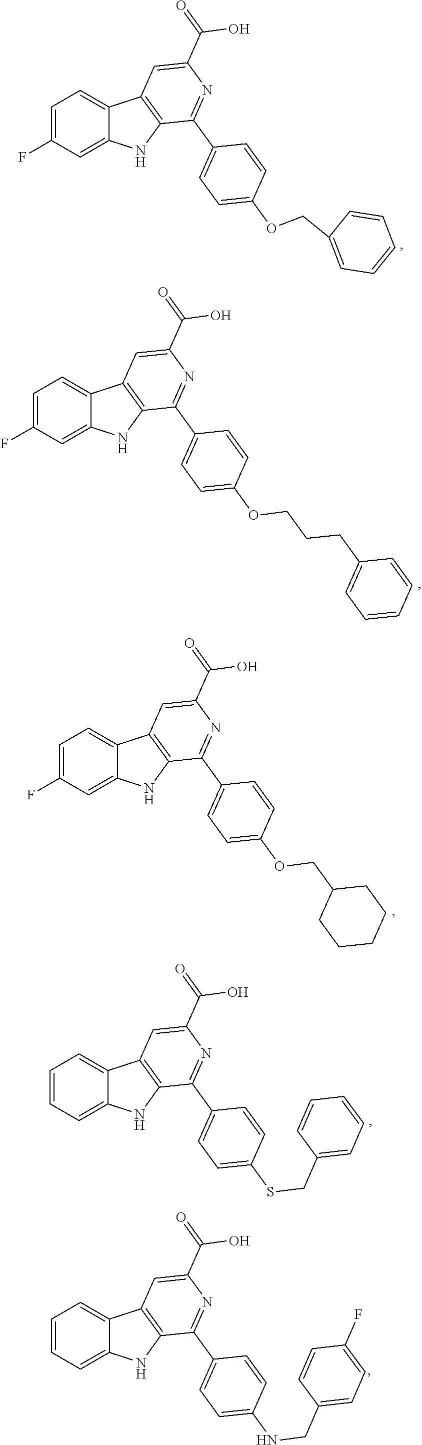 [SMILES: O=C(O)c1cc2c([nH]c3cc(F)ccc32)c(-c2ccc(OCc3ccccc3)cc2)n1 ; O=C(O)c1cc2c([nH]c3cc(F)ccc32)c(-c2ccc(OCCCc3ccccc3)cc2)n1 ; O=C(O)c1cc2c([nH]c3cc(F)ccc32)c(-c2ccc(OCC3CCCCC3)cc2)n1 ; O=C(O)c1cc2c([nH]c3ccccc32)c(-c2ccc(SCc3ccccc3)cc2)n1 ; O=C(O)c1cc2c([nH]c3ccccc32)c(-c2ccc(NCc3ccc(F)cc3)cc2)n1]
or a salt or an ester thereof.

22. A pharmaceutical composition comprising the compound of claim 1 and a pharmaceutically acceptable carrier.

23. A method for treating a disease or condition in an animal, or inhibiting aging in a subject, or inhibiting DNA damage or improving DNA repair activity in a subject, the method comprising administering to the animal or subject a compound of claim 1 or a pharmaceutical composition comprising same; optionally wherein the animal or subject is a mammal or human.

24. The method of claim 23, wherein the disease or condition is cancer.

25. The method of claim 24, wherein the disease or condition is breast cancer, colorectal cancer, lung cancer, or brain cancer; or a cancer that is resistant to one or more topoisomerase I-directed chemotherapy drugs, optionally wherein the cancer is resistant to camptothecin, irinotecan, and/or topotecan;
or
wherein the disease or condition is characterized by (1) increased CRL4A ubiquitin ligase expression or activity as compared to that of a normal, non-diseased subject, (2) increased CUL4A or CUL4B expression or activity as compared to that of a normal, non-diseased subject, (3) increased DDB1 expression or activity as compared to that of a normal, non-diseased subject, or (4) any combination thereof;
or
wherein the method selectively kills tumor cells with a high level of expression of CUL4A, CUL4B, or both; optionally wherein the method does not inhibit CUL1 and/or CUL 3 ubiquitin ligase.

26. (canceled)

27. (canceled)

28. (canceled)

29. (canceled)

30. (canceled)

31. (canceled)
